(19) United States
(12) Patent Application Publication
(10) Pub. No.: US 2022/0254448 A1
(43) Pub. Date: Aug. 11, 2022

(54) METHODS OF IDENTIFYING DOPAMINERGIC NEURONS AND PROGENITOR CELLS

(71) Applicants: Franz-Josef MÜLLER, Felde (DE); Bernhard M. SCHULDT, Düsseldorf (DE); The Scripps Research Institute, La Jolla, CA (US); Aspen Neuroscience, Inc., San Diego, CA (US)

(72) Inventors: Jeanne F. LORING, Del Mar, CA (US); Franz-Josef MÜLLER, Felde (DE); Roy WILLIAMS, San Diego, CA (US); Bernhard M. SCHULDT, Düsseldorf (DE); Andres BRATT-LEAL, San Diego, CA (US)

(21) Appl. No.: 17/629,766

(22) PCT Filed: Jul. 24, 2020
(86) PCT No.: PCT/US2020/043627

§ 371 Date: Jan. 24, 2022

Related U.S. Application Data
(60) Provisional application No. 62/878,701, filed on Jul. 25, 2019.

(51) Int. Cl. G16B 25/10; G16B 40/20; C12N 5/0793; A61K 35/30

(52) U.S. Cl. CPC G16B 25/10; A61K 35/30; C12N 5/0619; G16B 40/20

(57) ABSTRACT

Provided herein are, inter alia, methods of assaying neuronal progenitor cell populations derived from iPSCs, thereby providing for a user friendly molecular diagnostic tool for neuronal cell types, including dopaminergic neurons. The methods provided are valuable for the efficient and precise characterization of identity and functionality of iPSC-derived dopaminergic neurons prior to their clinical application such as the treatment of Parkinson's disease or Multiple Sclerosis.

### CROSS-REFERENCE TO RELATED APPLICATIONS

This application claims priority to U.S. provisional applications 62/878,701, filed Jul. 25, 2019, entitled “METHOD OF IDENTIFYING DOPAMINERGIC NEURONS AND PROGENITOR CELLS,” the contents of which are incorporated by reference in its entirety for all purposes.

### BACKGROUND

This invention includes the establishment of key statistical models and data processing steps that will enable the evaluation of expression data derived from cultured neurons derived from induced pluripotent stem cells. It compares test data to a reference set of data from, for example, previously characterized neurons, neuronal progenitor cells, pluripotent stem cells with known biological characteristics.

### BRIEF SUMMARY

In one aspect, a computer implemented method of identifying a determined dopaminergic precursor cell within an in vitro population of neuronal progenitor cells is provided. The method includes, receiving a test dataset including data including gene expression profile information for an in vitro population of neuronal progenitor cells; querying a gene expression reference database to compare the test dataset with the gene expression reference database, the gene expression reference database including gene expression profile information for a desirable determined dopaminergic precursor cell; and outputting a computed label classification including an indication of whether the in vitro population of neuronal progenitor cells includes a determined dopaminergic precursor cell.

Provided herein are computer implemented methods of classifying an in vitro population of neuronal progenitor cells, the methods comprising receiving a test dataset comprising gene expression levels and expression levels of one or more metagenes for a cell or a plurality of cells comprised in an in vitro population of neuronal progenitor cells, wherein the one or more metagenes are determined based on correlated gene expression levels of reference cells in a reference database, wherein the reference cells are neuronal cells at one or more different stages of differentiation; applying the expression levels of the one or more metagenes as input to a process configured to determine a probability of the cell or the plurality of cells having metagene expression levels of a determined dopaminergic precursor cell; determining a deviation score for the cell or the plurality of cells, wherein the deviation score indicates the degree to which the gene expression levels in the test dataset deviate from gene expression levels in one or more reference cells in the reference database, wherein the one or more reference cells are at a stage of differentiation indicating a determined dopaminergic precursor cell; and outputting, based on the probability and the deviation score, a computed label classification comprising an indication of whether said cell or said plurality of cells from the in vitro population of neuronal progenitor cells is a determined dopaminergic precursor cell.

In some embodiments, the process comprises a supervised classification model trained using (i) expression levels of the one or more metagenes of the reference cells in the reference database; and (ii) class labels indicating each of the one or more different stages of differentiation for reference cells in the reference database, to determine a probability of a cell or a plurality of cells having metagene expression levels of a determined dopaminergic precursor cell.

Also provided herein are computer implemented methods of training a process to determine a probability of a cell or a plurality of cells having metagene expression levels of a determined dopaminergic precursor cell, the methods comprising training a supervised classification model using (i) expression levels of one or more metagenes, wherein the one or more metagenes are determined based on correlated gene expression levels of reference cells in a reference database, wherein the reference cells are neuronal cells at one or more different stages of differentiation; and (ii) class labels indicating each of the one or more different stages of differentiation for reference cells in the reference database, to determine a probability of a cell or a plurality of cells having metagene expression levels of a determined dopaminergic precursor cell.

Also provided herein are computer implemented methods of classifying an in vitro population of neuronal progenitor cells, the methods comprising receiving a test dataset comprising gene expression levels and expression levels of one or more metagenes for a cell or a plurality of cells comprised in an in vitro population of neuronal progenitor cells, wherein the one or more metagenes are determined based on correlated gene expression levels of reference cells in a reference database, wherein the reference cells are neuronal cells at one or more different stages of differentiation; applying the expression levels of the one or more metagenes as input to a process, the process comprising a supervised classification model trained using (i) expression levels of the one or more metagenes of reference cells in the reference database; and (ii) class labels indicating each of the one or more different stages of differentiation of reference cells in the reference database, to determine a probability of a cell or a plurality of cells having metagene expression levels of a determined dopaminergic precursor cell; determining a deviation score for the cell or the plurality of cells, wherein the deviation score indicates the degree to which the gene expression levels in the test dataset deviate from gene expression levels in one or more reference cells in the reference database, wherein the one or more reference cells are at a stage of differentiation indicating a determined dopaminergic precursor cell; and outputting, based on the probability and the deviation score, a computed label classification comprising an indication of whether said cell or plurality of cells from the in vitro population of neuronal progenitor cells is a determined dopaminergic precursor cell.

In some of any of the preceding embodiments, the method comprises, based on the computed label classification, identifying the in vitro population of neuronal progenitor cells as a population comprising determined dopaminergic precursor cells.

In some of any of the preceding embodiments, the supervised classification model is a logistic regression model.

In some of any of the preceding embodiments, the reference cells are an in vitro population of neuronal progenitor cells. In some of any of the preceding embodiments, said in vitro population of neuronal progenitor cells is formed by culturing one or more induced pluripotent stem cells (iPSC) in vitro for a period of time under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neurons. In some embodiments, said iPSC is a human iPSC. In some embodiments, said human is a healthy subject. In some embodiments, said human is a subject with Parkinson's disease.

In some of any of the preceding embodiments, the culturing is for period of time that is between at or about 2 and at or about 25 days. In some of any of the preceding embodiments, said iPSC is cultured for, for about, or for at least 2 days. In some of any of the preceding embodiments, said iPSC is cultured for, for about, or for at least 5 days. In some of any of the preceding embodiments, said iPSC is cultured for, for about, or for at least 10 days. In some of any of the preceding embodiments, said iPSC is cultured for, for about, or for at least 13 days. In some of any of the preceding embodiments, said iPSC is cultured for, for about, or for at least 15 days. In some of any of the preceding embodiments, said iPSC is cultured for, for about, or for at least 18 days. In some of any of the preceding embodiments, said iPSC is cultured for, for about, or for at least 25 days.

In some of any of the preceding embodiments, the reference database comprises gene expression levels determined from one or more reference cell populations, wherein each of the one or more reference cell populations are formed by culturing one or more iPSC in vitro for a different period of time each under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neuron. In some embodiments, the different period of time is between 2 and 30 days. In some embodiments, the different period of time is between 11 and 25 days.

In some of any of the preceding embodiments, the one or more stages of differentiation of reference cells in the reference database are formed by culturing one or more iPSC in vitro for one or more different period of time under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neuron, wherein the different period of time is between about 11 days and about 25 days, optionally a period of time of at or about 13 days; a period of time of at or about 18 days; or a period of time of at or about 25 days. In some of any of the preceding embodiments, at least one of the one or more reference cell populations in the reference database comprises gene expression levels determined by culturing the iPSC for at or about day 13, 18, or 25 days.

In some of any of the preceding embodiments, the conditions capable of differentiating the one or more iPSCs to a neuronal progenitor cell comprises culturing the iPSCs by (a) a first incubation comprising exposing the cells to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling, optionally under conditions to differentiate the cells to floor plate midbrain progenitor cells, optionally wherein the first incubation is initiated on day 0 of the culturing; and (b) a second incubation of cells after the first incubation, wherein the second incubation comprises culturing the cells under conditions to neurally differentiate the cells, optionally wherein the second incubation is initiated at or about day 11 after the first incubation, and further optionally wherein the second incubation is for between at or about 11 and at or about 25 days. In some embodiments, the conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

In some of any of the preceding embodiments, at least one of the one or more reference cell populations in the reference database comprises gene expression levels determined by culturing the iPSC for at or about 13 days. In some of any of the preceding embodiments, at least one of the one or more reference cell populations comprises gene expression levels determined by culturing the iPSC for at or about 18 days. In some of any of the preceding embodiments, at least one of the one or more reference cell populations comprises gene expression levels determined by culturing the iPSC for at or about 25 days.

In some of any of the preceding embodiments, the one or more metagenes and the expression levels of the one or more metagenes are determined by using a dimensionality reduction technique on one or more reference cells of the one or more reference database. In some embodiments, the dimensionality reduction technique is used on a reference cell population comprising gene expression levels determined at or about 13 days of culturing iPSC in vitro under conditions to differentiate neuronal progenitor cells. In some of any of the preceding embodiments, the dimensionality reduction technique is used on a reference cell population comprising gene expression levels determined at or about 18 days of culturing iPSC in vitro under conditions to differentiate neuronal progenitor cells. In some of any of the preceding embodiments, the dimensionality reduction technique is used on a reference cell population comprising gene expression levels determined at or about 25 days of culturing iPSC in vitro under conditions to differentiate neuronal progenitor cells. In some of any of the preceding embodiments, the dimensionality reduction technique is used on each of a reference cell population comprising gene expression levels determined at or about 13 days of culturing iPSC in vitro under conditions to differentiate neuronal progenitor cells; a reference cell population comprising gene expression levels determined at or about 18 days of culturing iPSC in vitro under conditions to differentiate neuronal progenitor cells; and a reference cell population comprising gene expression levels determined at or about 25 days of culturing iPSC in vitro under conditions to differentiate neuronal progenitor cells.

In some of any of the preceding embodiments, the supervised classification model is trained using the expression levels of the one or more metagenes determined from the one or more reference cells. In some of any of the preceding embodiments, the supervised classification model is trained using the expression levels of the one or more metagenes determined from one or more reference cells comprising gene expression levels between 11 and 25 days of culturing iPSC in vitro under conditions to differentiate neuronal progenitor cells, optionally one or more of 13, 18, and 25 days of culturing iPSC in vitro under conditions to differentiate neuronal progenitor cells. In some of any of the preceding embodiments, the supervised classification model is trained using the expression levels of the one or more metagenes determined from the one or more reference cells comprising gene expression levels determined at or about 13 days of culturing iPSC in vitro under conditions to differentiate neuronal progenitor cells. In some of any of the preceding embodiments, the supervised classification model is trained using the expression levels of the one or more metagenes determined from the one or more reference cells comprising gene expression levels determined at or about 18 days of culturing iPSC in vitro under conditions to differentiate neuronal progenitor cells. In some of any of the preceding embodiments, the supervised classification model is trained using the expression levels of the one or more metagenes determined from the one or more reference cells comprising gene expression levels determined at or about 25 days of culturing iPSC in vitro under conditions to differentiate neuronal progenitor cells. In some of any of the preceding embodiments, the supervised classification model is trained using the expression levels of the one or more metagenes determined from each of a reference cell population comprising gene expression levels determined at or about 13 days of culturing iPSC in vitro under conditions to differentiate neuronal progenitor cells; a reference cell population comprising gene expression levels determined at or about 18 days of culturing iPSC in vitro under conditions to differentiate neuronal progenitor cells; and a reference cell population comprising gene expression levels determined at or about 25 days of culturing iPSC in vitro under conditions to differentiate neuronal progenitor cells.

In some of any of the preceding embodiments, the class label indicating each of the one or more different stages of differentiation of the reference cells is either a determined dopaminergic precursor cell or a not a determined dopaminergic precursor cell.

In some of any of the preceding embodiments, the class label indicating each of the one or more different stages of differentiation of the reference cells is determined using an in vivo method. In some embodiments, the in vivo method comprises transplanting the in vitro population of neuronal progenitor cells comprising a reference cell population into a brain region of an animal model of Parkinson's disease; assessing the occurrence of an outcome associated with a therapeutic effect of the transplantation on the animal model, optionally wherein the outcome is selected from innervation or engrafting with host cells, reduction of a brain lesion in the animal model, or reversal of a brain lesion in the animal model; and designating the class label as a determined dopaminergic precursor cell if the transplantation results in the occurrence of the outcome associated with a therapeutic effect; or designating the class label as not a determined dopaminergic precursor cell if the transplantation does not result in the occurrence of the outcome associated with a therapeutic effect. In some embodiments, the brain region is the substantia nigra. In some of any of the preceding embodiments, the in vivo method comprises a behavioral assay.

In some of any of the preceding embodiments, the class label indicating each of the one or more different stages of differentiation of the reference cells is determined using an in vitro method. In some embodiments, the in vitro method comprises assessing dopamine production levels of a reference cell population; and the class label is designated as a determined dopaminergic precursor cell if the dopamine production levels are increased relative to a pluripotent stem cell. In some of any of the preceding embodiments, assessment of dopamine production is by high performance liquid chromatography.

In some of any of the preceding embodiments, the in vitro method comprises assessing levels of Tyrosine Hydroxylase expression for a reference cell population; and the class label is designated as a not a determined dopaminergic precursor cell if the reference cell population expresses high Tyrosine Hydroxylase. In some embodiments, the levels of Tyrosine Hydroxylase expression are assessed using flow cytometry.

In some of any of the preceding embodiments, the reference database further comprises the class labels of the one or more reference cells.

In some of any of the preceding embodiments, the expression levels of the one or more metagenes in the test dataset is determined based on (i) the one or more metagenes determined from the one or more reference cells in the reference database and (ii) the gene expression levels in the test dataset. In some embodiments, the expression levels of the one or more metagenes in the test dataset is determined using regression analysis based on (i) the one or more metagenes determined from the one or more reference cells in the reference database and (ii) the gene expression levels in the test dataset. In some of any of the preceding embodiments, the expression levels of the one or more metagenes in the test dataset is determined by merging the gene expression levels in the test dataset with the reference database to create an updated reference database and applying the dimensionality reduction technique on the updated reference database.

In some of any of the preceding embodiments, the dimensionality reduction technique is conventional non-negative matrix factorization, discriminant non-negative matrix factorization, graph regularized non-negative matrix factorization, bootstrapping sparse non-negative matrix factorization, or regularized non-negative matrix factorization. In some of any of the preceding embodiments, the dimensionality reduction technique is conventional non-negative matrix factorization.

In some of any of the preceding embodiments, the number of the one or more metagenes is chosen based on the performance of the supervised classification model in determining a probability of a cell or a plurality of cells having metagene expression levels of a determined dopaminergic precursor cell. In some of any of the preceding embodiments, the number of the one or more metagenes is chosen based on evaluating one or more metrics determined from performing the dimensionality reduction technique using multiple candidate numbers of metagenes. In some embodiments, the one or more metrics comprise cophenetic distance, dispersion, residuals, residual sum of squares (RSS), silhouette, and/or sparseness values.

In some of any of the preceding embodiments, the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than a threshold probability value. In some embodiments, the threshold probability value is set such that a determined dopaminergic precursor cell is identified with greater than or greater than about 75%, 80%, 85%, 90%, or 95% sensitivity; and/or the threshold probability value is set such that a determined dopaminergic precursor cell is identified with greater than or greater than about 75%, 80%, 85%, 90%, or 95% specificity. In some embodiments, the threshold probability value is set such that a determined dopaminergic precursor cell is identified with greater than or greater than about 98% sensitivity and 100% specificity. In some of any of the preceding embodiments, the threshold probability value is determined by using the area under a receiver operator characteristic (ROC) curve based on the supervised classification model. In some of any of the preceding embodiments, the threshold probability value is between or between about 0.4 and 0.8 inclusive. In some of any of the preceding embodiments, the threshold probability value is or is about 0.4, 0.45, 0.5, 0.55, 0.6, 0.65, 0.7, 0.75, or 0.8.

In some of any of the preceding embodiments, the deviation score for the cell or the plurality of cells is determined using a single-gene deviation score for each of one or more genes in the test dataset. In some embodiments, the single-gene deviation scores are determined using differences between the gene expression levels of the test dataset and the gene expression levels in one or more reference cells in the reference database. In some embodiments, the differences are absolute differences. In some of any of the preceding embodiments, the single-gene deviation scores are determined using standard deviations of gene expression levels in one or more of the one or more reference cells. In some of any of the preceding embodiments, the single-gene deviation scores are z-scores determined using the differences between the gene expression levels of the test dataset and the gene expression levels in the one or more reference cells in the reference database; and the standard deviations of gene expression levels in one or more of the one or more reference cells of the reference database.

In some of any of the preceding embodiments, the gene expression levels in one or more reference cells in the reference database are determined based on average gene expression levels in one or more reference cells of the reference database. In some of any of the preceding embodiments, the gene expression levels in the one or more reference cells in the reference database are determined based on the expression levels of the one or more metagenes in the test dataset. In some embodiments, the gene expression levels in the one or more reference cells in the reference database are determined using regression analysis based on (i) the expression levels of the one or more metagenes in the test dataset and (ii) the gene expression levels in the test dataset.

In some of any of the preceding embodiments, the deviation score is a summary statistic based on all single-gene deviation scores. In some of any of the preceding embodiments, the deviation score is a summary statistic based on single-gene deviation scores for one or more marker genes. In some of any of the preceding embodiments, the summary statistic is a sum. In some of any of the preceding embodiments, the summary statistic is a weighted sum. In some embodiments, the single-gene deviation scores of the one or more marker genes have higher weight.

In some of any of the preceding embodiments, the summary statistic is a percentile value. In some embodiments, the percentile value is between or between about the 50% percentile and the 100% percentile; and/or the percentile value is or is about the 50%, 60%, 70%, 80%, 90%, or 95% percentile.

In some of any of the preceding embodiments, the marker genes comprise radial glial cell markers, early neuronal development genes, pluripotency specific markers, intermediate to late neuronal markers, neurofilament light polypeptide chain markers, neurofilament medium polypeptide chain markers, nestin filament markers, early patterning markers, neural progenitor cell markers, early migration markers, stage-specific transcription factors, genes required for normal development of neurons, genes controlling dopaminergic neuron development, genes regulating identity and fate of neuronal progenitor cells, dopaminergic neuron markers, astrocyte markers, forebrain markers, hindbrain markers, subthalamic nucleus markers, radial glial markers, cell cycle markers, or any combination of any of the foregoing. In some of any of the preceding embodiments, the marker genes are or comprise WNT1, VIM, TOP2A, TH, SOX2A, SLIT2, RFX4, POU5F1, PITX2, PAX6, OTX2, NR4A2, NHLH2, NEUROD4, NEUROD1, NES, NEFM, NEFL, NASP, MAP2, LMX1A, LIN28A, HOXA2, HMGB2, HES1, FOXG1, FOXA2, FABP7, DDC, DCX, BARHL2, BARJL1, ASPM, ALDH1A1, or any combination of any of the foregoing.

In some of any of the preceding embodiments, the computed label classification indicates that said cell or plurality of cells from the in vitro population of neuronal progenitor cells is a determined dopaminergic precursor cell if the deviation score indicates that at least or at least about 50%, 50%, 70%, 80%, 90%, or 95% of gene expression levels in the test dataset are no more than five standard deviations away from gene expression levels of the one or more reference cells in the reference database. In some of any of the preceding embodiments, the computed label classification indicates that said cell or plurality of cells from the in vitro population of neuronal progenitor cells is a determined dopaminergic precursor cell if the deviation score indicates that at least or at least about 95% of gene expression levels in the test dataset are no more than 10, 9, 8, 7, 6, or 5 standard deviations away from the gene expression levels of the one or more reference cells in the reference database. In some of any of the preceding embodiments, the computed label classification indicates that said cell or plurality of cells from the in vitro population of neuronal progenitor cells is a determined dopaminergic precursor cell if the deviation score indicates that at least or at least about 50%, 50%, 70%, 80%, 90%, or 95% of marker gene expression levels in the test dataset are no more than five standard deviations away from the gene expression levels of the one or more reference cells in the reference database. In some of any of the preceding embodiments, the computed label classification indicates that said cell or plurality of cells from the in vitro population of neuronal progenitor cells is a determined dopaminergic precursor cell if the deviation score indicates that at least or at least about 95% of marker gene expression levels in the test dataset are no more than 10, 9, 8, 7, 6, or 5 standard deviations away from the gene expression levels of the one or more reference cells in the reference database.

In some of any of the preceding embodiments, the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than the threshold probability value; and the deviation score indicates that at least or at least about 50%, 50%, 70%, 80%, 90%, or 95% of gene expression levels in the test dataset are no more than five standard deviations away from the gene expression levels of the one or more reference cells in the reference database. In some of any of the preceding embodiments, the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than the threshold probability value; and the deviation score indicates that at least or at least about 50%, 50%, 70%, 80%, 90%, or 95% of marker gene expression levels in the test dataset are no more than five standard deviations away from the gene expression levels of the one or more reference cells in the reference database. In some of any of the preceding embodiments, the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than the threshold probability value; the deviation score indicates that at least or at least about 50%, 50%, 70%, 80%, 90%, or 95% of gene expression levels in the test dataset are no more than five standard deviations away from the gene expression levels of the one or more reference cells in the reference database; the deviation score indicates that at least or at least about 50%, 50%, 70%, 80%, 90%, or 95% of marker gene expression levels in the test dataset are no more than five standard deviations away from the gene expression levels of the one or more reference cells in the reference database.

In some of any of the preceding embodiments, the computed label classification indicates that said cell or plurality of cells from the in vitro population of neuronal progenitor cells is a determined dopaminergic precursor cell if the differences in expression of the marker genes between the test dataset and reference cells of the reference database is statistically insignificant based on a multiple-comparison corrected significance level. In some embodiments, the multiple-comparison corrected significance level is a Bonferroni corrected significance level or a false discover rate corrected significance level. In some of any of the preceding embodiments, the multiple-comparison corrected significance level is 0.01, 0.05, or 0.1.

In some of any of the preceding embodiments, said gene expression levels are obtained from microarray analysis of cellular RNA, RNA sequencing, or both. In some of any of the preceding embodiments, said gene expression levels are obtained from RNA sequencing. In some of any of the preceding embodiments, the RNA sequencing is performed on bulk RNA from the plurality of cells or a plurality of reference cells. In some of any of the preceding embodiments, the RNA sequencing is performed on RNA from the single cells or a single reference cell. In some of any of the preceding embodiments, the gene expression levels of reference cells in the reference database comprises expression levels determined by RNA sequencing that is performed on bulk RNA from a plurality of reference cells and on RNA from a single reference cell.

In some of any of the preceding embodiments, receiving said test dataset comprises receiving input from an array analysis system. In some of any of the preceding embodiments, receiving the test dataset comprises receiving input via a computer network. In some of any of the preceding embodiments, said one or more reference databases forms part of a storage medium.

In some of any of the preceding embodiments, the method comprises repeating the receiving, applying, determining, and outputting steps if the computed label classification indicates that said cell or plurality of cells is not a determined dopaminergic neuronal cell, optionally wherein the steps are repeated the same or a different in vitro population of neuronal progenitor cells. In some embodiments, the receiving, applying, determining, and outputting steps are repeated or repeated about one, two, three, four, five, six, seven, eight, nine, or 10 days after the previous iteration of the method.

In some of any of the preceding embodiments, the method comprises repeating the receiving, applying, determining, and outputting steps if the computed label classification indicates that said cell or plurality of cells is not a determined dopaminergic neuronal cell, wherein the steps are repeated using different in vitro population of neuronal progenitor cells formed by culturing another iPSC clone under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neurons. In some embodiments, said different in vitro population of neuronal progenitor cells is formed from the same human subject as the previous iteration of the method.

In some of any of the preceding embodiments, the receiving, applying, determining, and outputting steps are repeated on in vitro population of neuronal progenitor cells formed by culture of iPSC for different periods of time and/or under different conditions capable of differentiating the one or more iPSCs to a neuronal progenitor cell, until an indication that said cell or said plurality of cells is a determined dopaminergic neuronal cell is output.

Also provided herein are populations of determined dopaminergic precursor cells identified by the method of some of any of the preceding embodiments.

Also provided herein are methods of treatment, the methods comprising administering to a subject having Parkinson's disease the population of determined dopaminergic precursor cells of some of any of the preceding embodiments. In some embodiments, the administering is by implanting the population of determined dopaminergic precursor cells into one or more brain regions of the subject. In some embodiments, the one or more brain regions comprise the substantia nigra.

In some of any of the preceding embodiments, the population of determined dopaminergic precursor cells is autologous to the subject. In some of any of the preceding embodiments, the population of determined dopaminergic precursor cells is allogeneic to the subject.

Also provided herein are methods of treating a subject having Parkinson's disease, the methods comprising implanting a population of determined dopaminergic precursor cells into a brain region of a subject having Parkinson's disease, wherein the population of determined dopaminergic precursor cells has been identified using the computer implemented method of some of any of the preceding embodiments.

In some embodiments, the population of determined dopaminergic precursor cells is autologous to the subject. In some of any of the preceding embodiments, the population of determined dopaminergic precursor cells is allogeneic to the subject. In some of any of the preceding embodiments, about or at least or 1×10⁶ cells are injected into the substantia nigra. In some of any of the preceding embodiments, the cells are injected into both the left and right hemispheres.

### BRIEF DESCRIPTION OF FIGURES

FIG. 1 shows the stages of development and when conventional biomarkers cannot be used for stage identification.

FIG. 2 shows an outline of NeuroTest showing key components and data flow. NeuroTest is a computer implemented method of identifying a determined dopaminergic precursor cell within an in vitro population of neuronal progenitor cells. The outline shown in FIG. 2 is an outline of exemplary components and data flow in NeuroTest. In this exemplary embodiment, RNA sequencing (RNAseq) data from an in vitro population of neuronal progenitor cells (test sample) is provided to NeuroTest. For each test sample, NeuroTest provides two parameters as output: a NeuroScore and a Novelty Score. Together, these parameters are used to determine if the test sample contains a determined dopaminergic precursor cell.

FIG. 3A-3C show example output of NeuroTest: (FIG. 3A) a table of the statistical scores, (FIG. 3B) as a histogram or (FIG. 3C) a scatter plot showing NeuroScore on the y-axis and Novelty on the x-axis. FIG. 3B and FIG. 3C show induced pluripotent stem cells (iPSC) and dopaminergic (DA) neurons failing and passing NeuroTest, respectively. FIG. 3B and FIG. 3C are displaying a NeuroScore on the y-axis which is rescaled to a percentage value. In FIG. 3C, the NeuroScore is referred to as “neuri,” and the Novelty Score is referred to as “deviation.”

FIG. 4 shows a scatter plot showing NeuroScores (y-axis) and novelty scores (x-axis) for the validation data set. Validating the NeuroTest model, initially trained on discriminating genes from the microarray data and supplemented with RNAseq based gene expression data. Here RNAseq data was used as validation since the model training was done with Illumina bead array data (by using 5 fold cross-validation). The validation RNAseq data was generated or downloaded from public data repositories. The samples in the upper left quadrant pass for both high NeuroScore and low novelty. The “Undiff” samples (mostly undifferentiated IPSC, diamonds) fail NeuroTest due to getting a low NeuroScore and having elevated levels of novelty compared to the reference data model. In FIG. 4, the NeuroScore is referred to as “N-score.”

FIG. 5 shows the NeuroTest result from the analysis of 86 publicly available neuronal RNAseq datasets. The datapoints highlighted with the black circles are specifically the data points from the challenge datasets. The solid background datapoints are from the Neurotest validation analysis of the 695 samples of validation data. These results provide context for the Neurotest challenge data. The spread of the challenge data, spanning the range from iPSC to cancer cells to neuronal reflects the input data. The tabular output reveals that NeuroTest gave a “pass” score to DA neuron cellular preparations. In FIG. 5, the NeuroScore is referred to as “N-score.”

FIG. 6 shows how NeuroTest uses gene expression as a phenotype to identify neuronal precursor cells.

FIG. 7 shows metagene expression levels (metagene contribution) for cell samples at day 18 of a dopaminergic neuron differentiation protocol. Metagenes and expression levels thereof were derived by applying conventional non-negative matrix factorization (NMF) on single-cell RNAseq (scRNAseq) data, scRNAseq data aggregated to approximate bulk RNAseq data (bulk from single cell), and bulk RNAseq data collected from each of four cell lines. For each sample collected from the cell lines, both scRNAseq and bulk RNAseq data were collected.

FIG. 8 shows a receiver operating characteristic (ROC) curve showing classification performance of a logistic regression model trained to identify a determined dopaminergic precursor cell within an in vitro population of neuronal progenitor cells.

FIG. 9 shows another exemplary workflow for building and using NeuroTest. In this exemplary workflow, gene expression data from publically available databases, scRNAseq datasets, and matched bulk RNAseq datasets are collected for in vitro populations of neuronal progenitor cells containing determined dopaminergic precursor cells. These datasets are supplied (circles 3 and 4) to a process that calculates metagenes and expression levels thereof. Metagene expression levels are supplied (circle 5) as training data to a classification model configured to determine the probability of a sample having metagene expression levels of a determined dopaminergic precursor cell. This model can be validated (circle 6) using additional data, for instance bulk RNAseq data not used in training the model. The trained model is then used as part of NeuroTest (circle 7) in order to test future test samples from other in vitro populations. Novelty Scores are also calculated per training sample, and these scores and the trained model are used to identify NeuroScore and Novelty Score thresholds (circle 8) that will be used to evaluate the future test samples. For future test samples, RNAseq data is subjected to sequence alignment using the Salmon pseudoaligner (circle 1). Next, the test RNAseq data is supplied to the trained model (circle 2), and a NeuroScore (circle 10) and Novelty Score (circle 11) are output for the test sample. These scores are compared to the previously determined thresholds in order to determine if the test sample should be transplanted, additionally screened, or discarded.

FIG. 10 shows gene expression deviation of an exemplary sample from an in vitro population of neural progenitor cells. Gene expression deviation is shown for several individual marker genes and is calculated as normalized residuals showing how far individual gene expression deviates from expected values, where the expected values are determined from cells with known identity (e.g., reference cells).

FIG. 11 shows the output of NeuroTest (NeuroScores and Novelty Scores) for cell samples at various stages (days) of a dopaminergic neuron differentiation protocol. The horizontal dashed line is at NeuroScore=0. The vertical dashed line is at Novelty Score=5. In this exemplary embodiment, samples with a Neuroscore >0 and a Novelty Score <5 are identified as containing determined dopaminergic precursor cells.

### DETAILED DESCRIPTION

Provided herein is a method of classifying whether an in vitro population of neuronal progenitor cells contains a particular differentiated neuronal cell type. In some embodiments, the provided methods classify whether an in vitro population of differentiated neuronal cells contains determined dopamingergic precursor cells. In some embodiments, the methods provided herein identify whether an in vitro population of neuronal cells contain determined dopaminergic precursor cells. In some embodiments, determined dopaminergic precursor cells are cells that differentiate into dopaminergic neurons and cannot differentiate into non-dopaminergic cells. A cell population that is classified according to the provided method can be used to identify cells of interest, for example, for therapeutic application. Thus, also provided are populations of determined dopaminergic precursor cells identified by the provide methods, and pharmaceutical compositions containing the same. In some embodiments, the determined dopaminergic precursor cells have therapeutic application in the treatment of neurodegenerative diseases, such as Parkinson's disease.

In provided methods, the methods include receiving a test dataset that includes (1) gene expression levels and (2) expression levels of one or more metagenes for a cell or a plurality of cells contained in an in vitro population of neuronal progenitor cells in which the one or more metagenes are determined based on correlated gene expression levels of reference cells in a reference database. In some embodiments, the in vitro population of neuronal progenitor cells is a population of cells that has been subjected to a process to differentiate pluripotent stem cells, such as induced pluripotent stem cells (iPSCs), into neuronal cells, such as dopaminergic neurons or a determined precursor of dopaminergic neurons. In some embodiments, the methods include applying the expression levels of the one or more metagenes as input to a process configured to determine a probability of the cell or the plurality of cells in the in vitro population of neuronal progenitor cells having metagene expression levels of a determined dopaminergic precursor cell. In some embodiments, the methods include also determining a deviation score for the cell or the plurality of cells in the in vitro population of neuronal progenitor cells in which the deviation score indicates the degree to which the gene expression levels in the test dataset deviate from gene expression levels in one or more reference cells in the reference database, wherein the one or more reference cells are at a stage of differentiation indicating a determined dopaminergic precursor cell. In some embodiments, the deviation score is determined using the gene expression levels in the test dataset and the gene expression levels in a reference database. In some embodiments, the methods include outputting, based on the probability and the deviation score, a computed label classification that provides an indication of whether said cell or said plurality of cells from the in vitro population of neuronal progenitor cells is a determined dopaminergic precursor cell, thereby classifying whether the in vitro population of neuronal progenitor cells is a population that is or contains determined dopaminergic precursor cell. In some embodiments, the methods thus can identify based on the classification whether the in vitro population of neuronal progenitor cells is a population that contains determined dopaminergic precursor cells.

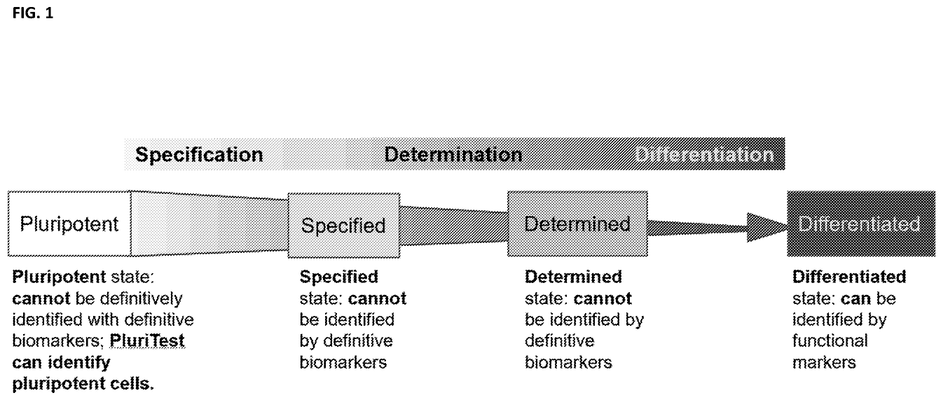

In some embodiments, certain differentiated neuronal cell populations differentiated from pluripotent stem cells, including determined dopaminergic precursor cells, may be cells in a stage of differentiation where the cells are not identifiable by one or a small number of features or characteristics. The methods provided herein allow for the determination of cell identity when a single or small number of features or characteristics, such as gene expression markers or functional properties, are unavailable (e.g., unknown) or cannot be practically used to determine cellular identity. For example, as shown in FIG. 1, cells undergoing differentiation enter stages where no definitive biomarker can be used to determine the identity of the cell. While pluripotent stem cells can be positively identified with definitive biomarkers, for instance the expression levels of specific genes, and differentiated cells can be positively identified based on functional markers, individual markers for the identification of cells at various transient stages throughout differentiation are unknown. Without such markers, there has been previous difficulty in characterizing, defining, and/or identifying pre-differentiated cells with particular cell phenotypes. In some aspects, the methods provided herein overcome the lack of a single or small number of features or characteristics (e.g., biomarkers) by examining groups of related genes and expression levels thereof. Such an approach does not rely on knowledge of individual marker genes and instead uses a whole transcriptome approach in characterizing and identifying determined dopaminergic precursor cells.

Induced pluripotent stem cells (iPSCs) are considered useful as a cell therapy for at least their ability to be differentiated into specialized cell types. For example, iPSCs, like pluripotent stem cells, can be differentiated into specific cell types that can be used to replace diseased or damaged tissue. In some cases, iPSCs that have been differentiated into a particular neuronal cell type or precursor may be used to treat neurodegenerative diseases, for example by differentiating iPSCs and implanting the differentiated neuronal cells into the brain of a subject having a neurodegenerative disease. The inability to determine the identity of the differentiated cells throughout the differentiation process can lead to uncertainty about the success of the process. For example, the differentiation process may need to be run to completion in order to determine if the differentiation process was successful. Thus, without the ability to determine whether differentiating cells are progressing through the transient stages as needed, the differentiation process becomes time consuming and inefficient, and can hinder treatment of the subject, for example when a differentiation process fails. Furthermore, in some cases, the therapeutic treatment can include administering (e.g., injecting) to the subject differentiated cells that have not entered a final differentiation stage.

In some embodiments, cells at an intermediate stage of differentiation cannot be, or cannot easily be, identified by definitive biomarkers. The methods provided herein allow for the identification of cells at stages of differentiation where no definitive features or characteristics are available or can be practically used to determine cell identity. In some embodiments, the methods provided herein improve the differentiation process, for example, by allowing a determination of cell identity throughout the stages of differentiation, which can be used to determine whether cells undergoing a differentiation process are differentiating appropriately and/or according to defined standards. If it is determined that the cells are not differentiating appropriately, in some embodiments, the process can be terminated and optionally reinitiated with different iPSC clones from the patient.

In some embodiments, the methods provided herein may be used in combination with a process that includes generating neuronal cells useful for the treatment of a neurodegenerative disease, such as Parkinson's disease, by differentiation from iPSCs. In some embodiments, the methods provided herein can be used to identify neuronal cells generated by a differentiation process, for example a process described in Section II, that are useful for the treatment of Parkinson's disease.

The methods provided herein can be used to determine if an in vitro population of cells comprises predetermined dopaminergic precursor cells. In some embodiments, the methods provided herein comprise determining metagenes and expression levels thereof of test cells comprised in the in vitro population. In some embodiments, the methods provided herein comprise determining the probability of the test cells having metagene expression levels of a determined dopaminergic precursor cell. In some embodiments, the probability is determined using a machine learning model. In some embodiments, the methods provided herein comprise determining a deviation score indicating the degree to which the gene expression levels of the test cells deviate from expected gene expression levels. In some embodiments, the expected gene expression levels are based on gene expression levels of reference cells that are known to be determined dopaminergic precursor cells. In some embodiments, the methods provided herein comprise outputting a computed label classification based on one or both of (i) the probability of the test cells having metagene expression levels of a determined dopaminergic precursor cell and (ii) the deviation score. In some embodiments, the deviation score is based on a subset of marker genes. In some embodiments, determining the probability of the test cells having metagene expression levels of a determined dopaminergic precursor cell allows for the identification of cells with the desired phenotype, said phenotypes lacking individual marker genes. In some embodiments, determining the deviation score allows for the identification of cells that may contain abnormalities, for instance in the expression of certain marker genes. Thus, the methods provided herein provide a multifaceted approach for determining suitable cells for treatment.

In the subsections below, exemplary features of provided methods of classifying whether an in vitro population of neuronal progenitor cells contains a particular differentiated neuronal cell type, and methods for identifying a particular differentiated neuronal cell type, are described. Related compositions and methods of production and uses thereof also are described.

### I. Methods of Determining a Determined Dopaminergic Cell

Provided herein are, inter alia, methods that use gene expression as a phenotype to identify dopaminergic precursors in an in vitro cell population of neuronal progenitor cells. The methods provided herein provide, inter alia, information whether a cell preparation (e.g., a population of neuronal progenitor cells) includes cells that are determined to differentiate into a specific functional cell type (e.g., a determined dopaminergic precursor cell) or whether the cell preparation includes cells from earlier stages (e.g. pluripotent stem cells, specified cells), other differentiating neuron types, and other differentiated cell types.

Thus, in one aspect, a computer implemented method of identifying a determined dopaminergic precursor cell within an in vitro population of neuronal progenitor cells is provided. The method includes, receiving a test dataset including data including gene expression profile information for an in vitro population of neuronal progenitor cells; querying a gene expression reference database to compare the test dataset with the gene expression reference database, the gene expression reference database including gene expression profile information for a desirable determined dopaminergic precursor cell; and outputting a computed label classification including an indication of whether the in vitro population of neuronal progenitor cells includes a determined dopaminergic precursor cell.

The methods provided herein may define a determined state of a cell and predict whether a cell preparation will differentiate into a specific cell type. The reference database provided herein may include gene expression profile information of two cell types. In embodiments, the cells identified with the methods provided herein are determined to differentiate into a specific functional cell type. Whether a cell is determined to differentiate into a specific functional cell type (e.g., a determined dopaminergic precursor cell) may further be demonstrated in vitro or in vivo by allowing the cells to fully differentiate. In embodiments, the cells identified with the methods provided herein are pluripotent stem cells, specified cells, differentiating neuron types other than dopaminergic precursors or other differentiated cell types.

In embodiments, the computer implemented method further includes a machine learning model trained to determine whether the in vitro population of neuronal progenitor cells includes the determined dopaminergic precursor cell, the machine learning model outputting the computed label classification. In embodiments, the in vitro population of neuronal progenitor cells are formed by allowing an induced pluripotent stem cell (iPSC) to differentiate in vitro. In embodiments, the iPSC is a human iPSC. In embodiments, the iPSC is cultured for at least 15 days under conditions for differentiation into a neuronal progenitor cell. In embodiments, the iPSC is cultured for about 18 days under conditions for differentiation into a neuronal progenitor cell. The in vitro cell population of neuronal progenitor cells provided herein may be formed by methods commonly known and used in the art to differentiate dopaminergic neurons from iPSCs. Exemplary methods of differentiation processes are described in Section II. Different timepoints of the process for differentiating dopaminergic neurons from iPCSs may result in cells that are at different stages of differention. Therefore, the term “d18” or “day 18” as provided herein refers to the 18^{th} day of the process of differentiating an iPSC to form a dopaminergic neuron. Likewise, the term “d0” or “day 0” refers to the day of the process of differentiating an iPSC to form a dopaminergic neuron is initiated. The provided methods can be used to classify, and thus identify, a differentiated population of neuronal cells that, based on classification labels in accord with the provided methods, is determined to contain a particular neuronal progenitor cell, such as a determined dopaminergic precursor cell.

In some embodiments, the computer implemented method includes a machine learning model trained to determine the probability of a cell or plurality of cells comprised in the in vitro population of neuronal progenitor cells as having metagene expression levels of a determined dopaminergic precursor cell. In embodiments, the machine learning model outputs the probability (also referred to herein as a Neuroscore) of the cell or plurality of cells having metagene expression levels of a determined dopaminergic precursor cell. In embodiments, the computer implemented method further includes determining a deviation score (also referred to herein as Novelty score) for the cell or plurality of cells, wherein the deviation score is indicative of the degree to which gene expression levels of the cell or plurality of cells deviates from expected gene expression levels. In some embodiments, the expected gene expression levels are based on gene expression levels of reference cells, e.g., reference cells that are known to be determined dopaminergic precursor cells. In some embodiments, the computer implemented method includes outputting based on the probability and the deviation score the computed label classification.

The methods, algorithms, and systems described herein are designed to produce a new way of defining a determined dopaminergic precursor cell or dopaminergic cell. This new way is called a computed definition and the previous types of definitions are referred to as biological definitions (functional, structural, genesis). The computed definition is related to a biological definition, but as discussed herein, the computed definition provides a more robust and accurate way of comparing two different cells and determining whether they are the same type of cell or different cell types. In some embodiments, the computed definition provides a more robust and accurate way of identifying a cell of unknown identity.

The computed definition refers to the use of computational analysis of information to arrive at the definition. Disclosed are databases of information about one or more cells. For example, some of the databases are reference databases. A reference database can comprise cell datasets that are produced from cell data for at least two known cell lines, tissues, or primary cells. By known cell line, tissue, or primary cell is meant a cell line for which some characteristic, such as phenotype, such as dopaminergic cell, a determined dopaminergic precursor cell, and has been identified by conventional biological assays, e.g. derivation method, source material, biochemical assays (e.g. enzyme activity, e.g. alkaline phosphatase activity) or markers like specific, identified proteins which are thought to be able to identify a specific cell type. In some embodiments, the cells for which some characteristics are known are referred to as reference cells. A computed phenotype can be defined by the global profiling methods, such as gene expression (or other molecular profiling method) which is then utilized in the methods disclosed herein. Biological phenotypes, such as whether a cell is a stem cell or differentiated cell, which have been determined using subsets of profiling data, such as a subset of markers or gene expression, can be used and incorporated into the methods in the form of labeled associated biological classes.

A. Reference Cells

The methods provided herein, in some aspects, include the use of reference cells and/or reference databases to identify (e.g., determine) the presence of determined dopaminergic precursor cells within an in vitro population of neuronal progenitor cells. The types of reference cells contemplated for use according to the methods provided herein include cells with known identity (e.g., labeled cell) and known characteristics, e.g., have characterized gene expression profiles. In some embodiments, the reference databases comprise reference cell labels and the corresponding reference cell characteristics from a plurality of reference cells. In some embodiments, the reference database can be used, e.g., according to the methods provided herein, to determine whether a cell of unknown identity (e.g., unlabeled) having certain characteristics, e.g., gene expression patterns, has a certain cellular identity.

In some embodiments, the reference cell is a pluripotent stem cell. In some embodiments, the pluripotent stem cell is an induced pluripotent stem cell (iPSC). In some embodiments, the iPSC is generated from fibroblasts collected from a healthy human subject. In some embodiments, the iPSC is generated from fibroblasts collected from a human subject having Parkinson's disease. In some embodiments, the iPSC is generated from fibroblasts collected from a human subject predisposed to developing Parkinson's disease. Exemplary methods for iPSC generation are described in Section II.

In some embodiments, the reference cell is a cell differentiated under conditions to become a neuronal progenitor cell, such as a floor plate midbrain progenitor cells, determined dopaminergic precursor cells, or a dopaminergic neuron. In some embodiments, the reference cell is a cell differentiated according to any of the methods described in Section II. In some embodiments, the reference cell is a determined dopaminergic precursor cell. In some embodiments, the reference cell is a dopaminergic neuron. In some embodiments, the differentiated cell, the determined dopaminergic cell, and/or the dopaminergic cell is derived from an iPSC, for example an iPSC as described above, that has been cultured under conditions to promote differentiation into a dopaminergic cell.

In some embodiments, the reference cell is a cell that is described, e.g., labelled, characterized, in a publically available database.

In some embodiments, the reference cell is of known identity. Thus, in some instances, the identity of the cell can be used as a label for the reference cell. In some embodiments, the reference cell label is indicative of a cellular phenotype. In some embodiments, the reference cell label is indicative of cellular characteristics, e.g., gene expression levels. In some embodiments, the reference cell label indicates if the reference cell is a pluripotent stem cell. In some embodiments, the reference cell label indicates if the reference cell is a determined dopaminergic precursor cell. In some embodiments, the reference cell label indicates if the reference cell is a dopaminergic neurons.

In some embodiments, the reference cell label indicates the differentiation stage of the reference cell. In some embodiments, the reference cell label indicates the period of time that the reference cell has been cultured under differentiation conditions. In some embodiments, the reference cell label indicates the period of time that the reference cell has been cultured under differentiation conditions to become a dopaminergic neuron, e.g., any of the periods of time described in Section II.

In some embodiments, the reference cell label is based on publically available annotations for the reference cell. In some embodiments, the reference cell label is based on the assessment of dopamine production levels of the reference cell. In some embodiments, dopamine production levels are assessed using high performance liquid chromatography (HPLC). In some embodiments, the reference cell label is based on the assessment of tyrosine hydroxylase (TH) expression in the reference cell. In some embodiments, TH expression is assessed using cell staining methods. In some embodiments, the reference cell label is based on the assessment of FOXA2 expression in the reference cell. In some embodiments, FOXA2 expression is assessed using cell staining methods. In some embodiments, TH expression is assessed using flow cytometry.

In some embodiments, a reference cell is characterized as a dopaminergic neuron if it expresses a marker of a midbrain dopaminergic neuron, such as expression of FOXA2 or tyrosine hydroxylase (TH). In some embodiments, a reference cell expresses TH (TH+). In some embodiments, the reference cell expresses FOXA2 (FOXA2+). In some embodiments, the reference cell expresses TH and FOXA2 (TH+FOXA2+).

In some embodiments, the reference cell is determined to or capable of becoming dopaminergic neuron, i.e. is a determined dopaminergic precursor cell, as ascertained based on one or more characteristics that indicate the reference cell is capable of having functional activity of a dopaminergic neuron but may not yet express a marker of a dopaminergic neuron or may not express it at a high level. For example, a reference cell may exhibit lower levels of TH than a dopaminergic neuron, yet still exhibits one or more characteristics of a determined dopaminergic precursor cell indicating the differentiated cell is capable of having functional activity of a dopaminergic neuron. In some embodiments, the one or more characteristics of the reference cell include activity to survive, engraft, and/or innervate other cells when administered in vivo, e.g. to an animal model. In some embodiments, the reference cells are capable of innervating host tissue upon transplantation into an animal or human subject.

In some embodiments, the reference cell is a cell with therapeutic effect to treat a neurodegenerative disease. In some embodiments, the reference cell when implanted ameliorates or reverses symptoms of a neurodegenerative disease. In some embodiments, the neurodegenerative disease is Parkinson's disease. In some embodiments, the reference cells when implanted in the substantia nigra of a subject, e.g., patient, in need thereof improves Parkinsonian symptoms.

In some embodiments, the reference cell is screened for its therapeutic effect to treat a neurodegenerative disease, such as determined in an animal model of a neurodegenerative disease. In some embodiments, the neurodegenerative disease is Parkinson's disease. In some embodiments, the reference cells are screened using an animal model of Parkinson's disease. Any known and available animal model of Parkinson's disease can be used for screening. In some embodiments, the animal model is a lesion model wherein animals received unilateral stereotaxic injection of 6-hydroxydopamine (6-OHDA) into the substantia nigra. In some embodiments, the animal model is a lesion model wherein animals received unilateral stereotaxic injection of 6-OHDA into the medial forebrain bundle. In some embodiments, the reference cells are implanted into the substantia nigra of the animal model. In some embodiments, a behavioral assay is performed to screen for therapeutic effects of the implantation on the animal model. In some embodiments, the behavioral assay comprises monitoring amphetamine-induced circling behavior. In some embodiments, the reference cell is determined to reduce, decrease or reverse a Parkinsonian model brain lesion in this model. In some embodiments, the reference cell may be a cell that does not reduce, decrease or reverse a Parkinsonian model brain lesion in this model. The reference database may include data from various reference cell populations that exhibit varied or different therapeutic effects to treat a neurodegenerative disease, such as in an animal model.

As described above, in some embodiments, any of a number of reference cell characteristics of a particular reference cell or cells can be determined, including any one or more characteristics, traits, features or attributes of a reference cell. In some embodiments, the reference cell characteristics can be used as data to characterize or describe a particular reference cell population. For instance, reference cell characteristics may include mRNA expression levels, microRNA expression levels, protein expression levels, post-translational protein modification levels, non-coding RNA expression profiles, DNA methylation levels, histone modification levels, transcription factor-DNA site binding profiles, DNA sequence profiles, or any other type of cell characteristic, or a combination of any of the foregoing. Any of the one or more of the reference cell characteristics can be used as data to input into or populate a reference cell database.

In some embodiments, reference cell characteristics include protein expression levels. In some embodiments, reference cell characteristics include post-translational protein modification levels. In some embodiments, reference cell characteristics include non-coding RNA expression profiles. In some embodiments, reference cell characteristics include epigenetic profiles. In some embodiments, reference cell characteristics include transcriptional profiles. In some embodiments, reference cell characteristics include gene expression levels. In some embodiments, the reference cell database can include information about any one or more of the above reference cell characteristics.

In some embodiments, the gene expression levels are obtained using microarray analysis. In some embodiments, the gene expression levels are obtained using RNA sequencing. In some embodiments, the gene expression levels are obtained using both microarray analysis and RNA sequencing. In some embodiments, the RNA sequencing is performed on bulk RNA from a plurality of cells. In some embodiments, the RNA sequencing is performed on single cells. In some embodiments, the RNA sequencing is performed on bulk RNA from a plurality of cells and on single cells.

In some aspects, a plurality of reference cells with known identities, e.g., labels, and known characteristics, e.g., gene expression levels, are used to populate a reference database. In some embodiments, the plurality of reference cells used to populate the reference database have different labels from one another. In some embodiments, a portion of the reference cells used to populate the reference database have the same label. In some embodiments, a portion of the reference cells used to populate the reference database have labels different from the other reference cells of the reference database. Thus, in some embodiments, the reference database may include a plurality of reference cells, some having the same label as other cells of the reference database and some having labels different from other cells in the reference database.

In some embodiments, the reference cell characteristics for particular reference cells are included in a reference database. In some embodiments, the reference database contains reference cell labels. In some embodiments, the reference database contains protein expression levels of reference cells. In some embodiments, the reference database contains epigenetic profiles of reference cells. In some embodiments, the reference database contains transcriptional profiles of reference cells. In some embodiments, the reference database contains gene expression levels of reference cells. In some embodiments, the reference database contains gene expression data from publically available databases. In some embodiments, the reference database contains microarray data. In some embodiments, the reference database contains RNA sequencing data. In some embodiments, the reference database contains microarray data and RNA sequencing data.

In some embodiments, the reference database contains bulk RNA sequencing data. In some embodiments, the bulk RNA sequencing data is obtained from a plurality of reference cells. In some embodiments, bulk RNA sequencing data is obtained from pooled RNA from the plurality of reference cells.

Any known and available methods for obtaining bulk RNA sequencing data can be used (for example, see Chao et al., 2019, BMC Genomics 20: 571, incorporated by reference herein in its entirety). For instance, total RNA from a sample, e.g., a plurality of reference cells from an in vitro population of cells, can be isolated using TRIZOL, treated with DNase I, and purified. Concentration and quality of isolated RNA can be measured and checked prior to library preparation for total RNA or mRNA. For library preparation, total RNA or mRNA are fragmented and converted to cDNA using reverse transcription. After construction, amplification, and optional barcoding of double-stranded cDNA, libraries can be processed for next generation sequencing using any known and available library preparation techniques, sequencing platforms, and genomic-alignment tools.

In some embodiments, the reference database includes single-cell RNA sequencing data. In some embodiments, the use of single-cell RNA sequencing data affords certain advantages. In some embodiments, the use of single-cell RNA sequencing data allows for characterization of subpopulations of cells, for instance of determined dopaminergic precursor cells within a larger in vitro population of cells. In some embodiments, the use of single-cell RNA sequencing data reduces the number of reference cells required for use in the methods provided herein. In some embodiments, the use of single-cell RNA sequencing data improves characterization of biological variability across reference cells. In some embodiments, the use of single-cell RNA sequencing data allows for easier validation and interpretation of gene expression levels.

Any known and available methods for single-cell RNA sequencing can be used (for example, see Zheng et al., 2017 (Nature Communications 8: 14049), and Haque et al., 2017 (Genome Medicine 9: 75, incorporated by reference herein in their entirety). For single-RNA sequencing, single cells from a sample, for instance an in vitro population of cells, can be isolated using flow cytometric cell-sorting, microfluidic platform, or droplet-based methods. Isolated cells are lysed to allow capture of RNA molecules. Poly[T]-primers can be used for the analysis of polyadenylated mRNA molecules specifically, and primed mRNA molecules are converted to cDNA using reverse transcription. In some instances, unique molecular identifiers can be used to mark single mRNA molecules based on cellular origin. The cDNA pool is then amplified, optionally barcoded, and sequenced, for instance using next-generation sequencing (NGS) and with library preparation techniques, sequencing platforms, and genomic-alignment tools similar to those used for bulk RNA samples. In some instances, unbiased cell-type classification within a mixed population of distinct cell types can be achieved with as few as 10,000 to 50,000 reads per cell, and single-cell libraries from various common protocols can be close to saturation when sequenced to a depth of 1,000,000 reads.

In some embodiments, the reference databases comprise bulk RNA sequencing data and single-cell RNA sequencing data. In some embodiments, the bulk RNA sequencing data and the single-cell RNA sequencing data are obtained from the same sample, e.g., in vitro population of cells. In some embodiments, the single-cell RNA sequencing data can be used to approximate the bulk RNA sequencing data obtained from the same sample, e.g., in vitro population of cells. In some embodiments, approximated bulk RNA sequencing data is obtained by averaging single-cell RNA sequencing data from reference cells comprised in the same sample, e.g., in vitro population of cells. In some embodiments, the reference database comprises approximated bulk RNA sequencing data.

In embodiments, the gene expression reference database includes transcriptional profiles of one or more dopaminergic neurons. In embodiments, the method includes classifying cells with the in vitro population of neuronal progenitor cells based at least in part on a computationally derived protein-protein network. In embodiments, the gene expression profile information includes a transcriptional profile. In embodiments, the gene expression profile information includes a transcriptional profile from a single cell. In embodiments, the gene expression reference database comprises known class labels.

The reference database is made up of cell datasets, and each cell dataset is made up of characteristic data. Characteristic data are output from, for example, mRNA expression analysis, microRNA expression analysis, protein expression analysis, post-translational protein modification analysis, non-coding RNA expression analysis, DNA methylation pattern analysis, histone modification analysis, transcription factor-DNA site binding analysis, DNA sequence analysis or any other type of cell characteristic.

B. Test Cells

In some aspects, the methods provided herein allow for determining whether a cell or plurality of cells of unknown identity are determined dopaminergic precursor cells. In some embodiments, the cell or plurality cells of unknown identity are test cells. In some embodiments, the test cells are an in vitro population of cells. In some embodiments, the test cells are contained in an in vitro population of neural progenitor cells. In some embodiments, the test cells include cells differentiated under conditions to become dopaminergic neurons. In some embodiments, the test cells include cells differentiated according to any of the methods described in Section II. In some embodiments, the test cells include cells differentiated under conditions to become dopaminergic neurons for any of the periods of time described in Section II. In some embodiments, the cells being differentiated are pluripotent stem cells. In some embodiments, the pluripotent stem cells are induced pluripotent stem cells (iPSCs). In some embodiments, the iPSCs are generated from fibroblasts collected from healthy human subjects. In some embodiments, the iPSCs are generated from fibroblasts collected from human subjects with Parkinson's disease. Exemplary methods for iPSC generation are described in Section II.

In some embodiments, the determination of the identity of the test cells, e.g., whether the test cells are determined dopaminergic precursor cells or not, indicates whether the in vitro population of cells contains a population of determined dopaminergic precursor cells or not.

In some embodiments, a test dataset is determined from the test cells. In some embodiments, the test dataset is used to determine whether the test cell is a determined dopaminergic precursor cell. In some embodiments, the test dataset is used to determine whether the test cells contain determined dopaminergic precursor cells.

A “test dataset” is a dataset that is produced from a cell (e.g., a neuronal progenitor cell) for which a computed definition is desired. It is produced from characteristic data for an unknown cell line, tissue, or primary cell. Unknown in this context means that a computed definition is desired. Typically the test dataset will be comprised of a global profile as discussed herein as it relates to the global profile of the reference database. The test dataset can be merged with the reference database forming an updated reference database. In certain embodiments this can be as simple as adding the data to an existing spreadsheet. Therefore, the test dataset including gene expression profile information for an in vitro population of neuronal progenitor cells may be included (merged) in the reference database after determining that the in vitro population of neuronal progenitor cells includes a determined dopaminergic precursor cell.

In some embodiments, the test data set includes characteristics of test cells. For example, in some cases, the test data set includes the same types of characteristics as those determined for reference cells. In some embodiments, the test dataset may include cell characteristics such as mRNA expression levels, microRNA expression levels, protein expression levels, post-translational protein modification levels, non-coding RNA expression profiles, DNA methylation levels, histone modification levels, transcription factor-DNA site binding profiles, DNA sequence profiles, or any other type of cell characteristic.

In some embodiments, the test dataset includes protein expression levels. In some embodiments, the test dataset includes post-translational protein modification levels. In some embodiments, the test dataset includes non-coding RNA expression profiles. In some embodiments, the test dataset includes epigenetic profiles. In some embodiments, the test dataset includes transcriptional profiles. In some embodiments, the test dataset includes gene expression levels.

In some embodiments, the gene expression levels are obtained using microarray analysis. In some embodiments, the gene expression levels are obtained using RNA sequencing. In some embodiments, the gene expression levels are obtained using both microarray analysis and RNA sequencing. In some embodiments, the RNA sequencing is performed on bulk RNA from a plurality of cells. In some embodiments, the RNA sequencing is performed on single cells. In some embodiments, the RNA sequencing is performed on bulk RNA from a plurality of cells and on single cells. Exemplary methods of extracting, preparing and analyzing bulk RNA and single-cell RNA are described in Section I.A above.

In some embodiments, the test cell characteristics are included in a test dataset. In some embodiments, the test dataset includes protein expression levels of test cells. In some embodiments, the test dataset includes epigenetic profiles of test cells. In some embodiments, the test dataset includes transcriptional profiles of test cells. In some embodiments, the test dataset includes gene expression levels of test cells. In some embodiments, the test dataset includes microarray data. In some embodiments, the test dataset includes RNA sequencing data. In some embodiments, the test dataset includes microarray data and RNA sequencing data. In some embodiments, the test dataset includes bulk RNA sequencing data. In some embodiments, the test dataset includes single-cell RNA sequencing data. In some embodiments, the test dataset includes bulk RNA sequencing data and single-cell RNA sequencing data. In some embodiments, the test dataset includes expression levels of one or more metagenes. Determination of metagenes and expression levels thereof is discussed in Section I.C.

C. Metagenes

In some aspects, the methods provided herein make use of metagenes and expression levels of metagenes for determining the identity of test cells. A metagene refers to a pattern of gene expression. For example, a metagene may be a group of genes with correlated gene expression. In some embodiments, a metagene combines information from multiple individual genes, and the expression level of the metagene is calculated based on the expression levels of the individual genes. Multiple metagenes and expression levels thereof can be determined based on individual gene expression levels. In some embodiments, metagene expression levels are based on combined individual gene expression levels, and the determination of said metagenes comprises determining the degree to which an individual gene's expression level contributes to the expression level of a metagene. For instance, metagene expression levels can be a weighted combination of individual gene expression levels, and the determination of said metagenes comprises determining for each metagene the weights of individual genes. In some embodiments, metagenes and expression levels thereof reflect correlated expression levels across individual genes. In some embodiments, metagenes and expression levels thereof reflect individual genes coexpressed by cells of the same phenotype (e.g., determined dopaminergic precursor cells). Exemplary coexpressed genes of determined dopaminergic precursor cells are discussed in Section III.

In some aspects, the methods provided herein use the expression levels of metagenes to determine if a cell contained in a population of cells is a determined dopaminergic precursor cell. In some embodiments, the expression levels of metagenes are used to determine whether a population of cells contained determined dopaminergic precursor cells. In some aspects, the use of metagenes reduces the number of features used in determining if a cell is a determined dopaminergic precursor cell or if a population of cells contains determined dopaminergic precursor cells. In some aspects, reducing the number of features makes such determination more computationally tractable. In some aspects, reducing the number of features improves the accuracy of such determination. For instance, the performance of a machine learning model trained using metagene expression levels may be higher than one trained on gene expression levels, particularly since metagenes combine and/or retain information from individual genes.

1. Metagene Determination

In some embodiments, metagenes are determined based on the gene expression levels of reference cells. In some embodiments, the gene expression levels of reference cells are contained in a reference database. Exemplary reference cells and reference databases are described in Section I.A. In some embodiments, a reference database containing microarray data is used to determine metagenes. In some embodiments, a reference database containing RNA sequencing data is used to determine metagenes. In some embodiments, a reference database containing microarray data and reference database containing RNA sequencing data are used to determine metagenes. In some embodiments, a reference database containing bulk RNA sequencing data is used to determine metagenes. In some embodiments, a reference database containing single-cell RNA sequencing data is used to determine metagenes. In some embodiments, a reference database containing bulk RNA sequencing data and a reference database containing single-cell RNA sequencing data are used to determine metagenes.

In some embodiments, metagenes are computationally determined. In some embodiments, metagenes are determined using a dimensionality reduction technique. A dimensionality reduction technique transforms data from a higher-dimensional space (e.g., individual genes) into a lower-dimensional space (e.g., metagenes) such that the lower-dimensional representation of the data still retains meaningful or informative properties of the original data. In some embodiments, metagenes are determined by applying a dimensionality reduction technique on a database.

In some embodiments, the dimensionality reduction technique is a linear technique. In some embodiments, the dimensionality reduction technique is factor analysis. In some embodiments, the dimensionality reduction technique is network component analysis. In some embodiments, the dimensionality reduction technique is linear discriminant analysis. In some embodiments, the dimensionality reduction technique is independent component analysis (ICA). In some embodiments, the dimensionality reduction technique is principal component analysis (PCA). In some embodiments, the dimensionality reduction technique is sparse PCA. In some embodiments, the dimensionality reduction technique is robust PCA.

In some embodiments, the dimensionality reduction technique is non-negative matrix factorization (NMF). Using NMF, a matrix can be factorized into two matrices such that all three matrices have no negative elements. This non-negativity can makes the resulting matrices easier to inspect, for instance when the original matrix itself contains only non-negative values. In some embodiments, the dimensionality reduction technique is conventional NMF. In some embodiments, the dimensionality reduction technique is discriminant NMF. In some embodiments, the dimensionality reduction technique is regularized NMF. In some embodiments, the dimensionality reduction technique is graph regularized NMF. In some embodiments, the dimensionality reduction technique is bootstrapping sparse NMF.

In some embodiments, the dimensionality reduction technique is a non-linear technique. In some embodiments, the dimensionality reduction technique is kernel PCA. In some embodiments, the dimensionality reduction technique is generalized discriminant analysis (GDA). In some embodiments, the dimensionality reduction technique is an autoencoder. In some embodiments, the dimensionality reduction technique is T-distributed Stochastic Neighbor Embedding (t-SNE). In some embodiments, the dimensionality reduction technique is a manifold learning technique. In some embodiments, the dimensionality reduction technique is Isomap. In some embodiments, the dimensionality reduction technique is locally linear embedding (LLE). In some embodiments, the dimensionality reduction technique is Hessian LLE. In some embodiments, the dimensionality reduction technique is Laplacian eigenmaps. In some embodiments, the dimensionality reduction technique is graph-based kernel PCA. In some embodiments, the dimensionality reduction technique is uniform manifold approximation and projection (UMAP).

In some embodiments, the dimensionality reduction technique is a clustering technique that can be used as a dimensionality reduction technique. In some embodiments, the dimensionality reduction technique is a connectivity-based clustering method. In some embodiments, the dimensionality reduction technique is hierarchical clustering. In some embodiments, the dimensionality reduction technique is a centroid-based clustering method. In some embodiments, the dimensionality reduction technique is k-means clustering. In some embodiments, the dimensionality reduction technique is a distribution-based clustering method. In some embodiments, the dimensionality reduction technique is Gaussian mixture modeling. In some embodiments, the dimensionality reduction technique is a density-based clustering method. In some embodiments, the dimensionality reduction technique is DBSCAN. In some embodiments, the dimensionality reduction technique is OPTICS. In some embodiments, the dimensionality reduction technique is a grid-based clustering method. In some embodiments, the dimensionality reduction technique is STING. In some embodiments, the dimensionality reduction technique is CLIQUE.

2. Metagene Expression Levels

In some embodiments, expression levels of the determined metagenes are calculated. In some embodiments, metagene expression levels are determined using the same reference database used to determine metagenes. In some embodiments, metagene expression levels are determined using a reference database not used to determine metagenes. In some embodiments, metagene expression levels are determined using test datasets (e.g., any test dataset described in Section I.B.). Determination of metagene expression levels is possible if expression levels of the same or similar sets of genes are included in the reference databases used to determine metagenes and the reference databases and/or test dataset used to determine metagene expression levels.

In some embodiments, metagene gene expression levels are determined using reference databases containing microarray data. In some embodiments, metagene gene expression levels are determined using a reference database containing RNA sequencing data. In some embodiments, metagene gene expression levels are determined using a reference database containing microarray data and reference databases comprising RNA sequencing data. In some embodiments, metagene gene expression levels are determined using reference database containing bulk RNA sequencing data. In some embodiments, metagene gene expression levels are determined using a reference database containing single-cell RNA sequencing data. In some embodiments, metagene gene expression levels are determined using a reference database containing bulk RNA sequencing data and a reference database containing single-cell RNA sequencing data.

In some embodiments, metagenes are determined using a reference database containing bulk RNA sequencing data, and metagene expression levels are determined using a reference database containing bulk RNA sequencing data. In some embodiments, metagenes are determined using a reference database containing bulk RNA sequencing data, and metagene expression levels are determined using a reference database containing single-cell RNA sequencing data. In some embodiments, metagenes are determined a reference database containing single-cell RNA sequencing data, and metagene expression levels are determined using a reference database containing bulk RNA sequencing data. In some embodiments, metagenes are determined using a reference database containing single-cell RNA sequencing data, and metagene expression levels are determined using a reference database containing single-cell RNA sequencing data. In some embodiments, metagenes are determined using a reference database containing bulk RNA sequencing data and a reference database containing single-cell RNA sequencing data, and metagene expression levels are determined a reference database containing bulk RNA sequencing data. In some embodiments, metagenes are determined using a reference database containing bulk RNA sequencing data and a reference database containing single-cell RNA sequencing data, and metagene expression levels are determined using a reference database containing single-cell RNA sequencing data.

In some embodiments, metagene gene expression levels are determined using a test dataset containing microarray data. In some embodiments, metagene gene expression levels are determined using a test dataset containing RNA sequencing data. In some embodiments, metagene gene expression levels are determined using a test dataset containing microarray data and RNA sequencing data. In some embodiments, metagene gene expression levels are determined using a test dataset containing bulk RNA sequencing data. In some embodiments, metagene gene expression levels are determined using a test dataset containing single-cell RNA sequencing data. In some embodiments, metagene gene expression levels are determined using a test dataset containing bulk RNA sequencing data and single-cell RNA sequencing data.

In some embodiments, metagenes are determined using a reference database containing bulk RNA sequencing data, and metagene expression levels are determined using a test dataset containing bulk RNA sequencing data. In some embodiments, metagenes are determined using a reference database containing bulk RNA sequencing data, and metagene expression levels are determined using a test dataset containing single-cell RNA sequencing data. In some embodiments, metagenes are determined using a reference database containing single-cell RNA sequencing data, and metagene expression levels are determined using a test dataset containing bulk RNA sequencing data. In some embodiments, metagenes are determined using a reference database containing single-cell RNA sequencing data, and metagene expression levels are determined using a test dataset containing single-cell RNA sequencing data. In some embodiments, metagenes are determined using a reference database containing bulk RNA sequencing data and reference databases containing single-cell RNA sequencing data, and metagene expression levels are determined using a test dataset containing bulk RNA sequencing data. In some embodiments, metagenes are determined using a reference database containing bulk RNA sequencing data and reference databases containing single-cell RNA sequencing data, and metagene expression levels are determined using a test dataset containing single-cell RNA sequencing data.

In some embodiments, metagenes are determined by applying a dimensionality reduction technique on one or more reference databases. In some embodiments, one or more outputs of the dimensionality reduction technique are used to determine metagene expression levels.

In some embodiments, one or more outputs of the dimensionality reduction technique and a reference database are used to determine metagene expression levels based on the reference database. In some embodiments, one or more outputs of the dimensionality reduction technique and a test dataset are used to determine metagene expression levels based on the test dataset.

In some embodiments, the one or more outputs of the dimensionality reduction technique includes information on how multiple individual genes are combined to form a metagene. In some embodiments, the one or more outputs of the dimensionality reduction technique includes information on the degree to which an individual gene's expression level contributes to the expression level of a metagene. In some embodiments, the one or more outputs of the dimensionality reduction technique includes the weights of individual genes, for instance when metagene expression levels are a weighted combination of individual gene expression levels.

In some embodiments, metagene expression levels are determined using regression analysis. In some embodiments, the regression analysis is linear regression. In some embodiments, regression analysis is performed using one or more outputs of the dimensionality reduction technique and the reference database. In some embodiments, regression analysis is used to approximate gene expression levels of the reference database using the one or more outputs of the dimensionality reduction technique (e.g., the weights of individual genes in contributing to a metagene). In some embodiments, regression analysis is used to approximate gene expression levels of the reference database as a weighted combination of the weights of individual genes in contributing to a metagene. In some embodiments, the weights estimated by regression analysis can be used as metagene expression levels for the reference database.

In some embodiments, regression analysis is performed using one or more outputs of the dimensionality reduction technique and the test dataset. In some embodiments, regression analysis is used to approximate gene expression levels of the test dataset using the one or more outputs of the dimensionality reduction technique (e.g., the weights of individual genes in contributing to a metagene). In some embodiments, regression analysis is used to approximate gene expression levels of the test dataset as a weighted combination of the weights of individual genes in contributing to a metagene. In some embodiments, the weights estimated by regression analysis can be used as metagene expression levels for the test dataset.

D. Probability Assessment (e.g. Neuroscore)

In some aspects, the methods provided herein include the use of a machine learning model. In some embodiments, the machine learning model is trained to determine the prospect of a cell or a plurality of cells having metagene expression levels of a determined dopaminergic precursor cell. In some embodiments, the machine learning model is trained to determine the probability of a cell or a plurality of cells having metagene expression levels of a determined dopaminergic precursor cell. In some embodiments, the machine learning model is trained to classify a cell or a plurality of cells as having metagene expression levels of a determined dopaminergic precursor cell or not.

In some embodiments, the machine learning model is trained on expression levels of one or more metagenes. In some embodiments, the machine learning model is trained on metagene expression levels determined based on reference databases (e.g., as determined using any of the reference databases described in Section I.A. and any of the methods described in Section I.C.).

In some embodiments, the machine learning model is a supervised classification model. In some embodiments, the machine learning model is trained using reference cell labels comprised in the reference databases. In some embodiments, the reference cell labels indicate if the corresponding reference cells are determined dopaminergic precursor cells. In some embodiments, the reference cell labels indicate the period of time that corresponding reference cells have differentiated under conditions to become dopaminergic neurons, e.g., any of the periods of time described in Section II. In some embodiments, the reference cell labels indicate if the period of time is at least or at least about 18 days. In some embodiments, the reference cell labels indicate if the period of time is between or between about 18 and 25 days.

In some embodiments, the supervised classification model is a logistic regression model. In some embodiments, the supervised classification model is a linear discriminant analysis (LDA) model. In some embodiments, the supervised classification model is a Naïve Bayes classifier. In some embodiments, the supervised classification model is a perceptron. In some embodiments, the supervised classification model is a support vector machine (SVM). In some embodiments, the supervised classification model is a quadratic classifier. In some embodiments, the supervised classification model is a decision tree. In some embodiments, the supervised classification model is a random forest. In some embodiments, the supervised classification model is a neural network. In some embodiments, the supervised classification model is an ensemble model comprising any of the foregoing models.

In embodiments, the machine learning model is a best fitting classification model identified by an algorithm as most stable to random perturbations. In embodiments, the best fitting classification model can cluster individual datasets such that each dataset within a cluster is indistinguishable from each other dataset within said cluster. In embodiments, the method includes identifying computationally derived class labels based only on biological characteristics. In embodiments, the method includes identifying differences in at least one dataset for at least one label between at least two samples in at least two clusters. In embodiments, the method includes filtering within a cluster for samples having a similar label profile. In embodiments, the method includes defining differentially regulated protein-protein networks. In embodiments, the method includes using the protein-protein networks to define a class membership, manipulate class membership, or define biological function of said neuronal progenitor cells. In embodiments, the best fitting classification model can cluster individual datasets such that each dataset within a cluster is different from each other individual dataset.

At some point after a reference database is received the methods can include performing unsupervised classification. This means that a new sorting of the data is performed, with no preconceptions about the results of the sorting. The sorting is typically performed multiple times, at least 5, 10, 20, 50, 100, 200, 300, 500, for example. The sorting results are analyzed for a result that is stable, meaning that the result of the sorting is providing the same result, or a similar result (at least 80%, 85%, 90%, 95%, 97%, 99% or 100% of the previous result). The re-sorting of the data can be performed completely de novo or it can start with certain assumptions.

In some embodiments, metagene expression levels for test cells are determined based on a test dataset (e.g., any of the test datasets described in Section I.B. and using any of the methods described in Section I.C.), and the metagene expression levels are applied as input to the trained machine learning model. In some embodiments, the machine learning model outputs a binary prediction of the test cells having metagene expression levels of a determined dopaminergic precursor cell. In some embodiments, the machine learning model outputs the prospect of the test cells having metagene expression levels of a determined dopaminergic precursor cell. In some embodiments, the machine learning model outputs the probability of the test cells having metagene expression levels of a determined dopaminergic precursor cell. The output (e.g., binary prediction, prospect, probability) is also referred to as a “Neuroscore” herein.

In some embodiments, the Neuroscore output for test cells, e.g. probability of the test cells having metagene expression levels of a determined dopaminergic precursor cell, is compared to a predetermined threshold. In some embodiments, the methods provided herein output a computed label classification, and the computed label classification indicates that the test cells comprise a determined dopaminergic precursor cell if the predetermined threshold is exceeded.

A variety of methods and criteria can be used to set a predetermined threshold for the Neuroscore. For instance, the predetermined threshold can be set in order to optimize specificity and/or sensitivity in predicting if test cells have metagene expression levels of a determined dopaminergic precursor cell. In some embodiments, the predetermined threshold is set such that test cells having metagene expression levels of a determined dopaminergic precursor cell are identified with greater than or greater than about 75%, 80%, 85%, 90%, 95%, 96%, 97%, 98%, or 99% sensitivity. In some embodiments, the predetermined threshold is set such that test cells having metagene expression levels of a determined dopaminergic precursor cell are identified with greater than or greater than about 75%, 80%, 85%, 90%, 95%, 96%, 97%, 98%, or 99% specificity. In some embodiments, the predetermined threshold is set such that test cells having metagene expression levels of a determined dopaminergic precursor cell are identified with greater than or greater than 98% sensitivity and 100% specificity.

In some embodiments, the predetermined threshold is set based on Neuroscores calculated based on reference databases. In some embodiments, the reference databases comprise gene expression levels of reference cells differentiated according to any of the methods described in Section II. In some embodiments, the predetermined threshold is set such that reference cells differentiated for at least or at least about 18 days have Neuroscores exceeding the predetermined threshold. In some embodiments, the predetermined threshold is set such that reference cells differentiated for between or between about 18 and 25 days have Neuroscores exceeding the predetermined threshold. In some embodiments, the predetermined threshold is set such that reference cells known to have a therapeutic effect, e.g., reduce or reverse symptoms of Parkinson's disease, have Neuroscores exceeding the predetermined threshold.

In some embodiments, the computed label classification indicates that the test cells are or contain a determined dopaminergic precursor cell if the test cells' Neuroscore indicates a probability greater than or greater than about 0.4 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.45 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.5 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.55 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.6 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.65 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.7 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.75 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.8 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.85 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.9 of the test cells' having metagene expression levels of a determined dopaminergic precursor cell. In some embodiments, the computed label classification indicates that the test cells are or contain a determined dopaminergic precursor cell if the test cells' Neuroscore indicates a probability greater than or greater than about 0.95 of the test cells' having metagene expression levels of a determined dopaminergic precursor cell.

In some embodiments, the computed label classification indicates that the test cells are or contain a determined dopaminergic precursor cell if the test cells' Neuroscore is greater than or greater than about a threshold probability value. In some embodiments, the threshold probability value is between or between about 0.4 and 1, inclusive. In some embodiments, the threshold probability value is between or between about 0.4 and 0.9, inclusive. In some embodiments, the threshold probability value is between or between about 0.4 and 0.8, inclusive. In some embodiments, the threshold probability value is between or between about 0.4 and 0.7, inclusive. In some embodiments, the threshold probability value is between or between about 0.4 and 0.6, inclusive. In some embodiments, the threshold probability value is between or between about 0.5 and 0.8, inclusive. In some embodiments, the threshold probability value is between or between about 0.5 and 0.7, inclusive. In some embodiments, the threshold probability value is between or between about 0.5 and 0.6, inclusive.

In some embodiments, the threshold probability value is or is about 0.4. In some embodiments, the threshold probability value is or is about 0.45. In some embodiments, the threshold probability value is or is about 0.5. In some embodiments, the threshold probability value is or is about 0.55. In some embodiments, the threshold probability value is or is about 0.6. In some embodiments, the threshold probability value is or is about 0.65. In some embodiments, the threshold probability value is or is about 0.7. In some embodiments, the threshold probability value is or is about 0.75. In some embodiments, the threshold probability value is or is about 0.8. In some embodiments, the threshold probability value is or is about 0.85. In some embodiments, the threshold probability value is or is about 0.9. In some embodiments, the threshold probability value is or is about 0.95.

E. Deviation Score (e.g. Novelty Score)

In some aspects, the methods provided herein comprise calculating a deviation score. The deviation score, also referred to herein as a Novelty Score, indicates the degree to which gene expression levels comprised in a test dataset (e.g., any described in Section I.B.) differ from expected gene expression levels. Expected gene expression values can be determined using a variety of methods. In some embodiments, expected gene expression levels are based on gene expression levels comprised in a reference database, for instance any exemplified in Section I.A. In some embodiments, expected gene expression levels are based on average gene expression levels in a reference database.

In some embodiments, expected gene expression levels are based on the expression levels of one or more metagenes determined for a test dataset, for instance determined using any of the exemplary methods described in Section I.C. herein. In some embodiments, expected gene expression levels are calculated based on gene expression levels in the test dataset and metagenes and expression levels thereof determined for the test dataset. Any method that can be used to calculate an expected value (e.g., expected gene expression level) based on the relationship between one or more predictors (e.g., metagene expression levels for the test dataset) and a dependent value (e.g., gene expression levels in the test dataset) can be used. In some embodiments, regression analysis is used to calculate expected gene expression levels for the test dataset.

In some embodiments, the deviation score is based on all genes whose expression levels are contained in the test dataset. In some embodiments, the deviation score is based on a subset of genes whose expression levels are contained in the test dataset.

In some embodiments, the deviation score is based on a set of preselected marker genes. In some embodiments, the marker genes are chosen based on their diagnostic capability, for instance if their expression levels can be used to distinguish between cell types (e.g., determined dopaminergic precursor cells and other cell types). In some embodiments, the marker genes comprise radial glial cell markers, early neuronal development genes, pluripotency specific markers, intermediate to late neuronal markers, neurofilament light polypeptide chain markers, neurofilament medium polypeptide chain markers, nestin filament markers, early patterning markers, neural progenitor cell markers, early migration markers, stage-specific transcription factors, genes required for normal development of neurons, genes controlling dopaminergic neuron development, genes regulating identity and fate of neuronal progenitor cells, dopaminergic neuron markers, astrocyte markers, forebrain markers, hindbrain markers, subthalamic nucleus markers, radial glial markers, cell cycle markers, or any combination of any of the foregoing. In some embodiments, the marker genes include genes not expected to be expressed by determined dopaminergic precursor cells. In some embodiments, the marker genes include one or more of any of the genes described in Table E1.

In some embodiments, preliminary deviation scores are calculated, and the maximum preliminary deviation score is output as the deviation score. In some embodiments, a first deviation score is calculated based on all genes whose expression levels are contained in the test dataset, and a second deviation score is calculated based on a subset of genes. In some embodiments, a first deviation score is calculated based on all genes whose expression levels are contained in the test dataset, and a second deviation score is calculated based on a set of preselected marker genes. In some embodiments, the deviation score is the maximum value of the preliminary deviation scores.

In some embodiments, the deviation of single genes is calculated as residuals (i.e., differences) between gene expression levels comprised in a test dataset and gene expression levels of one or more reference cells. In some embodiments, the one or more reference cells are at a stage of differentiation indicating a determined dopaminergic precursor cell. In some embodiments, the residuals are normalized. In some embodiments, the residuals are normalized by dividing by the variance of gene expression levels in a reference database, e.g., any of those described in Section I.A. In some embodiments, the residuals are normalized by dividing by the standard deviation of gene expression levels in the reference database.

In some embodiments, the deviation score is a summary statistic of the one or more single-gene deviation scores. Any known summary statistic can be used. In some embodiments, the deviation score is the average single-gene deviation score. In some embodiments, the deviation score is a sum of the single-gene deviation scores. In some embodiments, the deviation score is a weighted sum of the single-gene deviation scores. In some embodiments, single-gene deviation scores of particular genes (e.g., marker genes, for instance those described in Table E1 herein), are weighted more than single-gene deviation scores for other genes. In some embodiments, the deviation score is the single-gene deviation score corresponding to a percentile of one or more single-gene deviation scores. In some embodiments, the percentile is between or between about the 50% percentile and the 100% percentile. In some embodiments, the percentile is between or between about the 60% percentile and the 100% percentile. In some embodiments, the percentile is between or between about the 70% percentile and the 100% percentile. In some embodiments, the percentile is between or between about the 80% percentile and the 100% percentile. In some embodiments, the percentile is between or between about the 90% percentile and the 100% percentile. In some embodiments, the percentile is or is about the 95% percentile.

In some embodiments, the Novelty Score output for test cells is compared to a predetermined threshold. In some embodiments, the methods provided herein output a computed label classification, and the computed label classification indicates that the test cells are or contain a determined dopaminergic precursor cell if the predetermined threshold is not exceeded.

A variety of methods and criteria can be used to set a predetermined threshold for the Novelty Score. In some embodiments, the predetermined threshold is set based on Novelty Scores calculated based on a reference database. In some embodiments, the reference database includes gene expression levels of reference cells differentiated according to any of the methods described in Section II.

In some embodiments, the computed label classification indicates that the test cells are or contain a determined dopaminergic precursor cell if the test cells' Novelty Score indicates that at least or at least about 50% of gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 60% of gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 70% of gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 80% of gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 90% of gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than five standard deviations away from expected gene expression levels.

In some embodiments, the computed label classification indicates that the test cells are or contain a determined dopaminergic precursor cell if the test cells' Novelty Score indicates that at least or at least about 95% of gene expression levels in the test dataset are no more than 10 standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than 9 standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than 8 standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than 7 standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than 6 standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than five standard deviations away from expected gene expression levels.

In some embodiments, the computed label classification indicates that the test cells are or contain a determined dopaminergic precursor cell if the test cells' Novelty Score indicates that at least or at least about 50% of marker gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 60% of marker gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 70% of marker gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 80% of marker gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 90% of marker gene expression levels in the test dataset are no more than five standard deviations away from expected gene expression levels. In some embodiments, the computed label classification indicates that the test cells are or contain a determined dopaminergic precursor cell if the test cells' Novelty Score indicates that at least or at least about 95% of gene expression levels in the test dataset are no more than five standard deviations away from expected gene expression levels.

In some embodiments, the computed label classification indicates that the test cells are or contain a determined dopaminergic precursor cell if the test cells' Novelty Score indicates that at least or at least about 95% of marker gene expression levels in the test dataset are no more than 10 standard deviations away from expected gene expression levels. In some embodiments, the computed label classification indicates that the test cells are or contain a determined dopaminergic precursor cell if the test cells' Novelty Score indicates that at least or at least about 95% of marker gene expression levels in the test dataset are no more than 9 standard deviations away from expected gene expression levels. In some embodiments, the computed label classification indicates that the test cells are or contain a determined dopaminergic precursor cell if the test cells' Novelty Score indicates that at least or at least about 95% of marker gene expression levels in the test dataset are no more than 8 standard deviations away from expected gene expression levels. In some embodiments, the computed label classification indicates that the test cells are or contain a determined dopaminergic precursor cell if the test cells' Novelty Score indicates that at least or at least about 95% of marker gene expression levels in the test dataset are no more than 7 standard deviations away from expected gene expression levels. In some embodiments, the computed label classification indicates that the test cells are or contain a determined dopaminergic precursor cell if the test cells' Novelty Score indicates that at least or at least about 95% of marker gene expression levels in the test dataset are no more than 6 standard deviations away from expected gene expression levels. In some embodiments, the computed label classification indicates that the test cells are or contain a determined dopaminergic precursor cell if the test cells' Novelty Score indicates that at least or at least about 95% of marker gene expression levels in the test dataset are no more than five standard deviations away from expected gene expression levels.

In some embodiments, the computed label classification indicates that the test cells are or contain a determined dopaminergic precursor cell if the test cells' Novelty Score is less than less than about 10. In some embodiments, the computed label classification indicates that the test cells are or contain a determined dopaminergic precursor cell if the test cells' Novelty Score is less than less than about 9. In some embodiments, the computed label classification indicates that the test cells are or contain a determined dopaminergic precursor cell if the test cells' Novelty Score is less than less than about 8. In some embodiments, the computed label classification indicates that the test cells are or contain a determined dopaminergic precursor cell if the test cells' Novelty Score is less than less than about 7. In some embodiments, the computed label classification indicates that the test cells are or contain a determined dopaminergic precursor cell if the test cells' Novelty Score is less than less than about 6. In some embodiments, the computed label classification indicates that the test cells are or contain a determined dopaminergic precursor cell if the test cells' Novelty Score is less than less than about 5.

F. Exemplary Method

In some embodiments, the methods provided herein are used to determine if test cells, e.g. a population of neuronal progenitor cells produced by a differentiation process from iPSCs, are or contain determined dopaminergic precursor cells. In some embodiments, the ability to determine if a test cell population contains determined dopaminergic precursor cells according to any of the methods provided herein can validate release of the cells for use in subsequent applications. In some embodiments, subsequent applications can include therapeutic applications of the determined dopaminergic precursor cells, such as for use in treating a neurodegenerative disease. In some embodiments, the therapeutic applications include the implantation of the test cells for the treatment of a neurodegenerative disease. In some embodiments, the neurodegenerative disease is Parkinson's disease. In some embodiments, the test cells are implanted in the substantia nigra for treating the neurodegenerative disease, e.g. Parkinson's disease.

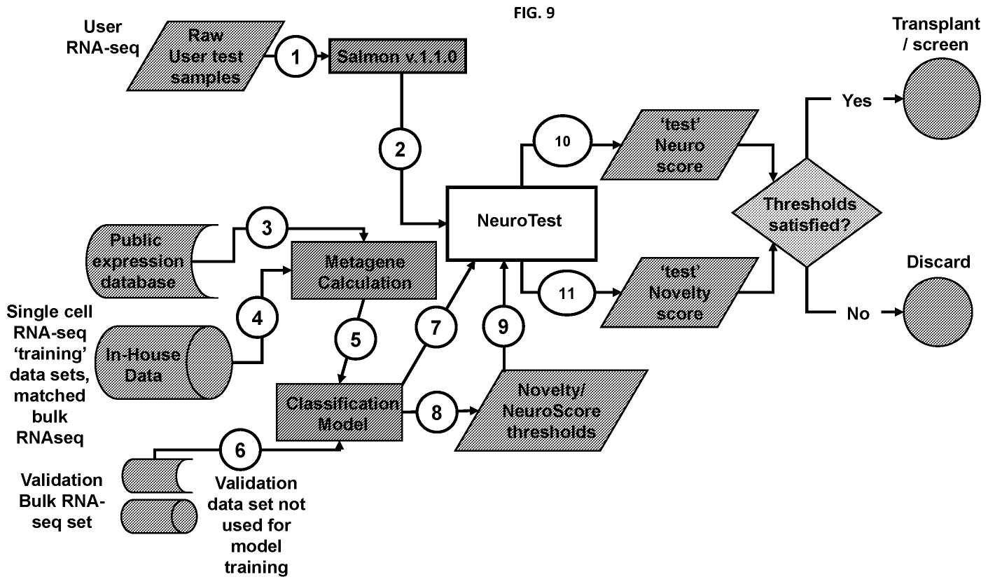

An exemplary process in accord with the provided methods is shown in FIG. 9. In some embodiments, a reference database containing gene expression levels from publically available databases are used. In some embodiments, a reference database containing gene expression levels obtained from single-cell RNA sequencing are used. In some embodiments, a reference database containing gene expression levels obtained from bulk RNA sequencing are used. In some embodiments, the reference database is used (circles 3 and 4) to determine metagenes. In some embodiments, metagene expression levels are calculated for the reference databases and used (circle 5) to train a machine learning model to determine the probability of test cells having metagene expression levels of a determined dopaminergic precursor cell. In some embodiments, the machine learning model can be validated (circle 6) using additional data, for instance bulk RNA sequencing data not used in training the model.

In some embodiments, the trained machine learning is used as part of the methods provided herein (circle 7) for classifying test cells. In some embodiments, Novelty Scores are calculated based on the reference databases. In some embodiments, the Novelty Scores based on the reference databases are used to identify NeuroScore and Novelty Score thresholds (circle 8).

In some embodiments, test cells are used to produce a test dataset including gene expression levels of the test cells. In some embodiments, the gene expression levels of the test cells are obtained using RNA sequencing. In some embodiments, the gene expression levels are subjected to sequencing alignment (circle 1). In some embodiments, the sequencing alignment is performed using a Salmon pseudoaligner. In some embodiments, the test dataset is supplied to the trained model (circle 2). In some embodiments, a NeuroScore (circle 10) and a Novelty Score (circle 11) are output for the test dataset. In some embodiments, the NeuroScore and the Novelty Score are compared to the previously determined NeuroScore and Novelty Score thresholds. In some embodiments, the test cells are transplanted and/or screened, for instance if both thresholds are met. In some embodiments, the test cells are discarded, for instance if neither threshold is met.

In some embodiments, reference cells and reference databases are produced, for instance according to any of the methods described in Sections I.A and II. In some embodiments, the reference cells are produced using iPSCs generated from subjects with Parkinson's disease. In some embodiments, the reference databases include gene expression levels of reference cells allowed to differentiate from iPSCs for various times in culture, such as for, for about, or for at least 13, 18, and 25 days under conditions to differentiate iPSCs into neuronal cells. In some embodiments, the reference database includes bulk RNA sequencing data. In some embodiments, the reference database includes single-cell RNA sequencing data. In some embodiments, the reference database includes reference cell labels indicating if reference cells exhibit features of determined dopaminergic precursor cells, for example, as determined by functional assays, such as using animal models of a neurodegenerative disease. In some embodiments, the reference database includes reference cell labels of a cell population differentiated into neuronal cells from iPSCs for, for about, or for at least 18 days. The methods of differentiation can include any as described in Section II.

In some embodiments, the reference database including single-cell RNA sequencing data is used to determine metagenes, for instance using any of the methods described in Section I.C.1. In some embodiments, and based on the determined metagenes, metagene expression levels are determined using a reference database including bulk RNA sequencing data, for instance using any of the methods described in Section I.C.2.

In some embodiments, the metagene expression levels are used to train a machine learning model, for instance any described in Section I.D. In some embodiments, the machine learning model is a supervised classification model. In some embodiments, the machine learning model is a logistic regression model. In some embodiments, the machine learning model is trained using reference cell labels comprised in the reference databases.

In some embodiments, test cells and test datasets are produced, for instance using any of the methods described in Sections I.B. and II. In some embodiments, the test cells are produced using iPSCs generated from a patient with Parkinson's disease. In some embodiments, the test dataset is used to determine metagene expression levels for the test cells, for instance using any of the methods described in Section I.C.2. In some embodiments, the test cells are contained in an in vitro population of cells. In some embodiments, the test cells are contained in an in vitro population of neuronal progenitor cells

In some embodiments, the metagene expression levels determined from the test dataset are supplied as input to the machine learning model. In some embodiments, the machine learning model outputs a Neuroscore (e.g., any exemplified in Section I.D.). In some embodiments, a Novelty Score is determined using the test dataset, for instance according to any of the methods described in Section I.E. In some embodiments, a Neuroscore and a Novelty Score are determined for the test cells.

In some embodiments, the test cells' Neuroscore is compared to a predetermined threshold (e.g., any described in Section I.D.). In some embodiments, the test cells' Novelty Score is compared to a predetermined threshold (e.g., any described in Section I.E.). In some embodiments, both the Neuroscore and the Novelty Score of the test cells are compared to predetermined thresholds.

In some embodiments, the methods provided herein include outputting a computed label classification comprising an indication of whether the test cells include a determined dopaminergic precursor cell. In some embodiments, the computed label classification is based on the Neuroscore and comparison thereof to its corresponding predetermined threshold. In some embodiments, the computed label classification is based on the Novelty Score and comparison thereof to its corresponding predetermined threshold. In some embodiments, the computed label classification is based on both the Neuroscore and comparison thereof to its corresponding predetermined threshold and on the Novelty Score and comparison thereof to its corresponding predetermined threshold.

In some embodiments, the computed label classification indicates that the test cells are or contain a determined dopaminergic precursor cell if the test cells' Neuroscore indicates a probability greater than or greater than about 0.5 of the test cells' having metagene expression levels of a predetermined dopaminergic precursor cell. In some embodiments, the computed label classification indicates that the test cells are or contain a determined dopaminergic precursor cell if the test cells' Novelty Score indicates that at least or at least about 95% of gene expression levels in the test dataset are no more than five standard deviations away from expected gene expression levels. In some embodiments, the computed label classification indicates that the test cells are or contain a determined dopaminergic precursor cell if (i) the test cells' Neuroscore indicates a probability greater than or greater than about 0.5 of the test cells' having metagene expression levels of a determined dopaminergic precursor cell and (ii) the test cells' Novelty Score indicates that at least or at least about 95% of gene expression levels in the test dataset are no more than five standard deviations away from expected gene expression levels.

In some embodiments, the test cells' computed label classification indicates that the test cells are or contain determined dopaminergic precursor cells. In some embodiments, the in vitro population of cells comprising the test cells identified as determined dopaminergic precursor cells is selected for use. In some embodiments, the in vitro population of cells containing the test cells identified as determined dopaminergic precursor cells is selected for transplant, for instance according to any of the methods described in Section V.

In some embodiments, the test cells' computed label classification indicates that the test cells do not contain determined dopaminergic precursor cells. In some embodiments, the test cells' Novelty Score indicates that less than or less than about 95% of gene expression levels in the test dataset were no more than five standard deviations away from expected gene expression levels. In some embodiments, the in vitro population of cells comprising the test cells not identified as determined dopaminergic precursor cells is no longer allowed to differentiate. In some embodiments, the in vitro population of cells containing the test cells not identified as determined dopaminergic precursor cells is discarded. In some embodiments, the methods provided herein are repeated by producing an additional set of test cells and another test dataset. In some embodiments, the additional set of test cells is produced from the same subject with Parkinson's disease. In some embodiments, the additional set of test cells is produced from the same population of iPSCs with which the first set of test cells was produced. In some embodiments, a computed label classification is output for the additional set of test cells.

In some embodiments, the test cells' computed label classification indicates that the test cells do not contain determined dopaminergic precursor cells. In some embodiments, the test cells' Neuroscore indicates that a probability less than or less than about 0.5 of the test cells' having metagene expression levels of a determined dopaminergic precursor cell. In some embodiments, the test cells' Novelty Score indicates that greater than or greater than about 95% of gene expression levels in the test dataset were no more than five standard deviations away from expected gene expression levels. In some embodiments, the in vitro population of cells containing the test cells not identified as determined dopaminergic precursor cells is allowed to continue differentiating. In some embodiments, an additional set of test cells and test dataset from the same in vitro population of cells is collected. In some embodiments, a computed label classification is output for the additional set of test cells.

In some embodiments, the additional set of test cells is collected and tested according to the methods provided herein between or between about one and 30 days after testing of the first set of test cells. In some embodiments, the additional set of test cells is collected and tested according to the methods provided herein between or between about one and 25 days after testing of the first set of test cells. In some embodiments, the additional set of test cells is collected and tested according to the methods provided herein between or between about one and 20 days after testing of the first set of test cells. In some embodiments, the additional set of test cells is collected and tested according to the methods provided herein between or between about one and 15 days after testing of the first set of test cells. In some embodiments, the additional set of test cells is collected and tested according to the methods provided herein between or between about one and 10 days after testing of the first set of test cells. In some embodiments, the additional set of test cells is collected and tested according to the methods provided herein between or between about one and 5 days after testing of the first set of test cells. In some embodiments, the additional set of test cells is collected and tested according to the methods provided herein between or between about one and 3 days after testing of the first set of test cells.

In some embodiments, the methods provided herein are repeated until a computed label classification is provided indicating that test cells produced from the subject are or contain determined dopaminergic precursor cells.

In embodiments, the computed label classification is an unsupervised classification of the updated reference database including clustering RNA, DNA and/or protein profiles. In embodiments, the gene expression profile information is obtained from microarray analysis of cellular RNA. In embodiments, the gene expression profile information is obtained from microarray analysis of cellular RNA derived from a single cell. In embodiments, the computed label classification is an unsupervised machine classification including a bootstrapping sparse non-negative matrix factorization.

In embodiments, the gene expression reference database forms part of a storage medium. In embodiments, receiving the test dataset includes receiving input from an array analysis system. In embodiments, receiving the test dataset includes receiving input via a computer network. In embodiments, the data in the reference database is associated with one or more labeled associated biological classes of the cells.

### II. Methods for Differentiating Cells

In some aspects, the methods provided herein include the use of reference cells and/or test cells that are the product of a method to differentiate a cell. In some embodiments, the reference cells and/or test cells described in Sections I.A. and I.B. are the product of a method to differentiate a pluripotent stem cell. Various sources of pluripotent stem cells can be used, including embryonic stem (ES) cells and induced pluripotent stem cells (iPSCs). In some embodiments, the cell is an iPSC. In some embodiments, the pluripotent stem cell is an iPSC. In some embodiments, the pluripotent stem cell is an iPSC, artificially derived from a non-pluripotent cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka Cell (2006) 126: 663-76.

In some embodiments, the cell is a pluripotent stem cell. In some embodiments, the cell is a pluripotent stem cell that was artificially derived from a non-pluripotent cell of a subject. In some embodiments, the non-pluripotent cell is a fibroblast. In some embodiments, the subject is a human. In some embodiments, the subject is a human with Parkinson's Disease. In some embodiments, the pluripotent stem cell is an iPSC.

A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphologies associated with pluripotent stem cells.

Methods for generating iPSCs are known. For example, mouse iPSCs were reported in 2006 (Takahashi and Yamanaka), and human iPSCs were reported in late 2007 (Takahashi et al. and Yu et al). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the reference cells and/or the test cells are neuronal cells that have been differentiated from a pluripotent stem cell. In some embodiments, the cells are differentiated using methods that differentiate cells, e.g., iPSCs, into any neural cell type using any available or known method for inducing the differentiation of cells. As is understood, the particular differentiation protocol and timing of the culture may result in different states of differentiated neuronal cells. In some embodiments, the differentiation is carried out by culture of pluripotent stem cells, e.g. iPSCs, under conditions to produce neuronal progenitor cells that are or include cells that are committed to being a neuronal cell. In some embodiments, the iPSCs are differentiated under conditions to result in floor plate midbrain progenitor cells, determined dopaminergic precursor cells, and/or dopamine (DA) neurons. In some embodiments, iPSCs are cultured under conditions to for differentiation into determined dopaminergic precursor cells. In some embodiments, the iPSCs are cultured under conditions to differentiate into dopaminergic neurons. Any available and known method for inducing differentiation of the cells, e.g., pluripotent stem cells, into floor plate midbrain progenitor cells, determined dopaminergic precursor cells, and/or dopamine (DA) neurons can be used. Exemplary methods of differentiating neural cells can be found, e.g., in WO2013104752, WO2010096496, WO2013067362, WO2014176606, WO2016196661, WO2015143342, US20160348070, the contents of which are hereby incorporated by reference in their entirety. In some embodiments, iPSCs are allowed to differentiate in culture as part of differentiation into neuronal cells. In some embodiments, the cells are cultured or incubated in the presence of one or more factors able to induce or promote the differentiation of iPSCs into neuronal cells. In some embodiments, the iPSCs are cultured in the presence of one or more of (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling. In some embodiments, the iPSCs are cultured in the presence of (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling. In some embodiments, the inhibitor of TGF-β/activing-Nodal signaling is 5B431542 (e.g. between about 1 μM and about 20 μM, such as 10 μM). In some embodiments, the at least one activator of SHH signaling is SHH (e.g. between about 10 ng/mL and about 500 ng/mL, such as 100 ng/mL) or purmorphamine (e.g. between about 0.1 μM and about 10 μM, such as 2 μM). In some embodiments, the at least one activator of SHH signaling includes SHH protein (e.g. between about 10 ng/mL and about 500 ng/mL, such as 100 ng/mL) and purmorphamine (e.g. between about 0.1 μM and about 10 μM, such as 2 μM). In some embodiments, the inhibitor of BMP signaling is LDN193189 (e.g. between about 0.01 μM and about 5 μM, such as 0.1 μM). In some embodiments, the inhibitor of GSK3β signaling is CHIR99021 (e.g. between about 0.1 μM and about 10 μM, such as 2 μM).

In some embodiments, the iPSCs are exposed to the one or more factors or agents at the initiation of the culturing or incubation (day 0). In some embodiments, the presence of the one or more of the factors or agents, each independently, may be maintained in the culture for the duration of the culture or for a portion of the culture. In some embodiments, the one or more factors or agents are, each independently, present in the culture for a time period to allow differentiation of the iPSCs into midbrain floor plate precursors, or until such cells exhibit characteristics of midbrain floor plate precursors as determined by a classification label according to the provided methods. In some embodiments, the one or more factors or agents are, each independently, present in the culture for up to day 5, up to day 6, up to day 7, up to day 8, up to day 9, up to day 10, up to day 11, up to day 12 or up to day 13 of the culture. For example, in an exemplary protocol, the culturing under conditions for differentiating iPSCs into neuronal cells includes initiating a first incubation on about day 0, wherein the first incubation includes culturing the pluripotent stem cells and exposing the cells to (i) an inhibitor of TGF-β/activing-Nodal signaling from day 0 through day 10, each day inclusive; (ii) at least one activator of Sonic Hedgehog (SHH) signaling from day 1 through day 6, each day inclusive; (iii) an inhibitor of bone morphogenetic protein (BMP) signaling from day 0 through day 10, each day inclusive; and (iv) an inhibitor of glycogen synthase kinase 3β (GSK3β) signaling from day 0 through day 12, each day inclusive.

In some embodiments, a second culture or incubation can be carried out on cells differentiated in the first culture, in which the second culture or incubation is carried out the presence of one or more additional agents or factors under conditions to further neurally differentiate the cells. In some embodiments, the second culture or initiation may be initiated at or about the time that the cells in the first culture have differentiated into midbrain floor plate precursors, or until such cells exhibit characteristics of midbrain floor plate precursors as determined by a classification label according to the provided methods. In some embodiments, the one or more additional agents or factors can include any one or more the one or more factors present in the first culture. In some embodiments, the one or more additional agents or factors can include one or more of (i) brain-derived neurotrophic factor (BDNF); (ii) ascorbic acid; (iii) glial cell-derived neurotrophic factor (GDNF); (iv) cyclic AMP (cAMP), e.g. dibutyryl cyclic AMP (dbcAMP); (v) transforming growth factor beta-3 (TGFβ3) (collectively, “BAGCT”); and (vi) an inhibitor of Notch. In some embodiments, the additional agents or factors include (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch. In some embodiments, the cells are exposed to a concentration of BDNF between about 1 ng/mL and 100 ng/mL (e.g. 20 ng/mL). In some embodiments, the cells are exposed to ascorbic acid at a concentration of between about 0.05 mM and 5 mM, e.g. 0.2 mM. In some embodiments, the cells are exposed to GDNF at a concentration of between 1 ng/mL and 100 ng/mL, e.g. 20 ng/mL. In some embodiments, the cells are exposed to cAMP, e.g. dibutyryl cyclic AMP (dbcAMP), at a concentration between about 0.05 mM and 5 mM, e.g. about 0.5 mM. In some embodiments, the cells are exposed to transforming growth factor beta 3 (TGFβ3) at a concentration of between about 0.1 ng/mL and 10 ng/mL, e.g. 1 ng/mL.

In some embodiments, the second culture or incubation can be carried out for a period of time to differentiate the cells into determined dopaminergic precursor cells, or until such cells exhibit characteristics of dopaminergic neurons as determined by a classification label according to the provided methods. In some embodiments the second culture or incubation can be carried out for a period of time to differentiate the cells into dopaminergic neurons, or until such cells exhibit characteristics of dopaminergic neurons as determined by a classification label according to the provided methods. In some embodiments, the second culture or incubation is carried out up until about day 30 after the initiation of the first culture or incubations. In some embodiments, the second culture or incubation is carried out up until about day 11 to day 25 after initiation of the first culture or incubations, such as from day 11, day 12, day 13, day 14, day 15, day 16, day 17, day 18, day 19, day 20, day 21, day 22, day 23, day 24 or day 25. In some embodiments, the second culture or incubation is carried out to at or about day 18 after initiation of the first culture. In some embodiments, the second culture is carried out to at or about day 25 after initiation of the first culture.

In some embodiments, cells of the culture are exposed to the one or more additional factors or agents for the duration of the culture or for a period of time. In some embodiments, the presence of the one or more of additional factors or agents, each independently, may be maintained in the culture for the duration of the culture or for a portion of the culture. In some embodiments, the one or more additional factors or agents are, each independently, present in the culture for a time period to differentiate the cells into determined dopaminergic precursor cells, or until such cells exhibit characteristics of dopaminergic neurons as determined by a classification label according to the provided methods. In some embodiments, the one or more additional factors or agents are, each independently, present in the culture for a time period to differentiate the cells into dopaminergic neurons, or until such cells exhibit characteristics of dopaminergic neurons as determined by a classification label in accord with the provided methods. In some embodiments, the second culture or incubation is carried out up until about day 30 after the initiation of the first culture or incubations. In some embodiments, the one or more additional agent or factor are, each independently, present in the culture from the initiation of the second culture until about day 11 to day 25 after initiation of the first culture or incubation, such as up until day 11, day 12, day 13, day 14, day 15, day 16, day 17, day 18, day 19, day 20, day 21, day 22, day 23, day 24 or day 25. In some embodiments, the one or more additional agent or factor are, each independently, present in the culture from the initiation of the second culture to at or about day 18 after initiation of the first culture. In some embodiments, the one or more additional agent or factor are, each independently, present in the culture from the initiation of the second culture until to at or about day 25 after initiation of the first culture. For example, in an exemplary protocol, the culturing under conditions for differentiating iPSCs into neuronal cells further includes a second incubation in which cells from the first incubation are further cultured by exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch, beginning on day 11. In some embodiments, the cells are exposed to BAGCT until harvest of the neurally differentiated cells, such as until day 18 or until day 25. In some embodiments, the second incubation may further include culture by exposing the cells to an inhibitor of GSK3β signaling from day 11 through day 12, each day inclusive.

In some embodiments, the incubation may include culture by exposing the cells to an inhibitor of Rho-associated protein kinase (ROCK) signaling at one or more times during the culturing, such as on about day 0, day 7, day 16 and/or day 20 from the initiation of the first culture. In some embodiments, the ROCK inhibitor is Y-27632 (e.g. between about 1 μM and about 20 μM, such as about 10 μM.

In some embodiments, the culturing of the iPSCs under conditions for differentiation into neuronal cells can be for a time period from the initiation of the culturing until harvest of differentiated cells that is between 10 days and 30 days. It is understood that the particular timing may be chosen based on the desired differentiation state of the cells, for example as determined empirically by a functional or other phenotypic assay or as determined based on classification label of the differentiated cells as determined in accord with the provided methods. In some embodiments, a reference cell is differentiated by culture for a certain or defined period of time. In some embodiments a reference cell is differentiated by culture for a total period of time in which the cell is determined to exhibit a desired functional or phenotypic attribute or feature, e.g. as described in Section I.A. In some embodiments, a test cell is differentiated by culture for a total period of time. In some embodiments, a test cell is differentiated by culture for a total period of time at which it is determined the test cell exhibits a desired classification label in accord with the provided methods. In some embodiments, the provided methods can be used to assess if a test cell has been cultured under conditions for its differentiation into a desired neuronal cell, e.g. determined dopaminergic precursor cell, by its classification label as determined in accord with any of the provided methods.

In embodiments, the iPSC is cultured for differentiation into a neuronal cell for at least 10 days. In embodiments, the iPSC is cultured for differentiation into a neuronal cell for at least 11 days. In embodiments, the iPSC is cultured for differentiation into a neuronal cell for at least 12 days. In embodiments, the iPSC is cultured for differentiation into a neuronal cell for at least 13 days. In embodiments, the iPSC is cultured for differentiation into a neuronal cell for at least 14 days. In embodiments, the iPSC is cultured for differentiation into a neuronal cell for at least 15 days. In embodiments, the iPSC is cultured for differentiation into a neuronal cell for at least 16 days. In embodiments, the iPSC is cultured for differentiation into a neuronal cell for at least 17 days. In embodiments, the iPSC is cultured for differentiation into a neuronal cell for at least 18 days. In embodiments, the iPSC is cultured for differentiation into a neuronal cell for at least 19 days. In embodiments, the iPSC is cultured for differentiation into a neuronal cell for at least 20 days.

In embodiments, the iPSC is cultured for differentiation into a neuronal cell for about 10 days. In embodiments, the iPSC is cultured for differentiation into a neuronal cell for about 11 days. In embodiments, the iPSC is cultured for differentiation into a neuronal cell for about 12 days. In embodiments, the iPSC is cultured for differentiation into a neuronal cell for about 13 days. In embodiments, the iPSC is cultured for differentiation into a neuronal cell for about 14 days. In embodiments, the iPSC is cultured for differentiation into a neuronal cell for about 15 days. In embodiments, the iPSC is cultured for differentiation into a neuronal cell for about 16 days. In embodiments, the iPSC is cultured for differentiation into a neuronal cell for about 17 days. In embodiments, the iPSC is cultured for differentiation into a neuronal cell for about 18 days. In embodiments, the iPSC is cultured for differentiation into a neuronal cell for about 19 days. In embodiments, the iPSC is cultured for differentiation into a neuronal cell for about 20 days. In embodiments, the iPSC is cultured for differentiation into a neuronal cell for about 21 days. In embodiments, the iPSC is cultured for differentiation into a neuronal cell for about 22 days. In embodiments, the iPSC is cultured for differentiation into a neuronal cell for about 23 days. In embodiments, the iPSC is cultured for differentiation into a neuronal cell for about 24 days. In embodiments, the iPSC is cultured for differentiation into a neuronal cell for about 25 days.

In some embodiments, reference cells, for example as described in Section I.A., undergo methods of differentiation as described herein. In some embodiments, test cells, for example as described in Section I.B., undergo methods of differentiation as described herein. In some embodiments, both reference cells and test cells undergo the same methods of differentiation as provided herein.

### III. Exemplary Features of a Determined Dopaminergic Neuron

In some embodiments, the determined dopaminergic precursor cells identified by the methods provided herein have certain increased and/or decreased gene expression levels relative to a pluripotent stem cell. In some embodiments, an in vitro population of neuronal progenitor cells having certain increased and/or decreased gene expression levels relative to a pluripotent stem cell is indicative of the in vitro population comprising desirable determined dopaminergic precursor cells.

In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies of Table 1.

In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies selected from the group consisting of gene ontologies of Table 1.

In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies of GO:0007399, GO:0120025, GO:0042995, GO:0032502, GO:0044767, GO:0048856, GO:0048731, GO:0022008, GO:0048699, GO:0007275, GO:0030030, GO:0032501, GO:0044707, GO:0050874, GO:0048468, GO:0120036, GO:0120038, GO:0044463, GO:0097458, GO:0045202, GO:0030182, GO:0030154, GO:0048869, GO:0051960, GO:0007156, GO:0005929, GO:0072372, GO:0035082, GO:0035083, GO:0035084, GO:0060284, GO:0050767, GO:0001578, GO:0016339, GO:0043005, GO:0044456, GO:0098742, GO:0045664, GO:0006928, GO:0099699, GO:0048666, GO:0003341, GO:0036142, GO:0005509, GO:0097060, GO:0031514, GO:0009434, GO:0031512, GO:0007155, GO:0098602, GO:0010975, GO:0098794, GO:0022610, GO:0030424, GO:0099240, GO:0032989, GO:0120035, GO:0000902, GO:0007148, GO:0045790, GO:0045791, GO:0048812, GO:0036477, GO:0031344, GO:0120039, GO:0061564, GO:0048858, GO:0099055, GO:0009653, GO:0098609, GO:0016337, GO:0031175, GO:0005930, GO:0035085, GO:0035086, GO:0010720, GO:0007416, GO:0097014, GO:0032990, GO:0098936, GO:0043025, GO:0050768, GO:0051962, GO:0050808, GO:0007409, GO:0007410, GO:2000026, GO:0045597, GO:0044441, GO:0044442, GO:0007417, GO:0048667, GO:0010721, GO:0044459, GO:0060322, GO:0045211, GO:0045666, GO:0032838, GO:0099056, GO:0051961, GO:0044297, GO:0007018, GO:0050769, GO:0040011, GO:0050793, GO:0051094, GO:0005874, GO:0000904, GO:0010976, GO:0045595, GO:0050770, GO:0099536, GO:0098889, GO:0051239, GO:0007420, GO:0099537, GO:0031346, GO:0007268, GO:0098916, GO:0097485, GO:0044782, GO:0031226, GO:0060285, GO:0071974, GO:0010769, GO:0001539, GO:0050804, GO:0099177, GO:0005887, GO:0098984, GO:0045665, GO:0050919, GO:0007411, GO:0008040, GO:0030425, GO:0061387, GO:0097447, GO:0050803, GO:0042734, GO:0042391, GO:0001764, GO:0032279, GO:0010770, GO:0021953, GO:0099572, GO:0098590, GO:0044447, GO:0098978, GO:0014069, GO:0097481, GO:0097483, GO:0033267, GO:0010977, GO:0007017, GO:0150034, GO:0034702, GO:0034703, GO:0050807, GO:0060271, GO:0042384, GO:0051240, GO:0050772, GO:0120031, GO:0007626, GO:0008092, GO:0005886, GO:0005904, GO:0007610, GO:0044708, GO:0098793, GO:0022604, GO:0007267, GO:0071944, GO:0099060, GO:0022836, GO:0030031, GO:0042220, GO:0019226, GO:0030516, GO:0035637, GO:0045596, GO:0021954, GO:0022832, GO:0005244, GO:1902495, GO:0050771, GO:0048513, GO:0022839, GO:0098948, GO:0001508, GO:0099568, GO:0008484, GO:0051966, GO:0003358, GO:0033602, GO:0005261, GO:0015281, GO:0015338, GO:0022603, GO:1990351, GO:0097729, GO:0015631, GO:0051270, GO:0005216, GO:0016043, GO:0044235, GO:0071842, GO:0031345, GO:0005856, GO:0022838, GO:0099061, GO:0098982, GO:0051674, GO:0048870, GO:0060294, GO:0072359, GO:0099634, GO:0015630, GO:0036126, GO:1990939, GO:0072347, GO:0015267, GO:0015249, GO:0015268, GO:0022803, GO:0022814, GO:0008045, GO:0098797, GO:0060160, GO:0099146, GO:0010771, GO:0000226, GO:0045503, GO:0005578, GO:0030334, GO:0044304, GO:0010463, GO:0010646, GO:0008574, GO:0043279 or any combination thereof.

In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies selected from the group consisting of GO:0007399, GO:0120025, GO:0042995, GO:0032502, GO:0044767, GO:0048856, GO:0048731, GO:0022008, GO:0048699, GO:0007275, GO:0030030, GO:0032501, GO:0044707, GO:0050874, GO:0048468, GO:0120036, GO:0120038, GO:0044463, GO:0097458, GO:0045202, GO:0030182, GO:0030154, GO:0048869, GO:0051960, GO:0007156, GO:0005929, GO:0072372, GO:0035082, GO:0035083, GO:0035084, GO:0060284, GO:0050767, GO:0001578, GO:0016339, GO:0043005, GO:0044456, GO:0098742, GO:0045664, GO:0006928, GO:0099699, GO:0048666, GO:0003341, GO:0036142, GO:0005509, GO:0097060, GO:0031514, GO:0009434, GO:0031512, GO:0007155, GO:0098602, GO:0010975, GO:0098794, GO:0022610, GO:0030424, GO:0099240, GO:0032989, GO:0120035, GO:0000902, GO:0007148, GO:0045790, GO:0045791, GO:0048812, GO:0036477, GO:0031344, GO:0120039, GO:0061564, GO:0048858, GO:0099055, GO:0009653, GO:0098609, GO:0016337, GO:0031175, GO:0005930, GO:0035085, GO:0035086, GO:0010720, GO:0007416, GO:0097014, GO:0032990, GO:0098936, GO:0043025, GO:0050768, GO:0051962, GO:0050808, GO:0007409, GO:0007410, GO:2000026, GO:0045597, GO:0044441, GO:0044442, GO:0007417, GO:0048667, GO:0010721, GO:0044459, GO:0060322, GO:0045211, GO:0045666, GO:0032838, GO:0099056, GO:0051961, GO:0044297, GO:0007018, GO:0050769, GO:0040011, GO:0050793, GO:0051094, GO:0005874, GO:0000904, GO:0010976, GO:0045595, GO:0050770, GO:0099536, GO:0098889, GO:0051239, GO:0007420, GO:0099537, GO:0031346, GO:0007268, GO:0098916, GO:0097485, GO:0044782, GO:0031226, GO:0060285, GO:0071974, GO:0010769, GO:0001539, GO:0050804, GO:0099177, GO:0005887, GO:0098984, GO:0045665, GO:0050919, GO:0007411, GO:0008040, GO:0030425, GO:0061387, GO:0097447, GO:0050803, GO:0042734, GO:0042391, GO:0001764, GO:0032279, GO:0010770, GO:0021953, GO:0099572, GO:0098590, GO:0044447, GO:0098978, GO:0014069, GO:0097481, GO:0097483, GO:0033267, GO:0010977, GO:0007017, GO:0150034, GO:0034702, GO:0034703, GO:0050807, GO:0060271, GO:0042384, GO:0051240, GO:0050772, GO:0120031, GO:0007626, GO:0008092, GO:0005886, GO:0005904, GO:0007610, GO:0044708, GO:0098793, GO:0022604, GO:0007267, GO:0071944, GO:0099060, GO:0022836, GO:0030031, GO:0042220, GO:0019226, GO:0030516, GO:0035637, GO:0045596, GO:0021954, GO:0022832, GO:0005244, GO:1902495, GO:0050771, GO:0048513, GO:0022839, GO:0098948, GO:0001508, GO:0099568, GO:0008484, GO:0051966, GO:0003358, GO:0033602, GO:0005261, GO:0015281, GO:0015338, GO:0022603, GO:1990351, GO:0097729, GO:0015631, GO:0051270, GO:0005216, GO:0016043, GO:0044235, GO:0071842, GO:0031345, GO:0005856, GO:0022838, GO:0099061, GO:0098982, GO:0051674, GO:0048870, GO:0060294, GO:0072359, GO:0099634, GO:0015630, GO:0036126, GO:1990939, GO:0072347, GO:0015267, GO:0015249, GO:0015268, GO:0022803, GO:0022814, GO:0008045, GO:0098797, GO:0060160, GO:0099146, GO:0010771, GO:0000226, GO:0045503, GO:0005578, GO:0030334, GO:0044304, GO:0010463, GO:0010646, GO:0008574, GO:0043279 and any combination thereof.

In embodiments, the first gene set includes about 1-500 increased genes within one or more of the first gene ontologies. In embodiments, the first gene set includes about 2-500 increased genes within one or more of the first gene ontologies. In embodiments, the first gene set includes about 3-500 increased genes within one or more of the first gene ontologies. In embodiments, the first gene set includes about 4-500 increased genes within one or more of the first gene ontologies. In embodiments, the first gene set includes about 5-500 increased genes within one or more of the first gene ontologies. In embodiments, the first gene set includes about 6-500 increased genes within one or more of the first gene ontologies. In embodiments, the first gene set includes about 7-500 increased genes within one or more of the first gene ontologies. In embodiments, the first gene set includes about 8-500 increased genes within one or more of the first gene ontologies. In embodiments, the first gene set includes about 9-500 increased genes within one or more of the first gene ontologies. In embodiments, the first gene set includes about 10-500 increased genes within one or more of the first gene ontologies.

In embodiments, the first gene set includes about 15-500 increased genes within one or more of the first gene ontologies. In embodiments, the first gene set includes about 20-500 increased genes within one or more of the first gene ontologies. In embodiments, the first gene set includes about 25-500 increased genes within one or more of the first gene ontologies. In embodiments, the first gene set includes about 30-500 increased genes within one or more of the first gene ontologies. In embodiments, the first gene set includes about 35-500 increased genes within one or more of the first gene ontologies. In embodiments, the first gene set includes about 40-500 increased genes within one or more of the first gene ontologies. In embodiments, the first gene set includes about 45-500 increased genes within one or more of the first gene ontologies. In embodiments, the first gene set includes about 50-500 increased genes within one or more of the first gene ontologies. In embodiments, the first gene set includes about 55-500 increased genes within one or more of the first gene ontologies. In embodiments, the first gene set includes about 60-500 increased genes within one or more of the first gene ontologies. In embodiments, the first gene set includes about 65-500 increased genes within one or more of the first gene ontologies. In embodiments, the first gene set includes about 70-500 increased genes within one or more of the first gene ontologies. In embodiments, the first gene set includes about 75-500 increased genes within one or more of the first gene ontologies. In embodiments, the first gene set includes about 80-500 increased genes within one or more of the first gene ontologies. In embodiments, the first gene set includes about 85-500 increased genes within one or more of the first gene ontologies. In embodiments, the first gene set includes about 90-500 increased genes within one or more of the first gene ontologies. In embodiments, the first gene set includes about 95-500 increased genes within one or more of the first gene ontologies. In embodiments, the first gene set includes about 100-500 increased genes within one or more of the first gene ontologies.

In embodiments, the first gene set includes about 105-500 increased genes within one or more of the first gene ontologies. In embodiments, the first gene set includes about 115-500 increased genes within one or more of the first gene ontologies. In embodiments, the first gene set includes about 120-500 increased genes within one or more of the first gene ontologies. In embodiments, the first gene set includes about 125-500 increased genes within one or more of the first gene ontologies. In embodiments, the first gene set includes about 130-500 increased genes within one or more of the first gene ontologies. In embodiments, the first gene set includes about 135-500 increased genes within one or more of the first gene ontologies. In embodiments, the first gene set includes about 140-500 increased genes within one or more of the first gene ontologies. In embodiments, the first gene set includes about 145-500 increased genes within one or more of the first gene ontologies. In embodiments, the first gene set includes about 150-500 increased genes within one or more of the first gene ontologies. In embodiments, the first gene set includes about 155-500 increased genes within one or more of the first gene ontologies. In embodiments, the first gene set includes about 160-500 increased genes within one or more of the first gene ontologies. In embodiments, the first gene set includes about 165-500 increased genes within one or more of the first gene ontologies. In embodiments, the first gene set includes about 170-500 increased genes within one or more of the first gene ontologies. In embodiments, the first gene set includes about 175-500 increased genes within one or more of the first gene ontologies. In embodiments, the first gene set includes about 180-500 increased genes within one or more of the first gene ontologies. In embodiments, the first gene set includes about 185-500 increased genes within one or more of the first gene ontologies. In embodiments, the first gene set includes about 190-500 increased genes within one or more of the first gene ontologies. In embodiments, the first gene set includes about 195-500 increased genes within one or more of the first gene ontologies. In embodiments, the first gene set includes about 200-500 increased genes within one or more of the first gene ontologies.

In embodiments, the first gene set includes about 205-500 increased genes within one or more of the first gene ontologies. In embodiments, the first gene set includes about 215-500 increased genes within one or more of the first gene ontologies. In embodiments, the first gene set includes about 220-500 increased genes within one or more of the first gene ontologies. In embodiments, the first gene set includes about 225-500 increased genes within one or more of the first gene ontologies. In embodiments, the first gene set includes about 230-500 increased genes within one or more of the first gene ontologies. In embodiments, the first gene set includes about 235-500 increased genes within one or more of the first gene ontologies. In embodiments, the first gene set includes about 240-500 increased genes within one or more of the first gene ontologies. In embodiments, the first gene set includes about 245-500 increased genes within one or more of the first gene ontologies. In embodiments, the first gene set includes about 250-500 increased genes within one or more of the first gene ontologies. In embodiments, the first gene set includes about 255-500 increased genes within one or more of the first gene ontologies. In embodiments, the first gene set includes about 260-500 increased genes within one or more of the first gene ontologies. In embodiments, the first gene set includes about 265-500 increased genes within one or more of the first gene ontologies. In embodiments, the first gene set includes about 270-500 increased genes within one or more of the first gene ontologies. In embodiments, the first gene set includes about 275-500 increased genes within one or more of the first gene ontologies. In embodiments, the first gene set includes about 280-500 increased genes within one or more of the first gene ontologies. In embodiments, the first gene set includes about 285-500 increased genes within one or more of the first gene ontologies. In embodiments, the first gene set includes about 290-500 increased genes within one or more of the first gene ontologies. In embodiments, the first gene set includes about 295-500 increased genes within one or more of the first gene ontologies. In embodiments, the first gene set includes about 300-500 increased genes within one or more of the first gene ontologies.

In embodiments, the first gene set includes about 305-500 increased genes within one or more of the first gene ontologies. In embodiments, the first gene set includes about 315-500 increased genes within one or more of the first gene ontologies. In embodiments, the first gene set includes about 320-500 increased genes within one or more of the first gene ontologies. In embodiments, the first gene set includes about 325-500 increased genes within one or more of the first gene ontologies. In embodiments, the first gene set includes about 330-500 increased genes within one or more of the first gene ontologies. In embodiments, the first gene set includes about 335-500 increased genes within one or more of the first gene ontologies. In embodiments, the first gene set includes about 340-500 increased genes within one or more of the first gene ontologies. In embodiments, the first gene set includes about 345-500 increased genes within one or more of the first gene ontologies. In embodiments, the first gene set includes about 350-500 increased genes within one or more of the first gene ontologies. In embodiments, the first gene set includes about 355-500 increased genes within one or more of the first gene ontologies. In embodiments, the first gene set includes about 360-500 increased genes within one or more of the first gene ontologies. In embodiments, the first gene set includes about 365-500 increased genes within one or more of the first gene ontologies. In embodiments, the first gene set includes about 370-500 increased genes within one or more of the first gene ontologies. In embodiments, the first gene set includes about 375-500 increased genes within one or more of the first gene ontologies. In embodiments, the first gene set includes about 380-500 increased genes within one or more of the first gene ontologies. In embodiments, the first gene set includes about 385-500 increased genes within one or more of the first gene ontologies. In embodiments, the first gene set includes about 390-500 increased genes within one or more of the first gene ontologies. In embodiments, the first gene set includes about 395-500 increased genes within one or more of the first gene ontologies. In embodiments, the first gene set includes about 400-500 increased genes within one or more of the first gene ontologies.

In embodiments, the first gene set includes about 405-500 increased genes within one or more of the first gene ontologies. In embodiments, the first gene set includes about 415-500 increased genes within one or more of the first gene ontologies. In embodiments, the first gene set includes about 420-500 increased genes within one or more of the first gene ontologies. In embodiments, the first gene set includes about 425-500 increased genes within one or more of the first gene ontologies. In embodiments, the first gene set includes about 430-500 increased genes within one or more of the first gene ontologies. In embodiments, the first gene set includes about 435-500 increased genes within one or more of the first gene ontologies. In embodiments, the first gene set includes about 440-500 increased genes within one or more of the first gene ontologies. In embodiments, the first gene set includes about 445-500 increased genes within one or more of the first gene ontologies. In embodiments, the first gene set includes about 450-500 increased genes within one or more of the first gene ontologies. In embodiments, the first gene set includes about 455-500 increased genes within one or more of the first gene ontologies. In embodiments, the first gene set includes about 460-500 increased genes within one or more of the first gene ontologies. In embodiments, the first gene set includes about 465-500 increased genes within one or more of the first gene ontologies. In embodiments, the first gene set includes about 470-500 increased genes within one or more of the first gene ontologies. In embodiments, the first gene set includes about 475-500 increased genes within one or more of the first gene ontologies. In embodiments, the first gene set includes about 480-500 increased genes within one or more of the first gene ontologies. In embodiments, the first gene set includes about 485-500 increased genes within one or more of the first gene ontologies. In embodiments, the first gene set includes about 490-500 increased genes within one or more of the first gene ontologies. In embodiments, the first gene set includes about 495-500 increased genes within one or more of the first gene ontologies.

In embodiments, the first gene set includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 increased genes within one or more of the first gene ontologies.

The gene expression profile information for the desirable determined dopaminergic precursor cell may include increased gene expression levels relative to a pluripotent stem cell for a first gene set, wherein the first gene set includes at least one increased gene within one or more first gene ontologies of Table 1. “One or more” as described herein in the context of first gene ontologies refers to at least one, for example, 1, 2, 3, 4, 5, 6, 7, 8, 9, 10, 11, 12, 13, 14, 15, 16, 17, 18, 19, etc. of first gene ontologies. In embodiments, the first gene set includes about 1-500 increased genes within 1-300 of the first gene ontologies. In embodiments, the first gene set includes about 1-500 increased genes within 10-300 of the first gene ontologies. In embodiments, the first gene set includes about 1-500 increased genes within 20-300 of the first gene ontologies. In embodiments, the first gene set includes about 1-500 increased genes within 30-300 of the first gene ontologies. In embodiments, the first gene set includes about 1-500 increased genes within 40-300 of the first gene ontologies. In embodiments, the first gene set includes about 1-500 increased genes within 50-300 of the first gene ontologies. In embodiments, the first gene set includes about 1-500 increased genes within 60-300 of the first gene ontologies. In embodiments, the first gene set includes about 1-500 increased genes within 70-300 of the first gene ontologies. In embodiments, the first gene set includes about 1-500 increased genes within 80-300 of the first gene ontologies. In embodiments, the first gene set includes about 1-500 increased genes within 90-300 of the first gene ontologies. In embodiments, the first gene set includes about 1-500 increased genes within 100-300 of the first gene ontologies. In embodiments, the first gene set includes about 1-500 increased genes within 110-300 of the first gene ontologies. In embodiments, the first gene set includes about 1-500 increased genes within 120-300 of the first gene ontologies. In embodiments, the first gene set includes about 1-500 increased genes within 130-300 of the first gene ontologies. In embodiments, the first gene set includes about 1-500 increased genes within 140-300 of the first gene ontologies. In embodiments, the first gene set includes about 1-500 increased genes within 150-300 of the first gene ontologies. In embodiments, the first gene set includes about 1-500 increased genes within 160-300 of the first gene ontologies. In embodiments, the first gene set includes about 1-500 increased genes within 170-300 of the first gene ontologies. In embodiments, the first gene set includes about 1-500 increased genes within 180-300 of the first gene ontologies. In embodiments, the first gene set includes about 1-500 increased genes within 190-300 of the first gene ontologies. In embodiments, the first gene set includes about 1-500 increased genes within 200-300 of the first gene ontologies. In embodiments, the first gene set includes about 1-500 increased genes within 210-300 of the first gene ontologies. In embodiments, the first gene set includes about 1-500 increased genes within 220-300 of the first gene ontologies. In embodiments, the first gene set includes about 1-500 increased genes within 230-300 of the first gene ontologies. In embodiments, the first gene set includes about 1-500 increased genes within 240-300 of the first gene ontologies. In embodiments, the first gene set includes about 1-500 increased genes within 250-300 of the first gene ontologies. In embodiments, the first gene set includes about 1-500 increased genes within 260-300 of the first gene ontologies. In embodiments, the first gene set includes about 1-500 increased genes within 270-300 of the first gene ontologies. In embodiments, the first gene set includes about 1-500 increased genes within 280-300 of the first gene ontologies. In embodiments, the first gene set includes about 1-500 increased genes within 290-300 of the first gene ontologies.

In embodiments, the first gene set includes about 1-500 increased genes within 1-290 of the first gene ontologies. In embodiments, the first gene set includes about 1-500 increased genes within 1-280 of the first gene ontologies. In embodiments, the first gene set includes about 1-500 increased genes within 1-270 of the first gene ontologies. In embodiments, the first gene set includes about 1-500 increased genes within 1-260 of the first gene ontologies. In embodiments, the first gene set includes about 1-500 increased genes within 1-250 of the first gene ontologies. In embodiments, the first gene set includes about 1-500 increased genes within 1-240 of the first gene ontologies. In embodiments, the first gene set includes about 1-500 increased genes within 1-230 of the first gene ontologies. In embodiments, the first gene set includes about 1-500 increased genes within 1-220 of the first gene ontologies. In embodiments, the first gene set includes about 1-500 increased genes within 1-210 of the first gene ontologies. In embodiments, the first gene set includes about 1-500 increased genes within 1-200 of the first gene ontologies. In embodiments, the first gene set includes about 1-500 increased genes within 1-190 of the first gene ontologies. In embodiments, the first gene set includes about 1-500 increased genes within 1-180 of the first gene ontologies. In embodiments, the first gene set includes about 1-500 increased genes within 1-170 of the first gene ontologies. In embodiments, the first gene set includes about 1-500 increased genes within 1-160 of the first gene ontologies. In embodiments, the first gene set includes about 1-500 increased genes within 1-150 of the first gene ontologies. In embodiments, the first gene set includes about 1-500 increased genes within 1-140 of the first gene ontologies. In embodiments, the first gene set includes about 1-500 increased genes within 1-130 of the first gene ontologies. In embodiments, the first gene set includes about 1-500 increased genes within 1-120 of the first gene ontologies. In embodiments, the first gene set includes about 1-500 increased genes within 1-110 of the first gene ontologies. In embodiments, the first gene set includes about 1-500 increased genes within 1-100 of the first gene ontologies. In embodiments, the first gene set includes about 1-500 increased genes within 1-90 of the first gene ontologies. In embodiments, the first gene set includes about 1-500 increased genes within 1-80 of the first gene ontologies. In embodiments, the first gene set includes about 1-500 increased genes within 1-70 of the first gene ontologies. In embodiments, the first gene set includes about 1-500 increased genes within 1-60 of the first gene ontologies. In embodiments, the first gene set includes about 1-500 increased genes within 1-50 of the first gene ontologies. In embodiments, the first gene set includes about 1-500 increased genes within 1-40 of the first gene ontologies. In embodiments, the first gene set includes about 1-500 increased genes within 1-30 of the first gene ontologies. In embodiments, the first gene set includes about 1-500 increased genes within 1-20 of the first gene ontologies. In embodiments, the first gene set includes about 1-500 increased genes within 1-10 of the first gene ontologies. In embodiments, the first gene set includes about 1-500 increased genes within 1-5 of the first gene ontologies.

In embodiments, the first gene set includes at least one increased gene within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, or 208 first gene ontologies of Table 1.

In embodiments, the first gene set includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307,308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 increased genes within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147,148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207 or 208 first gene ontologies of Table 1.

In embodiments, the first gene ontologies are any one of the gene ontologies listed in Table 1. In embodiments, the first gene ontologies are any one of GO:0007399, GO:0120025, GO:0042995, GO:0032502, GO:0044767, GO:0048856, GO:0048731, GO:0022008, GO:0048699, GO:0007275, GO:0030030, GO:0032501, GO:0044707, GO:0050874, GO:0048468, GO:0120036, GO:0120038, GO:0044463, GO:0097458, GO:0045202, GO:0030182, GO:0030154, GO:0048869, GO:0051960, GO:0007156, GO:0005929, GO:0072372, GO:0035082, GO:0035083, GO:0035084, GO:0060284, GO:0050767, GO:0001578, GO:0016339, GO:0043005, GO:0044456, GO:0098742, GO:0045664, GO:0006928, GO:0099699, GO:0048666, GO:0003341, GO:0036142, GO:0005509, GO:0097060, GO:0031514, GO:0009434, GO:0031512, GO:0007155, GO:0098602, GO:0010975, GO:0098794, GO:0022610, GO:0030424, GO:0099240, GO:0032989, GO:0120035, GO:0000902, GO:0007148, GO:0045790, GO:0045791, GO:0048812, GO:0036477, GO:0031344, GO:0120039, GO:0061564, GO:0048858, GO:0099055, GO:0009653, GO:0098609, GO:0016337, GO:0031175, GO:0005930, GO:0035085, GO:0035086, GO:0010720, GO:0007416, GO:0097014, GO:0032990, GO:0098936, GO:0043025, GO:0050768, GO:0051962, GO:0050808, GO:0007409, GO:0007410, GO:2000026, GO:0045597, GO:0044441, GO:0044442, GO:0007417, GO:0048667, GO:0010721, GO:0044459, GO:0060322, GO:0045211, GO:0045666, GO:0032838, GO:0099056, GO:0051961, GO:0044297, GO:0007018, GO:0050769, GO:0040011, GO:0050793, GO:0051094, GO:0005874, GO:0000904, GO:0010976, GO:0045595, GO:0050770, GO:0099536, GO:0098889, GO:0051239, GO:0007420, GO:0099537, GO:0031346, GO:0007268, GO:0098916, GO:0097485, GO:0044782, GO:0031226, GO:0060285, GO:0071974, GO:0010769, GO:0001539, GO:0050804, GO:0099177, GO:0005887, GO:0098984, GO:0045665, GO:0050919, GO:0007411, GO:0008040, GO:0030425, GO:0061387, GO:0097447, GO:0050803, GO:0042734, GO:0042391, GO:0001764, GO:0032279, GO:0010770, GO:0021953, GO:0099572, GO:0098590, GO:0044447, GO:0098978, GO:0014069, GO:0097481, GO:0097483, GO:0033267, GO:0010977, GO:0007017, GO:0150034, GO:0034702, GO:0034703, GO:0050807, GO:0060271, GO:0042384, GO:0051240, GO:0050772, GO:0120031, GO:0007626, GO:0008092, GO:0005886, GO:0005904, GO:0007610, GO:0044708, GO:0098793, GO:0022604, GO:0007267, GO:0071944, GO:0099060, GO:0022836, GO:0030031, GO:0042220, GO:0019226, GO:0030516, GO:0035637, GO:0045596, GO:0021954, GO:0022832, GO:0005244, GO:1902495, GO:0050771, GO:0048513, GO:0022839, GO:0098948, GO:0001508, GO:0099568, GO:0008484, GO:0051966, GO:0003358, GO:0033602, GO:0005261, GO:0015281, GO:0015338, GO:0022603, GO:1990351, GO:0097729, GO:0015631, GO:0051270, GO:0005216, GO:0016043, GO:0044235, GO:0071842, GO:0031345, GO:0005856, GO:0022838, GO:0099061, GO:0098982, GO:0051674, GO:0048870, GO:0060294, GO:0072359, GO:0099634, GO:0015630, GO:0036126, GO:1990939, GO:0072347, GO:0015267, GO:0015249, GO:0015268, GO:0022803, GO:0022814, GO:0008045, GO:0098797, GO:0060160, GO:0099146, GO:0010771, GO:0000226, GO:0045503, GO:0005578, GO:0030334, GO:0044304, GO:0010463, GO:0010646, GO:0008574, GO:0043279 or any combination thereof.

In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies selected from the group consisting of: GO0005509, GO0016339, GO0007416 and GO0048731. In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies of: GO0005509, GO0016339, GO0007416 or GO0048731. In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies selected from the group consisting of: GO0048699, GO0050767, GO0060160, GO0097458, GO0010975, GO0022008 and any combination thereof. In embodiments, the gene expression profile information for the desirable determined dopaminergic precursor cell includes increased gene expression levels relative to a pluripotent stem cell for a first gene set, wherein the first gene set includes at least one increased gene within one or more first gene ontologies of: GO0048699, GO0050767, GO0060160, GO0097458, GO0010975, GO0022008 or any combination thereof.

In embodiments, the first gene set includes at least one (e.g., 1, 2, 3, 4, 5, 6 etc.) increased gene of Table 2, Table 3, Table 4, Table 5 Table 6 or Table 7 or any combination thereof.

In embodiments, the first gene set includes at least one (e.g., 1, 2, 3, 4, 5, 6 etc.) increased gene of Table 2. In embodiments, the at least one (e.g., 1, 2, 3, 4, 5, 6 etc.) increased gene is GPM6A, DRD2, BMP7, EFNB3, SEMA3C, FSCN2, LGI1, SRCIN1, WNT4, SLIT2, NRG1, TTBK1, RNF165, CDH2, ELAVL4, ONECUT2, KREMEN1, SCRT1, KIAA1024, DSCAM, MAP2, FAT4, PAK3, NGF, SEMA6D, STMN2, ZFHX3, LRP2, APOA1, CAMK2B, MDGA1, ISLR2, SNAP25, NEUROD4, PHOX2B, DCX, MAGI2, PIK3R1, NCAM1, NTRK3, PITX3, MYT1L, AVIL, CDK5R2, INSM1, SOX21, IL6ST, KIFSC, SYNJ1, KALRN, GFRA1, TCTN1, CELSR1, IRX5, PMP22, RUNX1, DPYSL4, NRCAM, ZNF521, MDGA2, PROX1, ZNF536, MAP1A, NEGR1, PLXNA4, EPB41L3, GAP43, EPHA7, DLL3, VSTM2L, ID4, NRN1, SPOCK1, DUSP10, COL3A1, CX3CL1, SLIT3, MAPK8IP2, FAIM2, TCF12, BMP6, NRBP2, NCAM2, HIPK2, CDH11, ADGRL3, ZNF804A, ULK2, CCKAR, SARM1, PLXNA3, ENC1, ASCL1, UNCX, MEIS1, ARX, SRRM4, TRIM67, ALCAM, NTN1, ZNF365, GFI1, ADCYAP1, CNR1, ANKRD1, ALK, STMN4, MAPT, RUFY3, PLXNA2, PLXNC1, MAP1B, DPYSL5, PTPRO, FZD1 or DLX5.

In embodiments, the at least one (e.g., 1, 2, 3, 4, 5, 6 etc.) increased gene is selected from the group consisting of GPM6A, DRD2, BMP7, EFNB3, SEMA3C, FSCN2, LGI1, SRCIN1, WNT4, SLIT2, NRG1, TTBK1, RNF165, CDH2, ELAVL4, ONECUT2, KREMEN1, SCRT1, KIAA1024, DSCAM, MAP2, FAT4, PAK3, NGF, SEMA6D, STMN2, ZFHX3, LRP2, APOA1, CAMK2B, MDGA1, ISLR2, SNAP25, NEUROD4, PHOX2B, DCX, MAGI2, PIK3R1, NCAM1, NTRK3, PITX3, MYT1L, AVIL, CDK5R2, INSM1, SOX21, IL6ST, KIF5C, SYNJ1, KALRN, GFRA1, TCTN1, CELSR1, IRX5, PMP22, RUNX1, DPYSL4, NRCAM, ZNF521, MDGA2, PROX1, ZNF536, MAP1A, NEGR1, PLXNA4, EPB41L3, GAP43, EPHA7, DLL3, VSTM2L, ID4, NRN1, SPOCK1, DUSP10, COL3A1, CX3CL1, SLIT3, MAPK8IP2, FAIM2, TCF12, BMP6, NRBP2, NCAM2, HIPK2, CDH11, ADGRL3, ZNF804A, ULK2, CCKAR, SARM1, PLXNA3, ENC1, ASCL1, UNCX, MEIS1, ARX, SRRM4, TRIM67, ALCAM, NTN1, ZNF365, GFI1, ADCYAP1, CNR1, ANKRD1, ALK, STMN4, MAPT, RUFY3, PLXNA2, PLXNC1, MAP1B, DPYSL5, PTPRO, FZD1 and DLX5.

In embodiments, the first gene set includes at least one (e.g., 1, 2, 3, 4, 5, 6 etc.) increased gene of Table 3. In embodiments, the at least one (e.g., 1, 2, 3, 4, 5, 6 etc.) increased gene is DRD2, BMP7, EFNB3, SEMA3C, SRCIN1, SLIT2, NRG1, TTBK1, CDH2, KREMEN1, SCRT1, KIAA1024, DSCAM, MAP2, PAK3, NGF, SEMA6D, STMN2, ZFHX3, LRP2, CAMK2B, ISLR2, SNAP25, PHOX2B, MAGI2, NTRK3, PITX3, AVIL, IL6ST, SYNJ1, KALRN, PMP22, NRCAM, PROX1, ZNF536, NEGR1, PLXNA4, EPHA7, DLL3, ID4, SPOCK1, DUSP10, COL3A1, CX3CL1, TCF12, BMP6, ZNF804A, ULK2, SARM1, PLXNA3, ENC1, ASCL1, MEIS1, TRIM67, NTN1, ZNF365, GFI1, ADCYAP1, CNR1, ANKRD1, ALK, MAPT, RUFY3, PLXNA2, PLXNC1, MAP1B, PTPRO or FZD1.

In embodiments, the at least one (e.g., 1, 2, 3, 4, 5, 6 etc.) increased gene is selected from the group consisting of DRD2, BMP7, EFNB3, SEMA3C, SRCIN1, SLIT2, NRG1, TTBK1, CDH2, KREMEN1, SCRT1, KIAA1024, DSCAM, MAP2, PAK3, NGF, SEMA6D, STMN2, ZFHX3, LRP2, CAMK2B, ISLR2, SNAP25, PHOX2B, MAGI2, NTRK3, PITX3, AVIL, IL6ST, SYNJ1, KALRN, PMP22, NRCAM, PROX1, ZNF536, NEGR1, PLXNA4, EPHA7, DLL3, ID4, SPOCK1, DUSP10, COL3A1, CX3CL1, TCF12, BMP6, ZNF804A, ULK2, SARM1, PLXNA3, ENC1, ASCL1, MEIS1, TRIM67, NTN1, ZNF365, GFI1, ADCYAP1, CNR1, ANKRD1, ALK, MAPT, RUFY3, PLXNA2, PLXNC1, MAP1B, PTPRO and FZD1.

In embodiments, the first gene set includes at least one (e.g., 1, 2, 3, 4, 5, 6 etc.) increased gene of Table 4. In embodiments, the at least one (e.g., 1, 2, 3, 4, 5, 6 etc.) increased gene is DRD2, RGS4, or PALM.

In embodiments, the at least one (e.g., 1, 2, 3, 4, 5, 6 etc.) increased gene is selected from the group consisting of DRD2, RGS4, and PALM.

In embodiments, the first gene set includes at least one (e.g., 1, 2, 3, 4, 5, 6 etc.) increased gene of Table 5. In embodiments, the at least one (e.g., 1, 2, 3, 4, 5, 6 etc.) increased gene is GPM6A, KIFAP3, DRD2, EFNB3, FSCN2, SLC8A1, SCGN, SRCIN1, PACRG, TRIM9, NRG1, TTBK1, HTR2A, SLC18A1, CERKL, CDH2, PALMD, KREMEN1, TANC2, MAPK10, SCN3A, LRRC4, DSCAM, TGFB3, MAP2, ELFN1, PAK3, NGF, CPEB2, DDN, STMN2, LRP2, CAMK2B, SVOP, SRR, SNAP25, PPFIA2, KCNA2, SYT5, BAIAP3, CADM2, CHRM2, DCX, MAGI2, KLHL1, NTRK3, PITX3, P2RX3, ADGRA1, AVIL, CADM3, CDK5R2, IL6ST, KIFSC, SYNJ1, TSPOAP1, DRP2, TMPRSS3, SYBU, HMP19, SNAP91, SCN11A, PALM, SLC1A4, NRCAM, CACNG4, CNIH2, DGKI, CLSTN2, MAP1A, GLRA2, CUBN, SCN7A, EPB41L3, BSN, GAP43, EPHA7, VSTM2L, SPOCK1, CX3CL1, MAPK8IP2, CAMK2N1, PDE1C, NCAM2, SLC17A6, SLC18A3, KCNC1, ADGRL3, ZNF804A, SARM1, GRIK4, ENC1, ASCL1, DMTN, KNCN, TMEM163, CLDN5, KCND3, PCDHB13, GABRR2, ALCAM, SV2B, KCTD16, ADCYAP1, APBA1, CNR1, STMN4, CADPS, MAPT, RUFY3, TP63, NRSN1, MAP1B, PCSK2, DPYSL5, GRM3, SLC6A1, ABAT, CACNA1C, CACNG2, PTPRO, CHRNA5, or CDH10.

In embodiments, the first gene set includes at least one (e.g., 1, 2, 3, 4, 5, 6 etc.) increased gene of Table 5. In embodiments, the at least one (e.g., 1, 2, 3, 4, 5, 6 etc.) increased gene is selected from the group consisting of GPM6A, KIFAP3, DRD2, EFNB3, FSCN2, SLC8A1, SCGN, SRCIN1, PACRG, TRIM9, NRG1, TTBK1, HTR2A, SLC18A1, CERKL, CDH2, PALMD, KREMEN1, TANC2, MAPK10, SCN3A, LRRC4, DSCAM, TGFB3, MAP2, ELFN1, PAK3, NGF, CPEB2, DDN, STMN2, LRP2, CAMK2B, SVOP, SRR, SNAP25, PPFIA2, KCNA2, SYT5, BAIAP3, CADM2, CHRM2, DCX, MAGI2, KLHL1, NTRK3, PITX3, P2RX3, ADGRA1, AVIL, CADM3, CDK5R2, IL6ST, KIFSC, SYNJ1, TSPOAP1, DRP2, TMPRSS3, SYBU, HMP19, SNAP91, SCN11A, PALM, SLC1A4, NRCAM, CACNG4, CNIH2, DGKI, CLSTN2, MAP1A, GLRA2, CUBN, SCN7A, EPB41L3, BSN, GAP43, EPHA7, VSTM2L, SPOCK1, CX3CL1, MAPK8IP2, CAMK2N1, PDE1C, NCAM2, SLC17A6, SLC18A3, KCNC1, ADGRL3, ZNF804A, SARM1, GRIK4, ENC1, ASCL1, DMTN, KNCN, TMEM163, CLDN5, KCND3, PCDHB13, GABRR2, ALCAM, SV2B, KCTD16, ADCYAP1, APBA1, CNR1, STMN4, CADPS, MAPT, RUFY3, TP63, NRSN1, MAP1B, PCSK2, DPYSL5, GRM3, SLC6A1, ABAT, CACNA1C, CACNG2, PTPRO, CHRNA5, and CDH10.

In embodiments, the first gene set includes at least one (e.g., 1, 2, 3, 4, 5, 6 etc.) increased gene of Table 6. In embodiments, the at least one (e.g., 1, 2, 3, 4, 5, 6 etc.) increased gene is EFNB3, SEMA3C, SRCIN1, SLIT2, CDH2, KREMEN1, KIAA1024, DSCAM, MAP2, PAK3, NGF, SEMA6D, STMN2, CAMK2B, ISLR2, SNAP25, MAGI2, NTRK3, AVIL, KALRN, PMP22, NRCAM, NEGR1, PLXNA4, EPHA7, SPOCK1, CX3CL1, ZNF804A, ULK2, SARM1, PLXNA3, ENC1, TRIM67, NTN1, ZNF365, GFI1, ADCYAP1, CNR1, ANKRD1, MAPT, RUFY3, PLXNA2, PLXNC1, MAP1B, PTPRO or FZD1.

In embodiments, the at least one (e.g., 1, 2, 3, 4, 5, 6 etc.) increased gene is selected from the group consisting of EFNB3, SEMA3C, SRCIN1, SLIT2, CDH2, KREMEN1, KIAA1024, DSCAM, MAP2, PAK3, NGF, SEMA6D, STMN2, CAMK2B, ISLR2, SNAP25, MAGI2, NTRK3, AVIL, KALRN, PMP22, NRCAM, NEGR1, PLXNA4, EPHA7, SPOCK1, CX3CL1, ZNF804A, ULK2, SARM1, PLXNA3, ENC1, TRIM67, NTN1, ZNF365, GFI1, ADCYAP1, CNR1, ANKRD1, MAPT, RUFY3, PLXNA2, PLXNC1, MAP1B, PTPRO and FZD1.

In embodiments, the first gene set includes at least one (e.g., 1, 2, 3, 4, 5, 6 etc.) increased gene of Table 7. In embodiments, the at least one (e.g., 1, 2, 3, 4, 5, 6 etc.) increased gene is GPM6A, DRD2, BMP7, EFNB3, SEMA3C, FSCN2, LGI1, SRCIN1, WNT4, SLIT2, NAV3, NRG1, TTBK1, RNF165, PRDM16, CDH2, ELAVL4, ONECUT2, KREMEN1, SCRT1, KIAA1024, DSCAM, MAP2, PRDM8, FAT4, PAK3, NGF, SEMA6D, STMN2, ZFHX3, LRP2, APOA1, CAMK2B, MDGA1, ISLR2, SNAP25, NEUROD4, PHOX2B, DCX, MAGI2, PIK3R1, NCAM1, NTRK3, PITX3, MYT1L, AVIL, CDK5R2, INSM1, SOX21, IL6ST, KIFSC, SYNJ1, KALRN, GFRA1, TCTN1, CELSR1, IRX5, PMP22, SOX6, RUNX1, DPYSL4, NRCAM, ZNF521, MDGA2, PROX1, FGF5, ZNF536, MAP1A, DCHS1, NEGR1, PLXNA4, EPB41L3, GAP43, EPHA7, DLL3, VSTM2L, ID4, NRN1, SPOCK1, DUSP10, COL3A1, CX3CL1, SLIT3, MAPK8IP2, FAIM2, TCF12, BMP6, NRBP2, NCAM2, HIPK2, CDH11, ADGRL3, ZNF804A, ULK2, CCKAR, SARM1, PLXNA3, ENC1, ASCL1, UNCX, MEIS1, ARX, SRRM4, TRIM67, ALCAM, NTN1, ZNF365, GFI1, ADCYAP1, CNR1, ANKRD1, ALK, STMN4, MAPT, RUFY3, PLXNA2, PLXNC1, MAP1B, DPYSL5, PTPRO, FZD1 or DLX5.

In embodiments, the at least one (e.g., 1, 2, 3, 4, 5, 6 etc.) increased gene is selected from the group consisting of GPM6A, DRD2, BMP7, EFNB3, SEMA3C, FSCN2, LGI1, SRCIN1, WNT4, SLIT2, NAV3, NRG1, TTBK1, RNF165, PRDM16, CDH2, ELAVL4, ONECUT2, KREMEN1, SCRT1, KIAA1024, DSCAM, MAP2, PRDM8, FAT4, PAK3, NGF, SEMA6D, STMN2, ZFHX3, LRP2, APOA1, CAMK2B, MDGA1, ISLR2, SNAP25, NEUROD4, PHOX2B, DCX, MAGI2, PIK3R1, NCAM1, NTRK3, PITX3, MYT1L, AVIL, CDK5R2, INSM1, SOX21, IL6ST, KIFSC, SYNJ1, KALRN, GFRA1, TCTN1, CELSR1, IRX5, PMP22, SOX6, RUNX1, DPYSL4, NRCAM, ZNF521, MDGA2, PROX1, FGF5, ZNF536, MAP1A, DCHS1, NEGR1, PLXNA4, EPB41L3, GAP43, EPHA7, DLL3, VSTM2L, ID4, NRN1, SPOCK1, DUSP10, COL3A1, CX3CL1, SLIT3, MAPK8IP2, FAIM2, TCF12, BMP6, NRBP2, NCAM2, HIPK2, CDH11, ADGRL3, ZNF804A, ULK2, CCKAR, SARM1, PLXNA3, ENC1, ASCL1, UNCX, MEIS1, ARX, SRRM4, TRIM67, ALCAM, NTN1, ZNF365, GFI1, ADCYAP1, CNR1, ANKRD1, ALK, STMN4, MAPT, RUFY3, PLXNA2, PLXNC1, MAP1B, DPYSL5, PTPRO, FZD1 and DLX5.

In embodiments, the at least one increased gene is selected from the group consisting of: CAPN14, FAT3, FAT4, PCDHGC4, SLC8A1, SLIT2, CEMIP2, CDHR3, CDH2, DRD2, EPHB2, MAGI2, PCDHB11, PCDHB13, PCDHB14, PCDHB16, PCDHB2, ADGRG6, ELF5, EPHA7, FOXP1, GDF7, HOXA1, MINAR1, MSX1, NRBP2, NRIP1, PITX3, POU6F2, PTPRO, SLC35D1, TCF12, ZFHX3 and ZNF703. In embodiments, the at least one increased gene is CAPN14, FAT3, FAT4, PCDHGC4, SLC8A1, SLIT2, CEMIP2, CDHR3, CDH2, DRD2, EPHB2, MAGI2, PCDHB11, PCDHB13, PCDHB14, PCDHB16, PCDHB2, ADGRG6, ELF5, EPHA7, FOXP1, GDF7, HOXA1, MINAR1, MSX1, NRBP2, NRIP1, PITX3, POU6F2, PTPRO, SLC35D1, TCF12, ZFHX3 or ZNF703.

In embodiments, the increased expression levels are at least 4 times higher relative to a pluripotent stem cell. In embodiments, the increased expression levels are about 4 times higher relative to a pluripotent stem cell. In embodiments, the increased expression levels are at least 5 times higher relative to a pluripotent stem cell. In embodiments, the increased expression levels are about 5 times higher relative to a pluripotent stem cell. In embodiments, the increased expression levels are at least 6 times higher relative to a pluripotent stem cell. In embodiments, the increased expression levels are about 6 times higher relative to a pluripotent stem cell. In embodiments, the increased expression levels are at least 7 times higher relative to a pluripotent stem cell. In embodiments, the increased expression levels are about 7 times higher relative to a pluripotent stem cell. In embodiments, the increased expression levels are at least 8 times higher relative to a pluripotent stem cell. In embodiments, the increased expression levels are about 8 times higher relative to a pluripotent stem cell. In embodiments, the increased expression levels are at least 9 times higher relative to a pluripotent stem cell. In embodiments, the increased expression levels are about 9 times higher relative to a pluripotent stem cell. In embodiments, the increased expression levels are at least 10 times higher relative to a pluripotent stem cell. In embodiments, the increased expression levels are about 10 times higher relative to a pluripotent stem cell.

In embodiments, the increased expression levels are at least 11 times higher relative to a pluripotent stem cell. In embodiments, the increased expression levels are about 11 times higher relative to a pluripotent stem cell. In embodiments, the increased expression levels are at least 12 times higher relative to a pluripotent stem cell. In embodiments, the increased expression levels are about 12 times higher relative to a pluripotent stem cell. In embodiments, the increased expression levels are at least 13 times higher relative to a pluripotent stem cell. In embodiments, the increased expression levels are about 13 times higher relative to a pluripotent stem cell. In embodiments, the increased expression levels are at least 14 times higher relative to a pluripotent stem cell. In embodiments, the increased expression levels are about 14 times higher relative to a pluripotent stem cell. In embodiments, the increased expression levels are at least 15 times higher relative to a pluripotent stem cell. In embodiments, the increased expression levels are about 15 times higher relative to a pluripotent stem cell. In embodiments, the increased expression levels are at least 16 times higher relative to a pluripotent stem cell. In embodiments, the increased expression levels are about 16 times higher relative to a pluripotent stem cell. In embodiments, the increased expression levels are at least 17 times higher relative to a pluripotent stem cell. In embodiments, the increased expression levels are about 17 times higher relative to a pluripotent stem cell. In embodiments, the increased expression levels are at least 18 times higher relative to a pluripotent stem cell. In embodiments, the increased expression levels are about 18 times higher relative to a pluripotent stem cell. In embodiments, the increased expression levels are at least 19 times higher relative to a pluripotent stem cell. In embodiments, the increased expression levels are about 19 times higher relative to a pluripotent stem cell. In embodiments, the increased expression levels are at least 20 times higher relative to a pluripotent stem cell. In embodiments, the increased expression levels are about 20 times higher relative to a pluripotent stem cell.

In embodiments, the increased expression levels are about 4-100 times higher relative to a pluripotent stem cell. In embodiments, the increased expression levels are 4-100 times higher relative to a pluripotent stem cell. In embodiments, the increased expression levels are about 6-100 times higher relative to a pluripotent stem cell. In embodiments, the increased expression levels are 6-100 times higher relative to a pluripotent stem cell. In embodiments, the increased expression levels are about 8-100 times higher relative to a pluripotent stem cell. In embodiments, the increased expression levels are 8-100 times higher relative to a pluripotent stem cell. In embodiments, the increased expression levels are about 10-100 times higher relative to a pluripotent stem cell. In embodiments, the increased expression levels are 10-100 times higher relative to a pluripotent stem cell. In embodiments, the increased expression levels are about 20-100 times higher relative to a pluripotent stem cell. In embodiments, the increased expression levels are 20-100 times higher relative to a pluripotent stem cell. In embodiments, the increased expression levels are about 30-100 times higher relative to a pluripotent stem cell. In embodiments, the increased expression levels are 30-100 times higher relative to a pluripotent stem cell. In embodiments, the increased expression levels are about 40-100 times higher relative to a pluripotent stem cell. In embodiments, the increased expression levels are 40-100 times higher relative to a pluripotent stem cell. In embodiments, the increased expression levels are about 50-100 times higher relative to a pluripotent stem cell. In embodiments, the increased expression levels are 50-100 times higher relative to a pluripotent stem cell. In embodiments, the increased expression levels are about 60-100 times higher relative to a pluripotent stem cell. In embodiments, the increased expression levels are 60-100 times higher relative to a pluripotent stem cell. In embodiments, the increased expression levels are about 70-100 times higher relative to a pluripotent stem cell. In embodiments, the increased expression levels are 70-100 times higher relative to a pluripotent stem cell. In embodiments, the increased expression levels are about 80-100 times higher relative to a pluripotent stem cell. In embodiments, the increased expression levels are 80-100 times higher relative to a pluripotent stem cell. In embodiments, the increased expression levels are about 90-100 times higher relative to a pluripotent stem cell. In embodiments, the increased expression levels are 90-100 times higher relative to a pluripotent stem cell.

In embodiments, the increased expression levels are about 4-90 times higher relative to a pluripotent stem cell. In embodiments, the increased expression levels are 4-90 times higher relative to a pluripotent stem cell. In embodiments, the increased expression levels are about 4-80 times higher relative to a pluripotent stem cell. In embodiments, the increased expression levels are 4-80 times higher relative to a pluripotent stem cell. In embodiments, the increased expression levels are about 4-70 times higher relative to a pluripotent stem cell. In embodiments, the increased expression levels are 4-70 times higher relative to a pluripotent stem cell. In embodiments, the increased expression levels are about 4-60 times higher relative to a pluripotent stem cell. In embodiments, the increased expression levels are 4-60 times higher relative to a pluripotent stem cell. In embodiments, the increased expression levels are about 4-50 times higher relative to a pluripotent stem cell. In embodiments, the increased expression levels are 4-50 times higher relative to a pluripotent stem cell. In embodiments, the increased expression levels are about 4-40 times higher relative to a pluripotent stem cell. In embodiments, the increased expression levels are 4-40 times higher relative to a pluripotent stem cell. In embodiments, the increased expression levels are about 4-30 times higher relative to a pluripotent stem cell. In embodiments, the increased expression levels are 4-30 times higher relative to a pluripotent stem cell. In embodiments, the increased expression levels are about 4-20 times higher relative to a pluripotent stem cell. In embodiments, the increased expression levels are 4-20 times higher relative to a pluripotent stem cell. In embodiments, the increased expression levels are about 4-10 times higher relative to a pluripotent stem cell. In embodiments, the increased expression levels are 4-10 times higher relative to a pluripotent stem cell. In embodiments, the increased expression levels are about 4-8 times higher relative to a pluripotent stem cell. In embodiments, the increased expression levels are 4-8 times higher relative to a pluripotent stem cell. In embodiments, the increased expression levels are about 4-6 times higher relative to a pluripotent stem cell. In embodiments, the increased expression levels are 4-6 times higher relative to a pluripotent stem cell.

In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies of Table 8.

In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies selected from the group consisting of gene ontologies of Table 8.

In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies of GO:0044459, GO:0071944, GO:0005886, GO:0005904, GO:0031226, GO:0005887, GO:0042127, GO:0005576, GO:0044421, GO:0070887, GO:0034097, GO:0050896, GO:0051869, GO:0071345, GO:0048856, GO:0010033, GO:0044425, GO:0007166, GO:0032501, GO:0044707, GO:0050874, GO:0023052, GO:0023046, GO:0044700, GO:0031982, GO:0031988, GO:0032502, GO:0044767, GO:0007154, GO:0071310, GO:0005615, GO:0042221, GO:0031224, GO:0051049, GO:0019221, GO:0048583, GO:0008284, GO:0007275, GO:0023051, GO:0010646, GO:0048584, GO:0051239, GO:0032879, GO:0006954, GO:0007165, GO:0023033, GO:0043230, GO:0098771, GO:0055065, GO:0016021, GO:1903561, GO:0009966, GO:0035466, GO:0050801, GO:0010647, GO:0006811, GO:0065008, GO:0051240, GO:0098590, GO:0055082, GO:0055080, GO:0023056, GO:0006875, GO:0070062, GO:0051716, GO:0048878, GO:0043269, GO:0065009, GO:0051050, GO:0050865, GO:0098857, GO:0006873, GO:0048518, GO:0043119, GO:0030003, GO:0048731, GO:0042592, GO:0045121, GO:0006952, GO:0002217, GO:0042829, GO:0048522, GO:0051242, GO:0046903, GO:0005102, GO:0030154, GO:0019725, GO:0001775, GO:0009967, GO:0035468, GO:0002376, GO:0072503, GO:0045321, GO:0050863, GO:0050878, GO:0048869, GO:0002703, GO:0050670, GO:0022407, GO:0032944, GO:0016020, GO:1902533, GO:0010740, GO:0043270, GO:0045785, GO:0072507, GO:0009888, GO:0022409, GO:0042493, GO:0017035, GO:0002682, GO:0006874, GO:0032101, GO:0070663, GO:0007204, GO:1902531, GO:0010627, GO:1903039, GO:1903037, GO:0002694, GO:0031012, GO:0009605, GO:0044281, GO:2000021, GO:0055074, GO:0035296, GO:0097746, GO:0042312, GO:0044093, GO:0002685, GO:0098589, GO:0051480, GO:0003013, GO:0008015, GO:0070261, GO:1901700, GO:0007187, GO:0030155, GO:0003006, GO:0034220, GO:0050870, GO:0009611, GO:0002245, GO:0008217, GO:1903524, GO:0042129, GO:0033993, GO:0050880, GO:0007188, GO:0051704, GO:0051706, GO:0035150, GO:0030198, GO:0032103, GO:0043062, GO:0050867, GO:0040017, GO:0002687, GO:0022857, GO:0005386, GO:0015563, GO:0015646, GO:0022891, GO:0022892, GO:0048608, GO:0015267, GO:0015249, GO:0015268, GO:0002274, GO:0001890, GO:0048513, GO:0022803, GO:0022814, GO:0002684, GO:0050776, GO:0002819, GO:0045937, GO:0010562, GO:0002366, GO:0061458, GO:0051094, GO:0034762, GO:2000147, GO:0030141, GO:0002263, GO:0006955, GO:0015075, GO:0099503, GO:0000003, GO:0019952, GO:0050876, GO:0098772, GO:0002252, GO:0009653, GO:0050900, GO:1901701, GO:0042802, GO:0043085, GO:0048554, GO:0030335, GO:0005215, GO:0005478, GO:0022414, GO:0044702, GO:0051241, GO:0002696, GO:0046873, GO:0042060, GO:0003018, GO:0032940, GO:0031410, GO:0016023, GO:0002822, GO:0046394, GO:0051272, GO:0097708, GO:0009986, GO:0009928, GO:0009929, GO:0016053, GO:0051928, GO:0042327, GO:0031225, GO:0010469, GO:0009987, GO:0008151, GO:0044763, GO:0050875, GO:0006950, GO:0043207, GO:0002886, GO:0051249, GO:0098655, GO:0005575, GO:0008372, GO:0002697, GO:0019935, GO:0007267, GO:0032496, GO:0070160, GO:0005216, GO:0034765, GO:0006820, GO:0006822, GO:0005911, GO:0019933, GO:0004252, GO:0048545, GO:0051924, GO:0006812, GO:0006819, GO:0015674, GO:0019932, GO:0051707, GO:0009613, GO:0042828, GO:0001934, GO:0022838, GO:1902105, GO:0006636, GO:0071624, GO:0055085, GO:0010959, GO:0005923, GO:0030001, GO:0002237, GO:0009607, GO:0002699, GO:0005261, GO:0015281, GO:0015338, GO:1903522, GO:0043408, GO:0008324, GO:0015711, GO:0071622, GO:0070665, GO:0002683, GO:0010543, GO:0050730, GO:0007189, GO:0010579, GO:0010580, GO:0016338, GO:0050671, GO:0015318, GO:0050777, GO:0050793, GO:0030054, GO:0022610, GO:0032946, GO:0043300, GO:0042102, GO:0001817, GO:0002275, GO:0032844, GO:0060429, GO:0001653, GO:0031347, GO:0048646, GO:0042981, GO:0051345, GO:0002690, GO:0043302, GO:0098660, GO:0009719, GO:0048018, GO:0071884, GO:0009116, GO:0043168, GO:0002444, GO:0043296, GO:0065007, GO:0098662, GO:0043299, GO:0030193, GO:0042119, GO:0050921, GO:0002688, GO:0043410, GO:0022836, GO:0090022, GO:0002888, GO:0002821, GO:1900046, GO:0042509, GO:0042510, GO:0042513, GO:0042516, GO:0042519, GO:0042522, GO:0042525, GO:0042528, GO:0035295, GO:0043235, GO:0022839, GO:0090023, GO:0043065, GO:0046718, GO:0019063, GO:0043067, GO:0043070, GO:0030545, GO:0001816, GO:0003382, GO:0044409, GO:0051806, GO:0030260, GO:0051828, GO:0036230, GO:0010941, GO:0009725, GO:0002476, GO:0002526, GO:0051384, GO:0050790, GO:0048552, GO:0051247, GO:0008285, GO:0097755, GO:0045909, GO:0031960, GO:0070374, GO:0002824, GO:0030728, GO:0007155, GO:0098602, GO:0035556, GO:0007242, GO:0007243, GO:0023013, GO:0023034, GO:0010942, GO:0070372, GO:0051046, GO:0043068, GO:0043071, GO:1902107, GO:0002283, GO:0005509, GO:0050818, GO:0051336, GO:0009119, GO:0003073, GO:0036018, GO:0046635, GO:2000026, GO:0006082, GO:0001819, GO:0004175, GO:0016809, GO:0050764, GO:0043436, GO:0005201, GO:0097028, GO:0008528, GO:0045055, GO:0016477, GO:0030168, GO:0035239, GO:0070820, GO:0031349, GO:0001932, GO:0098797, GO:0045137, GO:0043312, GO:0002446, GO:0052547, GO:0048585, GO:0009070, GO:0009113, GO:0034764, GO:0022600, GO:0016323, GO:0045597, GO:0042803, GO:0016324, GO:0045177, GO:0008406, GO:0006887, GO:0016194, GO:0016195, GO:0008236, GO:0072358, GO:0001944, GO:0002521, GO:1902624, GO:0044283, GO:0048519, GO:0043118, GO:0045684, GO:0006690, GO:0010522, GO:0022890, GO:0015082, GO:0019752, GO:0071396, GO:0001525, GO:0050731, GO:0036017, GO:0042609, GO:0050817, GO:0070252, GO:0060670, GO:0019369, GO:0019229, GO:0009164, GO:0017171, GO:0045907, GO:0008289, GO:1902622, GO:0050920, GO:0051047, GO:0046649, GO:0032270, GO:0009991, GO:0033628, GO:0004715, GO:0045776, GO:0042454, GO:0005515, GO:0001948, GO:0045308, GO:0002706, GO:1903530, GO:1901657, GO:0030322, GO:0042270, GO:0045088, GO:0046717, GO:0016661, GO:0008584, GO:0002428, GO:1901568, GO:0042325, GO:0044433, GO:0044057, GO:0031638, GO:0006953, GO:0050729, GO:0046546, GO:0042531, GO:0042511, GO:0042515, GO:0042517, GO:0042520, GO:0042523, GO:0042526, GO:0042529, GO:0046850, GO:0005178, GO:0048514, GO:0045682, GO:0003674, GO:0005554, GO:0046634, GO:0061041, GO:0008016, GO:0043407, GO:0046456, GO:0007596, GO:0045606, GO:0014070, GO:0048870, GO:0051674, GO:0002704, GO:0007584, GO:0070228, GO:0002675, GO:0052548, GO:0001664, GO:0090330, GO:0045117, GO:0034340, GO:0044853, GO:0032587, GO:0007586, GO:0097529, GO:0045595, GO:0040012, GO:0050866, GO:0010035, GO:0034767, GO:0098801, GO:0015079, GO:0015388, GO:0022817, GO:0044706, GO:1901605, GO:0009636, GO:0007599, GO:0002705, GO:2000145, GO:0034103, GO:0032642, GO:0098805, GO:0051209, GO:1901137, GO:0090066, GO:0098641, GO:0032409, GO:0007589, GO:0046128, GO:0061134, GO:0015893, GO:0001726, GO:0001893, GO:0030334, GO:0042398 or any combination thereof.

In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies selected from the group consisting of GO:0044459, GO:0071944, GO:0005886, GO:0005904, GO:0031226, GO:0005887, GO:0042127, GO:0005576, GO:0044421, GO:0070887, GO:0034097, GO:0050896, GO:0051869, GO:0071345, GO:0048856, GO:0010033, GO:0044425, GO:0007166, GO:0032501, GO:0044707, GO:0050874, GO:0023052, GO:0023046, GO:0044700, GO:0031982, GO:0031988, GO:0032502, GO:0044767, GO:0007154, GO:0071310, GO:0005615, GO:0042221, GO:0031224, GO:0051049, GO:0019221, GO:0048583, GO:0008284, GO:0007275, GO:0023051, GO:0010646, GO:0048584, GO:0051239, GO:0032879, GO:0006954, GO:0007165, GO:0023033, GO:0043230, GO:0098771, GO:0055065, GO:0016021, GO:1903561, GO:0009966, GO:0035466, GO:0050801, GO:0010647, GO:0006811, GO:0065008, GO:0051240, GO:0098590, GO:0055082, GO:0055080, GO:0023056, GO:0006875, GO:0070062, GO:0051716, GO:0048878, GO:0043269, GO:0065009, GO:0051050, GO:0050865, GO:0098857, GO:0006873, GO:0048518, GO:0043119, GO:0030003, GO:0048731, GO:0042592, GO:0045121, GO:0006952, GO:0002217, GO:0042829, GO:0048522, GO:0051242, GO:0046903, GO:0005102, GO:0030154, GO:0019725, GO:0001775, GO:0009967, GO:0035468, GO:0002376, GO:0072503, GO:0045321, GO:0050863, GO:0050878, GO:0048869, GO:0002703, GO:0050670, GO:0022407, GO:0032944, GO:0016020, GO:1902533, GO:0010740, GO:0043270, GO:0045785, GO:0072507, GO:0009888, GO:0022409, GO:0042493, GO:0017035, GO:0002682, GO:0006874, GO:0032101, GO:0070663, GO:0007204, GO:1902531, GO:0010627, GO:1903039, GO:1903037, GO:0002694, GO:0031012, GO:0009605, GO:0044281, GO:2000021, GO:0055074, GO:0035296, GO:0097746, GO:0042312, GO:0044093, GO:0002685, GO:0098589, GO:0051480, GO:0003013, GO:0008015, GO:0070261, GO:1901700, GO:0007187, GO:0030155, GO:0003006, GO:0034220, GO:0050870, GO:0009611, GO:0002245, GO:0008217, GO:1903524, GO:0042129, GO:0033993, GO:0050880, GO:0007188, GO:0051704, GO:0051706, GO:0035150, GO:0030198, GO:0032103, GO:0043062, GO:0050867, GO:0040017, GO:0002687, GO:0022857, GO:0005386, GO:0015563, GO:0015646, GO:0022891, GO:0022892, GO:0048608, GO:0015267, GO:0015249, GO:0015268, GO:0002274, GO:0001890, GO:0048513, GO:0022803, GO:0022814, GO:0002684, GO:0050776, GO:0002819, GO:0045937, GO:0010562, GO:0002366, GO:0061458, GO:0051094, GO:0034762, GO:2000147, GO:0030141, GO:0002263, GO:0006955, GO:0015075, GO:0099503, GO:0000003, GO:0019952, GO:0050876, GO:0098772, GO:0002252, GO:0009653, GO:0050900, GO:1901701, GO:0042802, GO:0043085, GO:0048554, GO:0030335, GO:0005215, GO:0005478, GO:0022414, GO:0044702, GO:0051241, GO:0002696, GO:0046873, GO:0042060, GO:0003018, GO:0032940, GO:0031410, GO:0016023, GO:0002822, GO:0046394, GO:0051272, GO:0097708, GO:0009986, GO:0009928, GO:0009929, GO:0016053, GO:0051928, GO:0042327, GO:0031225, GO:0010469, GO:0009987, GO:0008151, GO:0044763, GO:0050875, GO:0006950, GO:0043207, GO:0002886, GO:0051249, GO:0098655, GO:0005575, GO:0008372, GO:0002697, GO:0019935, GO:0007267, GO:0032496, GO:0070160, GO:0005216, GO:0034765, GO:0006820, GO:0006822, GO:0005911, GO:0019933, GO:0004252, GO:0048545, GO:0051924, GO:0006812, GO:0006819, GO:0015674, GO:0019932, GO:0051707, GO:0009613, GO:0042828, GO:0001934, GO:0022838, GO:1902105, GO:0006636, GO:0071624, GO:0055085, GO:0010959, GO:0005923, GO:0030001, GO:0002237, GO:0009607, GO:0002699, GO:0005261, GO:0015281, GO:0015338, GO:1903522, GO:0043408, GO:0008324, GO:0015711, GO:0071622, GO:0070665, GO:0002683, GO:0010543, GO:0050730, GO:0007189, GO:0010579, GO:0010580, GO:0016338, GO:0050671, GO:0015318, GO:0050777, GO:0050793, GO:0030054, GO:0022610, GO:0032946, GO:0043300, GO:0042102, GO:0001817, GO:0002275, GO:0032844, GO:0060429, GO:0001653, GO:0031347, GO:0048646, GO:0042981, GO:0051345, GO:0002690, GO:0043302, GO:0098660, GO:0009719, GO:0048018, GO:0071884, GO:0009116, GO:0043168, GO:0002444, GO:0043296, GO:0065007, GO:0098662, GO:0043299, GO:0030193, GO:0042119, GO:0050921, GO:0002688, GO:0043410, GO:0022836, GO:0090022, GO:0002888, GO:0002821, GO:1900046, GO:0042509, GO:0042510, GO:0042513, GO:0042516, GO:0042519, GO:0042522, GO:0042525, GO:0042528, GO:0035295, GO:0043235, GO:0022839, GO:0090023, GO:0043065, GO:0046718, GO:0019063, GO:0043067, GO:0043070, GO:0030545, GO:0001816, GO:0003382, GO:0044409, GO:0051806, GO:0030260, GO:0051828, GO:0036230, GO:0010941, GO:0009725, GO:0002476, GO:0002526, GO:0051384, GO:0050790, GO:0048552, GO:0051247, GO:0008285, GO:0097755, GO:0045909, GO:0031960, GO:0070374, GO:0002824, GO:0030728, GO:0007155, GO:0098602, GO:0035556, GO:0007242, GO:0007243, GO:0023013, GO:0023034, GO:0010942, GO:0070372, GO:0051046, GO:0043068, GO:0043071, GO:1902107, GO:0002283, GO:0005509, GO:0050818, GO:0051336, GO:0009119, GO:0003073, GO:0036018, GO:0046635, GO:2000026, GO:0006082, GO:0001819, GO:0004175, GO:0016809, GO:0050764, GO:0043436, GO:0005201, GO:0097028, GO:0008528, GO:0045055, GO:0016477, GO:0030168, GO:0035239, GO:0070820, GO:0031349, GO:0001932, GO:0098797, GO:0045137, GO:0043312, GO:0002446, GO:0052547, GO:0048585, GO:0009070, GO:0009113, GO:0034764, GO:0022600, GO:0016323, GO:0045597, GO:0042803, GO:0016324, GO:0045177, GO:0008406, GO:0006887, GO:0016194, GO:0016195, GO:0008236, GO:0072358, GO:0001944, GO:0002521, GO:1902624, GO:0044283, GO:0048519, GO:0043118, GO:0045684, GO:0006690, GO:0010522, GO:0022890, GO:0015082, GO:0019752, GO:0071396, GO:0001525, GO:0050731, GO:0036017, GO:0042609, GO:0050817, GO:0070252, GO:0060670, GO:0019369, GO:0019229, GO:0009164, GO:0017171, GO:0045907, GO:0008289, GO:1902622, GO:0050920, GO:0051047, GO:0046649, GO:0032270, GO:0009991, GO:0033628, GO:0004715, GO:0045776, GO:0042454, GO:0005515, GO:0001948, GO:0045308, GO:0002706, GO:1903530, GO:1901657, GO:0030322, GO:0042270, GO:0045088, GO:0046717, GO:0016661, GO:0008584, GO:0002428, GO:1901568, GO:0042325, GO:0044433, GO:0044057, GO:0031638, GO:0006953, GO:0050729, GO:0046546, GO:0042531, GO:0042511, GO:0042515, GO:0042517, GO:0042520, GO:0042523, GO:0042526, GO:0042529, GO:0046850, GO:0005178, GO:0048514, GO:0045682, GO:0003674, GO:0005554, GO:0046634, GO:0061041, GO:0008016, GO:0043407, GO:0046456, GO:0007596, GO:0045606, GO:0014070, GO:0048870, GO:0051674, GO:0002704, GO:0007584, GO:0070228, GO:0002675, GO:0052548, GO:0001664, GO:0090330, GO:0045117, GO:0034340, GO:0044853, GO:0032587, GO:0007586, GO:0097529, GO:0045595, GO:0040012, GO:0050866, GO:0010035, GO:0034767, GO:0098801, GO:0015079, GO:0015388, GO:0022817, GO:0044706, GO:1901605, GO:0009636, GO:0007599, GO:0002705, GO:2000145, GO:0034103, GO:0032642, GO:0098805, GO:0051209, GO:1901137, GO:0090066, GO:0098641, GO:0032409, GO:0007589, GO:0046128, GO:0061134, GO:0015893, GO:0001726, GO:0001893, GO:0030334, GO:0042398 and any combination thereof.

In embodiments, the second gene set includes about 1-1000 decreased genes within one or more of the second gene ontologies. In embodiments, the second gene set includes about 2-1000 decreased genes within one or more of the second gene ontologies. In embodiments, the second gene set includes about 3-1000 decreased genes within one or more of the second gene ontologies. In embodiments, the second gene set includes about 4-1000 decreased genes within one or more of the second gene ontologies. In embodiments, the second gene set includes about 5-1000 decreased genes within one or more of the second gene ontologies. In embodiments, the second gene set includes about 6-1000 decreased genes within one or more of the second gene ontologies. In embodiments, the second gene set includes about 7-1000 decreased genes within one or more of the second gene ontologies. In embodiments, the second gene set includes about 8-1000 decreased genes within one or more of the second gene ontologies. In embodiments, the second gene set includes about 9-1000 decreased genes within one or more of the second gene ontologies. In embodiments, the second gene set includes about 10-1000 decreased genes within one or more of the second gene ontologies.

In embodiments, the second gene set includes about 15-1000 decreased genes within one or more of the second gene ontologies. In embodiments, the second gene set includes about 20-1000 decreased genes within one or more of the second gene ontologies. In embodiments, the second gene set includes about 25-1000 decreased genes within one or more of the second gene ontologies. In embodiments, the second gene set includes about 30-1000 decreased genes within one or more of the second gene ontologies. In embodiments, the second gene set includes about 35-1000 decreased genes within one or more of the second gene ontologies. In embodiments, the second gene set includes about 40-1000 decreased genes within one or more of the second gene ontologies. In embodiments, the second gene set includes about 45-1000 decreased genes within one or more of the second gene ontologies. In embodiments, the second gene set includes about 50-1000 decreased genes within one or more of the second gene ontologies. In embodiments, the second gene set includes about 55-1000 decreased genes within one or more of the second gene ontologies. In embodiments, the second gene set includes about 60-1000 decreased genes within one or more of the second gene ontologies. In embodiments, the second gene set includes about 65-1000 decreased genes within one or more of the second gene ontologies. In embodiments, the second gene set includes about 70-1000 decreased genes within one or more of the second gene ontologies. In embodiments, the second gene set includes about 75-1000 decreased genes within one or more of the second gene ontologies. In embodiments, the second gene set includes about 80-1000 decreased genes within one or more of the second gene ontologies. In embodiments, the second gene set includes about 85-1000 decreased genes within one or more of the second gene ontologies. In embodiments, the second gene set includes about 90-1000 decreased genes within one or more of the second gene ontologies. In embodiments, the second gene set includes about 95-1000 decreased genes within one or more of the second gene ontologies. In embodiments, the second gene set includes about 100-1000 decreased genes within one or more of the second gene ontologies.

In embodiments, the second gene set includes about 105-1000 decreased genes within one or more of the second gene ontologies. In embodiments, the second gene set includes about 115-1000 decreased genes within one or more of the second gene ontologies. In embodiments, the second gene set includes about 120-1000 decreased genes within one or more of the second gene ontologies. In embodiments, the second gene set includes about 125-1000 decreased genes within one or more of the second gene ontologies. In embodiments, the second gene set includes about 130-1000 decreased genes within one or more of the second gene ontologies. In embodiments, the second gene set includes about 135-1000 decreased genes within one or more of the second gene ontologies. In embodiments, the second gene set includes about 140-1000 decreased genes within one or more of the second gene ontologies. In embodiments, the second gene set includes about 145-1000 decreased genes within one or more of the second gene ontologies. In embodiments, the second gene set includes about 150-1000 decreased genes within one or more of the second gene ontologies. In embodiments, the second gene set includes about 155-1000 decreased genes within one or more of the second gene ontologies. In embodiments, the second gene set includes about 160-1000 decreased genes within one or more of the second gene ontologies. In embodiments, the second gene set includes about 165-1000 decreased genes within one or more of the second gene ontologies. In embodiments, the second gene set includes about 170-1000 decreased genes within one or more of the second gene ontologies. In embodiments, the second gene set includes about 175-1000 decreased genes within one or more of the second gene ontologies. In embodiments, the second gene set includes about 180-1000 decreased genes within one or more of the second gene ontologies. In embodiments, the second gene set includes about 185-1000 decreased genes within one or more of the second gene ontologies. In embodiments, the second gene set includes about 190-1000 decreased genes within one or more of the second gene ontologies. In embodiments, the second gene set includes about 195-1000 decreased genes within one or more of the second gene ontologies. In embodiments, the second gene set includes about 200-1000 decreased genes within one or more of the second gene ontologies.

In embodiments, the second gene set includes about 205-1000 decreased genes within one or more of the second gene ontologies. In embodiments, the second gene set includes about 215-1000 decreased genes within one or more of the second gene ontologies. In embodiments, the second gene set includes about 220-1000 decreased genes within one or more of the second gene ontologies. In embodiments, the second gene set includes about 225-1000 decreased genes within one or more of the second gene ontologies. In embodiments, the second gene set includes about 230-1000 decreased genes within one or more of the second gene ontologies. In embodiments, the second gene set includes about 235-1000 decreased genes within one or more of the second gene ontologies. In embodiments, the second gene set includes about 240-1000 decreased genes within one or more of the second gene ontologies. In embodiments, the second gene set includes about 245-1000 decreased genes within one or more of the second gene ontologies. In embodiments, the second gene set includes about 250-1000 decreased genes within one or more of the second gene ontologies. In embodiments, the second gene set includes about 255-1000 decreased genes within one or more of the second gene ontologies. In embodiments, the second gene set includes about 260-1000 decreased genes within one or more of the second gene ontologies. In embodiments, the second gene set includes about 265-1000 decreased genes within one or more of the second gene ontologies. In embodiments, the second gene set includes about 270-1000 decreased genes within one or more of the second gene ontologies. In embodiments, the second gene set includes about 275-1000 decreased genes within one or more of the second gene ontologies. In embodiments, the second gene set includes about 280-1000 decreased genes within one or more of the second gene ontologies. In embodiments, the second gene set includes about 285-1000 decreased genes within one or more of the second gene ontologies. In embodiments, the second gene set includes about 290-1000 decreased genes within one or more of the second gene ontologies. In embodiments, the second gene set includes about 295-1000 decreased genes within one or more of the second gene ontologies. In embodiments, the second gene set includes about 300-1000 decreased genes within one or more of the second gene ontologies.

In embodiments, the second gene set includes about 305-1000 decreased genes within one or more of the second gene ontologies. In embodiments, the second gene set includes about 315-1000 decreased genes within one or more of the second gene ontologies. In embodiments, the second gene set includes about 320-1000 decreased genes within one or more of the second gene ontologies. In embodiments, the second gene set includes about 325-1000 decreased genes within one or more of the second gene ontologies. In embodiments, the second gene set includes about 330-1000 decreased genes within one or more of the second gene ontologies. In embodiments, the second gene set includes about 335-1000 decreased genes within one or more of the second gene ontologies. In embodiments, the second gene set includes about 340-1000 decreased genes within one or more of the second gene ontologies. In embodiments, the second gene set includes about 345-1000 decreased genes within one or more of the second gene ontologies. In embodiments, the second gene set includes about 350-1000 decreased genes within one or more of the second gene ontologies. In embodiments, the second gene set includes about 355-1000 decreased genes within one or more of the second gene ontologies. In embodiments, the second gene set includes about 360-1000 decreased genes within one or more of the second gene ontologies. In embodiments, the second gene set includes about 365-1000 decreased genes within one or more of the second gene ontologies. In embodiments, the second gene set includes about 370-1000 decreased genes within one or more of the second gene ontologies. In embodiments, the second gene set includes about 375-1000 decreased genes within one or more of the second gene ontologies. In embodiments, the second gene set includes about 380-1000 decreased genes within one or more of the second gene ontologies. In embodiments, the second gene set includes about 385-1000 decreased genes within one or more of the second gene ontologies. In embodiments, the second gene set includes about 390-1000 decreased genes within one or more of the second gene ontologies. In embodiments, the second gene set includes about 395-1000 decreased genes within one or more of the second gene ontologies. In embodiments, the second gene set includes about 400-1000 decreased genes within one or more of the second gene ontologies.

In embodiments, the second gene set includes about 405-1000 decreased genes within one or more of the second gene ontologies. In embodiments, the second gene set includes about 415-1000 decreased genes within one or more of the second gene ontologies. In embodiments, the second gene set includes about 420-1000 decreased genes within one or more of the second gene ontologies. In embodiments, the second gene set includes about 425-1000 decreased genes within one or more of the second gene ontologies. In embodiments, the second gene set includes about 430-1000 decreased genes within one or more of the second gene ontologies. In embodiments, the second gene set includes about 435-1000 decreased genes within one or more of the second gene ontologies. In embodiments, the second gene set includes about 440-1000 decreased genes within one or more of the second gene ontologies. In embodiments, the second gene set includes about 445-1000 decreased genes within one or more of the second gene ontologies. In embodiments, the second gene set includes about 450-1000 decreased genes within one or more of the second gene ontologies. In embodiments, the second gene set includes about 455-1000 decreased genes within one or more of the second gene ontologies. In embodiments, the second gene set includes about 460-1000 decreased genes within one or more of the second gene ontologies. In embodiments, the second gene set includes about 465-1000 decreased genes within one or more of the second gene ontologies. In embodiments, the second gene set includes about 470-1000 decreased genes within one or more of the second gene ontologies. In embodiments, the second gene set includes about 475-1000 decreased genes within one or more of the second gene ontologies. In embodiments, the second gene set includes about 480-1000 decreased genes within one or more of the second gene ontologies. In embodiments, the second gene set includes about 485-1000 decreased genes within one or more of the second gene ontologies. In embodiments, the second gene set includes about 490-1000 decreased genes within one or more of the second gene ontologies. In embodiments, the second gene set includes about 495-1000 decreased genes within one or more of the second gene ontologies.

In embodiments, the second gene set includes about 500-1000 decreased genes within one or more of the second gene ontologies. In embodiments, the second gene set includes about 505-1000 decreased genes within one or more of the second gene ontologies. In embodiments, the second gene set includes about 510-1000 decreased genes within one or more of the second gene ontologies. In embodiments, the second gene set includes about 515-1000 decreased genes within one or more of the second gene ontologies. In embodiments, the second gene set includes about 520-1000 decreased genes within one or more of the second gene ontologies. In embodiments, the second gene set includes about 525-1000 decreased genes within one or more of the second gene ontologies. In embodiments, the second gene set includes about 530-1000 decreased genes within one or more of the second gene ontologies. In embodiments, the second gene set includes about 535-1000 decreased genes within one or more of the second gene ontologies. In embodiments, the second gene set includes about 540-1000 decreased genes within one or more of the second gene ontologies. In embodiments, the second gene set includes about 545-1000 decreased genes within one or more of the second gene ontologies. In embodiments, the second gene set includes about 550-1000 decreased genes within one or more of the second gene ontologies. In embodiments, the second gene set includes about 555-1000 decreased genes within one or more of the second gene ontologies. In embodiments, the second gene set includes about 565-1000 decreased genes within one or more of the second gene ontologies. In embodiments, the second gene set includes about 570-1000 decreased genes within one or more of the second gene ontologies. In embodiments, the second gene set includes about 575-1000 decreased genes within one or more of the second gene ontologies. In embodiments, the second gene set includes about 580-1000 decreased genes within one or more of the second gene ontologies. In embodiments, the second gene set includes about 585-1000 decreased genes within one or more of the second gene ontologies. In embodiments, the second gene set includes about 590-1000 decreased genes within one or more of the second gene ontologies. In embodiments, the second gene set includes about 595-1000 decreased genes within one or more of the second gene ontologies. In embodiments, the second gene set includes about 600-1000 decreased genes within one or more of the second gene ontologies.

In embodiments, the second gene set includes about 605-1000 decreased genes within one or more of the second gene ontologies. In embodiments, the second gene set includes about 615-1000 decreased genes within one or more of the second gene ontologies. In embodiments, the second gene set includes about 620-1000 decreased genes within one or more of the second gene ontologies. In embodiments, the second gene set includes about 625-1000 decreased genes within one or more of the second gene ontologies. In embodiments, the second gene set includes about 630-1000 decreased genes within one or more of the second gene ontologies. In embodiments, the second gene set includes about 635-1000 decreased genes within one or more of the second gene ontologies. In embodiments, the second gene set includes about 640-1000 decreased genes within one or more of the second gene ontologies. In embodiments, the second gene set includes about 645-1000 decreased genes within one or more of the second gene ontologies. In embodiments, the second gene set includes about 650-1000 decreased genes within one or more of the second gene ontologies. In embodiments, the second gene set includes about 655-1000 decreased genes within one or more of the second gene ontologies. In embodiments, the second gene set includes about 660-1000 decreased genes within one or more of the second gene ontologies. In embodiments, the second gene set includes about 665-1000 decreased genes within one or more of the second gene ontologies. In embodiments, the second gene set includes about 670-1000 decreased genes within one or more of the second gene ontologies. In embodiments, the second gene set includes about 675-1000 decreased genes within one or more of the second gene ontologies. In embodiments, the second gene set includes about 680-1000 decreased genes within one or more of the second gene ontologies. In embodiments, the second gene set includes about 685-1000 decreased genes within one or more of the second gene ontologies. In embodiments, the second gene set includes about 690-1000 decreased genes within one or more of the second gene ontologies. In embodiments, the second gene set includes about 695-1000 decreased genes within one or more of the second gene ontologies. In embodiments, the second gene set includes about 700-1000 decreased genes within one or more of the second gene ontologies.

In embodiments, the second gene set includes about 705-1000 decreased genes within one or more of the second gene ontologies. In embodiments, the second gene set includes about 715-1000 decreased genes within one or more of the second gene ontologies. In embodiments, the second gene set includes about 720-1000 decreased genes within one or more of the second gene ontologies. In embodiments, the second gene set includes about 725-1000 decreased genes within one or more of the second gene ontologies. In embodiments, the second gene set includes about 730-1000 decreased genes within one or more of the second gene ontologies. In embodiments, the second gene set includes about 735-1000 decreased genes within one or more of the second gene ontologies. In embodiments, the second gene set includes about 740-1000 decreased genes within one or more of the second gene ontologies. In embodiments, the second gene set includes about 745-1000 decreased genes within one or more of the second gene ontologies. In embodiments, the second gene set includes about 750-1000 decreased genes within one or more of the second gene ontologies. In embodiments, the second gene set includes about 755-1000 decreased genes within one or more of the second gene ontologies. In embodiments, the second gene set includes about 760-1000 decreased genes within one or more of the second gene ontologies. In embodiments, the second gene set includes about 765-1000 decreased genes within one or more of the second gene ontologies. In embodiments, the second gene set includes about 770-1000 decreased genes within one or more of the second gene ontologies. In embodiments, the second gene set includes about 775-1000 decreased genes within one or more of the second gene ontologies. In embodiments, the second gene set includes about 780-1000 decreased genes within one or more of the second gene ontologies. In embodiments, the second gene set includes about 785-1000 decreased genes within one or more of the second gene ontologies. In embodiments, the second gene set includes about 790-1000 decreased genes within one or more of the second gene ontologies. In embodiments, the second gene set includes about 795-1000 decreased genes within one or more of the second gene ontologies. In embodiments, the second gene set includes about 800-1000 decreased genes within one or more of the second gene ontologies.

In embodiments, the second gene set includes about 805-1000 decreased genes within one or more of the second gene ontologies. In embodiments, the second gene set includes about 815-1000 decreased genes within one or more of the second gene ontologies. In embodiments, the second gene set includes about 820-1000 decreased genes within one or more of the second gene ontologies. In embodiments, the second gene set includes about 825-1000 decreased genes within one or more of the second gene ontologies. In embodiments, the second gene set includes about 830-1000 decreased genes within one or more of the second gene ontologies. In embodiments, the second gene set includes about 835-1000 decreased genes within one or more of the second gene ontologies. In embodiments, the second gene set includes about 840-1000 decreased genes within one or more of the second gene ontologies. In embodiments, the second gene set includes about 845-1000 decreased genes within one or more of the second gene ontologies. In embodiments, the second gene set includes about 850-1000 decreased genes within one or more of the second gene ontologies. In embodiments, the second gene set includes about 855-1000 decreased genes within one or more of the second gene ontologies. In embodiments, the second gene set includes about 860-1000 decreased genes within one or more of the second gene ontologies. In embodiments, the second gene set includes about 865-1000 decreased genes within one or more of the second gene ontologies. In embodiments, the second gene set includes about 870-1000 decreased genes within one or more of the second gene ontologies. In embodiments, the second gene set includes about 875-1000 decreased genes within one or more of the second gene ontologies. In embodiments, the second gene set includes about 880-1000 decreased genes within one or more of the second gene ontologies. In embodiments, the second gene set includes about 885-1000 decreased genes within one or more of the second gene ontologies. In embodiments, the second gene set includes about 890-1000 decreased genes within one or more of the second gene ontologies. In embodiments, the second gene set includes about 895-1000 decreased genes within one or more of the second gene ontologies. In embodiments, the second gene set includes about 900-1000 decreased genes within one or more of the second gene ontologies.

In embodiments, the second gene set includes about 905-1000 decreased genes within one or more of the second gene ontologies. In embodiments, the second gene set includes about 915-1000 decreased genes within one or more of the second gene ontologies. In embodiments, the second gene set includes about 920-1000 decreased genes within one or more of the second gene ontologies. In embodiments, the second gene set includes about 925-1000 decreased genes within one or more of the second gene ontologies. In embodiments, the second gene set includes about 930-1000 decreased genes within one or more of the second gene ontologies. In embodiments, the second gene set includes about 935-1000 decreased genes within one or more of the second gene ontologies. In embodiments, the second gene set includes about 940-1000 decreased genes within one or more of the second gene ontologies. In embodiments, the second gene set includes about 945-1000 decreased genes within one or more of the second gene ontologies. In embodiments, the second gene set includes about 950-1000 decreased genes within one or more of the second gene ontologies. In embodiments, the second gene set includes about 955-1000 decreased genes within one or more of the second gene ontologies. In embodiments, the second gene set includes about 960-1000 decreased genes within one or more of the second gene ontologies. In embodiments, the second gene set includes about 965-1000 decreased genes within one or more of the second gene ontologies. In embodiments, the second gene set includes about 970-1000 decreased genes within one or more of the second gene ontologies. In embodiments, the second gene set includes about 975-1000 decreased genes within one or more of the second gene ontologies. In embodiments, the second gene set includes about 980-1000 decreased genes within one or more of the second gene ontologies. In embodiments, the second gene set includes about 985-1000 decreased genes within one or more of the second gene ontologies. In embodiments, the second gene set includes about 990-1000 decreased genes within one or more of the second gene ontologies. In embodiments, the second gene set includes about 995-1000 decreased genes within one or more of the second gene ontologies.

In embodiments, the second gene set includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511 512, 513, 514, 515, 516, 517, 518, 519, 520, 521, 522, 523, 524, 525, 526, 527, 528, 529, 530, 231, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 605, 603, 604, 605, 606, 607, 608, 609, 610, 611 612, 613, 614, 615 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707,708, 709, 710, 711 712, 713, 717, 715 716, 714, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 757, 755, 756, 754, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811 812, 813, 817, 815 816, 814, 818, 819, 820, 821, 822, 823, 824, 825, 826, 827, 828, 829, 830, 831, 832, 833, 834, 835, 836, 837, 838, 839, 840, 841, 842, 843, 845, 846, 847, 848, 849, 850, 851, 852, 853, 854, 855, 856, 854, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911 912, 913, 917, 915 916, 914, 918, 919, 920, 921, 922, 923, 924, 925, 926, 927, 928, 929, 930, 931, 932, 933, 934, 935, 936, 937, 938, 939, 940, 941, 942, 943, 945, 946, 947, 948, 949, 950, 951, 952, 953, 954, 955, 956, 954, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 decreased genes within one or more of the second gene ontologies.

The gene expression profile information for the desirable determined dopaminergic precursor cell may include decreased gene expression levels relative to a pluripotent stem cell for a second gene set, wherein the second gene set includes at least one decreased gene within one or more second gene ontologies of Table 8. “One or more” as described herein in the context of second gene ontologies refers to at least one, for example, 1, 2, 3, 4, 5, 6, 7, 8, 9, 10, 11, 12, 13, 14, 15, 16, 17, 18, 19, etc. of second gene ontologies.

In embodiments, the second gene set includes about 1-500 decreased genes within 1-1000 of the second gene ontologies. In embodiments, the second gene set includes about 1-500 decreased genes within 50-1000 of the second gene ontologies. In embodiments, the second gene set includes about 1-500 decreased genes within 100-1000 of the second gene ontologies. In embodiments, the second gene set includes about 1-500 decreased genes within 150-1000 of the second gene ontologies. In embodiments, the second gene set includes about 1-500 decreased genes within 200-1000 of the second gene ontologies. In embodiments, the second gene set includes about 250-500 decreased genes within 50-1000 of the second gene ontologies. In embodiments, the second gene set includes about 1-500 decreased genes within 300-1000 of the second gene ontologies. In embodiments, the second gene set includes about 1-500 decreased genes within 350-1000 of the second gene ontologies. In embodiments, the second gene set includes about 1-500 decreased genes within 400-1000 of the second gene ontologies. In embodiments, the second gene set includes about 1-500 decreased genes within 450-1000 of the second gene ontologies. In embodiments, the second gene set includes about 1-500 decreased genes within 500-1000 of the second gene ontologies. In embodiments, the second gene set includes about 1-500 decreased genes within 550-1000 of the second gene ontologies. In embodiments, the second gene set includes about 1-500 decreased genes within 600-1000 of the second gene ontologies. In embodiments, the second gene set includes about 1-500 decreased genes within 650-1000 of the second gene ontologies. In embodiments, the second gene set includes about 1-500 decreased genes within 700-1000 of the second gene ontologies. In embodiments, the second gene set includes about 1-500 decreased genes within 750-1000 of the second gene ontologies. In embodiments, the second gene set includes about 1-500 decreased genes within 800-1000 of the second gene ontologies. In embodiments, the second gene set includes about 1-500 decreased genes within 850-1000 of the second gene ontologies. In embodiments, the second gene set includes about 1-500 decreased genes within 900-1000 of the second gene ontologies. In embodiments, the second gene set includes about 1-500 decreased genes within 950-1000 of the second gene ontologies.

In embodiments, the second gene set includes about 1-500 decreased genes within 1-300 of the second gene ontologies. In embodiments, the second gene set includes about 1-500 decreased genes within 10-300 of the second gene ontologies. In embodiments, the second gene set includes about 1-500 decreased genes within 20-300 of the second gene ontologies. In embodiments, the second gene set includes about 1-500 decreased genes within 30-300 of the second gene ontologies. In embodiments, the second gene set includes about 1-500 decreased genes within 40-300 of the second gene ontologies. In embodiments, the second gene set includes about 1-500 decreased genes within 50-300 of the second gene ontologies. In embodiments, the second gene set includes about 1-500 decreased genes within 60-300 of the second gene ontologies. In embodiments, the second gene set includes about 1-500 decreased genes within 70-300 of the second gene ontologies. In embodiments, the second gene set includes about 1-500 decreased genes within 80-300 of the second gene ontologies. In embodiments, the second gene set includes about 1-500 decreased genes within 90-300 of the second gene ontologies. In embodiments, the second gene set includes about 1-500 decreased genes within 100-300 of the second gene ontologies. In embodiments, the second gene set includes about 1-500 decreased genes within 110-300 of the second gene ontologies. In embodiments, the second gene set includes about 1-500 decreased genes within 120-300 of the second gene ontologies. In embodiments, the second gene set includes about 1-500 decreased genes within 130-300 of the second gene ontologies. In embodiments, the second gene set includes about 1-500 decreased genes within 140-300 of the second gene ontologies. In embodiments, the second gene set includes about 1-500 decreased genes within 150-300 of the second gene ontologies. In embodiments, the second gene set includes about 1-500 decreased genes within 160-300 of the second gene ontologies. In embodiments, the second gene set includes about 1-500 decreased genes within 170-300 of the second gene ontologies. In embodiments, the second gene set includes about 1-500 decreased genes within 180-300 of the second gene ontologies. In embodiments, the second gene set includes about 1-500 decreased genes within 190-300 of the second gene ontologies. In embodiments, the second gene set includes about 1-500 decreased genes within 200-300 of the second gene ontologies. In embodiments, the second gene set includes about 1-500 decreased genes within 210-300 of the second gene ontologies. In embodiments, the second gene set includes about 1-500 decreased genes within 220-300 of the second gene ontologies. In embodiments, the second gene set includes about 1-500 decreased genes within 230-300 of the second gene ontologies. In embodiments, the second gene set includes about 1-500 decreased genes within 240-300 of the second gene ontologies. In embodiments, the second gene set includes about 1-500 decreased genes within 250-300 of the second gene ontologies. In embodiments, the second gene set includes about 1-500 decreased genes within 260-300 of the second gene ontologies. In embodiments, the second gene set includes about 1-500 decreased genes within 270-300 of the second gene ontologies. In embodiments, the second gene set includes about 1-500 decreased genes within 280-300 of the second gene ontologies. In embodiments, the second gene set includes about 1-500 decreased genes within 290-300 of the second gene ontologies.

In embodiments, the second gene set includes about 1-500 decreased genes within 1-290 of the second gene ontologies. In embodiments, the second gene set includes about 1-500 decreased genes within 1-280 of the second gene ontologies. In embodiments, the second gene set includes about 1-500 decreased genes within 1-270 of the second gene ontologies. In embodiments, the second gene set includes about 1-500 decreased genes within 1-260 of the second gene ontologies. In embodiments, the second gene set includes about 1-500 decreased genes within 1-250 of the second gene ontologies. In embodiments, the second gene set includes about 1-500 decreased genes within 1-240 of the second gene ontologies. In embodiments, the second gene set includes about 1-500 decreased genes within 1-230 of the second gene ontologies. In embodiments, the second gene set includes about 1-500 decreased genes within 1-220 of the second gene ontologies. In embodiments, the second gene set includes about 1-500 decreased genes within 1-210 of the second gene ontologies. In embodiments, the second gene set includes about 1-500 decreased genes within 1-200 of the second gene ontologies. In embodiments, the second gene set includes about 1-500 decreased genes within 1-190 of the second gene ontologies. In embodiments, the second gene set includes about 1-500 decreased genes within 1-180 of the second gene ontologies. In embodiments, the second gene set includes about 1-500 decreased genes within 1-170 of the second gene ontologies. In embodiments, the second gene set includes about 1-500 decreased genes within 1-160 of the second gene ontologies. In embodiments, the second gene set includes about 1-500 decreased genes within 1-150 of the second gene ontologies. In embodiments, the second gene set includes about 1-500 decreased genes within 1-140 of the second gene ontologies. In embodiments, the second gene set includes about 1-500 decreased genes within 1-130 of the second gene ontologies. In embodiments, the second gene set includes about 1-500 decreased genes within 1-120 of the second gene ontologies. In embodiments, the second gene set includes about 1-500 decreased genes within 1-110 of the second gene ontologies. In embodiments, the second gene set includes about 1-500 decreased genes within 1-100 of the second gene ontologies. In embodiments, the second gene set includes about 1-500 decreased genes within 1-90 of the second gene ontologies. In embodiments, the second gene set includes about 1-500 decreased genes within 1-80 of the second gene ontologies. In embodiments, the second gene set includes about 1-500 decreased genes within 1-70 of the second gene ontologies. In embodiments, the second gene set includes about 1-500 decreased genes within 1-60 of the second gene ontologies. In embodiments, the second gene set includes about 1-500 decreased genes within 1-50 of the second gene ontologies. In embodiments, the second gene set includes about 1-500 decreased genes within 1-40 of the second gene ontologies. In embodiments, the second gene set includes about 1-500 decreased genes within 1-30 of the second gene ontologies. In embodiments, the second gene set includes about 1-500 decreased genes within 1-20 of the second gene ontologies. In embodiments, the second gene set includes about 1-500 decreased genes within 1-10 of the second gene ontologies. In embodiments, the second gene set includes about 1-500 decreased genes within 1-5 of the second gene ontologies.

In embodiments, the second gene set includes at least one decreased gene within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407,408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, or 463 second gene ontologies of Table 8.

In embodiments, the second gene set includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511 512, 513, 514, 515, 516, 517, 518, 519, 520, 521, 522, 523, 524, 525, 526, 527, 528, 529, 530, 231, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 605, 603, 604, 605, 606, 607, 608, 609, 610, 611 612, 613, 614, 615 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711 712, 713, 717, 715 716, 714, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 757, 755, 756, 754, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811 812, 813, 817, 815 816, 814, 818, 819, 820, 821, 822, 823, 824, 825, 826, 827, 828, 829, 830, 831, 832, 833, 834, 835, 836, 837, 838, 839, 840, 841, 842, 843, 845, 846, 847, 848, 849, 850, 851, 852, 853, 854, 855, 856, 854, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911 912, 913, 917, 915 916, 914, 918, 919, 920, 921, 922, 923, 924, 925, 926, 927, 928, 929, 930, 931, 932, 933, 934, 935, 936, 937, 938, 939, 940, 941, 942, 943, 945, 946, 947, 948, 949, 950, 951, 952, 953, 954, 955, 956, 954, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 decreased genes within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311 312, 313, 314, 315 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 412, 413, 414, 415 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, or 463 second gene ontologies of Table 8.

In embodiments, the second gene ontologies are any one of the gene ontologies listed in Table 8. In embodiments, the second gene ontologies are any one of GO:0044459, GO:0071944, GO:0005886, GO:0005904, GO:0031226, GO:0005887, GO:0042127, GO:0005576, GO:0044421, GO:0070887, GO:0034097, GO:0050896, GO:0051869, GO:0071345, GO:0048856, GO:0010033, GO:0044425, GO:0007166, GO:0032501, GO:0044707, GO:0050874, GO:0023052, GO:0023046, GO:0044700, GO:0031982, GO:0031988, GO:0032502, GO:0044767, GO:0007154, GO:0071310, GO:0005615, GO:0042221, GO:0031224, GO:0051049, GO:0019221, GO:0048583, GO:0008284, GO:0007275, GO:0023051, GO:0010646, GO:0048584, GO:0051239, GO:0032879, GO:0006954, GO:0007165, GO:0023033, GO:0043230, GO:0098771, GO:0055065, GO:0016021, GO:1903561, GO:0009966, GO:0035466, GO:0050801, GO:0010647, GO:0006811, GO:0065008, GO:0051240, GO:0098590, GO:0055082, GO:0055080, GO:0023056, GO:0006875, GO:0070062, GO:0051716, GO:0048878, GO:0043269, GO:0065009, GO:0051050, GO:0050865, GO:0098857, GO:0006873, GO:0048518, GO:0043119, GO:0030003, GO:0048731, GO:0042592, GO:0045121, GO:0006952, GO:0002217, GO:0042829, GO:0048522, GO:0051242, GO:0046903, GO:0005102, GO:0030154, GO:0019725, GO:0001775, GO:0009967, GO:0035468, GO:0002376, GO:0072503, GO:0045321, GO:0050863, GO:0050878, GO:0048869, GO:0002703, GO:0050670, GO:0022407, GO:0032944, GO:0016020, GO:1902533, GO:0010740, GO:0043270, GO:0045785, GO:0072507, GO:0009888, GO:0022409, GO:0042493, GO:0017035, GO:0002682, GO:0006874, GO:0032101, GO:0070663, GO:0007204, GO:1902531, GO:0010627, GO:1903039, GO:1903037, GO:0002694, GO:0031012, GO:0009605, GO:0044281, GO:2000021, GO:0055074, GO:0035296, GO:0097746, GO:0042312, GO:0044093, GO:0002685, GO:0098589, GO:0051480, GO:0003013, GO:0008015, GO:0070261, GO:1901700, GO:0007187, GO:0030155, GO:0003006, GO:0034220, GO:0050870, GO:0009611, GO:0002245, GO:0008217, GO:1903524, GO:0042129, GO:0033993, GO:0050880, GO:0007188, GO:0051704, GO:0051706, GO:0035150, GO:0030198, GO:0032103, GO:0043062, GO:0050867, GO:0040017, GO:0002687, GO:0022857, GO:0005386, GO:0015563, GO:0015646, GO:0022891, GO:0022892, GO:0048608, GO:0015267, GO:0015249, GO:0015268, GO:0002274, GO:0001890, GO:0048513, GO:0022803, GO:0022814, GO:0002684, GO:0050776, GO:0002819, GO:0045937, GO:0010562, GO:0002366, GO:0061458, GO:0051094, GO:0034762, GO:2000147, GO:0030141, GO:0002263, GO:0006955, GO:0015075, GO:0099503, GO:0000003, GO:0019952, GO:0050876, GO:0098772, GO:0002252, GO:0009653, GO:0050900, GO:1901701, GO:0042802, GO:0043085, GO:0048554, GO:0030335, GO:0005215, GO:0005478, GO:0022414, GO:0044702, GO:0051241, GO:0002696, GO:0046873, GO:0042060, GO:0003018, GO:0032940, GO:0031410, GO:0016023, GO:0002822, GO:0046394, GO:0051272, GO:0097708, GO:0009986, GO:0009928, GO:0009929, GO:0016053, GO:0051928, GO:0042327, GO:0031225, GO:0010469, GO:0009987, GO:0008151, GO:0044763, GO:0050875, GO:0006950, GO:0043207, GO:0002886, GO:0051249, GO:0098655, GO:0005575, GO:0008372, GO:0002697, GO:0019935, GO:0007267, GO:0032496, GO:0070160, GO:0005216, GO:0034765, GO:0006820, GO:0006822, GO:0005911, GO:0019933, GO:0004252, GO:0048545, GO:0051924, GO:0006812, GO:0006819, GO:0015674, GO:0019932, GO:0051707, GO:0009613, GO:0042828, GO:0001934, GO:0022838, GO:1902105, GO:0006636, GO:0071624, GO:0055085, GO:0010959, GO:0005923, GO:0030001, GO:0002237, GO:0009607, GO:0002699, GO:0005261, GO:0015281, GO:0015338, GO:1903522, GO:0043408, GO:0008324, GO:0015711, GO:0071622, GO:0070665, GO:0002683, GO:0010543, GO:0050730, GO:0007189, GO:0010579, GO:0010580, GO:0016338, GO:0050671, GO:0015318, GO:0050777, GO:0050793, GO:0030054, GO:0022610, GO:0032946, GO:0043300, GO:0042102, GO:0001817, GO:0002275, GO:0032844, GO:0060429, GO:0001653, GO:0031347, GO:0048646, GO:0042981, GO:0051345, GO:0002690, GO:0043302, GO:0098660, GO:0009719, GO:0048018, GO:0071884, GO:0009116, GO:0043168, GO:0002444, GO:0043296, GO:0065007, GO:0098662, GO:0043299, GO:0030193, GO:0042119, GO:0050921, GO:0002688, GO:0043410, GO:0022836, GO:0090022, GO:0002888, GO:0002821, GO:1900046, GO:0042509, GO:0042510, GO:0042513, GO:0042516, GO:0042519, GO:0042522, GO:0042525, GO:0042528, GO:0035295, GO:0043235, GO:0022839, GO:0090023, GO:0043065, GO:0046718, GO:0019063, GO:0043067, GO:0043070, GO:0030545, GO:0001816, GO:0003382, GO:0044409, GO:0051806, GO:0030260, GO:0051828, GO:0036230, GO:0010941, GO:0009725, GO:0002476, GO:0002526, GO:0051384, GO:0050790, GO:0048552, GO:0051247, GO:0008285, GO:0097755, GO:0045909, GO:0031960, GO:0070374, GO:0002824, GO:0030728, GO:0007155, GO:0098602, GO:0035556, GO:0007242, GO:0007243, GO:0023013, GO:0023034, GO:0010942, GO:0070372, GO:0051046, GO:0043068, GO:0043071, GO:1902107, GO:0002283, GO:0005509, GO:0050818, GO:0051336, GO:0009119, GO:0003073, GO:0036018, GO:0046635, GO:2000026, GO:0006082, GO:0001819, GO:0004175, GO:0016809, GO:0050764, GO:0043436, GO:0005201, GO:0097028, GO:0008528, GO:0045055, GO:0016477, GO:0030168, GO:0035239, GO:0070820, GO:0031349, GO:0001932, GO:0098797, GO:0045137, GO:0043312, GO:0002446, GO:0052547, GO:0048585, GO:0009070, GO:0009113, GO:0034764, GO:0022600, GO:0016323, GO:0045597, GO:0042803, GO:0016324, GO:0045177, GO:0008406, GO:0006887, GO:0016194, GO:0016195, GO:0008236, GO:0072358, GO:0001944, GO:0002521, GO:1902624, GO:0044283, GO:0048519, GO:0043118, GO:0045684, GO:0006690, GO:0010522, GO:0022890, GO:0015082, GO:0019752, GO:0071396, GO:0001525, GO:0050731, GO:0036017, GO:0042609, GO:0050817, GO:0070252, GO:0060670, GO:0019369, GO:0019229, GO:0009164, GO:0017171, GO:0045907, GO:0008289, GO:1902622, GO:0050920, GO:0051047, GO:0046649, GO:0032270, GO:0009991, GO:0033628, GO:0004715, GO:0045776, GO:0042454, GO:0005515, GO:0001948, GO:0045308, GO:0002706, GO:1903530, GO:1901657, GO:0030322, GO:0042270, GO:0045088, GO:0046717, GO:0016661, GO:0008584, GO:0002428, GO:1901568, GO:0042325, GO:0044433, GO:0044057, GO:0031638, GO:0006953, GO:0050729, GO:0046546, GO:0042531, GO:0042511, GO:0042515, GO:0042517, GO:0042520, GO:0042523, GO:0042526, GO:0042529, GO:0046850, GO:0005178, GO:0048514, GO:0045682, GO:0003674, GO:0005554, GO:0046634, GO:0061041, GO:0008016, GO:0043407, GO:0046456, GO:0007596, GO:0045606, GO:0014070, GO:0048870, GO:0051674, GO:0002704, GO:0007584, GO:0070228, GO:0002675, GO:0052548, GO:0001664, GO:0090330, GO:0045117, GO:0034340, GO:0044853, GO:0032587, GO:0007586, GO:0097529, GO:0045595, GO:0040012, GO:0050866, GO:0010035, GO:0034767, GO:0098801, GO:0015079, GO:0015388, GO:0022817, GO:0044706, GO:1901605, GO:0009636, GO:0007599, GO:0002705, GO:2000145, GO:0034103, GO:0032642, GO:0098805, GO:0051209, GO:1901137, GO:0090066, GO:0098641, GO:0032409, GO:0007589, GO:0046128, GO:0061134, GO:0015893, GO:0001726, GO:0001893, GO:0030334, GO:0042398 or any combination thereof.

In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies selected from the group consisting of: GO0070887, GO0044459 and GO0044281. In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies of: GO0070887, GO0044459, or GO0044281. In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies selected from the group consisting of: GO0042127, GO006954, and GO0032502 and any combination thereof. In embodiments, the gene expression profile information for the desirable determined dopaminergic precursor cell includes decreased gene expression levels relative to a pluripotent stem cell for a second gene set, wherein the second gene set includes at least one decreased gene within one or more second gene ontologies of: GO0042127, GO006954, GO0032502 or any combination thereof.

In embodiments, the second gene set includes at least one (e.g., 1, 2, 3, 4, 5, 6 etc.) decreased gene of Table 9, Table 10, Table 11, or any combination thereof.

In embodiments, the second gene set includes at least one (e.g., 1, 2, 3, 4, 5, 6 etc.) decreased gene of Table 9. In embodiments, the at least one (e.g., 1, 2, 3, 4, 5, 6 etc.) decreased gene is DYSF, RASAL3, AKR1C3, CGREF1, SULT2B1, CAV2, IL12A, HMGA1, HHLA2, HMX2, CARD11, TSPO, IRF6, CEBPB, BCL11B, CASR, INPP5D, FGF21, NODAL, TNFRSF1B, HPSE, GRPR, TNMD, SPINT2, IER5, CAV1, JAML, SOX10, SFN, NPYSR, MYB, HMOX1, CDH5, HEY2, CLDN7, CXCR2, FGF2, APELA, FLT3LG, CD22, CDCA7L, NPM1, STYK1, SKOR2, LRRC32, HRG, CDH3, IL4R, TERT, ANG, RAB25, NRK, ADM, MARVELD3, DPP4, CD4, LTF, FGF4, ERBB3, IFITM1, P3H2, BAX, WNT11, CEBPA, AVPR1A, PTPRZ1, EIFSA, EPO, NPR1, NQO2, FGF16, EPHAl, CCL26, NR1D1, SYK, PTGES, TCIRG1, HCLS1, RAC2, NME2, TESC, HCK, FZD5, ETS1, APLN, TRIM71, ADA, MYC, GCNT2, SFRP1, FGFR4, EMX1, KDR, RARG, CD74, DRD3, PDPN, TRNP1, HPN, PLAU, TNFSF12, GAS6, SRPX, FGF19, PROK2, TSLP, SHMT2, PIM2, GHRHR, EBI3, ADORA1, NOS3, LIF, PINX1, TNFRSF8, FA2H, LECT1, CHRM1, NME1, SOX15, S100A11, NCCRP1, CD40, SERPINB3, RARRES3, LIN28A, TCL1A, ICOSLG, HYAL1, AIF1, LEP, EEF1E1, PRKCH, VIPR1, IL34, SH2B3, SPINT1, ESRP2, PYCARD, CLEC4G, MATK, EAF2, TACR1, EGFL7, CCNI2, GAL, FERMT1, SFRP5, PPP1R16B, MLXIPL, OVOL1, CD9, TNFSF9, KDF1, MST1R, IL23A, FLT1, FLT3, HLA-G, ADAMTS8, GUCY2C, MMP9, ALOX15B, VDR, SIX4, LGALS3, LAMC2, CCNE1, NPPC, CLC, APOE, MAP3K5, CCND1, XCL1, PTPN6, GLI1, TCL1B, PIM1, ARG2, LYN, NRARP, ELL3, TDGF1, FOSL1, CDCA7, NANOG, CCKBR, BNC1, PNP, TRIB1, HPGD, PRTN3, KIAA1462, HTR1A, BTK, FZD7, IFNLR1, JAK3, CD55, TFAP4, SLA, FBXO2, RBPMS2, OSMR, IL12RB2, EPCAM, IL6, IDO1, CHP2, PTAFR, CXCL1, SFRP2, PF4, CCDC88B, PRKCQ, CXCL5, TGFA, GJA1, FZD5, RPA3, TACSTD2, TNFRSF11A, CNN1, or PTGER2.

In embodiments, the at least one (e.g., 1, 2, 3, 4, 5, 6 etc.) decreased gene is selected from the group consisting of DYSF, RASAL3, AKR1C3, CGREF1, SULT2B1, CAV2, IL12A, HMGA1, HHLA2, HMX2, CARD11, TSPO, IRF6, CEBPB, BCL11B, CASR, INPP5D, FGF21, NODAL, TNFRSF1B, HPSE, GRPR, TNMD, SPINT2, IER5, CAV1, JAML, SOX10, SFN, NPYSR, MYB, HMOX1, CDH5, HEY2, CLDN7, CXCR2, FGF2, APELA, FLT3LG, CD22, CDCA7L, NPM1, STYK1, SKOR2, LRRC32, HRG, CDH3, IL4R, TERT, ANG, RAB25, NRK, ADM, MARVELD3, DPP4, CD4, LTF, FGF4, ERBB3, IFITM1, P3H2, BAX, WNT11, CEBPA, AVPR1A, PTPRZ1, EIFSA, EPO, NPR1, NQO2, FGF16, EPHAl, CCL26, NR1D1, SYK, PTGES, TCIRG1, HCLS1, RAC2, NME2, TESC, HCK, FZD5, ETS1, APLN, TRIM71, ADA, MYC, GCNT2, SFRP1, FGFR4, EMX1, KDR, RARG, CD74, DRD3, PDPN, TRNP1, HPN, PLAU, TNFSF12, GAS6, SRPX, FGF19, PROK2, TSLP, SHMT2, PIM2, GHRHR, EBI3, ADORA1, NOS3, LIF, PINX1, TNFRSF8, FA2H, LECT1, CHRM1, NME1, SOX15, S100A11, NCCRP1, CD40, SERPINB3, RARRES3, LIN28A, TCL1A, ICOSLG, HYAL1, AIF1, LEP, EEF1E1, PRKCH, VIPR1, IL34, SH2B3, SPINT1, ESRP2, PYCARD, CLEC4G, MATK, EAF2, TACR1, EGFL7, CCNI2, GAL, FERMT1, SFRP5, PPP1R16B, MLXIPL, OVOL1, CD9, TNFSF9, KDF1, MST1R, IL23A, FLT1, FLT3, HLA-G, ADAMTS8, GUCY2C, MMP9, ALOX15B, VDR, SIX4, LGALS3, LAMC2, CCNE1, NPPC, CLC, APOE, MAP3K5, CCND1, XCL1, PTPN6, GLI1, TCL1B, PIM1, ARG2, LYN, NRARP, ELL3, TDGF1, FOSL1, CDCA7, NANOG, CCKBR, BNC1, PNP, TRIB1, HPGD, PRTN3, KIAA1462, HTR1A, BTK, FZD7, IFNLR1, JAK3, CD55, TFAP4, SLA, FBXO2, RBPMS2, OSMR, IL12RB2, EPCAM, IL6, IDO1, CHP2, PTAFR, CXCL1, SFRP2, PF4, CCDC88B, PRKCQ, CXCL5, TGFA, GJA1, FZD9, RPA3, TACSTD2, TNFRSF11A, CNN1, and PTGER2.

In embodiments, the second gene set includes at least one (e.g., 1, 2, 3, 4, 5, 6 etc.) decreased gene of Table 10. In embodiments, the at least one (e.g., 1, 2, 3, 4, 5, 6 etc.) decreased gene is C3, AFAP1L2, PTGDR, CMKLR1, CEBPB, NFKBID, TNFRSF1B, SMPDL3B, F2RL1, HMOX1, CXCR2, FPR2, IL17RE, CHST4, IL4R, NFKBIZ, RELB, ADM, ALOX5, SPP1, SIGIRR, EPO, CCL26, SYK, PTGES, TFR2, AHCY, TCIRG1, CHI3L1, UGT1A1, NLRP10, HCK, RARRES2, KLKB1, CXCL2, F12, ALOX15, PROK2, ELF3, ADORA1, CXCL6, CD40, HYAL1, AIF1, ADGRE2, IL34, AHSG, THEMIS2, MMP25, PLSCR1, NMI, PYCARD, TACR1, LBP, GAL, F11R, LY75, IL23A, NRROS, XCL1, ASS1, LYN, BTK, TNFAIP6, IL6, IDO1, PTAFR, CXCL1, PF4, PRKCQ, IL17C, CXCL5, GJA1, CXCL3, PLA2G4C, ICAM1, ORM2, SDC1, PTGER2, or TLR3.

In embodiments, the at least one (e.g., 1, 2, 3, 4, 5, 6 etc.) decreased gene is selected from the group consisting of C3, AFAP1L2, PTGDR, CMKLR1, CEBPB, NFKBID, TNFRSF1B, SMPDL3B, F2RL1, HMOX1, CXCR2, FPR2, IL17RE, CHST4, IL4R, NFKBIZ, RELB, ADM, ALOX5, SPP1, SIGIRR, EPO, CCL26, SYK, PTGES, TFR2, AHCY, TCIRG1, CHI3L1, UGT1A1, NLRP10, HCK, RARRES2, KLKB1, CXCL2, F12, ALOX15, PROK2, ELF3, ADORA1, CXCL6, CD40, HYAL1, AIF1, ADGRE2, IL34, AHSG, THEMIS2, MMP25, PLSCR1, NMI, PYCARD, TACR1, LBP, GAL, F11R, LY75, IL23A, NRROS, XCL1, ASS1, LYN, BTK, TNFAIP6, IL6, IDO1, PTAFR, CXCL1, PF4, PRKCQ, IL17C, CXCL5, GJA1, CXCL3, PLA2G4C, ICAM1, ORM2, SDC1, PTGER2, and TLR3.

In embodiments, the second gene set includes at least one (e.g., 1, 2, 3, 4, 5, 6 etc.) decreased gene of Table 11. In embodiments, the at least one (e.g., 1, 2, 3, 4, 5, 6 etc.) decreased gene is C3, MOG, FOXI3, ACTN3, P2RX2, TWIST2, DYSF, MYBPC2, VSIG1, AKR1C3, CAV2, COL23A1, PTGDR, SLC2A4, RNF43, SHROOM1, BCAN, FGR, LFNG, KRTDAP, GCM2, SEMA4A, SYNGR3, COL13A1, SAMHD1, PDCD1, HMGA1, DSC3, GCNT4, FGF22, SNTG2, HMX2, CARD11, TSPO, IRF6, KLF15, ALAS2, KLK7, KCP, B3GNT2, CMKLR1, ACSBG1, CLDN3, MTHFD1, CEBPB, BCL11B, GDF3, CASR SLC29A1, POU2F3, TBX6, DAZAP1, TIMP4, PVALB, INPP5D, MAL2, NDP, ATXN3, MPZL2, NODAL, TNFRSF1B, BARX1, AFP, HPSE, SOCS1, DDX25, LAMB3, TNMD, BOLL, SPINT2, LPAR3, CAV1, IRF4, SOX10, SFN, NPYSR, MYB, F2RL1, MYBBP1A, HMOX1, TNFAIP2, CCDC85B, RASGRP4, CXCL14, CDH5, CA2, HEY2, ASB2, GNPNAT1, PADI2, RITZ, PCOLCE, CXCR2, FPR2, FGF2, HELLS, HACD1, APELA, LCTL, EVPL, GAB3, FLT3LG, RASAL1, ARC, ACTL8, NPM1, HSPE1, CDH1, SKOR2, ZNF488, RAP1GAP2, CR2, HRG, FABP5, CDH3, PSMB8, FOXD3, SP8, TERT, ANG, SPRR2F, RAMP3, UPK1B, JADE2, TJP2, ETV1, RYR2, RAB25, HSPA2, NRK, RELB, CTSC, INHBB, ANXA3, EPOR, ZFP57, BIK, ADM, DAZL, TM4SF1, PRKCD, CD4 ARTN, POU5F1, LTF, YBX2, SPRY4, EDA, FGF4, FOXA3, NR1I2, SPIB, STAR, FAM65B, ERBB3 ATIC, ARHGAP22, HAPLN3, FRAT2, MPZ, ZMYND15, ARHGAP4, NPAS1, DOCK2, RSPO4, ACAN, TCF15, COL14A1, MTHFD1L, BAX, WNT11, CEBPA, AVPR1A, PTPRZ1, SPP1, ADRA2C, HOOK1, CRYBA4, ANGPT4, SS18L2, BCL11A, CHMP4C, P2RY1, ZIC5, THOC6, NFE2 KRT17, EPO, RPS6KA1, UPK1A, FAM150B, LHCGR, FGF16, DPPA4, KRT7, EPHAl, CNFN, CLRN1, NR1D1, EPAS1, SYK, CHRNA9, PKP1, CLEC4D, PPARGC1B, GRID2, SEMA3G, RAPGEF3, SPTB, GJA5, RCN3, SP7, TCIRG1, CHI3L1, UGT1A1, HCLS1, SSH3, METTLE, RORC, KRTAP13-4, RAC2, KLK13, NME2, TESC, RRS1, HCK, FZD5, NPY1R, CATSPER4, PRRX2, ETS1, ALPL, APLN, ACP5, TRIM71, ADA, RARRES2, PRDX1, S1PR5, MYC, GCNT2, SFRP1, FGFR4, SHISA3, NPTX1, RP11-240B13.2, FOXI2, EMX1, KDR, VWDE, DNMT3B, ALDH1A3, ALDOC, RARG, CD74, TDRD5, FOXG1, DRD3, CDHR1, MFSD2A, PDPN, INSC, RTN4RL2, RAD54L, GABRA5, HESX1, WDR74, TRNP1, HPN, EIF4EBP1, DNAH11, FKBP4, DPPA5, ALOX15, SOHLH2, PHC1, LCP1, STC1, ATOH1, EPHA6, HES3, TNFSF12, GAS6, PKP3, FGF19, PROK2, PAQR5, CBR1, ELF3, M1AP, ITM2A, LAMC3, TEC, LHX6, PHOSPHO1, GHRHR, GJA4, PHLDA3, RGS14, VWA1, SEMG1, VENTX, OSCAR, LRRK1, NKX1-2, ECSCR, ADORAL, ITGAM, NOS3, SLC44A4, PFN1, MOV10L1, ALPK3, LIF, KLK8, TLL2, VILl, TULP1, PHGDH, FA2H, PCDH1, HSPD1, MGST1, ENPP1, LECT1, CHRM1, NME1, SOX15, PLA2G3, MMP17, VWA2, PCSK9, CPNE9, PPP1R13L, KRT15, ADCYAP1R1, PCK2, DOC2A, ARHGEF15, KRT18, ETV4, SRY, CTSV, LIN28A, AQP5, UNC5B, BBC3, GAS1, TCL1A, SLC34A2, NRN1L, NPTX2, HYAL1, AIF1, LEP, PRKCH, KCNQ1, TNNT2, IL34, SH2B3, AHSG, SPINT1, RASIP1, MMP25, P2RX5, GRB7, APRT, VAV1, TNNT1, ESRP2, SLC45A3, MATK, ESRP1, ITGB1BP2, CARMIL2, CLN8, CHAC1, EGFL7, TESMIN, SFRP5, SLC7A5, BATF2, PPP1R16B, TBX22, ADM2, FOXH1, MLXIPL, FOXS1, F11R, CDX4, OVOL1, VSX2, CD9, MME, GJC3, KDF1, FLT1, FLT3, CCDC63, HLA-G, HTR6, CLDN4, TRPC6, UNC13A, ACTN2, NRROS, GJB3, FAM150A, SLC2A14, JPH1, MMP9, ALOX15B, SH3GL3, VDR, SIX4, LGALS3, PRSS8, COL6A3, ZSCAN10, MAG, TRPM2, COL6A2, RAB38, LAMC2, CRABP1, HRH2, NPPC, CLC, MYLPF, KRTAP5-11, S100A4, ZIC2, APOE, LYAR, 0C90, CCND1, KLK4, RXFP1, MB21D1, PTGIS, INHBE, PTPN6, PLCG2, FBL, GLI1, ASST, PACSIN1, TMC1, PIM1, HPRT1, AK4, ARG2, LYN, NRARP, ELL3, TEX19, TDGF1, MESP2, MYOZ1, MT1G, GATA5, FOSL1, FUT9, TAF4B, NANOG, MEI1, CCKBR, ALOX12B, ST14, GNG8, BNC1, KCNJ10, PIWIL3, SYNE4, CCNBlIP1, DLX4, ASNS, TAF7L, SLC6A11, RORB, PAK1IP1, NOTO, HPGD, FOXL2, KRT19, LGR6, WIPF3, MFGE8, PRTN3, CD19, LTBR, FSTL4, FAM101B, MMP19, BTK, KLK5, UST, FZD7, CCM2L, ANOS1, HES2, JAK3, MKX, SLA, SORL1, PLPPR4, FRAS1, DUSP6, TRPV2, ITGB4, RP1-302G2.5, RBPMS2, YBX3, EPCAM, KLF1, IL6, SH2D2A, KREMEN2, THY1, CXCL1, PRDM14, CRYGD, SALL4, GRHL3, UTF1, DPPA3, OLFML3, AHSP, SYPL2, SFRP2, NOS1, TFAP2C, RNF112, LCK, PRKCQ, FHL2, UGT8, TDRD1, MREG, SOCS3, GH2, TGFA, TEAD4, GJA1, FZD9, FAM101A, COL4A1, HCN1, TACSTD2, UNC45B, SOCS2, ICAM1, PODXL, ZFP42, CST6, GAL3ST1, TNFRSF11A, ENG, TNNI3, CD79B, SDC1, TCF21, SPATA16, COL9A3, TLR3, DIAPH2, PREX2, ADAMTS4, TRIM54, or RAC3.

In embodiments, the at least one (e.g., 1, 2, 3, 4, 5, 6 etc.) decreased gene is selected from the group consisting of C3, MOG, FOXI3, ACTN3, P2RX2, TWIST2, DYSF, MYBPC2, VSIG1, AKR1C3, CAV2, COL23A1, PTGDR, SLC2A4, RNF43, SHROOM1, BCAN, FGR, LFNG, KRTDAP, GCM2, SEMA4A, SYNGR3, COL13A1, SAMHD1, PDCD1, HMGA1, DSC3, GCNT4, FGF22, SNTG2, HMX2, CARD11, TSPO, IRF6, KLF15, ALAS2, KLK7, KCP, B3GNT2, CMKLR1, ACSBG1, CLDN3, MTHFD1, CEBPB, BCL11B, GDF3, CASR SLC29A1, POU2F3, TBX6, DAZAP1, TIMP4, PVALB, INPP5D, MAL2, NDP, ATXN3, MPZL2, NODAL, TNFRSF1B, BARX1, AFP, HPSE, SOCS1, DDX25, LAMB3, TNMD, BOLL, SPINT2, LPAR3, CAV1, IRF4, SOX10, SFN, NPYSR, MYB, F2RL1, MYBBP1A, HMOX1, TNFAIP2, CCDC85B, RASGRP4, CXCL14, CDH5, CA2, HEY2, ASB2, GNPNAT1, PADI2, RITZ, PCOLCE, CXCR2, FPR2, FGF2, HELLS, HACD1, APELA, LCTL, EVPL, GAB3, FLT3LG, RASAL1, ARC, ACTL8, NPM1, HSPE1, CDH1, SKOR2, ZNF488, RAP1GAP2, CR2, HRG, FABP5, CDH3, PSMB8, FOXD3, SP8, TERT, ANG, SPRR2F, RAMP3, UPK1B, JADE2, TJP2, ETV1, RYR2, RAB25, HSPA2, NRK, RELB, CTSC, INHBB, ANXA3, EPOR, ZFP57, BIK, ADM, DAZL, TM4SF1, PRKCD, CD4 ARTN, POU5F1, LTF, YBX2, SPRY4, EDA, FGF4, FOXA3, NR1I2, SPIB, STAR, FAM65B, ERBB3 ATIC, ARHGAP22, HAPLN3, FRAT2, MPZ, ZMYND15, ARHGAP4, NPAS1, DOCK2, RSPO4, ACAN, TCF15, COL14A1, MTHFD1L, BAX, WNT11, CEBPA, AVPR1A, PTPRZ1, SPP1, ADRA2C, HOOK1, CRYBA4, ANGPT4, SS18L2, BCL11A, CHMP4C, P2RY1, ZIC5, THOC6, NFE2 KRT17, EPO, RPS6KA1, UPK1A, FAM150B, LHCGR, FGF16, DPPA4, KRT7, EPHAl, CNFN, CLRN1, NR1D1, EPAS1, SYK, CHRNA9, PKP1, CLEC4D, PPARGC1B, GRID2, SEMA3G, RAPGEF3, SPTB, GJA5, RCN3, SP7, TCIRG1, CHI3L1, UGT1A1, HCLS1, SSH3, METTLE, RORC, KRTAP13-4, RAC2, KLK13, NME2, TESC, RRS1, HCK, FZD5, NPY1R, CATSPER4, PRRX2, ETS1, ALPL, APLN, ACP5, TRIM71, ADA, RARRES2, PRDX1, S1PR5, MYC, GCNT2, SFRP1, FGFR4, SHISA3, NPTX1, RP11-240B13.2, FOXI2, EMX1, KDR, VWDE, DNMT3B, ALDH1A3, ALDOC, RARG, CD74, TDRD5, FOXG1, DRD3, CDHR1, MFSD2A, PDPN, INSC, RTN4RL2, RAD54L, GABRA5, HESX1, WDR74, TRNP1, HPN, EIF4EBP1, DNAH11, FKBP4, DPPA5, ALOX15, SOHLH2, PHC1, LCP1, STC1, ATOH1, EPHA6, HES3, TNFSF12, GAS6, PKP3, FGF19, PROK2, PAQR5, CBR1, ELF3, M1AP, ITM2A, LAMC3, TEC, LHX6, PHOSPHO1, GHRHR, GJA4, PHLDA3, RGS14, VWA1, SEMG1, VENTX, OSCAR, LRRK1, NKX1-2, ECSCR, ADORAL, ITGAM, NOS3, SLC44A4, PFN1, MOV10L1, ALPK3, LIF, KLK8, TLL2, VILl, TULP1, PHGDH, FA2H, PCDH1, HSPD1, MGST1, ENPP1, LECT1, CHRM1, NME1, SOX15, PLA2G3, MMP17, VWA2, PCSK9, CPNE9, PPP1R13L, KRT15, ADCYAP1R1, PCK2, DOC2A, ARHGEF15, KRT18, ETV4, SRY, CTSV, LIN28A, AQP5, UNC5B, BBC3, GAS1, TCL1A, SLC34A2, NRN1L, NPTX2, HYAL1, AIF1, LEP, PRKCH, KCNQ1, TNNT2, IL34, SH2B3, AHSG, SPINT1, RASIP1, MMP25, P2RX5, GRB7, APRT, VAV1, TNNT1, ESRP2, SLC45A3, MATK, ESRP1, ITGB1BP2, CARMIL2, CLN8, CHAC1, EGFL7, TESMIN, SFRP5, SLC7A5, BATF2, PPP1R16B, TBX22, ADM2, FOXH1, MLXIPL, FOXS1, F11R, CDX4, OVOL1, VSX2, CD9, MME, GJC3, KDF1, FLT1, FLT3, CCDC63, HLA-G, HTR6, CLDN4, TRPC6, UNC13A, ACTN2, NRROS, GJB3, FAM150A, SLC2A14, JPH1, MMP9, ALOX15B, SH3GL3, VDR, SIX4, LGALS3, PRSS8, COL6A3, ZSCAN10, MAG, TRPM2, COL6A2, RAB38, LAMC2, CRABP1, HRH2, NPPC, CLC, MYLPF, KRTAP5-11, S100A4, ZIC2, APOE, LYAR, 0C90, CCND1, KLK4, RXFP1, MB21D1, PTGIS, INHBE, PTPN6, PLCG2, FBL, GLI1, ASS1, PACSIN1, TMC1, PIM1, HPRT1, AK4, ARG2, LYN, NRARP, ELL3, TEX19, TDGF1, MESP2, MYOZ1, MT1G, GATA5, FOSL1, FUT9, TAF4B, NANOG, MEI1, CCKBR, ALOX12B, ST14, GNG8, BNC1, KCNJ10, PIWIL3, SYNE4, CCNB1IP1, DLX4, ASNS, TAF7L, SLC6A11, RORB, PAK1IP1, NOTO, HPGD, FOXL2, KRT19, LGR6, WIPF3, MFGE8, PRTN3, CD19, LTBR, FSTL4, FAM101B, MMP19, BTK, KLK5, UST, FZD7, CCM2L, ANOS1, HES2, JAK3, MKX, SLA, SORL1, PLPPR4, FRAS1, DUSP6, TRPV2, ITGB4, RP1-302G2.5, RBPMS2, YBX3, EPCAM, KLF1, IL6, SH2D2A, KREMEN2, THY1, CXCL1, PRDM14, CRYGD, SALL4, GRHL3, UTF1, DPPA3, OLFML3, AHSP, SYPL2, SFRP2, NOS1, TFAP2C, RNF112, LCK, PRKCQ, FHL2, UGT8, TDRD1, MREG, SOCS3, GH2, TGFA, TEAD4, GJA1, FZD9, FAM101A, COL4A1, HCN1, TACSTD2, UNC45B, SOCS2, ICAM1, PODXL, ZFP42, CST6, GAL3ST1, TNFRSF11A, ENG, TNNI3, CD79B, SDC1, TCF21, SPATA16, COL9A3, TLR3, DIAPH2, PREX2, ADAMTS4, TRIM54, or RAC3. C3, MOG, FOXI3, ACTN3, P2RX2, TWIST2, DYSF, MYBPC2, VSIG1, AKR1C3, CAV2, COL23A1, PTGDR, SLC2A4, RNF43, SHROOM1, BCAN, FGR, LFNG, KRTDAP, GCM2, SEMA4A, SYNGR3, COL13A1, SAMHD1, PDCD1, HMGA1, DSC3, GCNT4, FGF22, SNTG2, HMX2, CARD11, TSPO, IRF6, KLF15, ALAS2, KLK7, KCP, B3GNT2, CMKLR1, ACSBG1, CLDN3, MTHFD1, CEBPB, BCL11B, GDF3, CASR SLC29A1, POU2F3, TBX6, DAZAP1, TIMP4, PVALB, INPP5D, MAL2, NDP, ATXN3, MPZL2, NODAL, TNFRSF1B, BARX1, AFP, HPSE, SOCS1, DDX25, LAMB3, TNMD, BOLL, SPINT2, LPAR3, CAV1, IRF4, SOX10, SFN, NPY5R, MYB, F2RL1, MYBBP1A, HMOX1, TNFAIP2, CCDC85B, RASGRP4, CXCL14, CDH5, CA2, HEY2, ASB2, GNPNAT1, PADI2, RITZ, PCOLCE, CXCR2, FPR2, FGF2, HELLS, HACD1, APELA, LCTL, EVPL, GAB3, FLT3LG, RASAL1, ARC, ACTL8, NPM1, HSPE1, CDH1, SKOR2, ZNF488, RAP1GAP2, CR2, HRG, FABP5, CDH3, PSMB8, FOXD3, SP8, TERT, ANG, SPRR2F, RAMP3, UPK1B, JADE2, TJP2, ETV1, RYR2, RAB25, HSPA2, NRK, RELB, CTSC, INHBB, ANXA3, EPOR, ZFP57, BIK, ADM, DAZL, TM4SF1, PRKCD, CD4 ARTN, POU5F1, LTF, YBX2, SPRY4, EDA, FGF4, FOXA3, NR1I2, SPIB, STAR, FAM65B, ERBB3 ATIC, ARHGAP22, HAPLN3, FRAT2, MPZ, ZMYND15, ARHGAP4, NPAS1, DOCK2, RSPO4, ACAN, TCF15, COL14A1, MTHFD1L, BAX, WNT11, CEBPA, AVPR1A, PTPRZ1, SPP1, ADRA2C, HOOK1, CRYBA4, ANGPT4, SS18L2, BCL11A, CHMP4C, P2RY1, ZIC5, THOC6, NFE2 KRT17, EPO, RPS6KA1, UPK1A, FAM150B, LHCGR, FGF16, DPPA4, KRT7, EPHAl, CNFN, CLRN1, NR1D1, EPAS1, SYK, CHRNA9, PKP1, CLEC4D, PPARGC1B, GRID2, SEMA3G, RAPGEF3, SPTB, GJA5, RCN3, SP7, TCIRG1, CHI3L1, UGT1A1, HCLS1, SSH3, METTLE, RORC, KRTAP13-4, RAC2, KLK13, NME2, TESC, RRS1, HCK, FZD5, NPY1R, CATSPER4, PRRX2, ETS1, ALPL, APLN, ACP5, TRIM71, ADA, RARRES2, PRDX1, S1PR5, MYC, GCNT2, SFRP1, FGFR4, SHISA3, NPTX1, RP11-240B13.2, FOXI2, EMX1, KDR, VWDE, DNMT3B, ALDH1A3, ALDOC, RARG, CD74, TDRD5, FOXG1, DRD3, CDHR1, MFSD2A, PDPN, INSC, RTN4RL2, RAD54L, GABRA5, HESX1, WDR74, TRNP1, HPN, EIF4EBP1, DNAH11, FKBP4, DPPA5, ALOX15, SOHLH2, PHC1, LCP1, STC1, ATOH1, EPHA6, HES3, TNFSF12, GAS6, PKP3, FGF19, PROK2, PAQR5, CBR1, ELF3, M1AP, ITM2A, LAMC3, TEC, LHX6, PHOSPHO1, GHRHR, GJA4, PHLDA3, RGS14, VWA1, SEMG1, VENTX, OSCAR, LRRK1, NKX1-2, ECSCR, ADORAL, ITGAM, NOS3, SLC44A4, PFN1, MOV10L1, ALPK3, LIF, KLK8, TLL2, VILl, TULP1, PHGDH, FA2H, PCDH1, HSPD1, MGST1, ENPP1, LECT1, CHRM1, NME1, SOX15, PLA2G3, MMP17, VWA2, PCSK9, CPNE9, PPP1R13L, KRT15, ADCYAP1R1, PCK2, DOC2A, ARHGEF15, KRT18, ETV4, SRY, CTSV, LIN28A, AQP5, UNC5B, BBC3, GAS1, TCL1A, SLC34A2, NRN1L, NPTX2, HYAL1, AIF1, LEP, PRKCH, KCNQ1, TNNT2, IL34, SH2B3, AHSG, SPINT1, RASIP1, MMP25, P2RX5, GRB7, APRT, VAV1, TNNT1, ESRP2, SLC45A3, MATK, ESRP1, ITGB1BP2, CARMIL2, CLN8, CHAC1, EGFL7, TESMIN, SFRP5, SLC7A5, BATF2, PPP1R16B, TBX22, ADM2, FOXH1, MLXIPL, FOXS1, F11R, CDX4, OVOL1, VSX2, CD9, MME, GJC3, KDF1, FLT1, FLT3, CCDC63, HLA-G, HTR6, CLDN4, TRPC6, UNC13A, ACTN2, NRROS, GJB3, FAM150A, SLC2A14, JPH1, MMP9, ALOX15B, SH3GL3, VDR, SIX4, LGALS3, PRSS8, COL6A3, ZSCAN10, MAG, TRPM2, COL6A2, RAB38, LAMC2, CRABP1, HRH2, NPPC, CLC, MYLPF, KRTAP5-11, S100A4, ZIC2, APOE, LYAR, 0C90, CCND1, KLK4, RXFP1, MB21D1, PTGIS, INHBE, PTPN6, PLCG2, FBL, GLI1, ASS1, PACSIN1, TMC1, PIM1, HPRT1, AK4, ARG2, LYN, NRARP, ELL3, TEX19, TDGF1, MESP2, MYOZ1, MT1G, GATA5, FOSL1, FUT9, TAF4B, NANOG, MEI1, CCKBR, ALOX12B, ST14, GNG8, BNC1, KCNJ10, PIWIL3, SYNE4, CCNB1IP1, DLX4, ASNS, TAF7L, SLC6A11, RORB, PAK1IP1, NOTO, HPGD, FOXL2, KRT19, LGR6, WIPF3, MFGE8, PRTN3, CD19, LTBR, FSTL4, FAM101B, MMP19, BTK, KLK5, UST, FZD7, CCM2L, ANOS1, HES2, JAK3, MKX, SLA, SORL1, PLPPR4, FRAS1, DUSP6, TRPV2, ITGB4, RP1-302G2.5, RBPMS2, YBX3, EPCAM, KLF1, IL6, SH2D2A, KREMEN2, THY1, CXCL1, PRDM14, CRYGD, SALL4, GRHL3, UTF1, DPPA3, OLFML3, AHSP, SYPL2, SFRP2, NOS1, TFAP2C, RNF112, LCK, PRKCQ, FHL2, UGT8, TDRD1, MREG, SOCS3, GH2, TGFA, TEAD4, GJA1, FZD9, FAM101A, COL4A1, HCN1, TACSTD2, UNC45B, SOCS2, ICAM1, PODXL, ZFP42, CST6, GAL3ST1, TNFRSF11A, ENG, TNNI3, CD79B, SDC1, TCF21, SPATA16, COL9A3, TLR3, DIAPH2, PREX2, ADAMTS4, TRIM54, and RAC3.

In embodiments, the at least one decreased gene is selected from the group consisting of: ADCY8, AKR1C3, ALDH3A1, APRT, ASNS, BAX, BBC3, CCND1, CDH5, CH25H, CMKLR1, COL16A1, CXCL1, CXCL2, EDNRB, EEF1E1, RIPOR2, FGF10, FGF22, FZD7, GJA1, GNG8, GNPNAT1, HPGD, ICAM1, ITPR2, KLF1, KLF15, LEP, LPL, LRRC32, MAP3K5, MX1, MYC, NME1, NME2, NQO2, NR1D1, P2RY1, PCOLCE2, PDE4A, PDIA5, PFKP, PHGDH, PLK5, PPP1R14A, PRODH, PSMB8, PSMB9, PYCR1, RAPGEF3, RYR2, SCARB1, SHMT2, SIPA1, SPHK1, TRIM22, VDR, ADA, ADGRG3, ADGRL4, ANK1, ART3, CAll, CABP1, CDH15, CDHR1, COL13A1, EPHA6, CALHM6, GRID2IP, HS3ST3B1, ICAM5, JCAD, LGR6, LRRC38, NOXO1, PDPN, PLPPR5, PODXL, RAMP3, RGS7BP, RIMS4, RTBDN, RTN4RL2, S100A10, SEMA4A, SGCG, SH2D5, SHISA9, SHROOM1, SLC22A3, SLC24A2, SLC29A2, SLC6A11, SLC7A10, SLC7A5, SLCO2A1, STAC2, STYK1, TMC1, UNC13A, WWC1, ABCG2, ACSBG1, ACSS1, ACY1, AHCY, ALOX12B, AMD1, ARG2, ASS1, BCAT1, CHST2, CLN8, ENTPD2, FABP5, FADS3, FUT4, FUT9, GAL3ST3, GMDS, HACD1, HAS3, HPD, KYAT1, LDHD, MPP1, OGDHL, PDE4A, PGM1, PIPDX, PLAAT3, PLA2G4C, PLCB3, PNP, PSAT1, PTGES, REXO2, SCARB1, SLC27A6, SPHK1, STAB2, UAP1L1 and UCK2. In embodiments, the at least one decreased gene is ADCY8, AKR1C3, ALDH3A1, APRT, ASNS, BAX, BBC3, CCND1, CDH5, CH25H, CMKLR1, COL16A1, CXCL1, CXCL2, EDNRB, EEF1E1, RIPOR2, FGF10, FGF22, FZD7, GJA1, GNG8, GNPNAT1, HPGD, ICAM1, ITPR2, KLF1, KLF15, LEP, LPL, LRRC32, MAP3K5, MX1, MYC, NME1, NME2, NQO2, NR1D1, P2RY1, PCOLCE2, PDE4A, PDIA5, PFKP, PHGDH, PLK5, PPP1R14A, PRODH, PSMB8, PSMB9, PYCR1, RAPGEF3, RYR2, SCARB1, SHMT2, SIPA1, SPHK1, TRIM22, VDR, ADA, ADGRG3, ADGRL4, ANK1, ART3, CAll, CABP1, CDH15, CDHR1, COL13A1, EPHA6, CALHM6, GRID2IP, HS3ST3B1, ICAM5, JCAD, LGR6, LRRC38, NOXO1, PDPN, PLPPR5, PODXL, RAMP3, RGS7BP, RIMS4, RTBDN, RTN4RL2, S100A10, SEMA4A, SGCG, SH2D5, SHISA9, SHROOM1, SLC22A3, SLC24A2, SLC29A2, SLC6A11, SLC7A10, SLC7A5, SLCO2A1, STAC2, STYK1, TMC1, UNC13A, WWC1, ABCG2, ACSBG1, ACSS1, ACY1, AHCY, ALOX12B, AMD1, ARG2, ASS1, BCAT1, CHST2, CLN8, ENTPD2, FABP5, FADS3, FUT4, FUT9, GAL3ST3, GMDS, HACD1, HAS3, HPD, KYAT1, LDHD, MPP1, OGDHL, PDE4A, PGM1, PIPDX, PLAAT3, PLA2G4C, PLCB3, PNP, PSAT1, PTGES, REXO2, SCARB1, SLC27A6, SPHK1, STAB2, UAP1L1 or UCK2.

In embodiments, the decreased expression levels are at least 4 times lower relative to a pluripotent stem cell. In embodiments, the decreased expression levels are about 4 times lower relative to a pluripotent stem cell. In embodiments, the decreased expression levels are at least 5 times lower relative to a pluripotent stem cell. In embodiments, the decreased expression levels are about 5 times lower relative to a pluripotent stem cell. In embodiments, the decreased expression levels are at least 6 times lower relative to a pluripotent stem cell. In embodiments, the decreased expression levels are about 6 times lower relative to a pluripotent stem cell. In embodiments, the decreased expression levels are at least 7 times lower relative to a pluripotent stem cell. In embodiments, the decreased expression levels are about 7 times lower relative to a pluripotent stem cell. In embodiments, the decreased expression levels are at least 8 times lower relative to a pluripotent stem cell. In embodiments, the decreased expression levels are about 8 times lower relative to a pluripotent stem cell. In embodiments, the decreased expression levels are at least 9 times lower relative to a pluripotent stem cell. In embodiments, the decreased expression levels are about 9 times lower relative to a pluripotent stem cell. In embodiments, the decreased expression levels are at least 10 times lower relative to a pluripotent stem cell. In embodiments, the decreased expression levels are about 10 times lower relative to a pluripotent stem cell.

In embodiments, the decreased expression levels are at least 11 times lower relative to a pluripotent stem cell. In embodiments, the decreased expression levels are about 11 times lower relative to a pluripotent stem cell. In embodiments, the decreased expression levels are at least 12 times lower relative to a pluripotent stem cell. In embodiments, the decreased expression levels are about 12 times lower relative to a pluripotent stem cell. In embodiments, the decreased expression levels are at least 13 times lower relative to a pluripotent stem cell. In embodiments, the decreased expression levels are about 13 times lower relative to a pluripotent stem cell. In embodiments, the decreased expression levels are at least 14 times lower relative to a pluripotent stem cell. In embodiments, the decreased expression levels are about 14 times lower relative to a pluripotent stem cell. In embodiments, the decreased expression levels are at least 15 times lower relative to a pluripotent stem cell. In embodiments, the decreased expression levels are about 15 times lower relative to a pluripotent stem cell. In embodiments, the decreased expression levels are at least 16 times lower relative to a pluripotent stem cell. In embodiments, the decreased expression levels are about 16 times lower relative to a pluripotent stem cell. In embodiments, the decreased expression levels are at least 17 times lower relative to a pluripotent stem cell. In embodiments, the decreased expression levels are about 17 times lower relative to a pluripotent stem cell. In embodiments, the decreased expression levels are at least 18 times lower relative to a pluripotent stem cell. In embodiments, the decreased expression levels are about 18 times lower relative to a pluripotent stem cell. In embodiments, the decreased expression levels are at least 19 times lower relative to a pluripotent stem cell. In embodiments, the decreased expression levels are about 19 times lower relative to a pluripotent stem cell. In embodiments, the decreased expression levels are at least 20 times lower relative to a pluripotent stem cell. In embodiments, the decreased expression levels are about 20 times lower relative to a pluripotent stem cell.

In embodiments, the decreased expression levels are about 4-100 times lower relative to a pluripotent stem cell. In embodiments, the decreased expression levels are 4-100 times lower relative to a pluripotent stem cell. In embodiments, the decreased expression levels are about 6-100 times lower relative to a pluripotent stem cell. In embodiments, the decreased expression levels are 6-100 times lower relative to a pluripotent stem cell. In embodiments, the decreased expression levels are about 8-100 times lower relative to a pluripotent stem cell. In embodiments, the decreased expression levels are 8-100 times lower relative to a pluripotent stem cell. In embodiments, the decreased expression levels are about 10-100 times lower relative to a pluripotent stem cell. In embodiments, the decreased expression levels are 10-100 times lower relative to a pluripotent stem cell. In embodiments, the decreased expression levels are about 20-100 times lower relative to a pluripotent stem cell. In embodiments, the decreased expression levels are 20-100 times lower relative to a pluripotent stem cell. In embodiments, the decreased expression levels are about 30-100 times lower relative to a pluripotent stem cell. In embodiments, the decreased expression levels are 30-100 times lower relative to a pluripotent stem cell. In embodiments, the decreased expression levels are about 40-100 times lower relative to a pluripotent stem cell. In embodiments, the decreased expression levels are 40-100 times lower relative to a pluripotent stem cell. In embodiments, the decreased expression levels are about 50-100 times lower relative to a pluripotent stem cell. In embodiments, the decreased expression levels are 50-100 times lower relative to a pluripotent stem cell. In embodiments, the decreased expression levels are about 60-100 times lower relative to a pluripotent stem cell. In embodiments, the decreased expression levels are 60-100 times lower relative to a pluripotent stem cell. In embodiments, the decreased expression levels are about 70-100 times lower relative to a pluripotent stem cell. In embodiments, the decreased expression levels are 70-100 times lower relative to a pluripotent stem cell. In embodiments, the decreased expression levels are about 80-100 times lower relative to a pluripotent stem cell. In embodiments, the decreased expression levels are 80-100 times lower relative to a pluripotent stem cell. In embodiments, the decreased expression levels are about 90-100 times lower relative to a pluripotent stem cell. In embodiments, the decreased expression levels are 90-100 times lower relative to a pluripotent stem cell.

In embodiments, the decreased expression levels are about 4-90 times lower relative to a pluripotent stem cell. In embodiments, the decreased expression levels are 4-90 times lower relative to a pluripotent stem cell. In embodiments, the decreased expression levels are about 4-80 times lower relative to a pluripotent stem cell. In embodiments, the decreased expression levels are 4-80 times lower relative to a pluripotent stem cell. In embodiments, the decreased expression levels are about 4-70 times lower relative to a pluripotent stem cell. In embodiments, the decreased expression levels are 4-70 times lower relative to a pluripotent stem cell. In embodiments, the decreased expression levels are about 4-60 times lower relative to a pluripotent stem cell. In embodiments, the decreased expression levels are 4-60 times lower relative to a pluripotent stem cell. In embodiments, the decreased expression levels are about 4-50 times lower relative to a pluripotent stem cell. In embodiments, the decreased expression levels are 4-50 times lower relative to a pluripotent stem cell. In embodiments, the decreased expression levels are about 4-40 times lower relative to a pluripotent stem cell. In embodiments, the decreased expression levels are 4-40 times lower relative to a pluripotent stem cell. In embodiments, the decreased expression levels are about 4-30 times lower relative to a pluripotent stem cell. In embodiments, the decreased expression levels are 4-30 times lower relative to a pluripotent stem cell. In embodiments, the decreased expression levels are about 4-20 times lower relative to a pluripotent stem cell. In embodiments, the decreased expression levels are 4-20 times lower relative to a pluripotent stem cell. In embodiments, the decreased expression levels are about 4-10 times lower relative to a pluripotent stem cell. In embodiments, the decreased expression levels are 4-10 times lower relative to a pluripotent stem cell. In embodiments, the decreased expression levels are about 4-8 times lower relative to a pluripotent stem cell. In embodiments, the decreased expression levels are 4-8 times lower relative to a pluripotent stem cell. In embodiments, the decreased expression levels are about 4-6 times lower relative to a pluripotent stem cell. In embodiments, the decreased expression levels are 4-6 times lower relative to a pluripotent stem cell.

In embodiments, the gene expression profile information for the desirable determined dopaminergic precursor cell comprises an undesirable gene expression profile comprising one or more undesirable genes. In embodiments, the one or more undesirable genes is a cancer marker gene. In embodiments, the one or more undesirable genes is a tyrosine hydroxylase gene. An “undesirable gene” is a gene characterisitic for a non-dopaminergic cell or a non non-dopaminergic neuron. A “non-dopaminergic cell” or a “non-dopaminergic neuron” is a cell that lacks biological features of a dopaminergic neuron (e.g., does not express dopamine) Examples of non-dopaminergic neurons include without limitation, GABAergic cells, serotonergic neurons, non-A9 dopaminergic neurons, an ependymal cell, an astrocyte, a microglial cell or an oligodendrocyte. In embodiments, the non-dopaminergic neuron does not express detectable amounts of dopamine. In embodiments, the non-dopaminergic neuron expresses tyrosine hydroxylase.

### IV. Pharmaceutical Compositions and Formulations

Also provided herein are populations of cells identified as comprising a neuronal progenitor cell population identified based on the classification methods provided heren. For example, provided herein are populations of cells identified as comprising determined dopaminergic precursor cells (identified, e.g., by the methods provided herein). In some embodiments, a dose of such identified cells is provided as a composition or formulation, such as a pharmaceutical composition or formulation. In some embodiments, the dose of cells comprises differentiated cells, for instance cells differentiated according to any of the methods described in Section I.A.2. herein. In some embodiments, the dose of cells is identified as comprising determined dopaminergic precursor cells according to any of the methods described in Section I.F. herein.

Such compositions can be used in accord with the provided methods, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the substantia nigra.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g. the substantia nigra).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes

### V. Methods of Treatment

Also provided herein are methods of treating involving administration of a neuronal progenitor cell population identified based on the classification methods provided heren to a subject having a neurodegenerative disease in need of treatment thereof. In some embodiments, the a population of neuronal progenitor cells that are determined dopaminergic precursor cells are identified, (e.g., by the methods provided herein), and the method further includes administering the determined dopaminergic precursor cell to a subject in need thereof. Also provided herein are uses of any of the provided compositions or populations of neuronal progenitor cells, e.g. determined dopaminergic precursor cells, in such methods and treatments, and in the preparation of a medicament in order to carry out such therapeutic methods. In some embodiments, the methods thereby treat the neurodegenerative disease in the subject. Also provided herein are uses of any of the compositions, such as pharmaceutical compositions provided herein, for the treatment of a neurodegenerative disease. In embodiments, the subject suffers from a neurodegenerative disease. In embodiments, the subject suffers from Parkinson's Disease. In some embodiments, the determined dopaminergic precursor cells are differentiated from PSCs (e.g. iPSCs) autologous to the subject to be treated, i.e. the PSCs are derived from the same subject to whom the differentiated cells are administered.

In some embodiments, non-pluripotent cells (e.g., fibroblasts) derived from patients having Parkinson's disease (PD) are reprogrammed to become iPSCs, such as in accord with differentiation processes as described in Section II. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become iPSCs before differentiation into determined DA neuron progenitors cells and/or DA neurons, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous stem cell transplant. In some embodiments, the PSCs (e.g., iPSCs) are allogeneic to the subject to be treated, i.e. the PSCs are derived from a different individual than the subject to whom the differentiated cells will be administered. In some embodiments, non-pluripotent cells (e.g., fibroblasts) derived from another individual (e.g. an individual not having a neurodegenerative disorder, such as Parkinson's disease) are reprogrammed to become iPSCs before differentiation into determined DA neuron progenitor cells and/or DA neurons. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting differentiated cells are then administered to an individual who is not the same individual from whom the differentiated cells are derived (e.g. allogeneic cell therapy or allogeneic cell transplantation).

In some embodiments, the subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is Parkinsonism. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has Parkinson's Disease. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, the therapeutic composition comprising cells identified as comprising determined dopaminergic precursor cells is administered to treat a neurodegenerative disease, e.g., PD. In some embodiments, the dose of cells is a dose of a composition of cells, e.g., as described in Section III herein.

In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the substantia nigra of the subject. In some embodiments, the dose of cells is administered to one hemisphere of the subject's substantia nigra. In some embodiments, the dose of cells is administered to both hemispheres of the subject's substantia nigra.

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,000 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g. differentiated cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some aspects, the size of the dose is determined based on one or more criteria such as response of the subject to prior treatment, e.g. disease type and/or stage, and/or likelihood or incidence of the subject developing toxic outcomes, e.g., dyskinesia.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi nlm nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “gene ontology” or “gene ontologies” as provided herein are used according to their common meaning in the biological and bioinformatics arts, wherein a gene ontology is a representation of genes, gene expressions and gene properties and their relationships to each other. A gene ontology may include a cellular component (the parts of a cell or its extracellular environment), a molecular function (the elemental activities of a gene product at the molecular level, such as binding or catalysis) and a biological process (operations or sets of molecular events with a defined beginning and end, pertinent to the functioning of integrated living units such as cells, tissues, organs, and organisms). Each GO term within an ontology has a term name, which may be a word or string of words; a unique alphanumeric identifier; a definition with cited sources; and a namespace indicating the domain to which it belongs.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “isolated” may also refer to a cell or sample cells. An isolated cell or sample cells are a single cell type that is substantially free of many of the components which normally accompany the cells when they are in their native state or when they are initially removed from their native state. In certain embodiments, an isolated cell sample retains those components from its natural state that are required to maintain the cell in a desired state. In some embodiments, an isolated (e.g. purified, separated) cell or isolated cells, are cells that are substantially the only cell type in a sample. A purified cell sample may contain at least 60%, 70%, 75%, 80%, 85%, 90%, 95%, 96%, 97%, 98%, 99%, or 100% of one type of cell. An isolated cell sample may be obtained through the use of a cell marker or a combination of cell markers, either of which is unique to one cell type in an unpurified cell sample.

The term “purified” denotes that a nucleic acid or protein gives rise to essentially one band in an electrophoretic gel. In some embodiments, the nucleic acid or protein is at least 50% pure, optionally at least 65% pure, optionally at least 75% pure, optionally at least 85% pure, optionally at least 95% pure, and optionally at least 99% pure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

“Self renewal” refers to the ability of a cell to divide and generate at least one daughter cell with the self-renewing characteristics of the parent cell. The second daughter cell may commit to a particular differentiation pathway. For example, a self-renewing hematopoietic stem cell can divide and form one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Non-self-renewing cells refers to cells that undergo cell division to produce daughter cells, neither of which have the differentiation potential of the parent cell type, but instead generates differentiated daughter cells.

An adult stem cell is an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited ability to self renew and generate progeny of distinct cell types. Adult stem cells can include hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

The term “reprogramming” refers to the process of dedifferentiating a non-pluripotent cell into a cell exhibiting pluripotent stem cell characteristics.

A “cell culture” is an in vitro population of cells residing outside of an organism. The cell culture can be established from primary cells isolated from a cell bank or animal, or secondary cells that are derived from one of these sources and immortalized for long-term in vitro cultures.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. For example, in embodiments, the term “expand” refers to the differentiation of an iPSC in vitro. Cells are typically cultured/expanded in media, which can be changed during the course of the culture. The terms “medium,” “media” and “culture solution” refer to the cell culture milieu. Media is typically an isotonic solution, and can be liquid, gelatinous, or semisolid, e.g., to provide a matrix for cell adhesion or support. Media, as used herein, can include the components for nutritional, chemical, and structural support necessary for culturing a cell. The term “media” refers to a solution that includes various components including without limitation inorganic salts, amino acids, vitamins, growth factors, and other protein components. As used herein, “conditions to allow growth” in culture and the like refers to conditions of temperature (typically at about 37° C. for mammalian cells), humidity, CO2 (typically around 5%), in appropriate media (including salts, buffer, serum), such that the cells are able to undergo cell division or at least maintain viability for at least 24 hours, preferably longer (e.g., for days, weeks or months). The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

Where appropriate the expanding of iPSC may be subjected to a process of selection. A process of selection may include a selection marker introduced into an induced pluripotent stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected induced pluripotent stem cell the ability to expand in the presence of a toxin. Such a toxin typically inhibits cell expansion and/or causes cell death. Examples of such toxins include, but are not limited to, hygromycin, neomycin, puromycin and gentamycin. In embodiments, the toxin is hygromycin. Through the enzymatic activity of a selection maker a toxin may be converted to a non-toxin, which no longer inhibits expansion and causes cell death of a transfected induced pluripotent stem cell. Upon exposure to a toxin a cell lacking a selection marker may be eliminated and thereby precluded from expansion.

Identification of the induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics. The term “hiPSC-derived neuronal cell” refers to a neuronal progenitor cell (NPC) or a mature neuron that has been derived (e.g., differentiated) from a hiPSC cell in vitro. The hiPSCs can be differentiated by any appropriate method known in the art.

The development of an embryo can be described as self-assembly. The mother and fetus have closely associated blood vessels so that the fetus can be nourished during development, but the embryo develops by itself, through a series of cell-cell interactions that direct the fate of cells that then influence the fate of other cells. As the embryo develops, cells narrow their possible fates, until only one fate remains. During embryogenesis a pluripotent cell matures through specific stages that cumulatively commit it to a specific fate: first specification, then determination, and finally differentiation.

The term “specification” or “specified” as provided herein refers to the fate of a cell or tissue narrowed to a limited number of specific cell types. A specified cell can still change its specific fate until it reaches the determined state, in which it has only one choice of cell type it can differentiate into.

The term “determination” or “determined” as provided herein refers to a cell or tissue capable of differentiating autonomously even when placed into another region of the embryo or a cluster of differently specified cells in a petri dish.

The term “differentiation” or “differentiate” as provided herein refers to a cell or cells that have acquired a cell type-specific function.

A “specified state” as provided herein refers to cells that can be influenced by their environment but have limited fate options. For example, a bit of ectoderm can be transplanted to another part of the embryo and will interpret the surrounding signals in ectodermal terms and can form many types of neurons, glia, or skin.

A “determined state” as determined herein refers to a cell having a narrow range of fates. For example, determined ventral mesencephalic dopamine neuron precursors cannot make other types of neurons. They are not yet neurons themselves and may or may not express the definitive markers of specific cell types.

A “neuronal progenitor cell” is a cell that has a tendency to differentiate into a neuronal cell and does not have the pluripotent potential of a stem cell. A neuronal progenitor is a cell that is committed to the neuronal lineage and is characterized by expressing one or more marker genes that are specific for the neuronal lineage. Examples of neuronal lineage marker genes are N-CAM, the intermediate-filament protein nestin, SOX2, vimentin, A2B5, and the transcription factor PAX-6 for early stage neural markers (i.e. neural progenitors); NF-M, MAP-2AB, synaptosin, glutamic acid decarboxylase, β111-tubulin and tyrosine hydroxylase for later stage neural markers (i.e. differentiated neural cells). The terms “neural” and “neuronal” are used according to their common meaning in the art and can be used interchangeably throughout.

In embodiments, the neuronal progenitor cell includes an increased expression level of one or more genes within one or more gene ontologies of Table 1. In embodiments, the neuronal progenitor cell includes a decreased expression level of one or more genes within one or more gene ontologies of Table 8. Where the neuronal progenitor cell includes an increased expression level or a decreased expression level of one or more of the genes within one ore more gene ontologies of Table 1 or Table 8, respectively, the neuronal progenitor cell may be a determined dopaminergic precursor cell or a dopaminergic cell.

An “undesirable neuronal progenitor cell” is a cell that is unable to differentiate into a dopaminergic neuron. An undesirable neuronal progenitor cell is not a determined dopaminergic precursor cell or a dopaminergic cell. An undesirable neuronal progenitor cell may be a cell capable of differentiating into neuron types other than dopaminergic cells.

A “specified cell or “specified tissue” as used herein refers to a cell capable of differentiating autonomously (i.e., by itself) when placed in an environment that is neutral with respect to the developmental pathway, such as in a petri dish or test tube. At the stage of specification, cell commitment may still be capable of being altered. If a specified cell is transplanted to a population of differently specified cells, the fate of the transplant will be altered by its interactions with its new neighbors.

The term “determined dopaminergic precursor cell” as provided herein refers to a cell that differentiates into a dopaminergic neuron and cannot differentiate into a non-dopaminergic cell. The term “determined cell” as provided herein refers to a cell capable of differentiating autonomously when placed into a region of an embryo that is unrelated to said cell. For example, an unrelated region for a determined dopaminergic precursor cell is any other organ, tissue other than the brain. The term “determined cell” as provided herein further includes a cell capable of differentiating autonomously when placed into a cluster of differently specified cells in a petri dish. If a cell or tissue type is able to differentiate according to its specified fate even under these circumstances, the commitment is considered irreversible. Thus, a “determined dopaminergic precursor cell” is a cell capable to differentiate into a dopaminergic neuron independently of its environment. A determined dopaminergic precursor cell may express Foxa2 or Nurrl. A determined dopaminergic precursor cell may not express serotonin.

A “dopaminergic cell” or a “differentiated dopaminergic cell” as used herein refers to a cell capable of synthesizing the neurotransmitter dopamine. In embodiments, the dopaminergic cell is an A9 dopaminergic cell. The term “A9 dopaminergic cell” refers to the most densely packed group of dopaminergic cells in the human brain, which are located in the pars compacta of the substantia nigra in the midbrain of healthy, adult humans.

The term “sample” includes sections of tissues such as biopsy and autopsy samples, and frozen sections taken for histological purposes. Such samples include blood and blood fractions or products (e.g., bone marrow, serum, plasma, platelets, red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

A “global profile” as referred to herein is a profile of a characteristic, such as, but not limited to, expression of mRNA, microRNA, DNA methylation, DNA sequence, transcription factor binding, proteins, proteome-wide phospho-proteins, in which there is not a preselection of what genes, DNA sites or what proteins or what subset of the characteristic should be profiled with a specific technique (e.g. microarrays).

A “protein-protein network” as referred to herein is a list of pairwise interacting proteins. These interactions have been derived from previous studies where e.g. the binding of a protein “A” to protein “B” has been shown with biochemical, functional or other biological assays. This interaction can represent a physical covalent or non-covalent binding event of protein “A” with protein “B” or the transient binding of protein “A” to protein “B” in a short lived biochemical reaction such as when protein “A” phosphorylates protein “B”.

A “Stem Cell Matrix” as referred to herein is a collection or database of global profiling data, such as global molecular analysis profiles, which may be gene expression profiles, microRNA expression profiles, non-coding RNA profiles, DNA methylation profiles, transcription factor binding profiles, proteomic profiles, global proteome-wide phospho-protein profiles, DNA sequence profiles, or a combination of elements of the mentioned global profiles.

A “transcriptional profile” as referred to herein is the complete or partial set of data obtained from a cell or a population of cells that can be determined from a single time point or over a period of time, consisting of the RNA types that are transcribed from the genome. These RNA types include, but are not limited to, mRNA, microRNA (miRNA), PIWI-interacting RNAs (piRNAs), endogenous small interfering RNAs (e-siRNAs), TINY RNAs (tiRNA), long non coding RNAs or a combination of the mentioned RNA-types.

A “computer network” as referred to herein is one or more computers in operable communication with each other. Computer implemented refers to one or more steps being actions being performed by a computer, computer system, or computer network. A computer program product as referred to herein is a product which can be implemented and used on a computer, such as software.

An “unsupervised classification” as referred to herein is a computational, algorithm-based classification system, which builds models based on a set of inputs where not all labels for all samples are available or known or understood. As disclosed herein, what has been defined by others as semi-supervised machine learning, which combines both labeled and unlabeled examples to generate an appropriate function or classifier, as unsupervised classification system, can be used.

An “unsupervised cluster method” as referred to herein is an unsupervised machine learning approach to cluster transcriptional profiles of the cell preparations into stable groups. For example, consensus clustering (Monti, S., P. Tamayo, J. Mesirov and T. Golub (2003). “Consensus Clustering: A Resampling-Based Method for Class Discovery and Visualization of Gene Expression Microarray Data.” Machine Learning 52 (1-2): 91-118) outputs a sample-wise distance matrix where the distance between every sample to every other sample in the dataset is represented by a value set between 1 (indistinguishable similar in the context of the data set) and 0 (no similarity detectable in the context of the dataset). A cluster is defined in the consensus clustering framework of a set of samples with high similarity based on the sample-wise distance matrix based on a cutoff set by the consensus clustering algorithm individually for each model. Every other algorithm which outputs a fitting clustering model with and distance measure among all samples can be used instead of the consensus clustering algorithm.

A “similar label profile” as referred to herein may be a common regulatory biochemical or metabolic activity. A similar label profile could be labels from the reference data set (e.g. induced pluripotent stem cells), labels which were derived computationally (e.g. some or all samples belonging to one or more specified clusters) or a combination thereof (e.g. some or all induced pluripotent stem cells which also belong to one or more computationally derived clusters). This could be the identification of a set of marker genes, proteins or pathways different among computationally derived clusters, which can be identified in the future with other biochemical techniques and thus allow identification of computationally identified cluster members with a biochemical assay.

A “labeled associated biological class” as referred to herein is a class based upon a biological definition of a cell, such as by markers or expression, with the main characteristic being that the class is determined by a subset of the total possible profile information.

A “cell characteristic analysis system” as referred to herein is a system, which can assay a characteristic of a cell, such as gene expression, microRNA expression, or methylation patterning.

“Obtaining” as used in the context of data or values, such as characteristic data or values refers to acquiring this data or values. It can be acquired, by for example, collection, such as through a machine, such as a micro array analysis machine. It can also be acquired by downloading or getting data that has already been collected, and for example, stored in a way in which it can be retrieved at a later time.

“Outputting” as referred to herein means an analytical result after processing data by an algorithm. An “updated reference database” as referred to herein is a reference database which has had a dataset merged into it. A “cell dataset” refers to any collection of characteristic data. “Characteristic data” refers to any data of a cell, such as gene expression, microRNA expression, or for example, methylation patterning.

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXEMPLARY EMBODIMENTS

Among the provided embodiments are:
1. A computer implemented method of classifying an in vitro population of neuronal progenitor cells, the method comprising:
receiving a test dataset comprising gene expression levels and expression levels of one or more metagenes for a cell or a plurality of cells comprised in an in vitro population of neuronal progenitor cells, wherein the one or more metagenes are determined based on correlated gene expression levels of reference cells in a reference database, wherein the reference cells are neuronal cells at one or more different stages of differentiation;
applying the expression levels of the one or more metagenes as input to a process configured to determine a probability of the cell or the plurality of cells having metagene expression levels of a determined dopaminergic precursor cell;
determining a deviation score for the cell or the plurality of cells, wherein the deviation score indicates the degree to which the gene expression levels in the test dataset deviate from gene expression levels in one or more reference cells in the reference database, wherein the one or more reference cells are at a stage of differentiation indicating a determined dopaminergic precursor cell; and
outputting, based on the probability and the deviation score, a computed label classification comprising an indication of whether said cell or said plurality of cells from the in vitro population of neuronal progenitor cells is a determined dopaminergic precursor cell.
2. The computer implemented method of embodiment 1, wherein:
the process comprises a supervised classification model trained using (i) expression levels of the one or more metagenes of the reference cells in the reference database; and (ii) class labels indicating each of the one or more different stages of differentiation for reference cells in the reference database, to determine a probability of a cell or a plurality of cells having metagene expression levels of a determined dopaminergic precursor cell.
3. A computer implemented method of training a process to determine a probability of a cell or a plurality of cells having metagene expression levels of a determined dopaminergic precursor cell, the method comprising training a supervised classification model using (i) expression levels of one or more metagenes, wherein the one or more metagenes are determined based on correlated gene expression levels of reference cells in a reference database, wherein the reference cells are neuronal cells at one or more different stages of differentiation; and (ii) class labels indicating each of the one or more different stages of differentiation for reference cells in the reference database, to determine a probability of a cell or a plurality of cells having metagene expression levels of a determined dopaminergic precursor cell.
4. A computer implemented method of classifying an in vitro population of neuronal progenitor cells, the method comprising:
receiving a test dataset comprising gene expression levels and expression levels of one or more metagenes for a cell or a plurality of cells comprised in an in vitro population of neuronal progenitor cells, wherein the one or more metagenes are determined based on correlated gene expression levels of reference cells in a reference database, wherein the reference cells are neuronal cells at one or more different stages of differentiation;
applying the expression levels of the one or more metagenes as input to a process, the process comprising a supervised classification model trained using (i) expression levels of the one or more metagenes of reference cells in the reference database; and (ii) class labels indicating each of the one or more different stages of differentiation of reference cells in the reference database, to determine a probability of a cell or a plurality of cells having metagene expression levels of a determined dopaminergic precursor cell;
determining a deviation score for the cell or the plurality of cells, wherein the deviation score indicates the degree to which the gene expression levels in the test dataset deviate from gene expression levels in one or more reference cells in the reference database, wherein the one or more reference cells are at a stage of differentiation indicating a determined dopaminergic precursor cell; and
outputting, based on the probability and the deviation score, a computed label classification comprising an indication of whether said cell or plurality of cells from the in vitro population of neuronal progenitor cells is a determined dopaminergic precursor cell.
5. The method of any of embodiments 1, 2, and 4, further comprising, based on the computed label classification, identifying the in vitro population of neuronal progenitor cells as a population comprising determined dopaminergic precursor cells.
6. The computer implemented method of any of embodiments 2-5, wherein the supervised classification model is a logistic regression model.
7. The computer implemented method of any of embodiments 1-6, wherein the reference cells are an in vitro population of neuronal progenitor cells.
8. The computer implemented method of any of embodiments 1, 2, and 4-7, wherein said in vitro population of neuronal progenitor cells is formed by culturing one or more induced pluripotent stem cells (iPSC) in vitro for a period of time under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neurons.
9. The computer implemented method of embodiment 8, wherein said iPSC is a human iPSC.
10. The computer implemented method of embodiment 9, wherein said human is a healthy subject.
11. The computer implemented method of embodiment 9, wherein said human is a subject with Parkinson's disease.
12. The computer implemented method of any of embodiments 8-11 wherein the culturing is for period of time that is between at or about 2 and at or about 25 days.
13. The computer implemented method of any of embodiments 8-11, wherein said iPSC is cultured for, for about, or for at least 2 days.
14. The computer implemented method of any of embodiments 8-11, wherein said iPSC is cultured for, for about, or for at least 5 days.
15. The computer implemented method of any of embodiments 8-11, wherein said iPSC is cultured for, for about, or for at least 10 days.
16. The computer implemented method of any of embodiments 8-11, wherein said iPSC is cultured for, for about, or for at least 13 days.
17. The computer implemented method of any of embodiments 8-11, wherein said iPSC is cultured for, for about, or for at least 15 days.
18. The computer implemented method of any of embodiments 8-11, wherein said iPSC is cultured for, for about, or for at least 18 days.
19. The computer implemented method of any of embodiments 8-11, wherein said iPSC is cultured for, for about, or for at least 25 days.
20. The computer implemented method of any of embodiments 1-19, wherein the reference database comprises gene expression levels determined from one or more reference cell populations, wherein each of the one or more reference cell populations are formed by culturing one or more iPSC in vitro for a different period of time each under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neuron.
21. The computer implemented method of embodiment 20, wherein the different period of time is between 2 and 30 days.
22. The computer implemented method of embodiment 20, wherein the different period of time is between 11 and 25 days.
23. The computer implemented method of any of embodiments 1-28, wherein the one or more stages of differentiation of reference cells in the reference database are formed by culturing one or more iPSC in vitro for one or more different period of time under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neuron, wherein the different period of time is between about 11 days and about 25 days, optionally a period of time of at or about 13 days; a period of time of at or about 18 days; or a period of time of at or about 25 days.
24. The computer implemented method of any of embodiments 20-23, wherein at least one of the one or more reference cell populations in the reference database comprises gene expression levels determined by culturing the iPSC for at or about day 13, 18, or 25 days.
25. The computer implemented method of any of embodiments 8-24, wherein the conditions capable of differentiating the one or more iPSCs to a neuronal progenitor cell comprises culturing the iPSCs by:

(a) a first incubation comprising exposing the cells to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling, optionally under conditions to differentiate the cells to floor plate midbrain progenitor cells, optionally wherein the first incubation is initiated on day 0 of the culturing; and

(b) a second incubation of cells after the first incubation, wherein the second incubation comprises culturing the cells under conditions to neurally differentiate the cells, optionally wherein the second incubation is initiated at or about day 11 after the first incubation, and further optionally wherein the second incubation is for between at or about 11 and at or about 25 days.
26. The computer implemented method of embodiment 25, wherein the conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.
27. The computer implemented method of any of embodiments 20-26, wherein at least one of the one or more reference cell populations in the reference database comprises gene expression levels determined by culturing the iPSC for at or about 13 days.
28. The computer implemented method of any of embodiments 20-27, wherein at least one of the one or more reference cell populations comprises gene expression levels determined by culturing the iPSC for at or about 18 days.
29. The computer implemented method of any of embodiments 20-28, wherein at least one of the one or more reference cell populations comprises gene expression levels determined by culturing the iPSC for at or about 25 days.
30. The computer implemented method of any of embodiments 1-29, wherein the one or more metagenes and the expression levels of the one or more metagenes are determined by using a dimensionality reduction technique on one or more reference cells of the one or more reference database.
31. The computer implemented method of embodiment 30, wherein the dimensionality reduction technique is used on a reference cell population comprising gene expression levels determined at or about 13 days of culturing iPSC in vitro under conditions to differentiate neuronal progenitor cells.
32. The computer implemented method of embodiment 30 or embodiment 31, wherein the dimensionality reduction technique is used on a reference cell population comprising gene expression levels determined at or about 18 days of culturing iPSC in vitro under conditions to differentiate neuronal progenitor cells.
33. The computer implemented method of any of embodiments 30-32, wherein the dimensionality reduction technique is used on a reference cell population comprising gene expression levels determined at or about 25 days of culturing iPSC in vitro under conditions to differentiate neuronal progenitor cells.
34. The computer implemented method of any of embodiments 30-33, wherein the dimensionality reduction technique is used on each of:
a reference cell population comprising gene expression levels determined at or about 13 days of culturing iPSC in vitro under conditions to differentiate neuronal progenitor cells;
a reference cell population comprising gene expression levels determined at or about 18 days of culturing iPSC in vitro under conditions to differentiate neuronal progenitor cells; and
a reference cell population comprising gene expression levels determined at or about 25 days of culturing iPSC in vitro under conditions to differentiate neuronal progenitor cells.
35. The computer implemented method of any of embodiments 2-34, wherein the supervised classification model is trained using the expression levels of the one or more metagenes determined from the one or more reference cells.
36. The computer implemented method of any of embodiments 2-35, wherein the supervised classification model is trained using the expression levels of the one or more metagenes determined from one or more reference cells comprising gene expression levels between 11 and 25 days of culturing iPSC in vitro under conditions to differentiate neuronal progenitor cells, optionally one or more of 13, 18, and 25 days of culturing iPSC in vitro under conditions to differentiate neuronal progenitor cells.
37. The computer implemented method of any of embodiments 2-36, wherein the supervised classification model is trained using the expression levels of the one or more metagenes determined from the one or more reference cells comprising gene expression levels determined at or about 13 days of culturing iPSC in vitro under conditions to differentiate neuronal progenitor cells.
38. The computer implemented method of any of embodiments 2-37, wherein the supervised classification model is trained using the expression levels of the one or more metagenes determined from the one or more reference cells comprising gene expression levels determined at or about 18 days of culturing iPSC in vitro under conditions to differentiate neuronal progenitor cells.
39. The computer implemented method of any of embodiments 2-38, wherein the supervised classification model is trained using the expression levels of the one or more metagenes determined from the one or more reference cells comprising gene expression levels determined at or about 25 days of culturing iPSC in vitro under conditions to differentiate neuronal progenitor cells.
40. The computer implemented method of any of embodiments 2-39, wherein the supervised classification model is trained using the expression levels of the one or more metagenes determined from each of:
a reference cell population comprising gene expression levels determined at or about 13 days of culturing iPSC in vitro under conditions to differentiate neuronal progenitor cells;
a reference cell population comprising gene expression levels determined at or about 18 days of culturing iPSC in vitro under conditions to differentiate neuronal progenitor cells; and
a reference cell population comprising gene expression levels determined at or about 25 days of culturing iPSC in vitro under conditions to differentiate neuronal progenitor cells.
41. The computer implemented method of any of embodiments 2-40, wherein the class label indicating each of the one or more different stages of differentiation of the reference cells is either a determined dopaminergic precursor cell or a not a determined dopaminergic precursor cell.
42. The computer implemented method of any of embodiments 2-41, wherein the class label indicating each of the one or more different stages of differentiation of the reference cells is determined using an in vivo method.
43. The computer implemented method of embodiment 42, wherein the in vivo method comprises:
transplanting the in vitro population of neuronal progenitor cells comprising a reference cell population into a brain region of an animal model of Parkinson's disease;
assessing the occurrence of an outcome associated with a therapeutic effect of the transplantation on the animal model, optionally wherein the outcome is selected from innervation or engrafting with host cells, reduction of a brain lesion in the animal model, or reversal of a brain lesion in the animal model; and
designating the class label as a determined dopaminergic precursor cell if the transplantation results in the occurrence of the outcome associated with a therapeutic effect; or
designating the class label as not a determined dopaminergic precursor cell if the transplantation does not result in the occurrence of the outcome associated with a therapeutic effect.
44. The computer implemented method of embodiment 43, wherein the brain region is the substantia nigra.
45. The computer implemented method of embodiment 43 or embodiment 44, wherein the in vivo method comprises a behavioral assay.
46. The computer implemented method of any of embodiments 2-41, wherein the class label indicating each of the one or more different stages of differentiation of the reference cells is determined using an in vitro method.
47. The computer implemented method of embodiment 46, wherein:
the in vitro method comprises assessing dopamine production levels of a reference cell population; and
the class label is designated as a determined dopaminergic precursor cell if the dopamine production levels are increased relative to a pluripotent stem cell.
48. The computer implemented method of embodiment 46 or 47, wherein assessment of dopamine production is by high performance liquid chromatography.
49. The computer implemented method of any of embodiments 46-48, wherein:
the in vitro method comprises assessing levels of Tyrosine Hydroxylase expression for a reference cell population; and
the class label is designated as a not a determined dopaminergic precursor cell if the reference cell population expresses high Tyrosine Hydroxylase.
50. The computer implemented method of embodiment 49, wherein the levels of Tyrosine Hydroxylase expression are assessed using flow cytometry.
51. The computer implemented method of any of embodiments 2-50, wherein the reference database further comprises the class labels of the one or more reference cells.
52. The computer implemented method of any of embodiments 1, 2, and 4-51, wherein the expression levels of the one or more metagenes in the test dataset is determined based on (i) the one or more metagenes determined from the one or more reference cells in the reference database and (ii) the gene expression levels in the test dataset.
53. The computer implemented method of embodiment 52, wherein the expression levels of the one or more metagenes in the test dataset is determined using regression analysis based on (i) the one or more metagenes determined from the one or more reference cells in the reference database and (ii) the gene expression levels in the test dataset.
54. The computer implemented method of any of embodiments 1, 2, and 4-51, wherein the expression levels of the one or more metagenes in the test dataset is determined by merging the gene expression levels in the test dataset with the reference database to create an updated reference database and applying the dimensionality reduction technique on the updated reference database.
55. The computer implemented method of any of embodiments 30-54, wherein the dimensionality reduction technique is conventional non-negative matrix factorization, discriminant non-negative matrix factorization, graph regularized non-negative matrix factorization, bootstrapping sparse non-negative matrix factorization, or regularized non-negative matrix factorization.
56. The computer implemented method of any of embodiments 30-55, wherein the dimensionality reduction technique is conventional non-negative matrix factorization.
57. The computer implemented method of any of embodiments 2-56, wherein the number of the one or more metagenes is chosen based on the performance of the supervised classification model in determining a probability of a cell or a plurality of cells having metagene expression levels of a determined dopaminergic precursor cell.
58. The computer implemented method of any of embodiments 30-57, wherein the number of the one or more metagenes is chosen based on evaluating one or more metrics determined from performing the dimensionality reduction technique using multiple candidate numbers of metagenes.
59. The computer implemented method of embodiment 58, wherein the one or more metrics comprise cophenetic distance, dispersion, residuals, residual sum of squares (RSS), silhouette, and/or sparseness values.
60. The computer implemented method of any of embodiments 1, 2, and 4-59, wherein the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than a threshold probability value.
61. The computer implemented method of embodiment 60, wherein:
the threshold probability value is set such that a determined dopaminergic precursor cell is identified with greater than or greater than about 75%, 80%, 85%, 90%, or 95% sensitivity; and/or
the threshold probability value is set such that a determined dopaminergic precursor cell is identified with greater than or greater than about 75%, 80%, 85%, 90%, or 95% specificity.
62. The computer implemented method of embodiment 60, wherein the threshold probability value is set such that a determined dopaminergic precursor cell is identified with greater than or greater than about 98% sensitivity and 100% specificity.
63. The computer implemented method of any of embodiments 60-62, wherein the threshold probability value is determined by using the area under a receiver operator characteristic (ROC) curve based on the supervised classification model.
64. The computer implemented method of any of embodiments 60-63, wherein the threshold probability value is between or between about 0.4 and 0.8 inclusive.
65. The computer implemented method of any of embodiments 60-63, wherein the threshold probability value is or is about 0.4, 0.45, 0.5, 0.55, 0.6, 0.65, 0.7, 0.75, or 0.8.
66. The computer implemented method of any of embodiments 1, 2, and 4-65, wherein the deviation score for the cell or the plurality of cells is determined using a single-gene deviation score for each of one or more genes in the test dataset.
67. The computer implemented method of embodiment 66, wherein the single-gene deviation scores are determined using differences between the gene expression levels of the test dataset and the gene expression levels in one or more reference cells in the reference database.
68. The computer implemented method of embodiment 67, wherein the differences are absolute differences.
69. The computer implemented method of any of embodiments 66-68, wherein the single-gene deviation scores are determined using standard deviations of gene expression levels in one or more of the one or more reference cells.
70. The computer implemented method of any of embodiments 66-69, wherein the single-gene deviation scores are z-scores determined using:
the differences between the gene expression levels of the test dataset and the gene expression levels in the one or more reference cells in the reference database; and
the standard deviations of gene expression levels in one or more of the one or more reference cells of the reference database.
71. The computer implemented method of any of embodiments 1, 2, and 4-70, wherein the gene expression levels in one or more reference cells in the reference database are determined based on average gene expression levels in one or more reference cells of the reference database.
72. The computer implemented method of any of embodiments 1, 2, and 4-70, wherein the gene expression levels in the one or more reference cells in the reference database are determined based on the expression levels of the one or more metagenes in the test dataset.
73. The computer implemented method of embodiment 72, wherein the gene expression levels in the one or more reference cells in the reference database are determined using regression analysis based on (i) the expression levels of the one or more metagenes in the test dataset and (ii) the gene expression levels in the test dataset.
74. The computer implemented method of any of embodiments 66-73, wherein the deviation score is a summary statistic based on all single-gene deviation scores.
75. The computer implemented method of any of embodiments 66-73, wherein the deviation score is a summary statistic based on single-gene deviation scores for one or more marker genes.
76. The computer implemented method of embodiment 74 or embodiment 75, wherein the summary statistic is a sum.
77. The computer implemented method of embodiment 74 or embodiment 75, wherein the summary statistic is a weighted sum.
78. The computer implemented method of embodiment 77, wherein the single-gene deviation scores of the one or more marker genes have higher weight.
79. The computer implemented method of embodiment 74 or embodiment 75, wherein the summary statistic is a percentile value.
80. The computer implemented method of embodiment 79, wherein:
the percentile value is between or between about the 50% percentile and the 100% percentile; and/or
the percentile value is or is about the 50%, 60%, 70%, 80%, 90%, or 95% percentile.
81. The computer implemented method of any of embodiments 75-80, wherein the marker genes comprise radial glial cell markers, early neuronal development genes, pluripotency specific markers, intermediate to late neuronal markers, neurofilament light polypeptide chain markers, neurofilament medium polypeptide chain markers, nestin filament markers, early patterning markers, neural progenitor cell markers, early migration markers, stage-specific transcription factors, genes required for normal development of neurons, genes controlling dopaminergic neuron development, genes regulating identity and fate of neuronal progenitor cells, dopaminergic neuron markers, astrocyte markers, forebrain markers, hindbrain markers, subthalamic nucleus markers, radial glial markers, cell cycle markers, or any combination of any of the foregoing.
82. The computer implemented method of any of embodiments 75-81, wherein the marker genes are or comprise WNT1, VIM, TOP2A, TH, SOX2A, SLIT2, RFX4, POU5F1, PITX2, PAX6, OTX2, NR4A2, NHLH2, NEUROD4, NEUROD1, NES, NEFM, NEFL, NASP, MAP2, LMX1A, LIN28A, HOXA2, HMGB2, HES1, FOXG1, FOXA2, FABP7, DDC, DCX, BARHL2, BARJL1, ASPM, ALDH1A1, or any combination of any of the foregoing.
83. The computer implemented method of any of embodiments 1, 2, and 4-82, wherein the computed label classification indicates that said cell or plurality of cells from the in vitro population of neuronal progenitor cells is a determined dopaminergic precursor cell if the deviation score indicates that at least or at least about 50%, 50%, 70%, 80%, 90%, or 95% of gene expression levels in the test dataset are no more than five standard deviations away from gene expression levels of the one or more reference cells in the reference database.
84. The computer implemented method of any of embodiments 1, 2, and 4-82, wherein the computed label classification indicates that said cell or plurality of cells from the in vitro population of neuronal progenitor cells is a determined dopaminergic precursor cell if the deviation score indicates that at least or at least about 95% of gene expression levels in the test dataset are no more than 10, 9, 8, 7, 6, or 5 standard deviations away from the gene expression levels of the one or more reference cells in the reference database.
85. The computer implemented method of any of embodiments 1, 2, and 4-82, wherein the computed label classification indicates that said cell or plurality of cells from the in vitro population of neuronal progenitor cells is a determined dopaminergic precursor cell if the deviation score indicates that at least or at least about 50%, 50%, 70%, 80%, 90%, or 95% of marker gene expression levels in the test dataset are no more than five standard deviations away from the gene expression levels of the one or more reference cells in the reference database.
86. The computer implemented method of any of embodiments 1, 2, and 4-82, wherein the computed label classification indicates that said cell or plurality of cells from the in vitro population of neuronal progenitor cells is a determined dopaminergic precursor cell if the deviation score indicates that at least or at least about 95% of marker gene expression levels in the test dataset are no more than 10, 9, 8, 7, 6, or 5 standard deviations away from the gene expression levels of the one or more reference cells in the reference database.
87. The computer implemented method of any of embodiments 60-82, wherein the computed label classification indicates that said cell or plurality of cells from the in vitro population of neuronal progenitor cells is a determined dopaminergic precursor cell if:
the probability of the cell or the plurality of cells having metagene expression levels of the determined dopaminergic precursor cell is greater than the threshold probability value; and
   the deviation score indicates that at least or at least about 50%, 50%, 70%, 80%, 90%, or 95% of gene expression levels in the test dataset are no more than five standard deviations away from the gene expression levels of the one or more reference cells in the reference database.
88. The computer implemented method of any of embodiments 60-82, wherein the computed label classification indicates that said cell or plurality of cells from the in vitro population of neuronal progenitor cells is a determined dopaminergic precursor cell if:
the probability of the cell or the plurality of cells having metagene expression levels of the determined dopaminergic precursor cell is greater than the threshold probability value; and
the deviation score indicates that at least or at least about 50%, 50%, 70%, 80%, 90%, or 95% of marker gene expression levels in the test dataset are no more than five standard deviations away from the gene expression levels of the one or more reference cells in the reference database.
89. The computer implemented method of any of embodiments 60-82, wherein the computed label classification indicates that said cell or plurality of cells from the in vitro population of neuronal progenitor cells is a determined dopaminergic precursor cell if:
the probability of the cell or the plurality of cells having metagene expression levels of the determined dopaminergic precursor cell is greater than the threshold probability value;
the deviation score indicates that at least or at least about 50%, 50%, 70%, 80%, 90%, or 95% of gene expression levels in the test dataset are no more than five standard deviations away from the gene expression levels of the one or more reference cells in the reference database;
the deviation score indicates that at least or at least about 50%, 50%, 70%, 80%, 90%, or 95% of marker gene expression levels in the test dataset are no more than five standard deviations away from the gene expression levels of the one or more reference cells in the reference database.
90. The computer implemented method of any of embodiments 75-89, wherein the computed label classification indicates that said cell or plurality of cells from the in vitro population of neuronal progenitor cells is a determined dopaminergic precursor cell if the differences in expression of the marker genes between the test dataset and reference cells of the reference database is statistically insignificant based on a multiple-comparison corrected significance level.
91. The computer implemented method of embodiment 90, wherein the multiple-comparison corrected significance level is a Bonferroni corrected significance level or a false discover rate corrected significance level.
92. The computer implemented method of embodiment 90 or embodiment 91, wherein the multiple-comparison corrected significance level is 0.01, 0.05, or 0.1.
93. The computer implemented method of one of embodiments 1-92, wherein said gene expression levels are obtained from microarray analysis of cellular RNA, RNA sequencing, or both.
94. The computer implemented method of one of embodiments 1-93, wherein said gene expression levels are obtained from RNA sequencing.
95. The computer implemented method of embodiment 93 or embodiment 94, wherein the RNA sequencing is performed on bulk RNA from the plurality of cells or a plurality of reference cells.
96. The computer implemented method of embodiment 93 or embodiment 94, wherein the RNA sequencing is performed on RNA from the single cells or a single reference cell.
97. The computer implemented method of embodiment 93 or embodiment 94, wherein the gene expression levels of reference cells in the reference database comprises expression levels determined by RNA sequencing that is performed on bulk RNA from a plurality of reference cells and on RNA from a single reference cell.
98. The computer implemented method of any of embodiments 1, 2, and 4-97, wherein receiving said test dataset comprises receiving input from an array analysis system.
99. The computer implemented method of any of embodiments 1, 2, and 4-98, wherein receiving the test dataset comprises receiving input via a computer network.
100. The computer implemented method of any of embodiments 1, 2, and 4-99, wherein said one or more reference databases forms part of a storage medium.
101. The computer implemented method of any of embodiments 1, 2, and 4-100, comprising repeating the receiving, applying, determining, and outputting steps if the computed label classification indicates that said cell or plurality of cells is not a determined dopaminergic neuronal cell, optionally wherein the steps are repeated the same or a different in vitro population of neuronal progenitor cells.
102. The computer implemented method of embodiment 101, wherein the receiving, applying, determining, and outputting steps are repeated or repeated about one, two, three, four, five, six, seven, eight, nine, or 10 days after the previous iteration of the method.
103. The computer implemented method of any of embodiments 1, 2, and 4-102, comprising repeating the receiving, applying, determining, and outputting steps if the computed label classification indicates that said cell or plurality of cells is not a determined dopaminergic neuronal cell, wherein the steps are repeated using different in vitro population of neuronal progenitor cells formed by culturing another iPSC clone under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neurons.
104. The computer implemented method of embodiment 103, wherein said different in vitro population of neuronal progenitor cells is formed from the same human subject as the previous iteration of the method.
105. The computer implemented method of any of embodiments 101-104, wherein the receiving, applying, determining, and outputting steps are repeated on in vitro population of neuronal progenitor cells formed by culture of iPSC for different periods of time and/or under different conditions capable of differentiating the one or more iPSCs to a neuronal progenitor cell, until an indication that said cell or said plurality of cells is a determined dopaminergic neuronal cell is output.
106. A population of determined dopaminergic precursor cells identified by the method of any of embodiments 5-105.
107. A method of treatment, the method comprising administering to a subject having Parkinson's disease the population of determined dopaminergic precursor cells of embodiment 106.
108. The method of embodiment 107, wherein the administering is by implanting the population of determined dopaminergic precursor cells into one or more brain regions of the subject.
109. The method of embodiment 108, wherein the one or more brain regions comprise the substantia nigra.
110. The method of any of embodiments 107-109, wherein the population of determined dopaminergic precursor cells is autologous to the subject.
111. The method of any of embodiments 107-109, wherein the population of determined dopaminergic precursor cells is allogeneic to the subject.
112. A method of treating a subject having Parkinson's disease, the method comprising:
implanting a population of determined dopaminergic precursor cells into a brain region of a subject having Parkinson's disease, wherein the population of determined dopaminergic precursor cells has been identified using the computer implemented method of any of embodiments 5-105.
113. The method of embodiment 112, wherein the population of determined dopaminergic precursor cells is autologous to the subject.
114. The method of any of embodiments 112-113, wherein the population of determined dopaminergic precursor cells is allogeneic to the subject.
115. The method of any of embodiments 107-114, wherein about or at least or 1×10⁶ cells are injected into the substantia nigra.
116. The method of any of embodiments 107-115, wherein the cells are injected into both the left and right hemispheres.

Among the Provided Embodiments are:

1. A computer implemented method of identifying a determined dopaminergic precursor cell within an in vitro population of neuronal progenitor cells, the method comprising:

receiving a test dataset comprising data including gene expression profile information for an in vitro population of neuronal progenitor cells;

querying a gene expression reference database to compare said test dataset with said gene expression reference database, said gene expression reference database comprising gene expression profile information for a desirable determined dopaminergic precursor cell; and outputting a computed label classification comprising an indication of whether said in vitro population of neuronal progenitor cells copmrises a determined dopaminergic precursor cell.

2. The computer implemented method of embodiment 1, wherein said gene expression profile information for said desirable determined dopaminergic precursor cell comprises increased gene expression levels relative to a pluripotent stem cell for a first gene set, wherein said first gene set comprises at least one increased gene within one or more first gene ontologies selected from the group consisting of: GO0005509, GO0016339, GO0007416 and GO0048731.

3. The computer implemented method of embodiment 2, wherein said at least one increased gene is selected from the group consisting of: CAPN14, FAT3, FAT4, PCDHGC4, SLC8A1, SLIT2, CEMIP2, CDHR3, CDH2, DRD2, EPHB2, MAGI2, PCDHB11, PCDHB13, PCDHB14, PCDHB16, PCDHB2, ADGRG6, ELF5, EPHA7, FOXP1, GDF7, HOXA1, MINAR1, MSX1, NRBP2, NRIP1, PITX3, POU6F2, PTPRO, SLC35D1, TCF12, ZFHX3 and ZNF703.

4. The computer implemented method of one of embodiments 1 to 3, wherein said gene expression profile information for said desirable determined dopaminergic precursor cell comprises decreased gene expression levels relative to a pluripotent stem cell for a second gene set, wherein said second gene set comprises at least one decreased gene within one or more second gene ontologies selected from the group consisting of: GO0070887, GO0044459 and GO0044281.

5. The computer implemented method of embodiment 4, wherein said at least one decreased gene is selected from the group consisting of: ADCY8, AKR1C3, ALDH3A1, APRT, ASNS, BAX, BBC3, CCND1, CDH5, CH25H, CMKLR1, COL16A1, CXCL1, CXCL2, EDNRB, EEF1E1, RIPOR2, FGF10, FGF22, FZD7, GJA1, GNG8, GNPNAT1, HPGD, ICAM1, ITPR2, KLF1, KLF15, LEP, LPL, LRRC32, MAP3K5, MX1, MYC, NME1, NME2, NQO2, NR1D1, P2RY1, PCOLCE2, PDE4A, PDIA5, PFKP, PHGDH, PLK5, PPP1R14A, PRODH, PSMB8, PSMB9, PYCR1, RAPGEF3, RYR2, SCARB1, SHMT2, SIPA1, SPHK1, TRIM22, VDR, ADA, ADGRG3, ADGRL4, ANK1, ART3, CAll, CABP1, CDH15, CDHR1, COL13A1, EPHA6, CALHM6, GRID2IP, HS3ST3B1, ICAM5, JCAD, LGR6, LRRC38, NOXO1, PDPN, PLPPR5, PODXL, RAMP3, RGS7BP, RIMS4, RTBDN, RTN4RL2, S100A10, SEMA4A, SGCG, SH2D5, SHISA9, SHROOM1, SLC22A3, SLC24A2, SLC29A2, SLC6A11, SLC7A10, SLC7A5, SLCO2A1, STAC2, STYK1, TMC1, UNC13A, WWC1, ABCG2, ACSBG1, ACSS1, ACYL, AHCY, ALOX12B, AMD1, ARG2, ASST, BCAT1, CHST2, CLN8, ENTPD2, FABP5, FADS3, FUT4, FUT9, GAL3ST3, GMDS, HACD1, HAS3, HPD, KYAT1, LDHD, MPP1, OGDHL, PDE4A, PGM1, PIPDX, PLAAT3, PLA2G4C, PLCB3, PNP, PSAT1, PTGES, REXO2, SCARB1, SLC27A6, SPHK1, STAB2, UAP1L1 and UCK2.

6. The computer implemented method of one of embodiments 1 to 5, further comprising a machine learning model trained to determine whether said in vitro population of neuronal progenitor cells includes said determined dopaminergic precursor cell, said machine learning model outputting said computed label classification.

7. The computer implemented method of one of embodiments 1 to 6, wherein said in vitro population of neuronal progenitor cells are formed by allowing an induced pluripotent stem cell (iPSC) to expand in vitro.

8. The computer implemented method of one of embodiments 1 to 7, wherein said iPSC is a human iPSC.

9. The computer implemented method of one of embodiments 1 to 8, wherein said iPSC is allowed to expand for at least 15 days.

10. The computer implemented method of one of embodiments 1 to 9, wherein said iPSC is allowed to expand for about 18 days.

11. The computer implemented method of one of embodiments 1 to 10, wherein said gene expression profile information for said desirable determined dopaminergic precursor cell comprises an undesirable gene expression profile comprising one or more undesirable genes.

12. The computer implemented method of embodiment 11, wherein said one or more undesirable gene is a cancer marker gene.

13. The computer implemented method of embodiment 11, wherein said one or more undesirable genes is a tyrosine hydroxylase gene.

14. The computer implemented method of embodiment 6, wherein said machine learning model is a best fitting classification model identified by an algorithm as most stable to random perturbations.

15. The computer implemented method of embodiment 14, wherein said best fitting classification model can cluster individual datasets such that each dataset within a cluster is indistinguishable from each other dataset within said cluster.

16. The computer implemented method of one of embodiments 1-15, comprising identifying computationally derived class labels based only on biological characteristics.

17. The computer implemented method of one of embodiments 1-16, comprising identifying differences in at least one dataset for at least one label between at least two samples in at least two clusters.

18. The computer implemented method of one of embodiments 1-17, comprising filtering within a cluster for samples having a similar label profile.

19. The computer implemented method of one of embodiments 1-18, comprising defining differentially regulated protein-protein networks.

20. The computer implemented method of embodiment 19, comprising using said protein-protein networks to define a class membership, manipulate class membership, or define biological function of said neuronal progenitor cells.

21. The computer implemented method of embodiment 14, wherein said best fitting classification model can cluster individual datasets such that each dataset within a cluster is different from each other individual dataset.

22. The computer implemented method of one of embodiments 1-21, wherein said computed label classification is an unsupervised classification of said updated reference database comprising clustering RNA, DNA and/or protein profiles.

23. The computer implemented method of one of embodiments 1-22, wherein said gene expression profile information is obtained from microarray analysis of cellular RNA.

24. The computer implemented method of one of embodiments 1-23, wherein said computed label classification is an unsupervised machine classification comprising a bootstrapping sparse non-negative matrix factorization.

25. The computer implemented method of one of embodiments 1-24, wherein said gene expression reference database comprises transcriptional profiles of one or more dopaminergic neurons.

26. The computer implemented method of one of embodiments 1-25, further comprising classifying cells with said in vitro population of neuronal progenitor cells based at least in part on a computationally derived protein-protein network.

27. The method of one of embodiments 1-26, wherein said gene expression profile information comprises a transcriptional profile.

28. The computer implemented method of one of embodiments 1-27, wherein said gene expression reference database comprises known class labels.

29. The computer implemented method of one of embodiments 1-28, wherein said gene expression reference database forms part of a storage medium.

30. The computer implemented method of one of embodiments 1-29, wherein receiving said test dataset comprises receiving input from an array analysis system.

31. The computer implemented method of one of embodiments 1-29, wherein receiving the test dataset comprises receiving input via a computer network.

32. The computer implemented method of one of embodiments 1-29, wherein said data in said reference database is associated with one or more labeled associated biological classes of the cells.

### EXAMPLES

The following examples are included for illustrative purposes only and are not intended to limit the scope of the invention.

Methods of Identifying Dopaminergic Neurons and Progenitor Cells

### Example 1: Neurotest: Prediction of Dopaminergic Neuron Maturation and Function

The differentiation of induced pluripotent stem cells (iPSC) or embryonic stem cells (ESC) into neurons (Studer, 2012) is a developmental process which adheres to the principles of developmental biology.

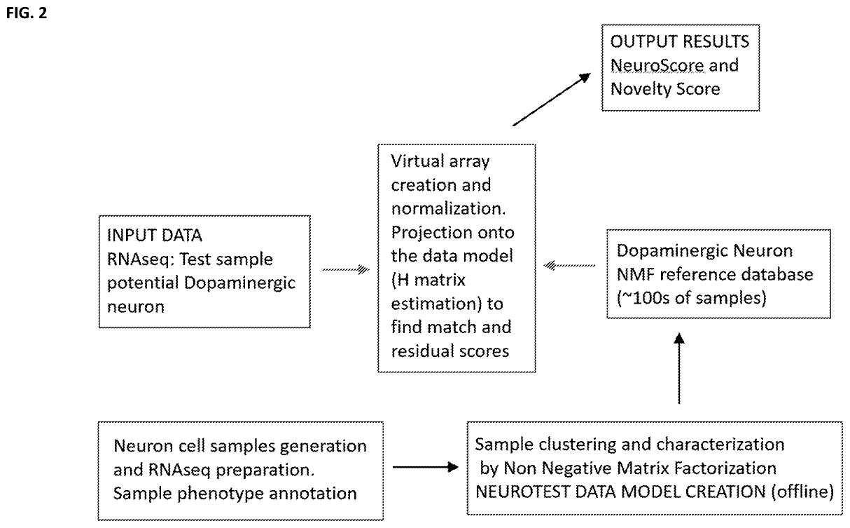

A method was developed for evaluating the whole cell phenotype of a cell type, for instance that of dopaminergic neurons, based on gene expression data collected during differentiation. An exemplary workflow for this method is shown in FIG. 2, and this workflow is referred to here in Example 1 as NeuroTest. Using the NeuroTest algorithm, two parameters were generated per developing neuronal preparation which, together, provided a concise description of the whole cell phenotype of the developing neuronal preparation (e.g., an in vitro population of neuronal progenitor cells). These two parameters were:

Parameter #1: a Neuroscore that was the result of a logistic regression model that measured the probability of a “test” developing neuronal cell preparation (e.g., an in vitro population of neuronal progenitor cells) being a phenotypic match to a reference developmentally-determined dopaminergic neuron (determined dopaminergic precursor cell). See FIG. 1 shows how an initially pluripotent cell would progress to a determined state before reaching a differentiated state. For example, the phenotype of interest could be the cellular developmental state occurring around day 18 (d18) of an in vitro dopaminergic neuron differentiation protocol.

Parameter #2: a Novelty score that indicated the phenotypic deviation of a “test” dopaminergic neuron preparation (in vitro population of neuronal progenitor cells) when compared to a known reference set of developmentally-determined dopaminergic neurons. The novelty score measured technical as well as biological variations in the data. Here larger Novelty score values indicated gene expression patterns usually not observed in the standard reference set. According to the NeuroTest algorithm, high quality determined day 18 dopaminergic lines (determined dopaminergic precursor cells) had a Neuroscore ≥500 and Novelty Score ≤0.48. These thresholds allowed for the labelling of a sample as a “pass” for having a high likelihood of continuing to mature into a therapeutically viable dopaminergic neuron as cellular development continues to day 25 and beyond.

This style of two parameter descriptor for evaluating the whole cell phenotype of a cell type is reminiscent of a different and distinct cell test called PluriTest. The new test procedure provided herein is focused on identifying a specific transitory developmental state of a cell type (e.g., a determined dopaminergic precursor cell), and then imputing a likelihood for its developmental end point. This was not the case for Pluritest, which was solely focused on identifying the stable cell state known as pluripotency (Muller et al., 2011).

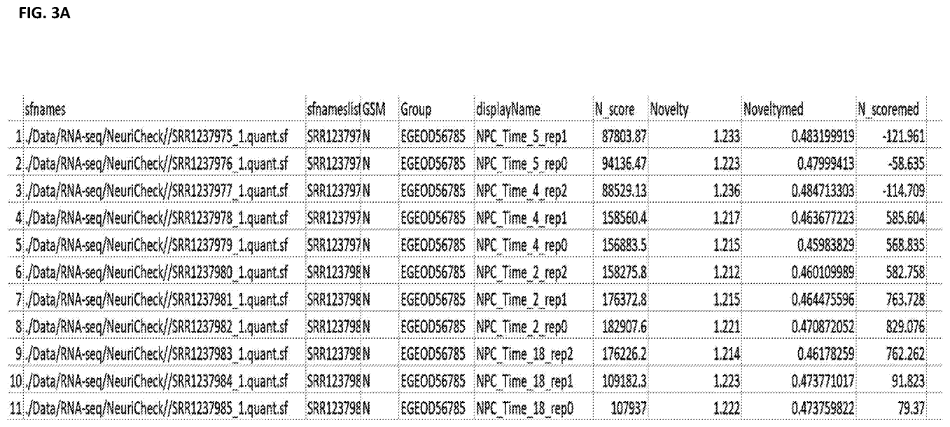

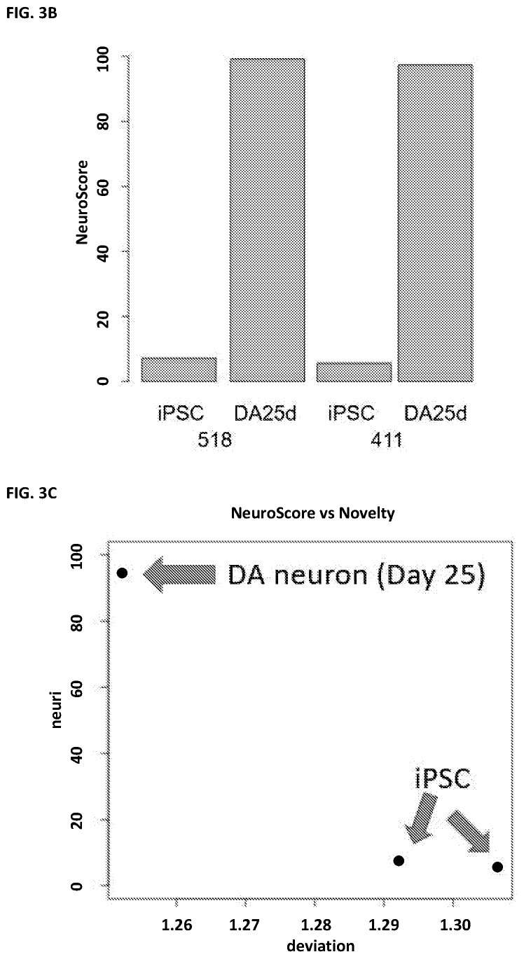

Underlying NeuroTest were two custom data analysis methods: [1] a reference-neuron data model, based on generated gene expression data and publicly available neuron gene expression data and [2] a computing method to compare RNAseq gene expression data coming from new neuronal test samples to the reference gene expression data summarized in the model. The exemplary workflow depicted in FIG. 2 shows how input RNAseq data from a test sample would be projected into and compared with the NeuroTest data model. The results from this comparison were communicated back to an end-user as a graph, illustrating the fit between the test sample and the reference data. FIG. 3A-3C show exemplary graphs that were provided to the end-user.

A. The Design and Construction of the NeuroTest Reference Set Data Model

To generate the reference datasets used in developing the NeuroTest model, dopaminergic neuron cellular samples were generated by differentiation of iPSCs in vitro and sampling of cell lines as they differentiated from d0 to d60, or beyond. Sample by sample, mRNA was extracted in bulk to enable the determination of the cell's gene expression pattern (Hrdlickova et al., 2017). The integration and analysis of these gene expression patterns was responsible for the creation of the developmentally-determined neuron data model used in NeuroTest.

To measure these gene expression patterns, total RNA was extracted from DA neurons using AllPrep DNA/RNA Mini Kit (QIAGEN) following the manufacturer's protocol. This was RNA quality was assessed based on RNA integrity number (RIN) using an Agilent Bioanalyzer. Any samples with RIN less than 7.5 were re-isolated. Paired end sequencing libraries were prepared using the Illumina PolyA+ TruSeq mRNA Library Prep kit V2 and sequenced using an Illumina HiSeq2500. Samples were sequenced to an average of 30 million paired end reads (Hrdlickova et al., 2017). The reads were converted into a table of gene expression data by aligning the reads to the transcriptome (Salmon version 0.7.2, (Patro et al., 2017)) and counting how many reads aligned to each gene. The summed counts directly reflected the concentration of a specific mRNA transcript in the cell at the time of the RNA extraction. Read counts were normalized to TPM (Transcripts Per Kilobase Million) values before analysis by Non Negative Matrix factorization (Brunet et al., 2004).

After sequencing, the RNAseq datasets as well as microarray datasets were included in the NeuroTest model and themselves included a variety of neuron focused gene expression datasets. Together, these reflected the discriminatory needs of the model and provided a perspective on intra- and inter-patient cell line variation, as well as sample to sample biological and technical variation present in DA neuron preparations. The datasets included:

6 RNAseq datasets from DA neurons used for a successful Rat neuron transplantation study (60 Rats in study), wherein transplantation led to reveral of the effect of a Parkinsonian model brain lesion. These were “gold standard” datasets which can be thought of as a dopaminergic neuron substitute for iPSC lines which have been “proven” pluripotent by passing the Teratoma assay (Daley et al., 2009). For this transplantation study, iPSCs were generated from six patients with Parkinson's disease (PD). First, punch biopsies were used to harvest skin fibroblasts from each patient. Tissue from the biopsies was minced with a scalpel and subjected to collagenase or trypsin treatment before being placed in culture. The fibroblasts were then reprogrammed to integration-free iPSCs using Sendai virus and frozen at passage 10.

After reprogramming, iPSCs were placed in an in vitro dopaminergic neuron differentiation protocol prior to being transplanted in a PD rat model. In this model, rats received unilateral stereotaxic injection of 6-hydroxydopamine (6-OHDA) into the substantia nigra or the medial forebrain bundle. This lesioning led to asymmetric dopamine discharge after amphetamine treatment (i.e., dopamine was discharged only from the unlesioned hemisphere) that caused lesioned rats to circle in one direction when moving. In this study, after baseline circling behavior was measured in lesioned rats, neural precursors at day 18 of the dopaminergic neuron differentiation protocol were transplanted into the lesioned hemisphere. Rats were then periodically tested for amphetamine-induced circling. Six to eight weeks after transplant, the net number of amphetamine-induced rotations was reduced to zero. This result showed that transplantation of developmentally determined dopaminergic precursor cells (i.e., neural precursors at day 18 of the dopaminergic neuron differentiation protocol) led to the reversal or amelioration of PD symptoms.

70 Microarray datasets from dopaminergic neuron preparations. These were quality controlled and annotated with an indication of final dopamine production levels. Microarray datasets included dopaminergic neuron preparations from day 25 of a dopaminergic neuron differentiation protocol, and iPSCs subjected to this protocol were generated from 12 PD patients.

47 RNAseq datasets from dopaminergic neuron preparations, annotated with quality control data for Tyrosine Hydroxylase staining followed by flow cytometry. Cell lines were sampled at day 0, day 13, day 18 and day 25 of a dopaminergic neuron differentiation protocol. These datasets were collected using iPSCs generated from the same PD patients as above as well as from healthy control subjects.

56 RNAseq datasets from dopaminergic neuron preparations originating from 7 individuals, each with biological replicate clones and sampled at day 0, day 13, day 18 and day 25 of a dopaminergic neuron differentiation protocol. These datasets were collected using iPSCs generated from the same PD patients as above as well as from a healthy control subject.

8 RNAseq spiked mixtures (0.1%, 1% spike) of dopaminrgic neurons with iPSC. These datasets were collected using iPSCs generated from the same PD patients as above as well as from healthy control subjects.

Some of these datasets contained samples with known and characterized imperfections, such as chromosome abnormalities. These imperfections can be labelled, and their inclusion enhances the discriminatory power of the NeuroTest model.

B. The NeuroTest Data Model and Non-Negative Matrix Factorization (NMF)

For training the NeuroTest data model, non-negative matrix factorization (NMF) was first applied to the reference datasets (RNAseq and microarray datasets) described in Section A above. In contrast to distance-based clustering algorithms, such as hierarchical clustering, NMF uses matrix factorization to detect relations between items (Brunet et al., 2004). The dataset was represented as a large matrix, called the V matrix, which contained N mRNAs, and M cells lines. Over many iterations, NMF computed two component matrices, the W matrix (an N×k matrix) and the H matrix (a k×M matrix), which when multiplied together approximated the complete matrix for the dataset. Initial values in the W and H matrices were chosen randomly, and each iteration attempted to minimize the distance between WH and V. Clustering of cell lines was read out from the H matrix, in which each entry was indexed to a cluster number and a cell line, and contained a value indicating how well the cell line fit in that cluster (Brunet et al., 2004).

The criteria that conventional NMF (V˜W×H) optimizes is quality of approximation of all samples in the V matrix with a given number of metagenes. The number of metagenes is equivalent to k; the W matrix reflects how each gene in the V matrix contributes to a metagene; and the H matrix reflects cell lines' expression levels of these metagenes. Sometimes, approximation of all samples in the V matrix can lead to inappropriate “placement” of metagenes/meta-samples, for example: (1) between determined and less constrained stages, or (2) closer to an easy to approximate, large, low heterogeneity subgroup such as day 0. Therefore, discriminant NMF (Zafeiriou et al., 2006) was selected, which used the class labels in the training of the NMF model for detecting developmentally-determined cell types. Class labels indicated whether or not a cell line was at day 18 or later of the dopaminergic neuron differentiation protocol. To increase tolerance towards platform specific technical artifacts, the model was pre-trained on an initial collection of Illumina Beadarray data and lifted via a virtual Array approach to the RNA-seq platform. Model lifting was accomplished by using DNA probe sequence matching and summing code, quantile normalization, and transfer filtering. The “novelty” detection used conventional NMF since all samples were considered to stem from the same class of determined dopaminergic neurons (determined dopaminergic precursor cells). In this example, a relatively low dimensionality of k=3 (i.e., number of metagenes) was used.

After NMF was performed, the NeuroTest data model was then trained based on the outputs of NMF. Specifically, a logistic regression model was trained using metagene expression levels (the H matrix) and the class labels indicating whether or not a cell line was at day 18 or later of the dopaminergic neuron differentiation protocol. The number and selection of metagenes used for training (rows of the H matrix) was chosen based on a systematic search procedure optimizing for high accuracy in predicting class labels. Metagenes highly expressed in the target class (i.e., dopaminergic differentiation day 18 or later) were used for training. Parameters were selected by 5-fold cross-validation (Hastie et al., 2009) and evaluated on an unused portion of the training dataset which had been set aside for this purpose. Defined mixtures were used to identify the sensitivity of the approach, and to define cut-off boundaries.

C. Method to Compare the Input Test Data with the NeuroTest Data Model

After training of the NeuroTest model, test samples containing RNAseq data from separate developing neuronal preparations were prepared for input. Specifically, a TPM (Transcripts Per Kilobase Million) based “virtual array” was constructed for each test sample from its RNAseq data. A “virtual array” probe set was generated by locating the exact match probe sequences from the HT12v4 Illumina array in the Gencode v25 transcriptome sequences. This “virtual array” probe set was pruned for probes with either no match in the Gencode v25 transcriptome, or that had large model errors. The error in the “virtual array” model was assessed by performing a t-test between the expression in pluripotent samples of the GSE53094 dataset (processed as described above) and the pluripotent samples in the original training dataset. Thus, probes with no hits in Gencode v25 or with a foldchange >0.5 and a p.value<0.05 according to the t-test were removed, leaving 10,079 probes. A sample “virtual-array” was created by summing the Salmon TPM for transcripts with matches to each of these 10,079 probe sequences. The data was then transformed into a standard R-lumiBatch object (Du et al., 2008), quantile normalized, and tested with the previously prepared NeuroTest predictive model.

Specifically, the test sample's gene expression data was first converted to that of the metagenes used in training the NeuroTest model. To do so, and using the W matrix generated by applying NMF to the reference databases, regression analysis was performed to solve for the weighted combination of W-matrix basis vectors that best reconstructed the test sample's gene expression data. These weights corresponded to metagene expression levels of the test sample. The logistic regression model was then tested with the metagene expression levels of the test sample, while the gene expression data of the test sample was compared to that of the reference datasets. This yielded the NeuroScore and Novelty Score, respectively, which together reflected how similar the “test sample” precursor dopaminergic neuron was to those in the original reference data model.

After determining the test sample's NeuroScore and Novelty Score, these values were compared to predetermined thresholds for each parameter. The NeuroScore and Novelty Score thresholds were previously set to separate high quality dopaminergic neuronal lines from those with quantifiable deviations from the dopaminergic neuron developmentally-determined phenotype (e.g. “Low quality, low dopamine producing” cell lines) with 98% sensitivity and 100% specificity. Specifically, NeuroScore and Novelty Score thresholds were set based upon empirical testing using age-specific gene expression patterns from various timepoints throughout cellular differentiation (Day 0 to Day 13, Day 18, and Day 25). Previously, high NeuroScores had been obtained using Day 18 and Day 25 gene expression patterns, while low scores had been obtained for Day 0 gene expression patterns. High Novelty Scores had been obtained for gene expression patterns not usually observed for determined dopaminergic precursor cells. To find appropriate thresholds that could classify determined dopaminergic precursor cells with the highest degree of accuracy, both NeuroScore and Novelty Score thresholds had been iteratively adjusted until the area under the receiver operator characteristic (ROC) curve was maximized Based on this analysis, test samples were classified as determined dopaminergic precursor cells if they displayed Neuroscore ≥500 and Novelty Score ≤0.48.

Preparations of precursor dopaminergic neurons that had unusually high Novelty Scores indicated that these test samples should be: (a) excluded from any downstream therapeutic applications and (b) evaluated for epigenetic or genetic abnormalities or unwanted differentiation. Cell lines that had NeuroScores just below the cutoff threshold would need further investigation to confirm the integrity of the precursor dopaminergic neuron developmentally-determined state. For cell lines not passing either threshold, they may need to be excluded from any downstream therapeutic applications and potentially examined to rule out genetic abnormalities. Dopaminergic neuron differentiation of failures can be examined to evaluate reasons for failing NeuroTest.

D. Computing Framework

The computing framework used to implement parts [1] and [2] of NeuroTest was written in the R statistical computing language (R Development Core Team, 2010). R may be used as well as other modern programming languages with tools for statistical analysis. Nucleic acid sequence alignment used the Salmon pseudo aligner (Patro et al., 2017). NeuroTest was deployed as a data analysis pipeline for Illumina short read sequencing data and used on a Linx based local server or a Linux based virtual machine running either locally, or in a remote “cloud” computing environment. The pipeline included sequence quality evaluation and verification steps, sequence alignment to the transcriptome, counting and summarization of all gene expression levels, statistical (quantile) normalization of gene expression counts, statistical comparison to the data in the model and preparation and plotting of graphical output.

E. The NeuroTest Model Validation Dataset

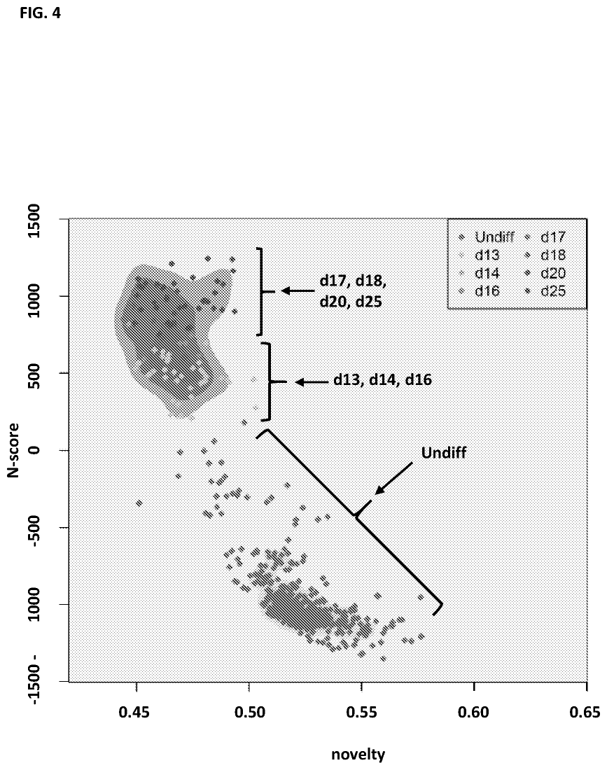

Additional RNAseq datasets were used to validate the NeuroTest model trained in Section B above. Before validation, these datasets were prepared for input as described in Section C above. As shown in FIG. 4, the NeuroTest model separated and discriminated between the undifferentiated, determined (˜day 14-day 18) and differentiated (˜day 20-day 25) neuronal cell types tested. The RNAseq validation dataset contained a total of 695 samples. The RNAseq gene expression data for differentiating dopaminergic neurons consisted of 37 sets of day 13, 1 set of day 14, 5 sets of day 16, 1 set day 17, 5 sets of day 18, 4 sets of day 20, and 35 sets of day 25. The remaining datasets were downloaded from public repositories.

Prior to validation, the NeuroTest model was initially trained on discriminating genes from the microarray data and supplemented with RNAseq based gene expression data. Then, RNAseq data was used as validation data since the model training was done with Illumina beadarray data by using 5 fold cross-validation. The validation RNAseq data was generated or downloaded from public data repositories. The samples in the upper left quadrant of FIG. 4 passed for both high NeuroScore and low Novelty Score. The “Undiff” samples (mostly undifferentiated IPSC, diamonds) failed NeuroTest due to getting a low NeuroScore and having elevated Novelty Scores compared to the reference data model.

F. The NeuroTest Challenge Dataset and Testing the Data Model

For further validation and to demonstrate that the model can distinguish between cell types expected to pass or fail NeuroTest, a test dataset was constructed with a set of predicted outcomes. The challenge dataset consisted of 86 publicly available RNAseq datasets, created from a variety of brain cell types (mainly astrocytes and various neurons). The RNAseq data were downloaded from The Gene Expression Omnibus (GEO-NCBI) https://www.ncbi.nlm.nih.gov/geo/.

Archival GEO GSE dataset numbers:

GSE116124 (di Domenico et al., 2019)

GSE117664 (Astrocytes, unpublished, but data released)

GSE99652 (Weissbein et al., 2017)

GSE120306 (unpublished, but data released for ipsc derived astrocytes)

GSE98289 (Hall et al., 2017)

GSE84684 (Kouroupi et al., 2017).

Challenging the NeuroTest model trained in Section B above with these new datasets revealed that the model could determine which samples matched to the phenotype of a dopaminergic neuron and which did not.

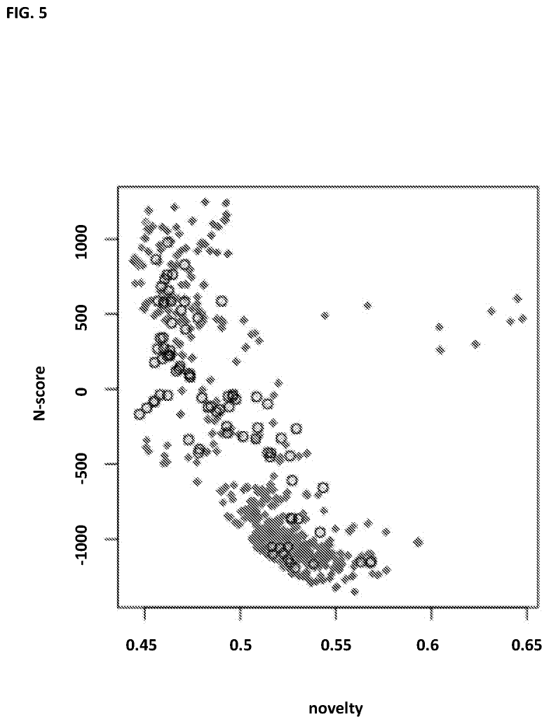

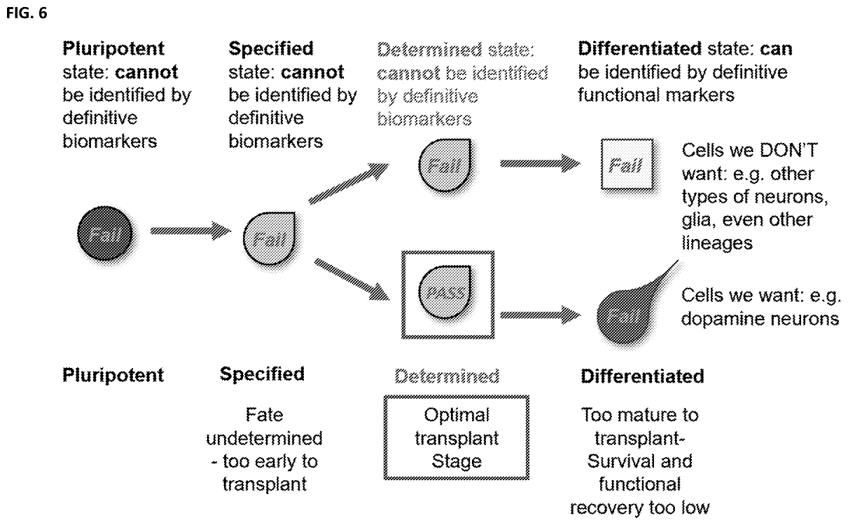

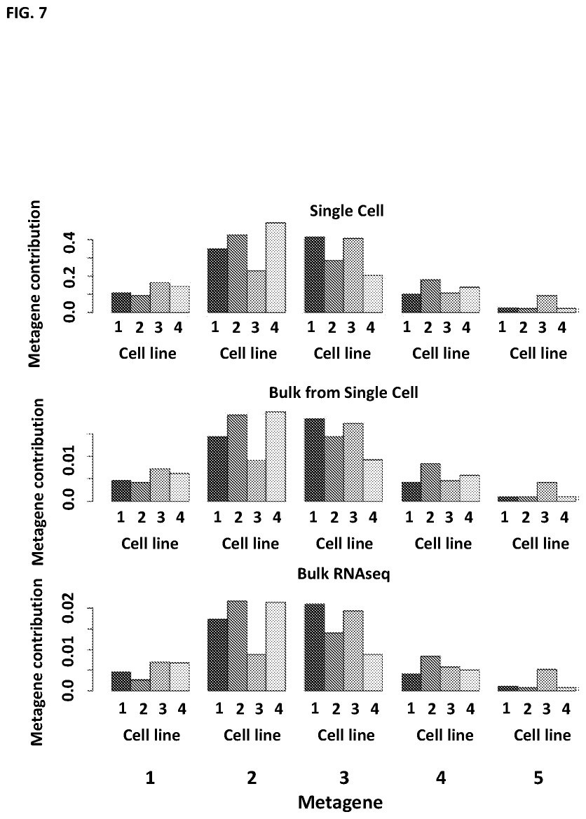

FIG. 5 shows the NeuroTest results from the analysis of the 86 publicly available neuronal RNAseq datasets. The datapoints highlighted with the black circles are specifically the data points from the challenge datasets. The colored background datapoints are from the NeuroTest validation analysis of the 695 samples of validation data. These results provide context for the NeuroTest challenge data. The spread of the challenge data, spanning the range from iPSC to cancer cells to neuronal reflected the input data. The tabular output revealed that NeuroTest gave a “pass” score to dopaminergic neuron cellular preparations.

G. R-Code Underlying the NeuroTest Core Functions

Example R-code which executes the statistical routine exemplified above for comparing the test sample to the reference data model is shown below. On the server, it functioned as a part of a larger data analysis pipeline. This routine could be envisaged and re-written in numerous different ways.

```
  CODE BELOW:
  NeurotestAllBatch1<−
function(working.lumi=working.lumi,normalize=“quantile”,transform=FALSE,Wneuro=Wneuro1,Wneu
roN=WneuroN1,target=targetNeuro,techIndex=c(1,1)){
    if( normalize==“quantile”){
      require(preprocessCore)
      if(transform==TRUE) working.lumi <− lumiT(working.lumi)
      exprs(working.lumi)<−normalize.quantiles.use.target(exprs(working.lumi),target =
drop((target)))
    }
    # corrected
    A <− fData(working.lumi)[, 1]
    sel.match <− match(colnames(Wneuro), A)
    sel <− match(rownames(Wneuro), fData(working.lumi)[, 1])
    V<−matrix(exprs(working.lumi)[sel]!is.na(sel)],],ncol=ncol(working.lumi) )
    HNeuro.new <− predictH(V, Wneuro[!is.na(sel), ])
    HNeuroN.new <− predictH(V, WneuroN[!is.na(sel), ])
    #resids <−exprs(working.lumi)[sel, ][!is.na(sel), ] - WnovCor[!is.na(sel), ] %*% H12.new
    resids<−matrix(0,ncol=ncol(working.lumi), nrow=nrow(WneuroN))
    resids[!is.na(sel),] <−V - WneuroN[!is.na(sel), ] %*% HNeuroN.new
    novel.new <− apply(resids\hat{ }2,2,mean )
    novel.new <− sqrt(novel.new)
  # print(novel.new)
    s.new <− drop(coefNeuro[1] + apply(coefNeuro[−c(1)] * HNeuro.new[, ],2,sum))
      #print(HNeuro.new)
    jpeg(file=“neuro1.jpg”)
    plot(logisticF(s.new)~novel.new, main=“neuroScore vs
Novelty”,ylab=“neuri”,xlab=“deviation”,xlim=c(0.3,1),ylim=c(0,100))
    dev.off( )
    jpeg(file=“neuro2.jpg”)
    barplot(logisticF(s.new),las=2, main=“neuroScore”,ylab=“Neuriscore”,ylim=c(0,100))
    dev.off( )
    write.csv2(data.frame(ID=sampleNames(working.lumi),neuriScore=logisticF(s.new),
neuriScoreRaw=s.new,NeuriNovel=novel.new),file=“neuritest.csv”)
    return(list(novelNeuro=novel.new,scoreNeuro=s.new))
  }
  CODE ENDS HERE
```

### Example 2: Using Single-Cell Rnaseq Data for Predicting Cell Phenotype

The use of single-cell RNAseq (scRNAseq) data was evaluated for use in the method for determining the whole cell phenotype of a cell type described in Example 1 herein. As above, NMF was used to derive metagenes (W matrix) and expression levels thereof (H matrix) from scRNAseq datasets. After performing NMF, metagenes derived from scRNAseq data were compared to those derived from corresponding bulk RNA data. Next, a logistic regression model was trained on metagene expression levels derived from scRNAseq data in order to predict the presence of determined dopaminergic neurons, and its performance on bulk RNAseq test samples was assessed.

To do so, neural precursor cells were generated as described above from the same PD patients and healthy control subjects. Single-cell RNA (scRNA) was isolated from these precursor cells at day 13, day 18, and day 25 of an in vitro dopaminergic neuron differentiation protocol using the isolation protocol illustrated in FIG. 1, Panel A of Zheng et al., 2017 (Nature Communications 8: 14049). Briefly, individual precursor cells were encapsulated into droplets alongside gel beads containing oligo(dT) primers with a unique cell barcode used to index the 3′ end of cDNA molecules during reverse transcription. In this manner, RNA transcripts were assigned to individual precursor cells during Illumina sequence analysis. In addition to isolating scRNA, bulk RNAseq data was also collected from the same samples of neural precursor cells, thus generating matched bulk RNAseq data.

A. Comparing Metagenes

Metagenes and expression levels thereof between different types of data (scRNAseq, bulk RNAseq) from the same samples were compared. Aggregrated scRNAseq data (i.e., bulk from single cell data) was also generated in order to approximate bulk RNAseq data, with aggregation achieved by taking the mean gene expression level across single cells within the same sample. Conventional NMF was performed on each dataset in order to determine each datasets' metagene composition and the expression levels of each metagene.

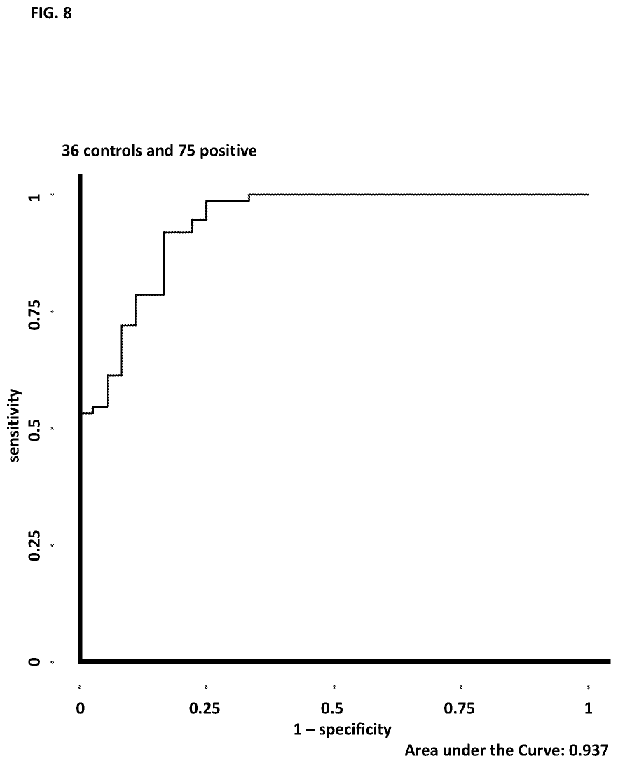

FIG. 7 shows a metagene comparison between scRNAseq, aggregated scRNAseq (i.e., bulk from single cell), and matched bulk RNAseq datasets. In FIG. 7, five metagenes for four cell lines at day 18 of differentiation are shown. Expression levels of the five metagenes were consistent across datasets (scRNAseq, aggregated scRNAseq, and matched bulk RNAseq) for each of the four cell lines. Thus, equivalent metagene compositions of the samples were reconstructed from both aggregated scRNAseq and bulk RNAseq datasets.

B. Comparing Model Performance and Output

To evaluate an scRNAseq-trained model used to predict the presence of a determined dopaminergic precursor cell, an NMF and model training procedure similar to that decribed in Example 1, Section B, herein was employed. Specifically, conventional NMF was first performed on scRNAseq data from precursor cells at day 25 of differentiation, thus producing a W matrix reflecting the contribution of each gene to a metagene. Next, scRNAseq gene expression data from each of several timepoints during differentiation was converted to metagene expression data. As above, this conversion was performed by using the W matrix and regression analysis to solve for each sample's metagene expression levels. Finally, a logistic regression model was trained using the metagene expression data and class labels indicating whether or not the cells were determined dopaminergic precursor cells.

To test for model performance, the scRNAseq-trained model was tested on 111 out-of-sample bulk RNAseq data points. Of these datapoints, 75 were from samples of determined dopaminergic precursor cells. As shown by the receiver operator characteristic (ROC) curve in FIG. 8, the scRNAseq-trained model achieved above-chance classification performance on bulk RNAseq data (AUC=0.937), even without explicit integration of bulk RNAseq data into the scRNAseq-trained model and optimization thereof.

Together, these results indicate that scRNAseq data could be incorporated into the method for determining the whole cell phenotype of a cell type described in Example 1 herein.

### Example 3: Using Single-Cell Rnaseq Data and Marker Genes for Predicting Cell Phenotype

Single-cell RNAseq data was incorporated into the method described in Example 1 herein. The evaluation of test samples' expression of various marker genes was also incorporated. FIG. 9 shows an exemplary workflow of the method, which used gene expression datasets from samples of neural precursor cells both (i) to train a model to predict the presence of determined dopaminergic precursor cells within a sample and (ii) to estimate baseline deviations in samples' single-gene expression levels and establish tolerated deviation levels for future test samples. Incorporating scRNAseq data improved definition of the cellular signatures in differentiating cultures of dopaminergic neurons. Use of the marker genes provided diagnostic insight into the quality of differentiating samples. In this manner, the ability to identify specific features that might impair the functionality of cell samples was improved.

A. Datasets for Model Training and Gene Deviation Estimation

Single-cell and bulk RNAseq datasets were generated as described in Examples 1 and 2 herein. Specifically, scRNA and bulk RNA were isolated from samples of precursor cells at day 13, day 18, and day 25 of an in vitro dopaminergic neuron differentiation protocol. After RNA sequencing, all scRNAseq data was pre-processed using a Seurat single-cell processing pipeline. This preprocessing was used to match single cells to their respective cell lines, remove data representing more than one cell (doublets), and filter out samples based on mitochondrial and ribosomal RNA content. Only genes with data available in all scRNAseq and bulk RNAseq datasets were included in subsequent processing.

B. Non-Negative Matrix Factorization (NMF) for Metagene Derivation

As in Example 1, metagenes were derived using NMF. Specifically, conventional NMF was performed for each scRNAseq dataset (day 13, day 18, day 25), in this manner deriving separate metagenes (W matrices) for each developmental timepoint. These metagene models described expected patterns of whole culture gene expression throughout differentiation. Initial W and H matrices were provided for each performance of NMF. For the initial W matrix, uniform manifold approximation and projection (UMAP) was performed on the scRNAseq dataset after preprocessing with principal component analysis (PCA). The cluster centroids output by UMAP, for which there were 5-6 clusters per scRNAseq dataset, were used as the initial W matrix. An initial H matrix was approximated from each scRNAseq dataset and its corresponding initial W matrix using non-negative least squares approximation.

C. Model Training

After NMF, the metagene expression levels (loadings) of the bulk RNAseq datasets were determined for all metagenes (i.e., those derived from each of the three scRNAseq datasets). First, the W matrices produced in Section B above were location- and scale-normalized. Next, a penalized regression model was used per sample in order to estimate each sample's bulk RNAseq data using each of the normalized W matrix (timepoint-specific metagenes). In this manner, samples' expression levels of metagenes derived throughout development were approximated, thus providing a time-resolved profile for each sample. Using these profiles, a logistic regression model was trained using the metagene expression levels for the bulk RNAseq datasets and class labels indicating whether or not the samples in the bulk RNAseq datasets were at day 18 or later of the dopaminergic neuron differentiation protocol. Thus, a model for predicting the presence of a determined (e.g., day 18 or later) dopaminergic precursor cell was generated, the output of the model providing an indication akin to the NeuroScore described in Example 1 herein. As the model was trained on bulk RNAseq data, key aspects related to cell population structure and important biological processes, such as cell cycle status, were captured in the model.

D. Deviation Score Calculation

Deviation scores similar to the Novelty Scores described in Example 1 herein were also calculated per bulk RNA sample. These deviation scores provided summary statitics of irregular pattrns of gene expression. To do so, single-gene expression level deviation was calculated per sample. Calculated deviations were specific to the timepoint at which each sample was collected (day 13, day 18, or day 25). First, and for optimal calculation of deviation given the count-based nature of bulk RNAseq data, a Limma-Voom counts-per-million (CPM) approach was used to convert bulk RNAseq data from units of TPM to CPM. Next, a linear model was used per sample in order to calculate estimated gene expression data based on the sample's metagene expression levels (estimated in Section B above). The residuals per gene (difference between the estimated gene expression data and the actual bulk RNAseq data in CPM) was then calculated.

To normalize residuals across genes, a set of genes with stable expression levels was first used to estimate typical deviation across samples. The median absolute deviation of stably expressed genes with log₂CPM values between four and 9.5 was used as an estimate of typical gene deviation across samples, and based on this analysis, a value of 0.5 was used as a baseline for residual standard deviation. Thus, residuals were normalized by dividing by either the standard deviation of gene expression across samples or 0.5 if such standard deviation was less than 0.5.

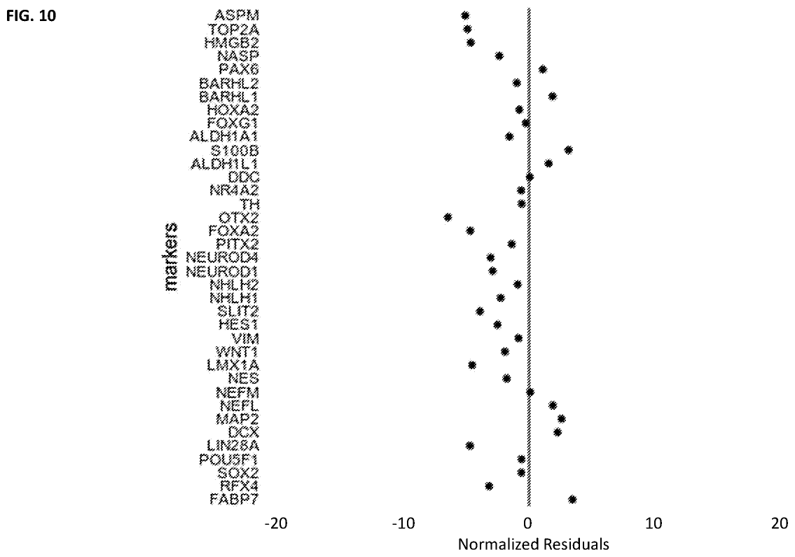

After normalization, two quantile values per sample were determined. First, the 95% quantile of the absolute normalized residuals was calculated. Second, the 95% quantile of absolute normalized residuals corresponding to ˜30 predefined marker genes was determined. These marker genes are shown in Table E1 below and were chosen based on their dynamic behavior through and impact on dopaminergic neuron differentiation. An exemplary sample's normalized residuals for these marker genes are shown in FIG. 10. Some markers, like astrocyte markers S100B and LDH1L1, should be absent or at very low levels in samples. The maximum quantile value between the two calculated values was then used as the overall deviation score for the sample, akin to the Novelty Score described in Example 1 and providing a conservative (worst case) picture of deviation in each sample.

TABLE E1
Marker Genes and Biological Significence

| Gene | Biological Significance |
|---|---|
| FABP7 | Radial glial cell marker |
| RFX4 | Early neuronal development gene, expressed until |
| | progenitor state only |
| SOX2 | Early neuronal development genes expressed until |
| | progenitor state only |
| POU5F1 | Pluripotency specific marker |
| LIN28A | Pluripotency specific marker |
| DCX | Intermediate to late neuronal marker, expressed |
| | in immature neurons |
| MAP2 | Intermediate to late neuronal marker |
| NEFL | Neurofilament light polypeptide chain marker |
| NEFM | Neurofilament medium polypeptide chain marker |
| NES | Nestin filament gene |
| LMX1A | Early patterning marker |
| WNT1 | Early patterning marker |
| VIM | Neural progenitor cell marker |
| HES1 | Neural progenitor cell marker |
| SLIT2 | Early migration marker, Robo-slit system, |
| NHLH1 | Stage specific transcription factor |
| NHLH2 | Stage specific transcription factor |
| NEUROD1 | Neuro-differentiation factor |
| NEUROD4 | Neuro-differentiation factor |
| PITX2 | Required for normal development of neurons |
| FOXA2 | Controls dopaminergic neuron development |
| OTX2 | Regulates identity and fate of neuronal progenitor |
| | cells |
| TH | Dopaminergic neuron marker |
| NR4A2 | Dopaminergic neuron marker |
| DDC | Dopaminergic neuron marker |
| ALDH1L1 | Dopaminergic neuron marker |
| S100B | Astrocyte marker |
| ALDH1A1 | Astrocyte marker |
| FOXG1 | Forebrain marker |
| HOXA2 | Hindbrain marker |
| BARHL1 | Subthalamic nucleus marker |
| BARHL2 | Subthalamic nucleus marker |
| PAX6 | Radial glial marker, region and time specific |
| NASP | Cell cycle S-phase marker |
| HMGB1 | G to M phase, proliferating progenitor marker |
| TOP2A | G to M-phase marker |
| ASPM | G to M-phase, neuron symmetric proliferation marker |

E. Thresholds for Model Output and Deviation Scores

To establish predetermined thresholds for evaluating test samples, model predictions (NeuroScores) and deviation scores (Novelty Scores) across samples were examined. As in Example 1, Section C herein, samples' bulk RNAseq data was converted using a linear model to expression levels of metagenes used to train the model produced in Example 3, Section C, and these converted metagene expression levels were provided to the trained model. Deviation scores were also calculated per sample as described in Section C above.

Such analysis indicated that samaples from day 18-25 of differentiation were likely to have model output greater than 0 (i.e., probability greater than 0.5 of the sample comprising a determined dopaminergic precursor cell), and it was determined that samples having a Novelty Score of less than 5 had acceptable gene deviation.

F. Model Validation

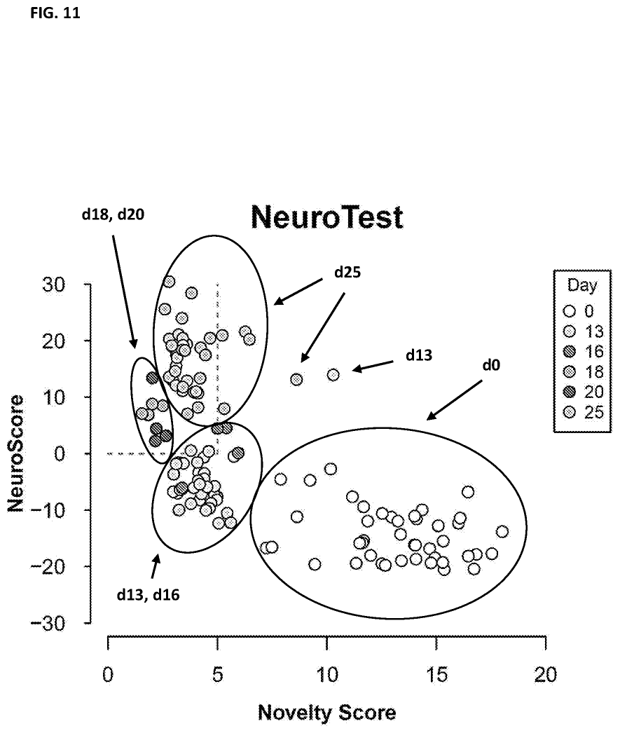

FIG. 11 shows model predictions (NeuroScores) and deviation scores (Novelty Scores) calculated across a collection of developing dopaminergic neurons and undifferentiated iPSCs. The cells were analysed by RNAseq at the differentiation timepoints shown in FIG. 11. FIG. 11 shows that based on threshold values described in Section D above, all samples from day 18-25 of differentiation exceeded the NeuroScore threshold, though some also had Novelty Scores higher than the predetermined threshold. All samples that were undifferentiated iPSCs (day 0) or at days 13-16 of differentiation did not meet one or both of the predetermined thresholds. These results indicate that the method was able to (i) predict with high specificity and sensitivity samples with determined dopaminergic precursor cells and (ii) identify samples with higher than expected or higher than tolerated deviation in gene expression levels.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Tables

TABLE 1
Exemplary gene ontologies including one or more
genes with 4 times increased gene expressior
levels relative to a pluripotent stem cell.

| GO ACCESSION | GO Term |
|---|---|
| GO:0007399 | nervous system development |
| GO:0120025 | plasma membrane bounded cell |
| | projection |
| GO:0042995 | cell projection |
| GO:0032502\|GO:0044767 | developmental process |
| GO:0048856 | anatomical structure development |
| GO:0048731 | system development |
| GO:0022008 | neurogenesis |
| GO:0048699 | generation of neurons |
| GO:0007275 | multicellular organism development |
| GO:0030030 | cell projection organization |
| GO:0032501\|GO:0044707\| | multicellular organismal process |
| GO:0050874 | |
| GO:0048468 | cell development |
| GO:0120036 | plasma membrane bounded cell |
| | projection organization |
| GO:0120038 | plasma membrane bounded cell |
| | projection part |
| GO:0044463 | cell projection part |
| GO:0097458 | neuron part |
| GO:0045202 | synapse |
| GO:0030182 | neuron differentiation |
| GO:0030154 | cell differentiation |
| GO:0048869 | cellular developmental process |
| GO:0051960 | regulation of nervous system |
| | development |
| GO:0007156 | homophilic cell adhesion via plasma |
| | membrane adhesion molecules |
| GO:0005929\|GO:0072372 | cilium |
| GO:0035082\|GO:0035083\| | axoneme assembly |
| GO:0035084 | |
| GO:0060284 | regulation of cell development |
| GO:0050767 | regulation of neurogenesis |
| GO:0001578 | microtubule bundle formation |
| GO:0016339 | calcium-dependent cell-cell adhesion via |
| | plasma membrane cell adhesion molecules |
| GO:0043005 | neuron projection |
| GO:0044456 | synapse part |
| GO:0098742 | cell-cell adhesion via plasma-membrane |
| | adhesion molecules |
| GO:0045664 | regulation of neuron differentiation |
| GO:0006928 | movement of cell or subcellular component |
| GO:0099699 | integral component of synaptic membrane |
| GO:0048666 | neuron development |
| GO:0003341\|GO:0036142 | cilium movement |
| GO:0005509 | calcium ion binding |
| GO:0097060 | synaptic membrane |
| GO:0031514\|GO:0009434\| | motile cilium |
| GO:0031512 | |
| GO:0007155\|GO:0098602 | cell adhesion |
| GO:0010975 | regulation of neuron projection |
| | development |
| GO:0098794 | postsynapse |
| GO:0022610 | biological adhesion |
| GO:0030424 | axon |
| GO:0099240 | intrinsic component of synaptic |
| | membrane |
| GO:0032989 | cellular component morphogenesis |
| GO:0120035 | regulation of plasma membrane bounded |
| | cell projection organization |
| GO:0000902\|GO:0007148\| | cell morphogenesis |
| GO:0045790\|GO:0045791 | |
| GO:0048812 | neuron projection morphogenesis |
| GO:0036477 | somatodendritic compartment |
| GO:0031344 | regulation of cell projection |
| | organization |
| GO:0120039 | plasma membrane bounded cell |
| | projection morphogenesis |
| GO:0061564 | axon development |
| GO:0048858 | cell projection morphogenesis |
| GO:0099055 | integral component of postsynaptic |
| | membrane |
| GO:0009653 | anatomical structure morphogenesis |
| GO:0098609\|GO:0016337 | cell-cell adhesion |
| GO:0031175 | neuron projection development |
| GO:0005930\|GO:0035085\| | axoneme |
| GO:0035086 | |
| GO:0010720 | positive regulation of cell development |
| GO:0007416 | synapse assembly |
| GO:0097014 | ciliary plasm |
| GO:0032990 | cell part morphogenesis |
| GO:0098936 | intrinsic component of postsynaptic |
| | membrane |
| GO:0043025 | neuronal cell body |
| GO:0050768 | negative regulation of neurogenesis |
| GO:0051962 | positive regulation of nervous |
| | system development |
| GO:0050808 | synapse organization |
| GO:0007409\|GO:0007410 | axonogenesis |
| GO:2000026 | regulation of multicellular organismal |
| | development |
| GO:0045597 | positive regulation of cell |
| | differentiation |
| GO:0044441\|GO:0044442 | ciliary part |
| GO:0007417 | central nervous system development |
| GO:0048667 | cell morphogenesis involved in |
| | neuron differentiation |
| GO:0010721 | negative regulation of cell development |
| GO:0044459 | plasma membrane part |
| GO:0060322 | head development |
| GO:0045211 | postsynaptic membrane |
| GO:0045666 | positive regulation of neuron |
| | differentiation |
| GO:0032838 | plasma membrane bounded cell |
| | projection cytoplasm |
| GO:0099056 | integral component of presynaptic |
| | membrane |
| GO:0051961 | negative regulation of nervous |
| | system development |
| GO:0044297 | cell body |
| GO:0007018 | microtubule-based movement |
| GO:0050769 | positive regulation of neurogenesis |
| GO:0040011 | locomotion |
| GO:0050793 | regulation of developmental process |
| GO:0051094 | positive regulation of developmental |
| | process |
| GO:0005874 | microtubule |
| GO:0000904 | cell morphogenesis involved in |
| | differentiation |
| GO:0010976 | positive regulation of neuron |
| | projection development |
| GO:0045595 | regulation of cell differentiation |
| GO:0050770 | regulation of axonogenesis |
| GO:0099536 | synaptic signaling |
| GO:0098889 | intrinsic component of presynaptic |
| | membrane |
| GO:0051239 | regulation of multicellular |
| | organismal process |
| GO:0007420 | brain development |
| GO:0099537 | trans-synaptic signaling |
| GO:0031346 | positive regulation of cell |
| | projection organization |
| GO:0007268 | chemical synaptic transmission |
| GO:0098916 | anterograde trans-synaptic signaling |
| GO:0097485 | neuron projection guidance |
| GO:0044782 | cilium organization |
| GO:0031226 | intrinsic component of plasma membrane |
| GO:0060285\|GO:0071974 | cilium-dependent cell motility |
| GO:0010769 | regulation of cell morphogenesis |
| | involved in differentiation |
| GO:0001539 | cilium or flagellum-dependent cell motility |
| GO:0050804 | modulation of chemical synaptic |
| | transmission |
| GO:0099177 | regulation of trans-synaptic signaling |
| GO:0005887 | integral component of plasma membrane |
| GO:0098984 | neuron to neuron synapse |
| GO:0045665 | negative regulation of neuron differentiation |
| GO:0050919 | negative chemotaxis |
| GO:0007411\|GO:0008040 | axon guidance |
| GO:0030425 | dendrite |
| GO:0061387 | regulation of extent of cell growth |
| GO:0097447 | dendritic tree |
| GO:0050803 | regulation of synapse structure or activity |
| GO:0042734 | presynaptic membrane |
| GO:0042391 | regulation of membrane potential |
| GO:0001764 | neuron migration |
| GO:0032279 | asymmetric synapse |
| GO:0010770 | positive regulation of cell morphogenesis |
| | involved in differentiation |
| GO:0021953 | central nervous system neuron differentiation |
| GO:0099572 | postsynaptic specialization |
| GO:0098590 | plasma membrane region |
| GO:0044447 | axoneme part |
| GO:0098978 | glutamatergic synapse |
| GO:0014069\|GO:0097481\| | postsynaptic density |
| GO:0097483 | |
| GO:0033267 | axon part |
| GO:0010977 | negative regulation of neuron projection |
| | development |
| GO:0007017 | microtubule-based process |
| GO:0150034 | distal axon |
| GO:0034702 | ion channel complex |
| GO:0034703 | cation channel complex |
| GO:0050807 | regulation of synapse organization |
| GO:0060271\|GO:0042384 | cilium assembly |
| GO:0051240 | positive regulation of multicellular |
| | organismal process |
| GO:0050772 | positive regulation of axonogenesis |
| GO:0120031 | plasma membrane bounded cell projection |
| | assembly |
| GO:0007626 | locomotory behavior |
| GO:0008092 | cytoskeletal protein binding |
| GO:0005886\|GO:0005904 | plasma membrane |
| GO:0007610\|GO:0044708 | behavior |
| GO:0098793 | presynapse |
| GO:0022604 | regulation of cell morphogenesis |
| GO:0007267 | cell-cell signaling |
| GO:0071944 | cell periphery |
| GO:0099060 | integral component of postsynaptic |
| | specialization membrane |
| GO:0022836 | gated channel activity |
| GO:0030031 | cell projection assembly |
| GO:0042220 | response to cocaine |
| GO:0019226 | transmission of nerve impulse |
| GO:0030516 | regulation of axon extension |
| GO:0035637 | multicellular organismal signaling |
| GO:0045596 | negative regulation of cell differentiation |
| GO:0021954 | central nervous system neuron development |
| GO:0022832 | voltage-gated channel activity |
| GO:0005244 | voltage-gated ion channel activity |
| GO:1902495 | transmembrane transporter complex |
| GO:0050771 | negative regulation of axonogenesis |
| GO:0048513 | animal organ development |
| GO:0022839 | ion gated channel activity |
| GO:0098948 | intrinsic component of postsynaptic |
| | specialization membrane |
| GO:0001508 | action potential |
| GO:0099568 | cytoplasmic region |
| GO:0008484 | sulfuric ester hydrolase activity |
| GO:0051966 | regulation of synaptic transmission, |
| | glutamatergic |
| GO:0003358 | noradrenergic neuron development |
| GO:0033602 | negative regulation of dopamine secretion |
| GO:0005261\|GO:0015281\| | cation channel activity |
| GO:0015338 | |
| GO:0022603 | regulation of anatomical structure |
| | morphogenesis |
| GO:1990351 | transporter complex |
| GO:0097729 | 9 + 2 motile cilium |
| GO:0015631 | tubulin binding |
| GO:0051270 | regulation of cellular component movement |
| GO:0005216 | ion channel activity |
| GO:0016043\|GO:0044235\| | cellular component organization |
| GO:0071842 | |
| GO:0031345 | negative regulation of cell projection |
| | organization |
| GO:0005856 | cytoskeleton |
| GO:0022838 | substrate-specific channel activity |
| GO:0099061 | integral component of postsynaptic |
| | density membrane |
| GO:0098982 | GABA-ergic synapse |
| GO:0051674 | localization of cell |
| GO:0048870 | cell motility |
| GO:0060294 | cilium movement involved in cell motility |
| GO:0072359 | circulatory system development |
| GO:0099634 | postsynaptic specialization membrane |
| GO:0015630 | microtubule cytoskeleton |
| GO:0036126 | sperm flagellum |
| GO:1990939 | ATP-dependent microtubule motor activity |
| GO:0072347 | response to anesthetic |
| GO:0015267\|GO:0015249\| | channel activity |
| GO:0015268 | |
| GO:0022803\|GO:0022814 | passive transmembrane transporter activity |
| GO:0008045 | motor neuron axon guidance |
| GO:0098797 | plasma membrane protein complex |
| GO:0060160 | negative regulation of dopamine |
| | receptor signaling pathway |
| GO:0099146 | intrinsic component of postsynaptic |
| | density membrane |
| GO:0010771 | negative regulation of cell morphogenesis |
| | involved in differentiation |
| GO:0000226 | microtubule cytoskeleton organization |
| GO:0045503 | dynein light chain binding |
| GO:0005578 | proteinaceous extracellular matrix |
| GO:0030334 | regulation of cell migration |
| GO:0044304 | main axon |
| GO:0010463 | mesenchymal cell proliferation |
| GO:0010646 | regulation of cell communication |
| GO:0008574 | ATP-dependent microtubule motor activity, |
| | plus-end-directed |
| GO:0043279 | response to alkaloid |

TABLE 2
Exemplary genes of gene ontology GO:0048699 with 4 times increased
gene expression levels relative to a pluripotent stem cell.

| | Gene |
|---|---|
| Gene ID | Symbol |
| ENSG00000150625.16 | GPM6A |
| ENSG00000149295.13 | DRD2 |
| ENSG00000101144.12 | BMP7 |
| ENSG00000108947.4 | EFNB3 |
| ENSG00000075223.13 | SEMA3C |
| ENSG00000186765.11 | FSCN2 |
| ENSG00000108231.12 | LGI1 |
| ENSG00000277363.4 | SRCIN1 |
| ENSG00000162552.14 | WNT4 |
| ENSG00000145147.19 | SLIT2 |
| ENSG00000157168.18 | NRG1 |
| ENSG00000146216.11 | TTBK1 |
| ENSG00000141622.13 | RNF165 |
| ENSG00000170558.8 | CDH2 |
| ENSG00000162374.16 | ELAVL4 |
| ENSG00000119547.5 | ONECUT2 |
| ENSG00000183762.12 | KREMEN1 |
| ENSG00000261678.2 | SCRT1 |
| ENSG00000169330.8 | KIAA1024 |
| ENSG00000171587.14 | DSCAM |
| ENSG00000078018.19 | MAP2 |
| ENSG00000196159.11 | FAT4 |
| ENSG00000077264.14 | PAK3 |
| ENSG00000134259.3 | NGF |
| ENSG00000137872.16 | SEMA6D |
| ENSG00000104435.13 | STMN2 |
| ENSG00000140836.14 | ZFHX3 |
| ENSG00000081479.12 | LRP2 |
| ENSG00000118137.9 | APOA1 |
| ENSG00000058404.19 | CAMK2B |
| ENSG00000112139.14 | MDGA1 |
| ENSG00000167178.15 | ISLR2 |
| ENSG00000132639.12 | SNAP25 |
| ENSG00000123307.3 | NEUROD4 |
| ENSG00000109132.6 | PHOX2B |
| ENSG00000077279.17 | DCX |
| ENSG00000187391.19 | MAGI2 |
| ENSG00000145675.14 | PIK3R1 |
| ENSG00000149294.16 | NCAM1 |
| ENSG00000140538.16 | NTRK3 |
| ENSG00000107859.9 | PITX3 |
| ENSG00000186487.17 | MYT1L |
| ENSG00000135407.10 | AVIL |
| ENSG00000171450.5 | CDK5R2 |
| ENSG00000173404.4 | INSM1 |
| ENSG00000125285.5 | SOX21 |
| ENSG00000134352.19 | IL6ST |
| ENSG00000168280.16 | KIF5C |
| ENSG00000159082.17 | SYNJ1 |
| ENSG00000160145.15 | KALRN |
| ENSG00000151892.14 | GFRA1 |
| ENSG00000204852.15 | TCTN1 |
| ENSG00000075275.16 | CELSR1 |
| ENSG00000176842.14 | IRX5 |
| ENSG00000109099.13 | PMP22 |
| ENSG00000159216.18 | RUNX1 |
| ENSG00000151640.12 | DPYSL4 |
| ENSG00000091129.19 | NRCAM |
| ENSG00000198795.10 | ZNF521 |
| ENSG00000139915.18 | MDGA2 |
| ENSG00000117707.15 | PROX1 |
| ENSG00000198597.8 | ZNF536 |
| ENSG00000166963.12 | MAP1A |
| ENSG00000172260.14 | NEGR1 |
| ENSG00000221866.9 | PLXNA4 |
| ENSG00000082397.17 | EPB41L3 |
| ENSG00000172020.12 | GAP43 |
| ENSG00000135333.13 | EPHA7 |
| ENSG00000090932.10 | DLL3 |
| ENSG00000132821.11 | VSTM2L |
| ENSG00000172201.11 | ID4 |
| ENSG00000124785.8 | NRN1 |
| ENSG00000152377.13 | SPOCK1 |
| ENSG00000143507.17 | DUSP10 |
| ENSG00000168542.13 | COL3A1 |
| ENSG00000006210.6 | CX3CL1 |
| ENSG00000184347.14 | SLIT3 |
| ENSG00000008735.13 | MAPK8IP2 |
| ENSG00000135472.8 | FAIM2 |
| ENSG00000140262.17 | TCF12 |
| ENSG00000153162.8 | BMP6 |
| ENSG00000185189.16 | NRBP2 |
| ENSG00000154654.14 | NCAM2 |
| ENSG00000064393.15 | HIPK2 |
| ENSG00000140937.13 | CDH11 |
| ENSG00000150471.16 | ADGRL3 |
| ENSG00000170396.7 | ZNF804A |
| ENSG00000083290.19 | ULK2 |
| ENSG00000163394.5 | CCKAR |
| ENSG00000004139.13 | SARM1 |
| ENSG00000130827.6 | PLXNA3 |
| ENSG00000171617.13 | ENC1 |
| ENSG00000139352.3 | ASCL1 |
| ENSG00000164853.8 | UNCX |
| ENSG00000143995.19 | MEIS1 |
| ENSG00000004848.7 | ARX |
| ENSG00000139767.8 | SRRM4 |
| ENSG00000119283.15 | TRIM67 |
| ENSG00000170017.12 | ALCAM |
| ENSG00000065320.8 | NTN1 |
| ENSG00000138311.15 | ZNF365 |
| ENSG00000162676.11 | GFI1 |
| ENSG00000141433.12 | ADCYAP1 |
| ENSG00000118432.12 | CNR1 |
| ENSG00000148677.6 | ANKRD1 |
| ENSG00000171094.15 | ALK |
| ENSG00000015592.16 | STMN4 |
| ENSG00000186868.15 | MAPT |
| ENSG00000018189.12 | RUFY3 |
| ENSG00000076356.6 | PLXNA2 |
| ENSG00000136040.8 | PLXNC1 |
| ENSG00000131711.14 | MAP1B |
| ENSG00000157851.16 | DPYSL5 |
| ENSG00000151490.13 | PTPRO |
| ENSG00000157240.3 | FZD1 |
| ENSG00000105880.4 | DLX5 |

TABLE 3
Exemplary genes of gene ontology GO:0050767 with 4 times increased
gene expression levels relative to a pluripotent stem cell.

| | Gene |
|---|---|
| Gene ID | Symbol |
| ENSG00000149295.13 | DRD2 |
| ENSG00000101144.12 | BMP7 |
| ENSG00000108947.4 | EFNB3 |
| ENSG00000075223.13 | SEMA3C |
| ENSG00000277363.4 | SRCIN1 |
| ENSG00000145147.19 | SLIT2 |
| ENSG00000157168.18 | NRG1 |
| ENSG00000146216.11 | TTBK1 |
| ENSG00000170558.8 | CDH2 |
| ENSG00000183762.12 | KREMEN1 |
| ENSG00000261678.2 | SCRT1 |
| ENSG00000169330.8 | KIAA1024 |
| ENSG00000171587.14 | DSCAM |
| ENSG00000078018.19 | MAP2 |
| ENSG00000077264.14 | PAK3 |
| ENSG00000134259.3 | NGF |
| ENSG00000137872.16 | SEMA6D |
| ENSG00000104435.13 | STMN2 |
| ENSG00000140836.14 | ZFHX3 |
| ENSG00000081479.12 | LRP2 |
| ENSG00000058404.19 | CAMK2B |
| ENSG00000167178.15 | ISLR2 |
| ENSG00000132639.12 | SNAP25 |
| ENSG00000109132.6 | PHOX2B |
| ENSG00000187391.19 | MAGI2 |
| ENSG00000140538.16 | NTRK3 |
| ENSG00000107859.9 | PITX3 |
| ENSG00000135407.10 | AVIL |
| ENSG00000134352.19 | IL6ST |
| ENSG00000159082.17 | SYNJ1 |
| ENSG00000160145.15 | KALRN |
| ENSG00000109099.13 | PMP22 |
| ENSG00000091129.19 | NRCAM |
| ENSG00000117707.15 | PROX1 |
| ENSG00000198597.8 | ZNF536 |
| ENSG00000172260.14 | NEGR1 |
| ENSG00000221866.9 | PLXNA4 |
| ENSG00000135333.13 | EPHA7 |
| ENSG00000090932.10 | DLL3 |
| ENSG00000172201.11 | ID4 |
| ENSG00000152377.13 | SPOCK1 |
| ENSG00000143507.17 | DUSP10 |
| ENSG00000168542.13 | COL3A1 |
| ENSG00000006210.6 | CX3CL1 |
| ENSG00000140262.17 | TCF12 |
| ENSG00000153162.8 | BMP6 |
| ENSG00000170396.7 | ZNF804A |
| ENSG00000083290.19 | ULK2 |
| ENSG00000004139.13 | SARM1 |
| ENSG00000130827.6 | PLXNA3 |
| ENSG00000171617.13 | ENC1 |
| ENSG00000139352.3 | ASCL1 |
| ENSG00000143995.19 | MEIS1 |
| ENSG00000119283.15 | TRIM67 |
| ENSG00000065320.8 | NTN1 |
| ENSG00000138311.15 | ZNF365 |
| ENSG00000162676.11 | GFI1 |
| ENSG00000141433.12 | ADCYAP1 |
| ENSG00000118432.12 | CNR1 |
| ENSG00000148677.6 | ANKRD1 |
| ENSG00000171094.15 | ALK |
| ENSG00000186868.15 | MAPT |
| ENSG00000018189.12 | RUFY3 |
| ENSG00000076356.6 | PLXNA2 |
| ENSG00000136040.8 | PLXNC1 |
| ENSG00000131711.14 | MAP1B |
| ENSG00000151490.13 | PTPRO |
| ENSG00000157240.3 | FZD1 |

TABLE 4
Exemplary genes of gene ontology GO:0060160 with 4 times increased
gene expression levels relative to a pluripotent stem cell.

| | Gene |
|---|---|
| Gene ID | Symbol |
| ENSG00000149295.13 | DRD2 |
| ENSG00000117152.13 | RGS4 |
| ENSG00000099864.17 | PALM |

TABLE 5
Exemplary genes of gene ontology GO:0097458 with 4 times increased
gene expression levels relative to a pluripotent stem cell.

| | Gene |
|---|---|
| Gene ID | Symbol |
| ENSG00000150625.16 | GPM6A |
| ENSG00000075945.12 | KIFAP3 |
| ENSG00000149295.13 | DRD2 |
| ENSG00000108947.4 | EFNB3 |
| ENSG00000186765.11 | FSCN2 |
| ENSG00000183023.18 | SLC8A1 |
| ENSG00000079689.13 | SCGN |
| ENSG00000277363.4 | SRCIN1 |
| ENSG00000112530.11 | PACRG |
| ENSG00000100505.13 | TRIM9 |
| ENSG00000157168.18 | NRG1 |
| ENSG00000146216.11 | TTBK1 |
| ENSG00000102468.10 | HTR2A |
| ENSG00000036565.14 | SLC18A1 |
| ENSG00000188452.13 | CERKL |
| ENSG00000170558.8 | CDH2 |
| ENSG00000099260.10 | PALMD |
| ENSG00000183762.12 | KREMEN1 |
| ENSG00000170921.14 | TANC2 |
| ENSG00000109339.18 | MAPK10 |
| ENSG00000153253.15 | SCN3A |
| ENSG00000128594.7 | LRRC4 |
| ENSG00000171587.14 | DSCAM |
| ENSG00000119699.7 | TGFB3 |
| ENSG00000078018.19 | MAP2 |
| ENSG00000225968.7 | ELFN1 |
| ENSG00000077264.14 | PAK3 |
| ENSG00000134259.3 | NGF |
| ENSG00000137449.15 | CPEB2 |
| ENSG00000181418.7 | DDN |
| ENSG00000104435.13 | STMN2 |
| ENSG00000081479.12 | LRP2 |
| ENSG00000058404.19 | CAMK2B |
| ENSG00000166111.9 | SVOP |
| ENSG00000167720.12 | SRR |
| ENSG00000132639.12 | SNAP25 |
| ENSG00000139220.16 | PPFIA2 |
| ENSG00000177301.13 | KCNA2 |
| ENSG00000129990.14 | SYT5 |
| ENSG00000007516.13 | BAIAP3 |
| ENSG00000175161.13 | CADM2 |
| ENSG00000181072.11 | CHRM2 |
| ENSG00000077279.17 | DCX |
| ENSG00000187391.19 | MAGI2 |
| ENSG00000150361.11 | KLHL1 |
| ENSG00000140538.16 | NTRK3 |
| ENSG00000107859.9 | PITX3 |
| ENSG00000109991.8 | P2RX3 |
| ENSG00000197177.15 | ADGRA1 |
| ENSG00000135407.10 | AVIL |
| ENSG00000162706.12 | CADM3 |
| ENSG00000171450.5 | CDK5R2 |
| ENSG00000134352.19 | IL6ST |
| ENSG00000168280.16 | KIF5C |
| ENSG00000159082.17 | SYNJ1 |
| ENSG00000005379.15 | TSPOAP1 |
| ENSG00000102385.12 | DRP2 |
| ENSG00000160183.13 | TMPRSS3 |
| ENSG00000147642.16 | SYBU |
| ENSG00000170091.10 | HMP19 |
| ENSG00000065609.14 | SNAP91 |
| ENSG00000168356.11 | SCN11A |
| ENSG00000099864.17 | PALM |
| ENSG00000115902.10 | SLC1A4 |
| ENSG00000091129.19 | NRCAM |
| ENSG00000075461.5 | CACNG4 |
| ENSG00000174871.10 | CNIH2 |
| ENSG00000157680.15 | DGKI |
| ENSG00000158258.16 | CLSTN2 |
| ENSG00000166963.12 | MAP1A |
| ENSG00000101958.13 | GLRA2 |
| ENSG00000107611.14 | CUBN |
| ENSG00000136546.13 | SCN7A |
| ENSG00000082397.17 | EPB41L3 |
| ENSG00000164061.4 | BSN |
| ENSG00000172020.12 | GAP43 |
| ENSG00000135333.13 | EPHA7 |
| ENSG00000132821.11 | VSTM2L |
| ENSG00000152377.13 | SPOCK1 |
| ENSG00000006210.6 | CX3CL1 |
| ENSG00000008735.13 | MAPK8IP2 |
| ENSG00000162545.5 | CAMK2N1 |
| ENSG00000154678.16 | PDE1C |
| ENSG00000154654.14 | NCAM2 |
| ENSG00000091664.7 | SLC17A6 |
| ENSG00000187714.6 | SLC18A3 |
| ENSG00000129159.6 | KCNC1 |
| ENSG00000150471.16 | ADGRL3 |
| ENSG00000170396.7 | ZNF804A |
| ENSG00000004139.13 | SARM1 |
| ENSG00000149403.11 | GRIK4 |
| ENSG00000171617.13 | ENC1 |
| ENSG00000139352.3 | ASCL1 |
| ENSG00000158856.17 | DMTN |
| ENSG00000162456.9 | KNCN |
| ENSG00000152128.13 | TMEM163 |
| ENSG00000184113.9 | CLDN5 |
| ENSG00000171385.9 | KCND3 |
| ENSG00000187372.11 | PCDHB13 |
| ENSG00000111886.10 | GABRR2 |
| ENSG00000170017.12 | ALCAM |
| ENSG00000185518.11 | SV2B |
| ENSG00000183775.10 | KCTD16 |
| ENSG00000141433.12 | ADCYAP1 |
| ENSG00000107282.7 | APBA1 |
| ENSG00000118432.12 | CNR1 |
| ENSG00000015592.16 | STMN4 |
| ENSG00000163618.17 | CADPS |
| ENSG00000186868.15 | MAPT |
| ENSG00000018189.12 | RUFY3 |
| ENSG00000073282.12 | TP63 |
| ENSG00000152954.11 | NRSN1 |
| ENSG00000131711.14 | MAP1B |
| ENSG00000125851.9 | PCSK2 |
| ENSG00000157851.16 | DPYSL5 |
| ENSG00000198822.10 | GRM3 |
| ENSG00000157103.10 | SLC6A1 |
| ENSG00000183044.11 | ABAT |
| ENSG00000151067.21 | CACNA1C |
| ENSG00000166862.6 | CACNG2 |
| ENSG00000151490.13 | PTPRO |
| ENSG00000169684.13 | CHRNA5 |
| ENSG00000040731.10 | CDH10 |

TABLE 6
Exemplary genes of gene ontology GO:0010975 with 4 times increased
gene expression levels relative to a pluripotent stem cell.

| | Gene |
|---|---|
| Gene ID | Symbol |
| ENSG00000108947.4 | EFNB3 |
| ENSG00000075223.13 | SEMA3C |
| ENSG00000277363.4 | SRCIN1 |
| ENSG00000145147.19 | SLIT2 |
| ENSG00000170558.8 | CDH2 |
| ENSG00000183762.12 | KREMEN1 |
| ENSG00000169330.8 | KIAA1024 |
| ENSG00000171587.14 | DSCAM |
| ENSG00000078018.19 | MAP2 |
| ENSG00000077264.14 | PAK3 |
| ENSG00000134259.3 | NGF |
| ENSG00000137872.16 | SEMA6D |
| ENSG00000104435.13 | STMN2 |
| ENSG00000058404.19 | CAMK2B |
| ENSG00000167178.15 | ISLR2 |
| ENSG00000132639.12 | SNAP25 |
| ENSG00000187391.19 | MAGI2 |
| ENSG00000140538.16 | NTRK3 |
| ENSG00000135407.10 | AVIL |
| ENSG00000160145.15 | KALRN |
| ENSG00000109099.13 | PMP22 |
| ENSG00000091129.19 | NRCAM |
| ENSG00000172260.14 | NEGR1 |
| ENSG00000221866.9 | PLXNA4 |
| ENSG00000135333.13 | EPHA7 |
| ENSG00000152377.13 | SPOCK1 |
| ENSG00000006210.6 | CX3CL1 |
| ENSG00000170396.7 | ZNF804A |
| ENSG00000083290.19 | ULK2 |
| ENSG00000004139.13 | SARM1 |
| ENSG00000130827.6 | PLXNA3 |
| ENSG00000171617.13 | ENC1 |
| ENSG00000119283.15 | TRIM67 |
| ENSG00000065320.8 | NTN1 |
| ENSG00000138311.15 | ZNF365 |
| ENSG00000162676.11 | GFI1 |
| ENSG00000141433.12 | ADCYAP1 |
| ENSG00000118432.12 | CNR1 |
| ENSG00000148677.6 | ANKRD1 |
| ENSG00000186868.15 | MAPT |
| ENSG00000018189.12 | RUFY3 |
| ENSG00000076356.6 | PLXNA2 |
| ENSG00000136040.8 | PLXNC1 |
| ENSG00000131711.14 | MAP1B |
| ENSG00000151490.13 | PTPRO |
| ENSG00000157240.3 | FZD1 |

TABLE 7
Exemplary genes of gene ontology GO:0022008 with 4 times increased
gene expression levels relative to a pluripotent stem cell.

| | Gene |
|---|---|
| Gene ID | Symbol |
| ENSG00000150625.16 | GPM6A |
| ENSG00000149295.13 | DRD2 |
| ENSG00000101144.12 | BMP7 |
| ENSG00000108947.4 | EFNB3 |
| ENSG00000075223.13 | SEMA3C |
| ENSG00000186765.11 | FSCN2 |
| ENSG00000108231.12 | LGI1 |
| ENSG00000277363.4 | SRCIN1 |
| ENSG00000162552.14 | WNT4 |
| ENSG00000145147.19 | SLIT2 |
| ENSG00000067798.14 | NAV3 |
| ENSG00000157168.18 | NRG1 |
| ENSG00000146216.11 | TTBK1 |
| ENSG00000141622.13 | RNF165 |
| ENSG00000142611.16 | PRDM16 |
| ENSG00000170558.8 | CDH2 |
| ENSG00000162374.16 | ELAVL4 |
| ENSG00000119547.5 | ONECUT2 |
| ENSG00000183762.12 | KREMEN1 |
| ENSG00000261678.2 | SCRT1 |
| ENSG00000169330.8 | KIAA1024 |
| ENSG00000171587.14 | DSCAM |
| ENSG00000078018.19 | MAP2 |
| ENSG00000152784.15 | PRDM8 |
| ENSG00000196159.11 | FAT4 |
| ENSG00000077264.14 | PAK3 |
| ENSG00000134259.3 | NGF |
| ENSG00000137872.16 | SEMA6D |
| ENSG00000104435.13 | STMN2 |
| ENSG00000140836.14 | ZFHX3 |
| ENSG00000081479.12 | LRP2 |
| ENSG00000118137.9 | APOA1 |
| ENSG00000058404.19 | CAMK2B |
| ENSG00000112139.14 | MDGA1 |
| ENSG00000167178.15 | ISLR2 |
| ENSG00000132639.12 | SNAP25 |
| ENSG00000123307.3 | NEUROD4 |
| ENSG00000109132.6 | PHOX2B |
| ENSG00000077279.17 | DCX |
| ENSG00000187391.19 | MAGI2 |
| ENSG00000145675.14 | PIK3R1 |
| ENSG00000149294.16 | NCAM1 |
| ENSG00000140538.16 | NTRK3 |
| ENSG00000107859.9 | PITX3 |
| ENSG00000186487.17 | MYT1L |
| ENSG00000135407.10 | AVIL |
| ENSG00000171450.5 | CDK5R2 |
| ENSG00000173404.4 | INSM1 |
| ENSG00000125285.5 | SOX21 |
| ENSG00000134352.19 | IL6ST |
| ENSG00000168280.16 | KIF5C |
| ENSG00000159082.17 | SYNJ1 |
| ENSG00000160145.15 | KALRN |
| ENSG00000151892.14 | GFRA1 |
| ENSG00000204852.15 | TCTN1 |
| ENSG00000075275.16 | CELSR1 |
| ENSG00000176842.14 | IRX5 |
| ENSG00000109099.13 | PMP22 |
| ENSG00000110693.16 | SOX6 |
| ENSG00000159216.18 | RUNX1 |
| ENSG00000151640.12 | DPYSL4 |
| ENSG00000091129.19 | NRCAM |
| ENSG00000198795.10 | ZNF521 |
| ENSG00000139915.18 | MDGA2 |
| ENSG00000117707.15 | PROX1 |
| ENSG00000138675.16 | FGF5 |
| ENSG00000198597.8 | ZNF536 |
| ENSG00000166963.12 | MAP1A |
| ENSG00000166341.7 | DCHS1 |
| ENSG00000172260.14 | NEGR1 |
| ENSG00000221866.9 | PLXNA4 |
| ENSG00000082397.17 | EPB41L3 |
| ENSG00000172020.12 | GAP43 |
| ENSG00000135333.13 | EPHA7 |
| ENSG00000090932.10 | DLL3 |
| ENSG00000132821.11 | VSTM2L |
| ENSG00000172201.11 | ID4 |
| ENSG00000124785.8 | NRN1 |
| ENSG00000152377.13 | SPOCK1 |
| ENSG00000143507.17 | DUSP10 |
| ENSG00000168542.13 | COL3A1 |
| ENSG00000006210.6 | CX3CL1 |
| ENSG00000184347.14 | SLIT3 |
| ENSG00000008735.13 | MAPK8IP2 |
| ENSG00000135472.8 | FAIM2 |
| ENSG00000140262.17 | TCF12 |
| ENSG00000153162.8 | BMP6 |
| ENSG00000185189.16 | NRBP2 |
| ENSG00000154654.14 | NCAM2 |
| ENSG00000064393.15 | HIPK2 |
| ENSG00000140937.13 | CDH11 |
| ENSG00000150471.16 | ADGRL3 |
| ENSG00000170396.7 | ZNF804A |
| ENSG00000083290.19 | ULK2 |
| ENSG00000163394.5 | CCKAR |
| ENSG00000004139.13 | SARM1 |
| ENSG00000130827.6 | PLXNA3 |
| ENSG00000171617.13 | ENC1 |
| ENSG00000139352.3 | ASCL1 |
| ENSG00000164853.8 | UNCX |
| ENSG00000143995.19 | MEIS1 |
| ENSG00000004848.7 | ARX |
| ENSG00000139767.8 | SRRM4 |
| ENSG00000119283.15 | TRIM67 |
| ENSG00000170017.12 | ALCAM |
| ENSG00000065320.8 | NTN1 |
| ENSG00000138311.15 | ZNF365 |
| ENSG00000162676.11 | GFI1 |
| ENSG00000141433.12 | ADCYAP1 |
| ENSG00000118432.12 | CNR1 |
| ENSG00000148677.6 | ANKRD1 |
| ENSG00000171094.15 | ALK |
| ENSG00000015592.16 | STMN4 |
| ENSG00000186868.15 | MAPT |
| ENSG00000018189.12 | RUFY3 |
| ENSG00000076356.6 | PLXNA2 |
| ENSG00000136040.8 | PLXNC1 |
| ENSG00000131711.14 | MAP1B |
| ENSG00000157851.16 | DPYSL5 |
| ENSG00000151490.13 | PTPRO |
| ENSG00000157240.3 | FZD1 |
| ENSG00000105880.4 | DLX5 |

TABLE 8
Exemplary gene ontologies including one or more with 4 times decreased
gene expression levels relative to a pluripotent stem cell.

| GO ACCESSION | GO Term |
|---|---|
| GO:0044459 | plasma membrane part |
| GO:0071944 | cell periphery |
| GO:0005886\|GO:0005904 | plasma membrane |
| GO:0031226 | intrinsic component of plasma |
| | membrane |
| GO:0005887 | integral component of plasma |
| | membrane |
| GO:0042127 | regulation of cell proliferation |
| GO:0005576 | extracellular region |
| GO:0044421 | extracellular region part |
| GO:0070887 | cellular response to chemical |
| | stimulus |
| GO:0034097 | response to cytokine |
| GO:0050896\|GO:0051869 | response to stimulus |
| GO:0071345 | cellular response to cytokine |
| | stimulus |
| GO:0048856 | anatomical structure |
| | development |
| GO:0010033 | response to organic substance |
| GO:0044425 | membrane part |
| GO:0007166 | cell surface receptor signaling |
| | pathway |
| GO:0032501\|GO:0044707\| | multicellular organismal process |
| GO:0050874 | |
| GO:0023052\|GO:0023046\| | signaling |
| GO:0044700 | |
| GO:0031982\|GO:0031988 | vesicle |
| GO:0032502\|GO:0044767 | developmental process |
| GO:0007154 | cell communication |
| GO:0071310 | cellular response to organic |
| | substance |
| GO:0005615 | extracellular space |
| GO:0042221 | response to chemical |
| GO:0031224 | intrinsic component of |
| | membrane |
| GO:0051049 | regulation of transport |
| GO:0019221 | cytokine-mediated signaling |
| | pathway |
| GO:0048583 | regulation of response to |
| | stimulus |
| GO:0008284 | positive regulation of cell |
| | proliferation |
| GO:0007275 | multicellular organism |
| | development |
| GO:0023051 | regulation of signaling |
| GO:0010646 | regulation of cell communication |
| GO:0048584 | positive regulation of response to |
| | stimulus |
| GO:0051239 | regulation of multicellular |
| | organismal process |
| GO:0032879 | regulation of localization |
| GO:0006954 | inflammatory response |
| GO:0007165\|GO:0023033 | signal transduction |
| GO:0043230 | extracellular organelle |
| GO:0098771 | inorganic ion homeostasis |
| GO:0055065 | metal ion homeostasis |
| GO:0016021 | integral component of membrane |
| GO:1903561 | extracellular vesicle |
| GO:0009966\|GO:0035466 | regulation of signal transduction |
| GO:0050801 | ion homeostasis |
| GO:0010647 | positive regulation of cell |
| | communication |
| GO:0006811 | ion transport |
| GO:0065008 | regulation of biological quality |
| GO:0051240 | positive regulation of |
| | multicellular organismal process |
| GO:0098590 | plasma membrane region |
| GO:0055082 | cellular chemical homeostasis |
| GO:0055080 | cation homeostasis |
| GO:0023056 | positive regulation of signaling |
| GO:0006875 | cellular metal ion homeostasis |
| GO:0070062 | extracellular exosome |
| GO:0051716 | cellular response to stimulus |
| GO:0048878 | chemical homeostasis |
| GO:0043269 | regulation of ion transport |
| GO:0065009 | regulation of molecular function |
| GO:0051050 | positive regulation of transport |
| GO:0050865 | regulation of cell activation |
| GO:0098857 | membrane microdomain |
| GO:0006873 | cellular ion homeostasis |
| GO:0048518\|GO:0043119 | positive regulation of biological |
| | process |
| GO:0030003 | cellular cation homeostasis |
| GO:0048731 | system development |
| GO:0042592 | homeostatic process |
| GO:0045121 | membrane raft |
| GO:0006952\|GO:0002217\| | defense response |
| GO:0042829 | |
| GO:0048522\|GO:0051242 | positive regulation of cellular |
| | process |
| GO:0046903 | secretion |
| GO:0005102 | receptor binding |
| GO:0030154 | cell differentiation |
| GO:0019725 | cellular homeostasis |
| GO:0001775 | cell activation |
| GO:0009967\|GO:0035468 | positive regulation of signal |
| | transduction |
| GO:0002376 | immune system process |
| GO:0072503 | cellular divalent inorganic cation |
| | homeostasis |
| GO:0045321 | leukocyte activation |
| GO:0050863 | regulation of T cell activation |
| GO:0050878 | regulation of body fluid levels |
| GO:0048869 | cellular developmental process |
| GO:0002703 | regulation of leukocyte mediated |
| | immunity |
| GO:0050670 | regulation of lymphocyte |
| | proliferation |
| GO:0022407 | regulation of cell-cell adhesion |
| GO:0032944 | regulation of mononuclear cell |
| | proliferation |
| GO:0016020 | membrane |
| GO:1902533\|GO:0010740 | positive regulation of |
| | intracellular signal transduction |
| GO:0043270 | positive regulation of ion |
| | transport |
| GO:0045785 | positive regulation of cell |
| | adhesion |
| GO:0072507 | divalent inorganic cation |
| | homeostasis |
| GO:0009888 | tissue development |
| GO:0022409 | positive regulation of cell-cell |
| | adhesion |
| GO:0042493\|GO:0017035 | response to drug |
| GO:0002682 | regulation of immune system |
| | process |
| GO:0006874 | cellular calcium ion homeostasis |
| GO:0032101 | regulation of response to |
| | external stimulus |
| GO:0070663 | regulation of leukocyte |
| | proliferation |
| GO:0007204 | positive regulation of cytosolic |
| | calcium ion concentration |
| GO:1902531\|GO:0010627 | regulation of intracellular signal |
| | transduction |
| GO:1903039 | positive regulation of leukocyte |
| | cell-cell adhesion |
| GO:1903037 | regulation of leukocyte cell-cell |
| | adhesion |
| GO:0002694 | regulation of leukocyte |
| | activation |
| GO:0031012 | extracellular matrix |
| GO:0009605 | response to external stimulus |
| GO:0044281 | small molecule metabolic |
| | process |
| GO:2000021 | regulation of ion homeostasis |
| GO:0055074 | calcium ion homeostasis |
| GO:0035296 | regulation of tube diameter |
| GO:0097746\|GO:0042312 | regulation of blood vessel |
| | diameter |
| GO:0044093 | positive regulation of molecular |
| | function |
| GO:0002685 | regulation of leukocyte |
| | migration |
| GO:0098589 | membrane region |
| GO:0051480 | regulation of cytosolic calcium |
| | ion concentration |
| GO:0003013 | circulatory system process |
| GO:0008015\|GO:0070261 | blood circulation |
| GO:1901700 | response to oxygen-containing |
| | compound |
| GO:0007187 | G-protein coupled receptor |
| | signaling pathway, coupled to |
| | cyclic nucleotide second |
| | messenger |
| GO:0030155 | regulation of cell adhesion |
| GO:0003006 | developmental process involved |
| | in reproduction |
| GO:0034220 | ion transmembrane transport |
| GO:0050870 | positive regulation of T cell |
| | activation |
| GO:0009611\|GO:0002245 | response to wounding |
| GO:0008217 | regulation of blood pressure |
| GO:1903524 | positive regulation of blood |
| | circulation |
| GO:0042129 | regulation of T cell proliferation |
| GO:0033993 | response to lipid |
| GO:0050880 | regulation of blood vessel size |
| GO:0007188 | adenylate cyclase-modulating G- |
| | protein coupled receptor |
| | signaling pathway |
| GO:0051704\|GO:0051706 | multi-organism process |
| GO:0035150 | regulation of tube size |
| GO:0030198 | extracellular matrix organization |
| GO:0032103 | positive regulation of response to |
| | external stimulus |
| GO:0043062 | extracellular structure |
| | organization |
| GO:0050867 | positive regulation of cell |
| | activation |
| GO:0040017 | positive regulation of |
| | locomotion |
| GO:0002687 | positive regulation of leukocyte |
| | migration |
| GO:0022857\|GO:0005386\| | transmembrane transporter |
| GO:0015563\|GO:0015646\| | activity |
| GO:0022891\|GO:0022892 | |
| GO:0048608 | reproductive structure |
| | development |
| GO:0015267\|GO:0015249\| | channel activity |
| GO:0015268 | |
| GO:0002274 | myeloid leukocyte activation |
| GO:0001890 | placenta development |
| GO:0048513 | animal organ development |
| GO:0022803\|GO:0022814 | passive transmembrane |
| | transporter activity |
| GO:0002684 | positive regulation of immune |
| | system process |
| GO:0050776 | regulation of immune response |
| GO:0002819 | regulation of adaptive immune |
| | response |
| GO:0045937 | positive regulation of phosphate |
| | metabolic process |
| GO:0010562 | positive regulation of |
| | phosphorus metabolic process |
| GO:0002366 | leukocyte activation involved in |
| | immune response |
| GO:0061458 | reproductive system |
| | development |
| GO:0051094 | positive regulation of |
| | developmental process |
| GO:0034762 | regulation of transmembrane |
| | transport |
| GO:2000147 | positive regulation of cell |
| | motility |
| GO:0030141 | secretory granule |
| GO:0002263 | cell activation involved in |
| | immune response |
| GO:0006955 | immune response |
| GO:0015075 | ion transmembrane transporter |
| | activity |
| GO:0099503 | secretory vesicle |
| GO:0000003\|GO:0019952\| | reproduction |
| GO:0050876 | |
| GO:0098772 | molecular function regulator |
| GO:0002252 | immune effector process |
| GO:0009653 | anatomical structure |
| | morphogenesis |
| GO:0050900 | leukocyte migration |
| GO:1901701 | cellular response to oxygen- |
| | containing compound |
| GO:0042802 | identical protein binding |
| GO:0043085\|GO:0048554 | positive regulation of catalytic |
| | activity |
| GO:0030335 | positive regulation of cell |
| | migration |
| GO:0005215\|GO:0005478 | transporter activity |
| GO:0022414\|GO:0044702 | reproductive process |
| GO:0051241 | negative regulation of |
| | multicellular organismal process |
| GO:0002696 | positive regulation of leukocyte |
| | activation |
| GO:0046873 | metal ion transmembrane |
| | transporter activity |
| GO:0042060 | wound healing |
| GO:0003018 | vascular process in circulatory |
| | system |
| GO:0032940 | secretion by cell |
| GO:0031410\|GO:0016023 | cytoplasmic vesicle |
| GO:0002822 | regulation of adaptive immune |
| | response based on somatic |
| | recombination of immune |
| | receptors built from |
| | immunoglobulin superfamily |
| | domains |
| GO:0046394 | carboxylic acid biosynthetic |
| | process |
| GO:0051272 | positive regulation of cellular |
| | component movement |
| GO:0097708 | intracellular vesicle |
| GO:0009986\|GO:0009928\| | cell surface |
| GO:0009929 | |
| GO:0016053 | organic acid biosynthetic process |
| GO:0051928 | positive regulation of calcium |
| | ion transport |
| GO:0042327 | positive regulation of |
| | phosphorylation |
| GO:0031225 | anchored component of |
| | membrane |
| GO:0010469 | regulation of receptor activity |
| GO:0009987\|GO:0008151 \| | cellular process |
| GO:0044763\|GO:0050875 | |
| GO:0006950 | response to stress |
| GO:0043207 | response to external biotic |
| | stimulus |
| GO:0002886 | regulation of myeloid leukocyte |
| | mediated immunity |
| GO:0051249 | regulation of lymphocyte |
| | activation |
| GO:0098655 | cation transmembrane transport |
| GO:0005575\|GO:0008372 | cellular_component |
| GO:0002697 | regulation of immune effector |
| | process |
| GO:0019935 | cyclic-nucleotide-mediated |
| | signaling |
| GO:0007267 | cell-cell signaling |
| GO:0032496 | response to lipopolysaccharide |
| GO:0070160 | occluding junction |
| GO:0005216 | ion channel activity |
| GO:0034765 | regulation of ion transmembrane |
| | transport |
| GO:0006820\|GO:0006822 | anion transport |
| GO:0005911 | cell-cell junction |
| GO:0019933 | cAMP-mediated signaling |
| GO:0004252 | serine-type endopeptidase |
| | activity |
| GO:0048545 | response to steroid hormone |
| GO:0051924 | regulation of calcium ion |
| | transport |
| GO:0006812\|GO:0006819\| | cation transport |
| GO:0015674 | |
| GO:0019932 | second-messenger-mediated |
| | signaling |
| GO:0051707\|GO:0009613\| | response to other organism |
| GO:0042828 | |
| GO:0001934 | positive regulation of protein |
| | phosphorylation |
| GO:0022838 | substrate-specific channel |
| | activity |
| GO:1902105 | regulation of leukocyte |
| | differentiation |
| GO:0006636 | unsaturated fatty acid |
| | biosynthetic process |
| GO:0071624 | positive regulation of |
| | granulocyte chemotaxis |
| GO:0055085 | transmembrane transport |
| GO:0010959 | regulation of metal ion transport |
| GO:0005923 | bicellular tight junction |
| GO:0030001 | metal ion transport |
| GO:0002237 | response to molecule of bacterial |
| | origin |
| GO:0009607 | response to biotic stimulus |
| GO:0002699 | positive regulation of immune |
| | effector process |
| GO:0005261\|GO:0015281\| | cation channel activity |
| GO:0015338 | |
| GO:1903522 | regulation of blood circulation |
| GO:0043408 | regulation of MAPK cascade |
| GO:0008324 | cation transmembrane |
| | transporter activity |
| GO:0015711 | organic anion transport |
| GO:0071622 | regulation of granulocyte |
| | chemotaxis |
| GO:0070665 | positive regulation of leukocyte |
| | proliferation |
| GO:0002683 | negative regulation of immune |
| | system process |
| GO:0010543 | regulation of platelet activation |
| GO:0050730 | regulation of peptidyl-tyrosine |
| | phosphorylation |
| GO:0007189\|GO:0010579\| | adenylate cyclase-activating G- |
| GO:0010580 | protein coupled receptor |
| | signaling pathway |
| GO:0016338 | calcium-independent cell-cell |
| | adhesion via plasma membrane |
| | cell-adhesion molecules |
| GO:0050671 | positive regulation of |
| | lymphocyte proliferation |
| GO:0015318 | inorganic molecular entity |
| | transmembrane transporter |
| | activity |
| GO:0050777 | negative regulation of immune |
| | response |
| GO:0050793 | regulation of developmental |
| | process |
| GO:0030054 | cell junction |
| GO:0022610 | biological adhesion |
| GO:0032946 | positive regulation of |
| | mononuclear cell proliferation |
| GO:0043300 | regulation of leukocyte |
| | degranulation |
| GO:0042102 | positive regulation of T cell |
| | proliferation |
| GO:0001817 | regulation of cytokine |
| | production |
| GO:0002275 | myeloid cell activation involved |
| | in immune response |
| GO:0032844 | regulation of homeostatic |
| | process |
| GO:0060429 | epithelium development |
| GO:0001653 | peptide receptor activity |
| GO:0031347 | regulation of defense response |
| GO:0048646 | anatomical structure formation |
| | involved in morphogenesis |
| GO:0042981 | regulation of apoptotic process |
| GO:0051345 | positive regulation of hydrolase |
| | activity |
| GO:0002690 | positive regulation of leukocyte |
| | chemotaxis |
| GO:0043302 | positive regulation of leukocyte |
| | degranulation |
| GO:0098660 | inorganic ion transmembrane |
| | transport |
| GO:0009719 | response to endogenous stimulus |
| GO:0048018\|GO:0071884 | receptor ligand activity |
| GO:0009116 | nucleoside metabolic process |
| GO:0043168 | anion binding |
| GO:0002444 | myeloid leukocyte mediated |
| | immunity |
| GO:0043296 | apical junction complex |
| GO:0065007 | biological regulation |
| GO:0098662 | inorganic cation transmembrane |
| | transport |
| GO:0043299 | leukocyte degranulation |
| GO:0030193 | regulation of blood coagulation |
| GO:0042119 | neutrophil activation |
| GO:0050921 | positive regulation of |
| | chemotaxis |
| GO:0002688 | regulation of leukocyte |
| | chemotaxis |
| GO:0043410 | positive regulation of MAPK |
| | cascade |
| GO:0022836 | gated channel activity |
| GO:0090022 | regulation of neutrophil |
| | chemotaxis |
| GO:0002888 | positive regulation of myeloid |
| | leukocyte mediated immunity |
| GO:0002821 | positive regulation of adaptive |
| | immune response |
| GO:1900046 | regulation of hemostasis |
| GO:0042509\|GO:0042510\| | regulation of tyrosine |
| GO:0042513\|GO:0042516\| | phosphorylation of STAT |
| GO:0042519\|GO:0042522\| | protein |
| GO:0042525\|GO:0042528 | |
| GO:0035295 | tube development |
| GO:0043235 | receptor complex |
| GO:0022839 | ion gated channel activity |
| GO:0090023 | positive regulation of neutrophil |
| | chemotaxis |
| GO:0043065 | positive regulation of apoptotic |
| | process |
| GO:0046718\|GO:0019063 | viral entry into host cell |
| GO:0043067\|GO:0043070 | regulation of programmed cell |
| | death |
| GO:0030545 | receptor regulator activity |
| GO:0001816 | cytokine production |
| GO:0003382 | epithelial cell morphogenesis |
| GO:0044409 | entry into host |
| GO:0051806 | entry into cell of other organism |
| | involved in symbiotic interaction |
| GO:0030260 | entry into host cell |
| GO:0051828 | entry into other organism |
| | involved in symbiotic interaction |
| GO:0036230 | granulocyte activation |
| GO:0010941 | regulation of cell death |
| GO:0009725 | response to hormone |
| GO:0002476 | antigen processing and |
| | presentation of endogenous |
| | peptide antigen via MHC class |
| | lb |
| GO:0002526 | acute inflammatory response |
| GO:0051384 | response to glucocorticoid |
| GO:0050790\|GO:0048552 | regulation of catalytic activity |
| GO:0051247 | positive regulation of protein |
| | metabolic process |
| GO:0008285 | negative regulation of cell |
| | proliferation |
| GO:0097755\|GO:0045909 | positive regulation of blood |
| | vessel diameter |
| GO:0031960 | response to corticosteroid |
| GO:0070374 | positive regulation of ERK1 and |
| | ERK2 cascade |
| GO:0002824 | positive regulation of adaptive |
| | immune response based on |
| | somatic recombination of |
| | immune receptors built from |
| | immunoglobulin superfamily |
| | domains |
| GO:0030728 | ovulation |
| GO:0007155\|GO:0098602 | cell adhesion |
| GO:0035556\|GO:0007242\| | intracellular signal transduction |
| GO:0007243\|GO:0023013\| | |
| GO:0023034 | |
| GO:0010942 | positive regulation of cell death |
| GO:0070372 | regulation of ERK1 and ERK2 |
| | cascade |
| GO:0051046 | regulation of secretion |
| GO:0043068\|GO:0043071 | positive regulation of |
| | programmed cell death |
| GO:1902107 | positive regulation of leukocyte |
| | differentiation |
| GO:0002283 | neutrophil activation involved in |
| | immune response |
| GO:0005509 | calcium ion binding |
| GO:0050818 | regulation of coagulation |
| GO:0051336 | regulation of hydrolase activity |
| GO:0009119 | ribonucleoside metabolic process |
| GO:0003073 | regulation of systemic arterial |
| | blood pressure |
| GO:0036018 | cellular response to |
| | erythropoietin |
| GO:0046635 | positive regulation of alpha-beta |
| | T cell activation |
| GO:2000026 | regulation of multicellular |
| | organismal development |
| GO:0006082 | organic acid metabolic process |
| GO:0001819 | positive regulation of cytokine |
| | production |
| GO:0004175\|GO:0016809 | endopeptidase activity |
| GO:0050764 | regulation of phagocytosis |
| GO:0043436 | oxoacid metabolic process |
| GO:0005201 | extracellular matrix structural |
| | constituent |
| GO:0097028 | dendritic cell differentiation |
| GO:0008528 | G-protein coupled peptide |
| | receptor activity |
| GO:0045055 | regulated exocytosis |
| GO:0016477 | cell migration |
| GO:0030168 | platelet activation |
| GO:0035239 | tube morphogenesis |
| GO:0070820 | tertiary granule |
| GO:0031349 | positive regulation of defense |
| | response |
| GO:0001932 | regulation of protein |
| | phosphorylation |
| GO:0098797 | plasma membrane protein |
| | complex |
| GO:0045137 | development of primary sexual |
| | characteristics |
| GO:0043312 | neutrophil degranulation |
| GO:0002446 | neutrophil mediated immunity |
| GO:0052547 | regulation of peptidase activity |
| GO:0048585 | negative regulation of response |
| | to stimulus |
| GO:0009070 | serine family amino acid |
| | biosynthetic process |
| GO:0009113 | purine nucleobase biosynthetic |
| | process |
| GO:0034764 | positive regulation of |
| | transmembrane transport |
| GO:0022600 | digestive system process |
| GO:0016323 | basolateral plasma membrane |
| GO:0045597 | positive regulation of cell |
| | differentiation |
| GO:0042803 | protein homodimerization |
| | activity |
| GO:0016324 | apical plasma membrane |
| GO:0045177 | apical part of cell |
| GO:0008406 | gonad development |
| GO:0006887\|GO:0016194\| | exocytosis |
| GO:0016195 | |
| GO:0008236 | serine-type peptidase activity |
| GO:0072358 | cardiovascular system |
| | development |
| GO:0001944 | vasculature development |
| GO:0002521 | leukocyte differentiation |
| GO:1902624 | positive regulation of neutrophil |
| | migration |
| GO:0044283 | small molecule biosynthetic |
| | process |
| GO:0048519\|GO:0043118 | negative regulation of biological |
| | process |
| GO:0045684 | positive regulation of epidermis |
| | development |
| GO:0006690 | icosanoid metabolic process |
| GO:0010522 | regulation of calcium ion |
| | transport into cytosol |
| GO:0022890\|GO:0015082 | inorganic cation transmembrane |
| | transporter activity |
| GO:0019752 | carboxylic acid metabolic |
| | process |
| GO:0071396 | cellular response to lipid |
| GO:0001525 | angiogenesis |
| GO:0050731 | positive regulation of peptidyl- |
| | tyrosine phosphorylation |
| GO:0036017 | response to erythropoietin |
| GO:0042609 | CD4 receptor binding |
| GO:0050817 | coagulation |
| GO:0070252 | actin-mediated cell contraction |
| GO:0060670 | branching involved in |
| | labyrinthine layer |
| | morphogenesis |
| GO:0019369 | arachidonic acid metabolic |
| | process |
| GO:0019229 | regulation of vasoconstriction |
| GO:0009164 | nucleoside catabolic process |
| GO:0017171 | serine hydrolase activity |
| GO:0045907 | positive regulation of |
| | vasoconstriction |
| GO:0008289 | lipid binding |
| GO:1902622 | regulation of neutrophil |
| | migration |
| GO:0050920 | regulation of chemotaxis |
| GO:0051047 | positive regulation of secretion |
| GO:0046649 | lymphocyte activation |
| GO:0032270 | positive regulation of cellular |
| | protein metabolic process |
| GO:0009991 | response to extracellular |
| | stimulus |
| GO:0033628 | regulation of cell adhesion |
| | mediated by integrin |
| GO:0004715 | non-membrane spanning protein |
| | tyrosine kinase activity |
| GO:0045776 | negative regulation of blood |
| | pressure |
| GO:0042454 | ribonucleoside catabolic process |
| GO:0005515\|GO:0001948\| | protein binding |
| GO:0045308 | |
| GO:0002706 | regulation of lymphocyte |
| | mediated immunity |
| GO:1903530 | regulation of secretion by cell |
| GO:1901657 | glycosyl compound metabolic |
| | process |
| GO:0030322 | stabilization of membrane |
| | potential |
| GO:0042270 | protection from natural killer cell |
| | mediated cytotoxicity |
| GO:0045088 | regulation of innate immune |
| | response |
| GO:0046717 | acid secretion |
| GO:0016661 | oxidoreductase activity, acting |
| | on other nitrogenous compounds |
| | as donors |
| GO:0008584 | male gonad development |
| GO:0002428 | antigen processing and |
| | presentation of peptide antigen |
| | via MHC class Ib |
| GO:1901568 | fatty acid derivative metabolic |
| | process |
| GO:0042325 | regulation of phosphorylation |
| GO:0044433 | cytoplasmic vesicle part |
| GO:0044057 | regulation of system process |
| GO:0031638 | zymogen activation |
| GO:0006953 | acute-phase response |
| GO:0050729 | positive regulation of |
| | inflammatory response |
| GO:0046546 | development of primary male |
| | sexual characteristics |
| GO:0042531\|GO:0042511\| | positive regulation of tyrosine |
| GO:0042515\|GO:0042517\| | phosphorylation of STAT |
| GO:0042520\|GO:0042523\| | protein |
| GO:0042526\|GO:0042529 | |
| GO:0046850 | regulation of bone remodeling |
| GO:0005178 | integrin binding |
| GO:0048514 | blood vessel morphogenesis |
| GO:0045682 | regulation of epidermis |
| | development |
| GO:0003674\|GO:0005554 | molecular_function |
| GO:0046634 | regulation of alpha-beta T cell |
| | activation |
| GO:0061041 | regulation of wound healing |
| GO:0008016 | regulation of heart contraction |
| GO:0043407 | negative regulation of MAP |
| | kinase activity |
| GO:0046456 | icosanoid biosynthetic process |
| GO:0007596 | blood coagulation |
| GO:0045606 | positive regulation of epidermal |
| | cell differentiation |
| GO:0014070 | response to organic cyclic |
| | compound |
| GO:0048870 | cell motility |
| GO:0051674 | localization of cell |
| GO:0002704 | negative regulation of leukocyte |
| | mediated immunity |
| GO:0007584 | response to nutrient |
| GO:0070228 | regulation of lymphocyte |
| | apoptotic process |
| GO:0002675 | positive regulation of acute |
| | inflammatory response |
| GO:0052548 | regulation of endopeptidase |
| | activity |
| GO:0001664 | G-protein coupled receptor |
| | binding |
| GO:0090330 | regulation of platelet aggregation |
| GO:0045117 | azole transport |
| GO:0034340 | response to type I interferon |
| GO:0044853 | plasma membrane raft |
| GO:0032587 | ruffle membrane |
| GO:0007586 | digestion |
| GO:0097529 | myeloid leukocyte migration |
| GO:0045595 | regulation of cell differentiation |
| GO:0040012 | regulation of locomotion |
| GO:0050866 | negative regulation of cell |
| | activation |
| GO:0010035 | response to inorganic substance |
| GO:0034767 | positive regulation of ion |
| | transmembrane transport |
| GO:0098801 | regulation of renal system |
| | process |
| GO:0015079\|GO:0015388\| | potassium ion transmembrane |
| GO:0022817 | transporter activity |
| GO:0044706 | multi-multicellular organism |
| | process |
| GO:1901605 | alpha-amino acid metabolic |
| | process |
| GO:0009636 | response to toxic substance |
| GO:0007599 | hemostasis |
| GO:0002705 | positive regulation of leukocyte |
| | mediated immunity |
| GO:2000145 | regulation of cell motility |
| GO:0034103 | regulation of tissue remodeling |
| GO:0032642 | regulation of chemokine |
| | production |
| GO:0098805 | whole membrane |
| GO:0051209 | release of sequestered calcium |
| | ion into cytosol |
| GO:1901137 | carbohydrate derivative |
| | biosynthetic process |
| GO:0090066 | regulation of anatomical |
| | structure size |
| GO:0098641 | cadherin binding involved in |
| | cell-cell adhesion |
| GO:0032409 | regulation of transporter activity |
| GO:0007589 | body fluid secretion |
| GO:0046128 | purine ribonucleoside metabolic |
| | process |
| GO:0061134 | peptidase regulator activity |
| GO:0015893 | drug transport |
| GO:0001726 | ruffle |
| GO:0001893 | maternal placenta development |
| GO:0030334 | regulation of cell migration |
| GO:0042398 | cellular modified amino acid |
| | biosynthetic process |

TABLE 9
Exemplary genes of gene ontology GO:0042127 with 4 times decreased
gene expression levels relative to a pluripotent stem cell.

| | Gene |
|---|---|
| Gene ID | Symbol |
| ENSG00000135636.13 | DYSF |
| ENSG00000105122.12 | RASAL3 |
| ENSG00000196139.13 | AKR1C3 |
| ENSG00000138028.14 | CGREF1 |
| ENSG00000088002.11 | SULT2B1 |
| ENSG00000105971.14 | CAV2 |
| ENSG00000168811.6 | IL12A |
| ENSG00000137309.19 | HMGA1 |
| ENSG00000114455.13 | HHLA2 |
| ENSG00000188816.3 | HMX2 |
| ENSG00000198286.9 | CARD11 |
| ENSG00000100300.17 | TSPO |
| ENSG00000117595.10 | IRF6 |
| ENSG00000172216.5 | CEBPB |
| ENSG00000127152.17 | BCL11B |
| ENSG00000036828.14 | CASR |
| ENSG00000168918.13 | INPP5D |
| ENSG00000105550.8 | FGF21 |
| ENSG00000156574.9 | NODAL |
| ENSG00000028137.17 | TNFRSF1B |
| ENSG00000173083.14 | HPSE |
| ENSG00000126010.5 | GRPR |
| ENSG00000000005.5 | TNMD |
| ENSG00000167642.12 | SPINT2 |
| ENSG00000162783.10 | IER5 |
| ENSG00000105974.11 | CAV1 |
| ENSG00000160593.17 | JAML |
| ENSG00000100146.16 | SOX10 |
| ENSG00000175793.11 | SFN |
| ENSG00000164129.11 | NPY5R |
| ENSG00000118513.18 | MYB |
| ENSG00000100292.16 | HMOX1 |
| ENSG00000179776.17 | CDH5 |
| ENSG00000135547.8 | HEY2 |
| ENSG00000181885.18 | CLDN7 |
| ENSG00000180871.7 | CXCR2 |
| ENSG00000138685.12 | FGF2 |
| ENSG00000248329.5 | APELA |
| ENSG00000090554.12 | FLT3LG |
| ENSG00000012124.15 | CD22 |
| ENSG00000164649.19 | CDCA7L |
| ENSG00000181163.13 | NPM1 |
| ENSG00000060140.8 | STYK1 |
| ENSG00000215474.7 | SKOR2 |
| ENSG00000137507.11 | LRRC32 |
| ENSG00000113905.4 | HRG |
| ENSG00000062038.13 | CDH3 |
| ENSG00000077238.13 | IL4R |
| ENSG00000164362.18 | TERT |
| ENSG00000214274.9 | ANG |
| ENSG00000132698.14 | RAB25 |
| ENSG00000123572.16 | NRK |
| ENSG00000148926.9 | ADM |
| ENSG00000140832.9 | MARVELD3 |
| ENSG00000197635.9 | DPP4 |
| ENSG00000010610.9 | CD4 |
| ENSG00000012223.12 | LTF |
| ENSG00000075388.3 | FGF4 |
| ENSG00000065361.14 | ERBB3 |
| ENSG00000185885.15 | IFITM1 |
| ENSG00000090530.9 | P3H2 |
| ENSG00000087088.19 | BAX |
| ENSG00000085741.12 | WNT11 |
| ENSG00000245848.2 | CEBPA |
| ENSG00000166148.3 | AVPR1A |
| ENSG00000106278.11 | PTPRZ1 |
| ENSG00000132507.17 | EIF5A |
| ENSG00000130427.2 | EPO |
| ENSG00000169418.9 | NPR1 |
| ENSG00000124588.19 | NQO2 |
| ENSG00000196468.7 | FGF16 |
| ENSG00000146904.8 | EPHA1 |
| ENSG00000006606.8 | CCL26 |
| ENSG00000126368.5 | NR1D1 |
| ENSG00000165025.14 | SYK |
| ENSG00000148344.10 | PTGES |
| ENSG00000110719.9 | TCIRG1 |
| ENSG00000180353.10 | HCLS1 |
| ENSG00000128340.14 | RAC2 |
| ENSG00000243678.11 | NME2 |
| ENSG00000088992.17 | TESC |
| ENSG00000101336.12 | HCK |
| ENSG00000163251.3 | FZD5 |
| ENSG00000134954.14 | ETS1 |
| ENSG00000171388.11 | APLN |
| ENSG00000206557.5 | TRIM71 |
| ENSG00000196839.12 | ADA |
| ENSG00000136997.15 | MYC |
| ENSG00000111846.15 | GCNT2 |
| ENSG00000104332.11 | SFRP1 |
| ENSG00000160867.14 | FGFR4 |
| ENSG00000135638.13 | EMX1 |
| ENSG00000128052.8 | KDR |
| ENSG00000172819.16 | RARG |
| ENSG00000019582.14 | CD74 |
| ENSG00000151577.12 | DRD3 |
| ENSG00000162493.16 | PDPN |
| ENSG00000253368.3 | TRNP1 |
| ENSG00000105707.13 | HPN |
| ENSG00000122861.15 | PLAU |
| ENSG00000239697.10 | TNFSF12 |
| ENSG00000183087.14 | GAS6 |
| ENSG00000101955.14 | SRPX |
| ENSG00000162344.3 | FGF19 |
| ENSG00000163421.8 | PROK2 |
| ENSG00000145777.14 | TSLP |
| ENSG00000182199.10 | SHMT2 |
| ENSG00000102096.9 | PIM2 |
| ENSG00000106128.18 | GHRHR |
| ENSG00000105246.5 | EBI3 |
| ENSG00000163485.15 | ADORA1 |
| ENSG00000164867.10 | NOS3 |
| ENSG00000128342.4 | LIF |
| ENSG00000254093.8 | PINX1 |
| ENSG00000120949.14 | TNFRSF8 |
| ENSG00000103089.8 | FA2H |
| ENSG00000136110.12 | LECT1 |
| ENSG00000168539.3 | CHRM1 |
| ENSG00000239672.7 | NME1 |
| ENSG00000129194.7 | SOX15 |
| ENSG00000163191.5 | S100A11 |
| ENSG00000188505.4 | NCCRP1 |
| ENSG00000101017.13 | CD40 |
| ENSG00000057149.15 | SERPINB3 |
| ENSG00000133321.10 | RARRES3 |
| ENSG00000131914.10 | LIN28A |
| ENSG00000100721.10 | TCL1A |
| ENSG00000160223.16 | ICOSLG |
| ENSG00000114378.16 | HYAL1 |
| ENSG00000204472.12 | AIF1 |
| ENSG00000174697.4 | LEP |
| ENSG00000124802.11 | EEF1E1 |
| ENSG00000027075.13 | PRKCH |
| ENSG00000114812.12 | VIPR1 |
| ENSG00000157368.10 | IL34 |
| ENSG00000111252.10 | SH2B3 |
| ENSG00000166145.14 | SPINT1 |
| ENSG00000103067.12 | ESRP2 |
| ENSG00000103490.13 | PYCARD |
| ENSG00000182566.13 | CLEC4G |
| ENSG00000007264.14 | MATK |
| ENSG00000145088.8 | EAF2 |
| ENSG00000115353.10 | TACR1 |
| ENSG00000172889.15 | EGFL7 |
| ENSG00000205089.7 | CCNI2 |
| ENSG00000069482.6 | GAL |
| ENSG00000101311.15 | FERMT1 |
| ENSG00000120057.4 | SFRP5 |
| ENSG00000101445.9 | PPP1R16B |
| ENSG00000009950.15 | MLXIPL |
| ENSG00000172818.9 | OVOL1 |
| ENSG00000010278.12 | CD9 |
| ENSG00000125657.4 | TNFSF9 |
| ENSG00000175707.8 | KDF1 |
| ENSG00000164078.12 | MST1R |
| ENSG00000110944.8 | IL23A |
| ENSG00000102755.10 | FLT1 |
| ENSG00000122025.14 | FLT3 |
| ENSG00000204632.11 | HLA-G |
| ENSG00000134917.9 | ADAMTS8 |
| ENSG00000070019.4 | GUCY2C |
| ENSG00000100985.7 | MMP9 |
| ENSG00000179593.15 | ALOX15B |
| ENSG00000111424.10 | VDR |
| ENSG00000100625.8 | SIX4 |
| ENSG00000131981.15 | LGALS3 |
| ENSG00000058085.14 | LAMC2 |
| ENSG00000105173.13 | CCNE1 |
| ENSG00000163273.3 | NPPC |
| ENSG00000105205.6 | CLC |
| ENSG00000130203.9 | APOE |
| ENSG00000197442.9 | MAP3K5 |
| ENSG00000110092.3 | CCND1 |
| ENSG00000143184.4 | XCL1 |
| ENSG00000111679.16 | PTPN6 |
| ENSG00000111087.9 | GLI1 |
| ENSG00000213231.12 | TCL1B |
| ENSG00000137193.13 | PIM1 |
| ENSG00000081181.7 | ARG2 |
| ENSG00000254087.7 | LYN |
| ENSG00000198435.3 | NRARP |
| ENSG00000128886.11 | ELL3 |
| ENSG00000241186.8 | TDGF1 |
| ENSG00000175592.8 | FOSL1 |
| ENSG00000144354.13 | CDCA7 |
| ENSG00000111704.10 | NANOG |
| ENSG00000110148.9 | CCKBR |
| ENSG00000169594.13 | BNC1 |
| ENSG00000198805.11 | PNP |
| ENSG00000173334.3 | TRIB1 |
| ENSG00000164120.13 | HPGD |
| ENSG00000196415.9 | PRTN3 |
| ENSG00000165757.8 | KIAA1462 |
| ENSG00000178394.4 | HTR1A |
| ENSG00000010671.15 | BTK |
| ENSG00000155760.2 | FZD7 |
| ENSG00000185436.11 | IFNLR1 |
| ENSG00000105639.18 | JAK3 |
| ENSG00000196352.14 | CD55 |
| ENSG00000090447.11 | TFAP4 |
| ENSG00000155926.13 | SLA |
| ENSG00000116661.9 | FBXO2 |
| ENSG00000166831.8 | RBPMS2 |
| ENSG00000145623.12 | OSMR |
| ENSG00000081985.10 | IL12RB2 |
| ENSG00000119888.10 | EPCAM |
| ENSG00000136244.11 | IL6 |
| ENSG00000131203.12 | IDO1 |
| ENSG00000166869.2 | CHP2 |
| ENSG00000169403.11 | PTAFR |
| ENSG00000163739.4 | CXCL1 |
| ENSG00000145423.4 | SFRP2 |
| ENSG00000163737.3 | PF4 |
| ENSG00000168071.21 | CCDC88B |
| ENSG00000065675.14 | PRKCQ |
| ENSG00000163735.6 | CXCL5 |
| ENSG00000163235.15 | TGFA |
| ENSG00000152661.7 | GJA1 |
| ENSG00000188763.4 | FZD9 |
| ENSG00000106399.11 | RPA3 |
| ENSG00000184292.6 | TACSTD2 |
| ENSG00000141655.15 | TNFRSF11A |
| ENSG00000130176.7 | CNN1 |
| ENSG00000125384.6 | PTGER2 |

TABLE 10
Exemplary genes of gene ontology GO:0006954 with 4 times decreased
gene expression levels relative to a pluripotent stem cell.

| | Gene |
|---|---|
| Gene ID | Symbol |
| ENSG00000125730.16 | C3 |
| ENSG00000169129.14 | AFAP1L2 |
| ENSG00000168229.3 | PTGDR |
| ENSG00000174600.13 | CMKLR1 |
| ENSG00000172216.5 | CEBPB |
| ENSG00000167604.13 | NFKBID |
| ENSG00000028137.17 | TNFRSF1B |
| ENSG00000130768.14 | SMPDL3B |
| ENSG00000164251.4 | F2RL1 |
| ENSG00000100292.16 | HMOX1 |
| ENSG00000180871.7 | CXCR2 |
| ENSG00000171049.8 | FPR2 |
| ENSG00000163701.18 | IL17RE |
| ENSG00000140835.9 | CHST4 |
| ENSG00000077238.13 | IL4R |
| ENSG00000144802.11 | NFKBIZ |
| ENSG00000104856.13 | RELB |
| ENSG00000148926.9 | ADM |
| ENSG00000012779.10 | ALOX5 |
| ENSG00000118785.13 | SPP1 |
| ENSG00000185187.12 | SIGIRR |
| ENSG00000130427.2 | EPO |
| ENSG00000006606.8 | CCL26 |
| ENSG00000165025.14 | SYK |
| ENSG00000148344.10 | PTGES |
| ENSG00000106327.12 | TFR2 |
| ENSG00000101444.12 | AHCY |
| ENSG00000110719.9 | TCIRG1 |
| ENSG00000133048.12 | CHI3L1 |
| ENSG00000241635.7 | UGT1A1 |
| ENSG00000182261.3 | NLRP10 |
| ENSG00000101336.12 | HCK |
| ENSG00000106538.9 | RARRES2 |
| ENSG00000164344.15 | KLKB1 |
| ENSG00000081041.8 | CXCL2 |
| ENSG00000131187.9 | F12 |
| ENSG00000161905.12 | ALOX15 |
| ENSG00000163421.8 | PROK2 |
| ENSG00000163435.15 | ELF3 |
| ENSG00000163485.15 | ADORA1 |
| ENSG00000124875.9 | CXCL6 |
| ENSG00000101017.13 | CD40 |
| ENSG00000114378.16 | HYAL1 |
| ENSG00000204472.12 | AIF1 |
| ENSG00000127507.17 | ADGRE2 |
| ENSG00000157368.10 | IL34 |
| ENSG00000145192.12 | AHSG |
| ENSG00000130775.15 | THEMIS2 |
| ENSG00000008516.16 | MMP25 |
| ENSG00000188313.12 | PLSCR1 |
| ENSG00000123609.10 | NMI |
| ENSG00000103490.13 | PYCARD |
| ENSG00000115353.10 | TACR1 |
| ENSG00000129988.5 | LBP |
| ENSG00000069482.6 | GAL |
| ENSG00000158769.17 | F11R |
| ENSG00000054219.10 | LY75 |
| ENSG00000110944.8 | IL23A |
| ENSG00000174004.5 | NRROS |
| ENSG00000143184.4 | XCL1 |
| ENSG00000130707.17 | ASS1 |
| ENSG00000254087.7 | LYN |
| ENSG00000010671.15 | BTK |
| ENSG00000123610.4 | TNFAIP6 |
| ENSG00000136244.11 | IL6 |
| ENSG00000131203.12 | IDO1 |
| ENSG00000169403.11 | PTAFR |
| ENSG00000163739.4 | CXCL1 |
| ENSG00000163737.3 | PF4 |
| ENSG00000065675.14 | PRKCQ |
| ENSG00000124391.4 | IL17C |
| ENSG00000163735.6 | CXCL5 |
| ENSG00000152661.7 | GJA1 |
| ENSG00000163734.4 | CXCL3 |
| ENSG00000105499.13 | PLA2G4C |
| ENSG00000090339.8 | ICAM1 |
| ENSG00000228278.3 | ORM2 |
| ENSG00000115884.10 | SDC1 |
| ENSG00000125384.6 | PTGER2 |
| ENSG00000164342.12 | TLR3 |

TABLE 11
Exemplary genes of gene ontology GO:0032502 with 4 times decreased
gene expression levels relative to a pluripotent stem cell.

| | Gene |
|---|---|
| Gene ID | Symbol |
| ENSG00000125730.16 | C3 |
| ENSG00000204655.11 | MOG |
| ENSG00000214336.4 | FOXI3 |
| ENSG00000248746.5 | ACTN3 |
| ENSG00000187848.12 | P2RX2 |
| ENSG00000233608.3 | TWIST2 |
| ENSG00000135636.13 | DYSF |
| ENSG00000086967.9 | MYBPC2 |
| ENSG00000101842.13 | VSIG1 |
| ENSG00000196139.13 | AKR1C3 |
| ENSG00000105971.14 | CAV2 |
| ENSG00000050767.15 | COL23A1 |
| ENSG00000168229.3 | PTGDR |
| ENSG00000181856.14 | SLC2A4 |
| ENSG00000108387.14 | 4-Sep |
| ENSG00000108375.12 | RNF43 |
| ENSG00000164403.14 | SHROOM1 |
| ENSG00000132692.18 | BCAN |
| ENSG00000000938.12 | FGR |
| ENSG00000106003.12 | LFNG |
| ENSG00000188508.10 | KRTDAP |
| ENSG00000124827.6 | GCM2 |
| ENSG00000196189.12 | SEMA4A |
| ENSG00000127561.14 | SYNGR3 |
| ENSG00000197467.13 | COL13A1 |
| ENSG00000101347.8 | SAMHD1 |
| ENSG00000188389.10 | PDCD1 |
| ENSG00000137309.19 | HMGA1 |
| ENSG00000134762.16 | DSC3 |
| ENSG00000176928.5 | GCNT4 |
| ENSG00000070388.11 | FGF22 |
| ENSG00000172554.11 | SNTG2 |
| ENSG00000188816.3 | HMX2 |
| ENSG00000198286.9 | CARD11 |
| ENSG00000100300.17 | TSPO |
| ENSG00000117595.10 | IRF6 |
| ENSG00000163884.3 | KLF15 |
| ENSG00000158578.18 | ALAS2 |
| ENSG00000169035.11 | KLK7 |
| ENSG00000135253.13 | KCP |
| ENSG00000170340.10 | B3GNT2 |
| ENSG00000174600.13 | CMKLR1 |
| ENSG00000103740.9 | ACSBG1 |
| ENSG00000165215.6 | CLDN3 |
| ENSG00000100714.15 | MTHFD1 |
| ENSG00000172216.5 | CEBPB |
| ENSG00000127152.17 | BCL11B |
| ENSG00000184344.3 | GDF3 |
| ENSG00000036828.14 | CASR |
| ENSG00000112759.16 | SLC29A1 |
| ENSG00000137709.9 | POU2F3 |
| ENSG00000149922.10 | TBX6 |
| ENSG00000071626.16 | DAZAP1 |
| ENSG00000157150.4 | TIMP4 |
| ENSG00000100362.12 | PVALB |
| ENSG00000168918.13 | INPP5D |
| ENSG00000147676.13 | MAL2 |
| ENSG00000124479.8 | NDP |
| ENSG00000066427.21 | ATXN3 |
| ENSG00000149573.8 | MPZL2 |
| ENSG00000156574.9 | NODAL |
| ENSG00000028137.17 | TNFRSF1B |
| ENSG00000131668.13 | BARX1 |
| ENSG00000081051.7 | AFP |
| ENSG00000173083.14 | HPSE |
| ENSG00000185338.4 | SOCS1 |
| ENSG00000109832.13 | DDX25 |
| ENSG00000196878.13 | LAMB3 |
| ENSG00000000005.5 | TNMD |
| ENSG00000152430.17 | BOLL |
| ENSG00000167642.12 | SPINT2 |
| ENSG00000171517.5 | LPAR3 |
| ENSG00000105974.11 | CAV1 |
| ENSG00000137265.14 | IRF4 |
| ENSG00000100146.16 | SOX10 |
| ENSG00000175793.11 | SFN |
| ENSG00000164129.11 | NPY5R |
| ENSG00000118513.18 | MYB |
| ENSG00000164251.4 | F2RL1 |
| ENSG00000132382.14 | MYBBP1A |
| ENSG00000100292.16 | HMOX1 |
| ENSG00000185215.8 | TNFAIP2 |
| ENSG00000175602.3 | CCDC85B |
| ENSG00000171777.15 | RASGRP4 |
| ENSG00000145824.12 | CXCL14 |
| ENSG00000179776.17 | CDH5 |
| ENSG00000104267.9 | CA2 |
| ENSG00000135547.8 | HEY2 |
| ENSG00000100628.11 | ASB2 |
| ENSG00000100522.8 | GNPNAT1 |
| ENSG00000117115.12 | PADI2 |
| ENSG00000152214.12 | RIT2 |
| ENSG00000106333.12 | PCOLCE |
| ENSG00000180871.7 | CXCR2 |
| ENSG00000171049.8 | FPR2 |
| ENSG00000138685.12 | FGF2 |
| ENSG00000119969.14 | HELLS |
| ENSG00000165996.13 | HACD1 |
| ENSG00000248329.5 | APELA |
| ENSG00000188501.11 | LCTL |
| ENSG00000167880.7 | EVPL |
| ENSG00000160219.11 | GAB3 |
| ENSG00000090554.12 | FLT3LG |
| ENSG00000111344.11 | RASAL1 |
| ENSG00000198576.3 | ARC |
| ENSG00000117148.7 | ACTL8 |
| ENSG00000181163.13 | NPM1 |
| ENSG00000115541.10 | HSPE1 |
| ENSG00000039068.18 | CDH1 |
| ENSG00000215474.7 | SKOR2 |
| ENSG00000265763.3 | ZNF488 |
| ENSG00000132359.14 | RAP1GAP2 |
| ENSG00000117322.16 | CR2 |
| ENSG00000113905.4 | HRG |
| ENSG00000164687.10 | FABP5 |
| ENSG00000062038.13 | CDH3 |
| ENSG00000204264.8 | PSMB8 |
| ENSG00000187140.5 | FOXD3 |
| ENSG00000164651.16 | SP8 |
| ENSG00000164362.18 | TERT |
| ENSG00000214274.9 | ANG |
| ENSG00000244094.1 | SPRR2F |
| ENSG00000122679.8 | RAMP3 |
| ENSG00000114638.7 | UPK1B |
| ENSG00000043143.20 | JADE2 |
| ENSG00000119139.17 | TJP2 |
| ENSG00000006468.13 | ETV1 |
| ENSG00000198626.15 | RYR2 |
| ENSG00000132698.14 | RAB25 |
| ENSG00000126803.9 | HSPA2 |
| ENSG00000123572.16 | NRK |
| ENSG00000104856.13 | RELB |
| ENSG00000109861.15 | CTSC |
| ENSG00000163083.5 | INHBB |
| ENSG00000138772.12 | ANXA3 |
| ENSG00000187266.13 | EPOR |
| ENSG00000204644.9 | ZFP57 |
| ENSG00000100290.2 | BIK |
| ENSG00000148926.9 | ADM |
| ENSG00000092345.13 | DAZL |
| ENSG00000169908.11 | TM4SF1 |
| ENSG00000163932.13 | PRKCD |
| ENSG00000010610.9 | CD4 |
| ENSG00000117407.16 | ARTN |
| ENSG00000204531.16 | POU5F1 |
| ENSG00000012223.12 | LTF |
| ENSG00000006047.12 | YBX2 |
| ENSG00000187678.8 | SPRY4 |
| ENSG00000158813.17 | EDA |
| ENSG00000075388.3 | FGF4 |
| ENSG00000170608.2 | FOXA3 |
| ENSG00000144852.16 | NR1I2 |
| ENSG00000269404.6 | SPIB |
| ENSG00000147465.11 | STAR |
| ENSG00000111913.16 | FAM65B |
| ENSG00000065361.14 | ERBB3 |
| ENSG00000138363.14 | ATIC |
| ENSG00000128805.14 | ARHGAP22 |
| ENSG00000140511.11 | HAPLN3 |
| ENSG00000181274.6 | FRAT2 |
| ENSG00000158887.15 | MPZ |
| ENSG00000141497.13 | ZMYND15 |
| ENSG00000089820.15 | ARHGAP4 |
| ENSG00000130751.9 | NPAS1 |
| ENSG00000134516.15 | DOCK2 |
| ENSG00000101282.8 | RSPO4 |
| ENSG00000157766.15 | ACAN |
| ENSG00000125878.6 | TCF15 |
| ENSG00000187955.11 | COL14A1 |
| ENSG00000120254.15 | MTHFD1L |
| ENSG00000087088.19 | BAX |
| ENSG00000085741.12 | WNT11 |
| ENSG00000245848.2 | CEBPA |
| ENSG00000166148.3 | AVPR1A |
| ENSG00000106278.11 | PTPRZ1 |
| ENSG00000118785.13 | SPP1 |
| ENSG00000184160.7 | ADRA2C |
| ENSG00000134709.10 | HOOK1 |
| ENSG00000196431.3 | CRYBA4 |
| ENSG00000101280.7 | ANGPT4 |
| ENSG00000008324.10 | SS18L2 |
| ENSG00000119866.20 | BCL11A |
| ENSG00000164695.4 | CHMP4C |
| ENSG00000169860.6 | P2RY1 |
| ENSG00000139800.8 | ZIC5 |
| ENSG00000131652.13 | THOC6 |
| ENSG00000123405.13 | NFE2 |
| ENSG00000128422.15 | KRT17 |
| ENSG00000130427.2 | EPO |
| ENSG00000117676.13 | RPS6KA1 |
| ENSG00000105668.7 | UPK1A |
| ENSG00000189292.15 | FAM150B |
| ENSG00000138039.14 | LHCGR |
| ENSG00000196468.7 | FGF16 |
| ENSG00000121570.12 | DPPA4 |
| ENSG00000135480.14 | KRT7 |
| ENSG00000146904.8 | EPHA1 |
| ENSG00000105427.9 | CNFN |
| ENSG00000163646.10 | CLRN1 |
| ENSG00000126368.5 | NR1D1 |
| ENSG00000116016.13 | EPAS1 |
| ENSG00000165025.14 | SYK |
| ENSG00000174343.5 | CHRNA9 |
| ENSG00000081277.12 | PKP1 |
| ENSG00000166527.7 | CLEC4D |
| ENSG00000155846.16 | PPARGC1B |
| ENSG00000152208.12 | GRID2 |
| ENSG00000010319.6 | SEMA3G |
| ENSG00000079337.15 | RAPGEF3 |
| ENSG00000070182.18 | SPTB |
| ENSG00000265107.2 | GJA5 |
| ENSG00000142552.7 | RCN3 |
| ENSG00000170374.5 | SP7 |
| ENSG00000110719.9 | TCIRG1 |
| ENSG00000133048.12 | CHI3L1 |
| ENSG00000241635.7 | UGT1A1 |
| ENSG00000180353.10 | HCLS1 |
| ENSG00000172830.12 | SSH3 |
| ENSG00000123600.18 | METTL8 |
| ENSG00000143365.16 | RORC |
| ENSG00000186971.3 | KRTAP13-4 |
| ENSG00000128340.14 | RAC2 |
| ENSG00000167759.12 | KLK13 |
| ENSG00000243678.11 | NME2 |
| ENSG00000088992.17 | TESC |
| ENSG00000179041.3 | RRS1 |
| ENSG00000101336.12 | HCK |
| ENSG00000163251.3 | FZD5 |
| ENSG00000164128.6 | NPY1R |
| ENSG00000188782.8 | CATSPER4 |
| ENSG00000167157.10 | PRRX2 |
| ENSG00000134954.14 | ETS1 |
| ENSG00000162551.13 | ALPL |
| ENSG00000171388.11 | APLN |
| ENSG00000102575.10 | ACP5 |
| ENSG00000206557.5 | TRIM71 |
| ENSG00000196839.12 | ADA |
| ENSG00000106538.9 | RARRES2 |
| ENSG00000117450.13 | PRDX1 |
| ENSG00000180739.13 | S1PR5 |
| ENSG00000136997.15 | MYC |
| ENSG00000111846.15 | GCNT2 |
| ENSG00000104332.11 | SFRP1 |
| ENSG00000160867.14 | FGFR4 |
| ENSG00000178343.4 | SHISA3 |
| ENSG00000171246.5 | NPTX1 |
| ENSG00000258417.3 | RP11-240B13.2 |
| ENSG00000186766.7 | FOXI2 |
| ENSG00000135638.13 | EMX1 |
| ENSG00000128052.8 | KDR |
| ENSG00000146530.11 | VWDE |
| ENSG00000088305.18 | DNMT3B |
| ENSG00000184254.16 | ALDH1A3 |
| ENSG00000109107.13 | ALDOC |
| ENSG00000172819.16 | RARG |
| ENSG00000019582.14 | CD74 |
| ENSG00000162782.15 | TDRD5 |
| ENSG00000176165.10 | FOXG1 |
| ENSG00000151577.12 | DRD3 |
| ENSG00000148600.14 | CDHR1 |
| ENSG00000168389.17 | MFSD2A |
| ENSG00000162493.16 | PDPN |
| ENSG00000188487.11 | INSC |
| ENSG00000186907.7 | RTN4RL2 |
| ENSG00000085999.11 | RAD54L |
| ENSG00000186297.11 | GABRA5 |
| ENSG00000163666.8 | HESX1 |
| ENSG00000133316.15 | WDR74 |
| ENSG00000253368.3 | TRNP1 |
| ENSG00000105707.13 | HPN |
| ENSG00000187840.4 | EIF4EBP1 |
| ENSG00000105877.17 | DNAH11 |
| ENSG00000004478.7 | FKBP4 |
| ENSG00000203909.3 | DPPA5 |
| ENSG00000161905.12 | ALOX15 |
| ENSG00000120669.15 | SOHLH2 |
| ENSG00000111752.10 | PHC1 |
| ENSG00000136167.13 | LCP1 |
| ENSG00000159167.11 | STC1 |
| ENSG00000172238.4 | ATOH1 |
| ENSG00000080224.17 | EPHA6 |
| ENSG00000173673.7 | HES3 |
| ENSG00000239697.10 | TNFSF12 |
| ENSG00000183087.14 | GAS6 |
| ENSG00000184363.9 | PKP3 |
| ENSG00000162344.3 | FGF19 |
| ENSG00000163421.8 | PROK2 |
| ENSG00000137819.13 | PAQR5 |
| ENSG00000159228.12 | CBR1 |
| ENSG00000163435.15 | ELF3 |
| ENSG00000159374.17 | M1AP |
| ENSG00000078596.10 | ITM2A |
| ENSG00000050555.17 | LAMC3 |
| ENSG00000135605.12 | TEC |
| ENSG00000106852.15 | LHX6 |
| ENSG00000173868.11 | PHOSPHO1 |
| ENSG00000106128.18 | GHRHR |
| ENSG00000187513.8 | GJA4 |
| ENSG00000174307.6 | PHLDA3 |
| ENSG00000169220.17 | RGS14 |
| ENSG00000179403.11 | VWA1 |
| ENSG00000124233.11 | SEMG1 |
| ENSG00000151650.7 | VENTX |
| ENSG00000170909.13 | OSCAR |
| ENSG00000154237.12 | LRRK1 |
| ENSG00000229544.8 | NKX1-2 |
| ENSG00000249751.3 | ECSCR |
| ENSG00000163485.15 | ADORA1 |
| ENSG00000169896.16 | ITGAM |
| ENSG00000164867.10 | NOS3 |
| ENSG00000204385.10 | SLC44A4 |
| ENSG00000108518.7 | PFN1 |
| ENSG00000073146.15 | MOV10L1 |
| ENSG00000136383.6 | ALPK3 |
| ENSG00000128342.4 | LIF |
| ENSG00000129455.15 | KLK8 |
| ENSG00000095587.8 | TLL2 |
| ENSG00000127831.10 | VIL1 |
| ENSG00000112041.12 | TULP1 |
| ENSG00000092621.11 | PHGDH |
| ENSG00000103089.8 | FA2H |
| ENSG00000156453.13 | PCDH1 |
| ENSG00000144381.16 | HSPD1 |
| ENSG00000008394.12 | MGST1 |
| ENSG00000197594.11 | ENPP1 |
| ENSG00000136110.12 | LECT1 |
| ENSG00000168539.3 | CHRM1 |
| ENSG00000239672.7 | NME1 |
| ENSG00000129194.7 | SOX15 |
| ENSG00000100078.3 | PLA2G3 |
| ENSG00000198598.6 | MMP17 |
| ENSG00000165816.12 | VWA2 |
| ENSG00000169174.10 | PCSK9 |
| ENSG00000144550.12 | CPNE9 |
| ENSG00000104881.15 | PPP1R13L |
| ENSG00000171346.14 | KRT15 |
| ENSG00000078549.14 | ADCYAP1R1 |
| ENSG00000100889.11 | PCK2 |
| ENSG00000149927.17 | DOC2A |
| ENSG00000198844.10 | ARHGEF15 |
| ENSG00000111057.10 | KRT18 |
| ENSG00000175832.12 | ETV4 |
| ENSG00000184895.7 | SRY |
| ENSG00000136943.10 | CTSV |
| ENSG00000131914.10 | LIN28A |
| ENSG00000161798.6 | AQP5 |
| ENSG00000107731.12 | UNC5B |
| ENSG00000105327.16 | BBC3 |
| ENSG00000180447.6 | GAS1 |
| ENSG00000100721.10 | TCL1A |
| ENSG00000157765.11 | SLC34A2 |
| ENSG00000188038.7 | NRN1L |
| ENSG00000106236.3 | NPTX2 |
| ENSG00000114378.16 | HYAL1 |
| ENSG00000204472.12 | AIF1 |
| ENSG00000174697.4 | LEP |
| ENSG00000027075.13 | PRKCH |
| ENSG00000053918.15 | KCNQ1 |
| ENSG00000118194.18 | TNNT2 |
| ENSG00000157368.10 | IL34 |
| ENSG00000111252.10 | SH2B3 |
| ENSG00000145192.12 | AHSG |
| ENSG00000166145.14 | SPINT1 |
| ENSG00000105538.9 | RASIP1 |
| ENSG00000008516.16 | MMP25 |
| ENSG00000083454.21 | P2RX5 |
| ENSG00000141738.13 | GRB7 |
| ENSG00000198931.10 | APRT |
| ENSG00000141968.7 | VAV1 |
| ENSG00000105048.16 | TNNT1 |
| ENSG00000103067.12 | ESRP2 |
| ENSG00000158715.5 | SLC45A3 |
| ENSG00000007264.14 | MATK |
| ENSG00000104413.15 | ESRP1 |
| ENSG00000147166.10 | ITGB1BP2 |
| ENSG00000159753.13 | CARMIL2 |
| ENSG00000182372.8 | CLN8 |
| ENSG00000128965.11 | CHAC1 |
| ENSG00000172889.15 | EGFL7 |
| ENSG00000132749.10 | TESMIN |
| ENSG00000120057.4 | SFRP5 |
| ENSG00000103257.8 | SLC7A5 |
| ENSG00000168062.9 | BATF2 |
| ENSG00000101445.9 | PPP1R16B |
| ENSG00000122145.14 | TBX22 |
| ENSG00000128165.8 | ADM2 |
| ENSG00000160973.7 | FOXH1 |
| ENSG00000009950.15 | MLXIPL |
| ENSG00000179772.7 | FOXS1 |
| ENSG00000158769.17 | F11R |
| ENSG00000131264.3 | CDX4 |
| ENSG00000172818.9 | OVOL1 |
| ENSG00000119614.2 | VSX2 |
| ENSG00000010278.12 | CD9 |
| ENSG00000196549.10 | MME |
| ENSG00000176402.5 | GJC3 |
| ENSG00000175707.8 | KDF1 |
| ENSG00000102755.10 | FLT1 |
| ENSG00000122025.14 | FLT3 |
| ENSG00000173093.12 | CCDC63 |
| ENSG00000204632.11 | HLA-G |
| ENSG00000158748.3 | HTR6 |
| ENSG00000189143.9 | CLDN4 |
| ENSG00000137672.12 | TRPC6 |
| ENSG00000130477.15 | UNCI3A |
| ENSG00000077522.12 | ACTN2 |
| ENSG00000174004.5 | NRROS |
| ENSG00000188910.7 | GJB3 |
| ENSG00000196711.8 | FAM150A |
| ENSG00000173262.11 | SLC2A14 |
| ENSG00000104369.4 | JPH1 |
| ENSG00000100985.7 | MMP9 |
| ENSG00000179593.15 | ALOX15B |
| ENSG00000140600.16 | SH3GL3 |
| ENSG00000111424.10 | VDR |
| ENSG00000100625.8 | SIX4 |
| ENSG00000131981.15 | LGALS3 |
| ENSG00000052344.15 | PRSS8 |
| ENSG00000163359.15 | COL6A3 |
| ENSG00000130182.7 | ZSCAN10 |
| ENSG00000105695.14 | MAG |
| ENSG00000142185.16 | TRPM2 |
| ENSG00000142173.14 | COL6A2 |
| ENSG00000123892.11 | RAB38 |
| ENSG00000058085.14 | LAMC2 |
| ENSG00000166426.7 | CRABP1 |
| ENSG00000113749.7 | HRH2 |
| ENSG00000163273.3 | NPPC |
| ENSG00000105205.6 | CLC |
| ENSG00000180209.11 | MYLPF |
| ENSG00000204571.5 | KRTAP5-11 |
| ENSG00000196154.11 | S100A4 |
| ENSG00000043355.11 | ZIC2 |
| ENSG00000130203.9 | APOE |
| ENSG00000145220.13 | LYAR |
| ENSG00000253117.4 | OC90 |
| ENSG00000110092.3 | CCND1 |
| ENSG00000167749.11 | KLK4 |
| ENSG00000171509.15 | RXFP1 |
| ENSG00000164430.15 | MB21D1 |
| ENSG00000124212.5 | PTGIS |
| ENSG00000139269.2 | INHBE |
| ENSG00000111679.16 | PTPN6 |
| ENSG00000197943.9 | PLCG2 |
| ENSG00000105202.7 | FBL |
| ENSG00000111087.9 | GLI1 |
| ENSG00000130707.17 | ASS1 |
| ENSG00000124507.10 | PACSIN1 |
| ENSG00000165091.15 | TMC1 |
| ENSG00000137193.13 | PIM1 |
| ENSG00000165704.14 | HPRT1 |
| ENSG00000162433.14 | AK4 |
| ENSG00000081181.7 | ARG2 |
| ENSG00000254087.7 | LYN |
| ENSG00000198435.3 | NRARP |
| ENSG00000128886.11 | ELL3 |
| ENSG00000182459.4 | TEX19 |
| ENSG00000241186.8 | TDGF1 |
| ENSG00000188095.4 | MESP2 |
| ENSG00000177791.11 | MYOZ1 |
| ENSG00000125144.13 | MT1G |
| ENSG00000130700.6 | GATA5 |
| ENSG00000175592.8 | FOSL1 |
| ENSG00000172461.10 | FUT9 |
| ENSG00000141384.12 | TAF4B |
| ENSG00000111704.10 | NANOG |
| ENSG00000167077.12 | MEI1 |
| ENSG00000110148.9 | CCKBR |
| ENSG00000179477.9 | ALOX12B |
| ENSG00000149418.10 | STU |
| ENSG00000167414.4 | GNG8 |
| ENSG00000169594.13 | BNC1 |
| ENSG00000177807.7 | KCNJ10 |
| ENSG00000184571.13 | PIWIL3 |
| ENSG00000181392.14 | SYNE4 |
| ENSG00000100814.17 | CCNB1IP1 |
| ENSG00000108813.10 | DLX4 |
| ENSG00000070669.16 | ASNS |
| ENSG00000102387.15 | TAF7L |
| ENSG00000132164.9 | SLC6A11 |
| ENSG00000198963.10 | RORB |
| ENSG00000111845.4 | PAK1IP1 |
| ENSG00000214513.3 | NOTO |
| ENSG00000164120.13 | HPGD |
| ENSG00000183770.5 | FOXL2 |
| ENSG00000171345.13 | KRT19 |
| ENSG00000133067.17 | LGR6 |
| ENSG00000122574.10 | WIPF3 |
| ENSG00000140545.14 | MFGE8 |
| ENSG00000196415.9 | PRTN3 |
| ENSG00000177455.12 | CD19 |
| ENSG00000111321.10 | LTBR |
| ENSG00000053108.16 | FSTL4 |
| ENSG00000183688.4 | FAM101B |
| ENSG00000123342.15 | MMP19 |
| ENSG00000010671.15 | BTK |
| ENSG00000167754.12 | KLK5 |
| ENSG00000111962.7 | UST |
| ENSG00000155760.2 | FZD7 |
| ENSG00000101331.15 | CCM2L |
| ENSG00000011201.11 | ANOS1 |
| ENSG00000069812.11 | HES2 |
| ENSG00000105639.18 | JAK3 |
| ENSG00000150051.13 | MKX |
| ENSG00000155926.13 | SLA |
| ENSG00000137642.12 | SORL1 |
| ENSG00000117600.12 | PLPPR4 |
| ENSG00000138759.17 | FRAS1 |
| ENSG00000139318.7 | DUSP6 |
| ENSG00000187688.14 | TRPV2 |
| ENSG00000132470.13 | ITGB4 |
| ENSG00000262179.2 | RP1-302G2.5 |
| ENSG00000166831.8 | RBPMS2 |
| ENSG00000060138.12 | YBX3 |
| ENSG00000119888.10 | EPCAM |
| ENSG00000105610.4 | KLF1 |
| ENSG00000136244.11 | IL6 |
| ENSG00000027869.11 | SH2D2A |
| ENSG00000131650.13 | KREMEN2 |
| ENSG00000154096.13 | THY1 |
| ENSG00000163739.4 | CXCL1 |
| ENSG00000147596.3 | PRDM14 |
| ENSG00000118231.4 | CRYGD |
| ENSG00000101115.12 | SALL4 |
| ENSG00000158055.15 | GRHL3 |
| ENSG00000171794.3 | UTF1 |
| ENSG00000187569.2 | DPPA3 |
| ENSG00000116774.11 | OLFML3 |
| ENSG00000169877.9 | AHSP |
| ENSG00000143028.8 | SYPL2 |
| ENSG00000145423.4 | SFRP2 |
| ENSG00000125354.22 | 6-Sep |
| ENSG00000089250.18 | NOS1 |
| ENSG00000087510.6 | TFAP2C |
| ENSG00000128482.15 | RNF112 |
| ENSG00000182866.16 | LCK |
| ENSG00000065675.14 | PRKCQ |
| ENSG00000115641.18 | FHL2 |
| ENSG00000174607.10 | UGT8 |
| ENSG00000095627.9 | TDRD1 |
| ENSG00000118242.15 | MREG |
| ENSG00000184557.4 | SOCS3 |
| ENSG00000136487.17 | GH2 |
| ENSG00000163235.15 | TGFA |
| ENSG00000197905.8 | TEAD4 |
| ENSG00000152661.7 | GJA1 |
| ENSG00000188763.4 | FZD9 |
| ENSG00000178882.14 | FAM101A |
| ENSG00000187498.14 | COL4A1 |
| ENSG00000164588.6 | HCN1 |
| ENSG00000184292.6 | TACSTD2 |
| ENSG00000141161.11 | UNC45B |
| ENSG00000120833.13 | SOCS2 |
| ENSG00000090339.8 | ICAM1 |
| ENSG00000128567.16 | PODXL |
| ENSG00000179059.9 | ZFP42 |
| ENSG00000175315.2 | CST6 |
| ENSG00000128242.12 | GAL3ST1 |
| ENSG00000141655.15 | TNFRSF11A |
| ENSG00000106991.13 | ENG |
| ENSG00000129991.12 | TNNI3 |
| ENSG00000007312.12 | CD79B |
| ENSG00000115884.10 | SDC1 |
| ENSG00000118526.6 | TCF21 |
| ENSG00000144962.6 | SPATA16 |
| ENSG00000092758.15 | COL9A3 |
| ENSG00000164342.12 | TLR3 |
| ENSG00000147202.17 | DIAPH2 |
| ENSG00000046889.18 | PREX2 |
| ENSG00000158859.9 | ADAMTS4 |
| ENSG00000138100.13 | TRIM54 |
| ENSG00000169750.8 | RAC3 |

### REFERENCES

Brunet, J. P., Tamayo, P., Golub, T. R., and Mesirov, J. P. (2004). Metagenes and molecular pattern discovery using matrix factorization. Proc Natl Acad Sci USA 101, 4164-4169.
Daley, G. Q., Lensch, M. W., Jaenisch, R., Meissner, A., Plath, K., and Yamanaka, S. (2009). Broader implications of defining standards for the pluripotency of iPSCs. Cell Stem Cell 4, 200-201; author reply 202.
di Domenico, A., Carola, G., Calatayud, C., Pons-Espinal, M., Munoz, J. P., Richaud-Patin, Y., Fernandez-Carasa, I., Gut, M., Faella, A., Parameswaran, J., et al. (2019). Patient-Specific iPSC-Derived Astrocytes Contribute to Non-Cell-Autonomous Neurodegeneration in Parkinson's Disease. Stem Cell Reports 12, 213-229.
Kibbe, W. A., and Lin, S. M. (2008). lumi: a pipeline for processing Illumina microarray. Bioinformatics 24, 1547-1548.
Hall, C. E., Yao, Z., Choi, M., Tyzack, G. E., Serio, A., Luisier, R., Harley, J., Preza, E., Arber, C., Crisp, S. J., et al. (2017). Progressive Motor Neuron Pathology and the Role of Astrocytes in a Human Stem Cell Model of VCP-Related ALS. Cell Rep 19, 1739-1749.
Hastie, T., Tibshirani, R., and Friedman, J. H. (2009). The elements of statistical learning: data mining, inference, and prediction, 2nd edn (New York, N.Y.: Springer).
Hrdlickova, R., Toloue, M., and Tian, B. (2017). RNA-Seq methods for transcriptome analysis. Wiley Interdiscip Rev RNA 8.
Kouroupi, G., Taoufik, E., Vlachos, I. S., Tsioras, K., Antoniou, N., Papastefanaki, F., Chroni-Tzartou, D., Wrasidlo, W., Bohl, D., Stellas, D., et al. (2017). Defective synaptic connectivity and axonal neuropathology in a human iPSC-based model of familial Parkinson's disease. Proc Natl Acad Sci USA 114, E3679-e3688.
Muller, F. J., Schuldt, B. M., Williams, R., Mason, D., Altun, G., Papapetrou, E. P., Danner, S., Goldmann, J. E., Herbst, A., Schmidt, N. O., et al. (2011). A bioinformatic assay for pluripotency in human cells. Nat Methods 8, 315-317.
Patro, R., Duggal, G., Love, M. I., Irizarry, R. A., and Kingsford, C. (2017). Salmon provides fast and bias-aware quantification of transcript expression. Nat Methods 14, 417-419.
R Development Core Team (2010). R: A language and environment for statistical computing (Vienna, Austria: R Foundation for Statistical Computing).
Studer, L. (2012). Derivation of dopaminergic neurons from pluripotent stem cells. Prog Brain Res 200, 243-263.
Weissbein, U., Plotnik, O., Vershkov, D., and Benvenisty, N. (2017). Culture-induced recurrent epigenetic aberrations in human pluripotent stem cells. PLoS Genet 13, e1006979.
Zafeiriou, S., Tefas, A., Buciu, I., and Pitas, I. (2006). Exploiting discriminant information in nonnegative matrix factorization with application to frontal face verification. IEEE Trans Neural Netw 17, 683-695.

1. A computer implemented method of classifying an in vitro population of neuronal progenitor cells, the method comprising:
receiving a test dataset comprising (a) gene expression levels, and (b) expression levels of one or more metagenes for a cell or a plurality of cells comprised in an in vitro population of neuronal progenitor cells, wherein the one or more metagenes are determined based on correlated gene expression levels of reference cells in a reference database, wherein the reference cells are neuronal cells at one or more different stages of differentiation;
applying the expression levels of the one or more metagenes as input to a process configured to determine a probability of the cell or the plurality of cells having metagene expression levels of a determined dopaminergic precursor cell;
determining a deviation score for the cell or the plurality of cells, wherein the deviation score indicates the degree to which the gene expression levels in the test dataset deviate from gene expression levels in one or more reference cells in the reference database, wherein the one or more reference cells are at a stage of differentiation indicating a determined dopaminergic precursor cell; and
outputting, based on the probability and the deviation score, a computed label classification comprising an indication of whether said cell or said plurality of cells from the in vitro population of neuronal progenitor cells is a determined dopaminergic precursor cell.

2. The computer implemented method of claim 1, wherein:
the process comprises a supervised classification model trained using (i) expression levels of the one or more metagenes of the reference cells in the reference database; and (ii) class labels indicating each of the one or more different stages of differentiation for reference cells in the reference database, to determine a probability of a cell or a plurality of cells having metagene expression levels of a determined dopaminergic precursor cell.

3. A computer implemented method of training a process to determine a probability of a cell or a plurality of cells having metagene expression levels of a determined dopaminergic precursor cell, the method comprising training a supervised classification model using (i) expression levels of one or more metagenes, wherein the one or more metagenes are determined based on correlated gene expression levels of reference cells in a reference database, wherein the reference cells are neuronal cells at one or more different stages of differentiation; and (ii) class labels indicating each of the one or more different stages of differentiation for reference cells in the reference database, to determine a probability of a cell or a plurality of cells having metagene expression levels of a determined dopaminergic precursor cell.

4-6. (canceled)

7. The computer implemented method of claim 1, wherein the reference cells are an in vitro population of neuronal progenitor cells.

8. The computer implemented method of claim 1, wherein said in vitro population of neuronal progenitor cells is formed by culturing one or more induced pluripotent stem cells (iPSC) in vitro for a period of time under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neurons.

9-11. (canceled)

12. The computer implemented method of claim 8, wherein the culturing is for period of time that is between at or about 2 and at or about 25 days.

13-19. (canceled)

20. The computer implemented method of claim 1, wherein the reference database comprises gene expression levels determined from one or more reference cell populations, wherein each of the one or more reference cell populations are formed by culturing one or more iPSC in vitro for a different period of time each under conditions capable of differentiating the one or more iPSCs to a neuronal progenitor cell, optionally wherein the neuronal progenitor cell is one or more of a floor plate midbrain progenitor cells, determined dopaminergic precursor cells, or dopamine (DA) neuron.

21-29. (canceled)

30. The computer implemented method of claim 1, wherein the one or more metagenes and the expression levels of the one or more metagenes are determined by using a dimensionality reduction technique on one or more reference cells of the one or more reference database.

31-41. (canceled)

42. The computer implemented method of claim 2, wherein the class label indicating each of the one or more different stages of differentiation of the reference cells is determined using an in vivo method.

43. The computer implemented method of claim 42, wherein the in vivo method comprises:
transplanting the in vitro population of neuronal progenitor cells comprising a reference cell population into a brain region of an animal model of Parkinson's disease;
assessing the occurrence of an outcome associated with a therapeutic effect of the transplantation on the animal model, optionally wherein the outcome is selected from innervation or engrafting with host cells, reduction of a brain lesion in the animal model, or reversal of a brain lesion in the animal model; and
designating the class label as a determined dopaminergic precursor cell if the transplantation results in the occurrence of the outcome associated with a therapeutic effect; or
designating the class label as not a determined dopaminergic precursor cell if the transplantation does not result in the occurrence of the outcome associated with a therapeutic effect.

44-45. (canceled)

46. The computer implemented method of claim 2, wherein the class label indicating each of the one or more different stages of differentiation of the reference cells is determined using an in vitro method.

47. The computer implemented method of claim 46, wherein:
the in vitro method comprises assessing dopamine production levels of a reference cell population; and
the class label is designated as a determined dopaminergic precursor cell if the dopamine production levels are increased relative to a pluripotent stem cell.

48-51. (canceled)

52. The computer implemented method of claim 1, wherein the expression levels of the one or more metagenes in the test dataset is determined based on (i) the one or more metagenes determined from the one or more reference cells in the reference database and (ii) the gene expression levels in the test dataset.

53. The computer implemented method of claim 52, wherein the expression levels of the one or more metagenes in the test dataset is determined using regression analysis based on (i) the one or more metagenes determined from the one or more reference cells in the reference database and (ii) the gene expression levels in the test dataset.

54. The computer implemented method of claim 30, wherein the expression levels of the one or more metagenes in the test dataset is determined by merging the gene expression levels in the test dataset with the reference database to create an updated reference database and applying the dimensionality reduction technique on the updated reference database.

55-57. (canceled)

58. The computer implemented method of claim 30, wherein the number of the one or more metagenes is chosen based on evaluating one or more metrics determined from performing the dimensionality reduction technique using multiple candidate numbers of metagenes.

59. (canceled)

60. The computer implemented method of claim 1, wherein the computed label classification indicates that said cell or plurality of cells from the in vitro population of neuronal progenitor cells is a determined dopaminergic precursor cell if the probability of the cell or the plurality of cells having metagene expression levels of the determined dopaminergic precursor cell is greater than a threshold probability value.

61. The computer implemented method of claim 60, wherein:
the threshold probability value is set such that a determined dopaminergic precursor cell is identified with greater than or greater than about 75%, 80%, 85%, 90%, or 95% sensitivity;
and/or the threshold probability value is set such that a determined dopaminergic precursor cell is identified with greater than or greater than about 75%, 80%, 85%, 90%, or 95% specificity.

62-65. (canceled)

66. The computer implemented method of claim 1, wherein the deviation score for the cell or the plurality of cells is determined using a single-gene deviation score for each of one or more genes in the test dataset.

67. The computer implemented method of claim 66, wherein the single-gene deviation scores are determined using differences between the gene expression levels of the test dataset and the gene expression levels in one or more reference cells in the reference database.

68. (canceled)

69. The computer implemented method of claim 66, any of wherein the single-gene deviation scores are determined using standard deviations of gene expression levels in one or more of the one or more reference cells.

70. The computer implemented method of claim 66, wherein the single-gene deviation scores are z-scores determined using:
differences between the gene expression levels of the test dataset and the gene expression levels in the one or more reference cells in the reference database; and
standard deviations of gene expression levels in one or more of the one or more reference cells of the reference database.

71-72. (canceled)

73. The computer implemented method of claim 1, wherein the gene expression levels in the one or more reference cells in the reference database are determined using regression analysis based on (i) the expression levels of the one or more metagenes in the test dataset and (ii) the gene expression levels in the test dataset.

74. The computer implemented method of claim 66, wherein the deviation score is a summary statistic based on all single-gene deviation scores.

75. The computer implemented method of claim 66, wherein the deviation score is a summary statistic based on single-gene deviation scores for one or more marker genes.

76. The computer implemented method of claim 74, wherein the summary statistic is a sum or a percentile value.

77-79. (canceled)

80. The computer implemented method of claim 76, wherein:
the percentile value is between or between about the 50% percentile and the 100% percentile; and/or
the percentile value is or is about the 50%, 60%, 70%, 80%, 90%, or 95% percentile.

81. The computer implemented method of claim 75, wherein the marker genes comprise radial glial cell markers, early neuronal development genes, pluripotency specific markers, intermediate to late neuronal markers, neurofilament light polypeptide chain markers, neurofilament medium polypeptide chain markers, nestin filament markers, early patterning markers, neural progenitor cell markers, early migration markers, stage-specific transcription factors, genes required for normal development of neurons, genes controlling dopaminergic neuron development, genes regulating identity and fate of neuronal progenitor cells, dopaminergic neuron markers, astrocyte markers, forebrain markers, hindbrain markers, subthalamic nucleus markers, radial glial markers, cell cycle markers, or any combination of any of the foregoing.

82. The computer implemented method of claim 75, wherein the marker genes are or comprise WNT1, VIM, TOP2A, TH, SOX2A, SLIT2, RFX4, POU5F1, PITX2, PAX6, OTX2, NR4A2, NHLH2, NEUROD4, NEUROD1, NES, NEFM, NEFL, NASP, MAP2, LMX1A, LIN28A, HOXA2, HMGB2, HES1, FOXG1, FOXA2, FABP7, DDC, DCX, BARHL2, BARJL1, ASPM, ALDH1A1, or any combination of any of the foregoing.

83. The computer implemented method of claim 1, wherein the computed label classification indicates that said cell or plurality of cells from the in vitro population of neuronal progenitor cells is a determined dopaminergic precursor cell if the deviation score indicates that at least or at least about 50%, 50%, 70%, 80%, 90%, or 95% of gene expression levels in the test dataset are no more than five standard deviations away from gene expression levels of the one or more reference cells in the reference database.

84. The computer implemented method of claim 1, wherein the computed label classification indicates that said cell or plurality of cells from the in vitro population of neuronal progenitor cells is a determined dopaminergic precursor cell if the deviation score indicates that at least or at least about 95% of gene expression levels in the test dataset are no more than 10, 9, 8, 7, 6, or 5 standard deviations away from the gene expression levels of the one or more reference cells in the reference database.

85. The computer implemented method of claim 60, wherein the computed label classification indicates that said cell or plurality of cells from the in vitro population of neuronal progenitor cells is a determined dopaminergic precursor cell if:
the probability of the cell or the plurality of cells having metagene expression levels of the determined dopaminergic precursor cell is greater than the threshold probability value; and
the deviation score indicates that at least or at least about 50%, 60%, 70%, 80%, 90%, or 95% of gene expression levels in the test dataset are no more than five standard deviations away from the gene expression levels of the one or more reference cells in the reference database.

86-89. (canceled)

90. The computer implemented method of claim 75, wherein the computed label classification indicates that said cell or plurality of cells from the in vitro population of neuronal progenitor cells is a determined dopaminergic precursor cell if the differences in expression of the marker genes between the test dataset and reference cells of the reference database is statistically insignificant based on a multiple-comparison corrected significance level.

91. The computer implemented method of claim 90, wherein the multiple-comparison corrected significance level is a Bonferroni corrected significance level or a false discover rate corrected significance level.

92. (canceled)

93. The computer implemented method of claim 1, wherein said gene expression levels are obtained from microarray analysis of cellular RNA, RNA sequencing, or both.

94. (canceled)

95. The computer implemented method of claim 93, wherein the RNA sequencing is performed on bulk RNA from the plurality of cells or a plurality of reference cells.

96. The computer implemented method of claim 93, wherein the RNA sequencing is performed on RNA from the single cells or a single reference cell.

97. (canceled)

98. The computer implemented method of claim 1, wherein receiving said test dataset comprises receiving input from an array analysis system.

99. (canceled)

100. The computer implemented method of claim 1, wherein said one or more reference databases forms part of a storage medium.

101. The computer implemented method of claim 1, comprising repeating the receiving, applying, determining, and outputting steps if the computed label classification indicates that said cell or plurality of cells is not a determined dopaminergic neuronal cell, optionally wherein the steps are repeated using the same or a different in vitro population of neuronal progenitor cells.

102-105. (canceled)

106. A population of determined dopaminergic precursor cells identified by the method of claim 1.

107. A method of treatment, the method comprising administering to a subject having Parkinson's disease the population of determined dopaminergic precursor cells of claim 106.

108. The method of claim 107, wherein the administering is by implanting the population of determined dopaminergic precursor cells into one or more brain regions of the subject.

109. (canceled)

110. The method of claim 107, wherein the population of determined dopaminergic precursor cells is autologous to the subject.

111. The method of claim 107, wherein the population of determined dopaminergic precursor cells is allogeneic to the subject.

112. A method of treating a subject having Parkinson's disease, the method comprising:
implanting a population of determined dopaminergic precursor cells into a brain region of a subject having Parkinson's disease, wherein the population of determined dopaminergic precursor cells has been identified using the computer implemented method of claim 1.

113. The method of claim 112, wherein the population of determined dopaminergic precursor cells is autologous to the subject.

114. The method of claim 112, wherein the population of determined dopaminergic precursor cells is allogeneic to the subject.

116-117. (canceled)
